(19) United States
(12) Patent Application Publication
(10) Pub. No.: US 2014/0155389 A1
(43) Pub. Date: Jun. 5, 2014

(54) BICYCLIC INHIBITORS OF ALK

(71) Applicants: Anil Vasudevan, Union Grove, WI (US); Thomas Dale Penning, Elmhurst, IL (US); Huanming Chen, Irvine, CA (US); Bo Liang, Beijing (CN); Shaohui Wang, Beijing (CN); Zhongqiang Zhao, Beijing (CN); Dikun Chai, Beijing (CN); Leifu Yang, Beijing (CN); Yingxiang Gao, Beijing (CN); Marina Pliushchev, Vernon Hills, IL (US)

(72) Inventors: Anil Vasudevan, Union Grove, WI (US); Thomas Dale Penning, Elmhurst, IL (US); Huanming Chen, Irvine, CA (US); Bo Liang, Beijing (CN); Shaohui Wang, Beijing (CN); Zhongqiang Zhao, Beijing (CN); Dikun Chai, Beijing (CN); Leifu Yang, Beijing (CN); Yingxiang Gao, Beijing (CN); Marina Pliushchev, Vernon Hills, IL (US)

(73) Assignee: ABBVIE INC., North Chicago, IL (US)

(21) Appl. No.: 13/979,389

(22) PCT Filed: Jan. 20, 2012
(86) PCT No.: PCT/CN12/00101

§ 371 Date: Feb. 19, 2014

(30) Foreign Application Priority Data
Jan. 21, 2011 (CH) PCTCN2011000110

(51) Int. Cl. C07D 471/04; C07D 401/12

(52) U.S. Cl. CPC C07D 471/04; C07D 401/12
USPC 514/221; 544/279; 514/252.16; 540/502; 544/236; 514/248; 514/249; 544/237

(57) ABSTRACT

The present invention relates to compounds of formula (1) or pharmaceutical acceptable salts, wherein R¹, X, Y, Z, A, B, G¹, and n are defined in the description. The present invention relates also to compositions containing said compounds which are useful for inhibiting kinases such as ALK and methods of treating diseases such as cancer.

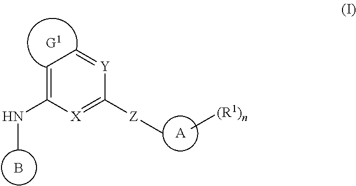

### FIELD OF THE INVENTION

This invention pertains to compounds which inhibit the activity of anaphastic lymphoma kinase (ALK), methods of making the compounds, compositions containing the compounds, and methods of treatment using the compounds.

### BACKGROUND OF THE INVENTION

Signaling through receptor tyrosine kinases (RTKs) regulates and fine-tunes many processes including cell growth, proliferation, differentiation, and apoptosis. The improper activation of RTKs is involved in the pathogenesis, growth, and metastasis of many cancers. The receptor tyrosine kinase ALK (Anaplastic Lymphoma Kinase) is a member of the insulin receptor superfamily that was initially identified from the t(2:5)(p23:q35) translocation in anaplastic large cell lymphoma (ALCL) (Fischer, P., et al. Blood, 72: 234-240. (1988)). The protein product of this translocation is ALK fused to nucleophosmin (NPM) (Morris et al. 1994). When fused to ALK, the dimerization domain of NPM results in constitutive dimerization and activation of ALK (reviewed in Chiarle, R., Nature reviews, 8:11-23 (2008)). Once activated, ALK recruits several adaptor proteins and stimulates multiple signaling pathways known to mediate tumor cell growth and survival including STAT3, PLC-γ, RAS-ERK1, 2, and PI3K-AKT (Bai. R. Y. et al. Molecular and cellular biology 18: 6951-6961 (1998): Bai, R. Y. et al. Blood 96:4319-4327 (2000); Chiarle, R., et al. Nature medicine 11:623-629 (2005): Pulford, K., et al. Journal of cellular physiology 199:330-358 (2004)). The dysregulation of ALK is highly oncogenic, as it is sufficient to induce cell transformation in a several immortalized cell lines (Bischof, D., et al. Molecular and cellular biology 17:2312-2325 (1997); Fujimoto, J., et al. Proceedings of the National Academy of Sciences of the United States of America 93: 4181-4186 (1996)) and to form tumors in animal models (Chiarle, R., et al. Blood 101: 1919-1927 (2003); Kuefer, M. U., et al. Blood 90: 2901-2910 (1997)). Moreover, NPM-ALK drives tumor formation, proliferation and survival in ALCL (reviewed in (Duyster, J., et al. Oncogene 20: 5623-5637 (2001)).

More recently, ALK translocations have been detected in ˜5% of non-small cell lung cancers (NSCLC). Similar to ALK translocations in ALCL, the fusion proteins in NSCLC display constitutive ALK activity and drive tumor growth and survival (Soda et al., Nature 448: 561-566 (2007); Soda et al., Proceedings of the National Academy of Sciences of the United States of America 105: 19893-19897 (2008)). NSCLC tumors harboring ALK translocations are mutually exclusive from K-Ras or EGFR aberrations and predominantly occur in younger patients that are non-smokers (Rodig et al., Clin Cancer Res 15: 5216-5223 (2009); Shaw et al., J Clin Oncol 27: 4247-4253 (2009); Wong et al., Cancer 115: 1723-1733 (2009)). In addition to chromosomal rearrangements, activating point mutations and amplifications have been reported in a subset of sporadic and familial neuroblastomas, further expanding the spectrum of tumors dependent on ALK activity (Chen et al., Nature 455: 971-974 (2008); George et al., Nature 455: 975-978 (2008); Janoueix-Lerosey et al., Nature 455: 967-970 (2008); Mosse et al., Nature 455: 930-935 (2008)). Neuroblastomas with ALK genetic aberrations also are dependent on ALK for proliferation and survival, and cells expressing ALK containing activating mutations form tumors in animal models.

Inhibitors of RTKs have the potential to cause lethality in cancerous cells that are reliant on deregulated RTK activity while sparing normal tissues. Thus, small molecule inhibitors of ALK would be beneficial for therapeutic intervention in ALCL, NSCLC, neuroblastoma, and other cancers that are dependent on ALK for growth and survival.

### SUMMARY OF THE INVENTION

The present invention has numerous embodiments. One embodiment of this invention, therefore, pertains to compounds that have formula (I)

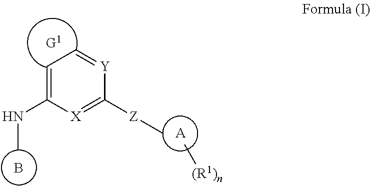

wherein R¹, n, X, Y, Z, A, B, and G¹ are as defined below and subsets therein.

Also provided are pharmaceutically acceptable compositions, comprising a therapeutically effective amount of a compound of formula (I) and a pharmaceutically acceptable salt in combination with a pharmaceutically suitable carrier.

One embodiment is directed to a method of treating cancer in a mammal comprising administering thereto a therapeutically acceptable amount of a compound or pharmaceutically acceptable salt of formula (I). Another embodiment pertains to a method of decreasing tumor volume in a mammal comprising administering thereto a therapeutically acceptable amount of a compound or pharmaceutically acceptable salt of formula (I).

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### ABBREVIATIONS AND DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. In this application, the use of “or” means “and/or” unless stated otherwise. Furthermore, the use of the term “including”, as well as other forms, such as “includes” and “included”, is not limiting. With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below. For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds which can be isolated in a useful degree of purity from a reaction mixture.

It is meant to be understood that proper valences are maintained for all combinations herein, that monovalent moieties having more than one atom are attached through their left ends, and that divalent moieties are drawn from left to right.

As used in the specification and the appended claims, unless specified to the contrary, the following terms have the meaning indicated:

The term “alkyl” (alone or in combination with another term(s)) means a straight- or branched-chain saturated hydrocarbyl substituent typically containing from 1 to about 10 carbon atoms; or in another embodiment, from 1 to about 8 carbon atoms; in another embodiment, from 1 to about 6 carbon atoms; and in another embodiment, from 1 to about 4 carbon atoms. Examples of such substituents include methyl, ethyl, n-propyl, isopropyl, n-butyl, isobutyl, sec-butyl, tert-butyl, pentyl, iso-amyl, and hexyl and the like.

The term “alkenyl” (alone or in combination with another term(s)) means a straight- or branched-chain hydrocarbyl substituent containing one or more double bonds and typically from 2 to about 10 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethenyl (vinyl), 2-propenyl, 3-propenyl, 1,4-pentadienyl, 1,4-butadienyl, 1-butenyl, 2-butenyl, and 3-butenyl and the like.

The term “alkynyl” (alone or in combination with another term(s)) means a straight- or branched-chain hydrocarbyl substituent containing one or more triple bonds and typically from 2 to about 10 carbon atoms; or in another embodiment, from 2 to about 8 carbon atoms: in another embodiment, from 2 to about 6 carbon atoms; and in another embodiment, from 2 to about 4 carbon atoms. Examples of such substituents include ethynyl, 2-propynyl, 3-propynyl, 2-butynyl, and 3-butynyl and the like.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent). A carbocyclyl may be a single-ring (monocyclic) or polycyclic ring structure.

A carbocyclyl may be a single ring structure, which typically contains from 3 to 8 ring atoms, more typically from 3 to 6 ring atoms, and even more typically 5 to 6 ring atoms. Examples of such single-ring carbocyclyls include cyclopropyl (cyclopropanyl), cyclobutyl (cyclobutanyl), cyclopentyl (cyclopentanyl), cyclopentenyl, cyclopentadienyl, cyclohexyl (cyclohexanyl), cyclohexenyl, cyclohexadienyl, and phenyl. A carbocyclyl may alternatively be polycyclic (i.e., may contain more than one ring). Examples of polycyclic carbocyclyls include bridged, fused, and spirocyclic carbocyclyls. In a spirocyclic carbocyclyl, one atom is common to two different rings. An example of a spirocyclic carbocyclyl is spiropentanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl.

The term “cycloalkyl” (alone or in combination with another term(s)) means a saturated cyclic hydrocarbyl substituent containing from 3 to 14 carbon ring atoms. A cycloalkyl may be a single carbon ring, which typically contains from 3 to 8 carbon ring atoms and more typically from 3 to 6 ring atoms. Examples of single-ring cycloalkyls include cyclopropyl, cyclobutyl, cyclopentyl, and cyclohexyl. A cycloalkyl may alternatively be polycyclic or contain more than one ring. Examples of polycyclic cycloalkyls include bridged, fused, and spirocyclic carbocyclyls.

The term “aryl” (alone or in combination with another term(s)) means an aromatic carbocyclyl containing from 6 to 14 carbon ring atoms. An aryl may be monocyclic or polycyclic (i.e., may contain more than one ring). In the case of polycyclic aromatic rings, only one ring the polycyclic system is required to be unsaturated while the remaining ring(s) may be saturated, partially saturated or unsaturated. Examples of aryls include phenyl, naphthalenyl, indenyl, indanyl, and tetrahydronapthyl.

In some instances, the number of carbon atoms in a hydrocarbyl substituent (e.g., alkyl, alkenyl, alkynyl, or cycloalkyl) is indicated by the prefix “Cₓ-C_{y}—”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₈-cycloalkyl means a saturated hydrocarbyl ring containing from 3 to 8 carbon ring atoms.

The term “hydrogen” (alone or in combination with another term(s)) means a hydrogen radical, and may be depicted as —H.

The term “hydroxy” (alone or in combination with another term(s)) means —OH.

The term “carboxy” (alone or in combination with another term(s)) means —C(O)—OH.

The term “amino” (alone or in combination with another term(s)) means —NH₂.

The term “halogen” or “halo” (alone or in combination with another term(s)) means a fluorine radical (which may be depicted as —F), chlorine radical (which may be depicted as —Cl), bromine radical (which may be depicted as —Br), or iodine radical (which may be depicted as —I).

If a substituent is described as being “substituted”, a non-hydrogen radical is in the place of hydrogen radical on a carbon or nitrogen of the substituent. Thus, for example, a substituted alkyl substituent is an alkyl substituent in which at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitution on a substituent, each non-hydrogen radical may be identical or different (unless otherwise stated).

If a substituent is described as being “optionally substituted”, the substituent may be either (1) not substituted or (2) substituted. If a substituent is described as being optionally substituted with up to a particular number of non-hydrogen radicals, that substituent may be either (1) not substituted: or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

This patent application uses the terms “substituent” and “radical” interchangeably.

The prefix “halo” indicates that the substituent to which the prefix is attached is substituted with one or more independently selected halogen radicals. For example, haloalkyl means an alkyl substituent in which at least one hydrogen radical is replaced with a halogen radical. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical.

The term “carbonyl” (alone or in combination with another term(s)) means —C(O)—.

The term “aminocarbonyl” (alone or in combination with another term(s)) means —C(O)—NH₂.

The term “oxo” (alone or in combination with another term(s)) means (═O). The term “oxy” (alone or in combination with another term(s)) means an ether substituent, and may be depicted as —O—.

The term “alkylhydroxy” (alone or in combination with another term(s)) means -alkyl-OH.

The term “alkylamino” (alone or in combination with another term(s)) means -alkyl-NH₂.

The term “alkyloxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, and tert-butoxy.

The term “alkylcarbonyl” (alone or in combination with another term(s)) means —C(O)-alkyl.

The term “aminoalkylcarbonyl” (alone or in combination with another term(s)) means —C(O)-alkyl-NH₂.

The term “alkyloxycarbonyl” (alone or in combination with another term(s)) means —C(O)—O-alkyl.

The term “carbocyclylcarbonyl” (alone or in combination with another term(s)) means —C(O)-carbocyclyl.

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl.

Similarly, the term “heterocyclylalkylcarbonyl” (alone or in combination with another term(s)) means —C(O)-alkyl-heterocyclyl.

The term “carbocyclyloxyycarbonyl” (alone or in combination with another term(s)) means —C(O)—O-carbocyclyl.

The term “carbocyclylalkyloxycarbonyl” (alone or in combination with another term(s)) means —C(O)—O-alkyl-carbocyclyl.

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-5-alkyl (alkyl-sulfanyl-alkyl).

The term “thiol” or “sulfhydryl” (alone or in combination with another term(s)) means a sulfhydryl substituent, and may be depicted as —SH.

The term “(thiocarbonyl)” (alone or in combination with another term(s)) means a carbonyl wherein the oxygen atom has been replaced with a sulfur. Such a substituent may be depicted as —C(S)—.

The term “sulfonyl” (alone or in combination with another term(s)) means —S(O)₂—.

The term “aminosulfonyl” (alone or in combination with another term(s)) means —S(O)₂—NH₂.

The term “sulfinyl” or “sulfoxido” (alone or in combination with another term(s)) means —S(O)—.

The term “heterocyclyl” (alone or in combination with another term(s)) means a saturated (i.e. “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocyclyl may be a single-ring (monocyclic) or polycyclic ring structure.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofuranyl, tetrahydrofuranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl(furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl, dihydropyranyl, thiopyranyl, tetrahydrothiopyranyl, pyridinyl(azinyl), piperidinyl, diazinyl (including pyridazinyl (1,2-diazinyl), pyrimidinyl (1,3-diazinyl), or pyrazinyl (1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, and diazepinyl.

A heterocyclyl may alternatively be polycyclic (i.e., may contain more than one ring). Examples of polycyclic heterocyclyls include bridged, fused, and spirocyclic heterocyclyls. In a spirocyclic heterocyclyl, one atom is common to two different rings. In a bridged heterocyclyl, the rings share at least two common non-adjacent atoms. In a fused-ring heterocyclyl, two or more rings may be fused together, such that two rings share one common bond. Examples of fused ring heterocyclyls containing two or three rings include indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), and benzisoxazinyl (including 1,2-benzisoxazinyl or 1,4-benzisoxazinyl).

The term “heterocycloalkyl” (alone or in combination with another term(s)) means a saturated heterocyclyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, pyridazinyl, and 1,3,5-, 1,2,4- or 1,2,3-triazinyl; 5-membered ring substituents such as imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl.

A prefix attached to a multi-component substituent only applies to the first component. To illustrate, the term “alkylcycloalkyl” contains two components: alkyl and cycloalkyl. Thus, the C₁-C₆-prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkyloxyalkyl indicates that only the alkyloxy component of the alkyloxyalkyl substituent is substituted with one or more halogen radicals. If halogen substitution may alternatively or additionally occur on the alkyl component, the substituent would instead be described as “halogen-substituted alkyloxyalkyl” rather than “haloalkyloxyalkyl.” And finally, if the halogen substitution may only occur on the alkyl component, the substituent would instead be described as “alkyloxyhaloalkyl.”

The terms “treat”, “treating” and “treatment” refer to a method of alleviating or abrogating a disease and/or its attendant symptoms.

The terms “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The term “therapeutically effective amount” refers to that amount of the compound being administered sufficient to prevent development of or alleviate to some extent one or more of the symptoms of the condition or disorder being treated.

The term “modulate” refers to the ability of a compound to increase or decrease the function, or activity, of a kinase. “Modulation”, as used herein in its various forms, is intended to encompass antagonism, agonism, partial antagonism and/or partial agonism of the activity associated with kinase. Kinase inhibitors are compounds that, e.g., bind to, partially or totally block stimulation, decrease, prevent, delay activation, inactivate, desensitize, or down regulate signal transduction. Kinase activators are compounds that, e.g., bind to, stimulate, increase, open, activate, facilitate, enhance activation, sensitize or up regulate signal transduction.

The term “composition” as used herein is intended to encompass a product comprising the specified ingredients in the specified amounts, as well as any product which results, directly or indirectly, from combination of the specified ingredients in the specified amounts.

By “pharmaceutically acceptable” it is meant the carrier, diluent or excipient must be compatible with the other ingredients of the formulation and not deleterious to the recipient thereof.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

### Compounds

### Embodiments of Formula (I)

In one embodiment, the present invention is directed, in part, to a class of compounds having a structure of Formula I

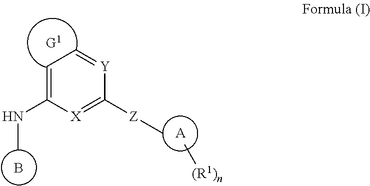

wherein

G¹ is

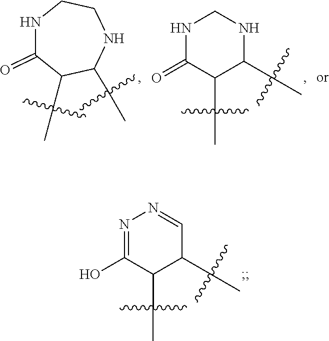 [SMILES: *C1NCCNC(=O)C1* ; *C1NCNC(=O)C1* ; *C1C=NN=C(O)C1*]

X is CH or N;

Y is CH or N;

wherein at least one of X and Y is N;

A is phenyl, naphthyl, indenyl, C₃₋₈ cycloalkyl, 4-7 membered heterocycloalkyl, 5-7 membered heterocycloalkenyl, or 5-7 membered heteroaryl;

B is

(a) phenyl, naphthyl, tetrahydronaphthyl, indenyl, or indanyl, wherein the phenyl, naphthyl, tetrahydronaphthyl, indenyl, or indanyl is optionally substituted with one, two, three, or four R² and is substituted with R³; or

(b) 5-16 membered monocyclic, bicyclic, or tricyclic heterocyclyl, wherein the heterocyclyl is optionally substituted with one, two, three, four, or five R⁴;

Z is a bond, C₁₋₆ alkylene, C₂₋₆ alkenylene, —O— or —NR⁵(CH₂)ₚ—;

R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁶, SR⁶, C(O)R⁶, C(O)NR⁷R⁸, C(O)OR⁶, OC(O)R⁶, OC(O)NR⁷R⁸, NR⁷R⁸, NR⁷C(O)R⁶, S(O)R⁶., S(O)NR⁷R⁸, S(O)₂R⁶, NR⁷S(O)₂R⁶, and S(O)₂NR⁷R⁸; wherein the C₃₋₈ cycloalkyl, aryl, heterocycloalkyl, and heteroaryl are optionally substituted with 1, 2, or 3 substituents independently selected from halo, C₁₋₆ alkyl, C₁₋₆ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c};

R², at each occurrence, is independently selected from the group consisting of halo, CN, OH, C₁₋₆ alkyl, C₁₋₆-haloalkyl, C₁₋₆ alkoxy, C₁₋₆ haloalkoxy, C₁₋₆-thioalkoxy, amino, C₁₋₆ alkylamino, and C₁₋₆ dialkylamino;

R³ is selected from the group consisting of aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁹, C(O)R⁹, —C₁₋₆-alkyl-C(O)R⁹, C(O)NR¹⁰R¹¹, C(O)OR⁹, OC(O)R⁹, OC(O)NR¹⁰R¹¹, NR¹⁰R¹¹, NR¹⁰C(O)R⁹, S(O)R⁹, S(O)NR¹⁰R¹¹, S(O)₂R⁹, NR¹⁰S(O)₂R⁹, and S(O)₂NR¹⁰R¹¹, wherein the C₃₋₈ cycloalkyl, aryl, heterocycloalkyl, and heteroaryl, alone or part of another moiety, are optionally substituted with one, two, or three R¹².

R⁴ is CN, NO₂, halo, C₁₋₆-alkyl, C₁₋₆-haloalkyl, OR^{d}, SR^{d}, C(O)R^{d}, C(O)NR^{e}R^{f}, C(O)OR^{d}, NR^{e}R^{f}, NR^{e}C(O)R^{d}, S(O)₂R^{d}, NR^{e}S(O)₂R^{d}, or S(O)₂NR^{e}R^{f};

R⁵ is H or C₁₋₆-alkyl;

R⁶, R⁷, and R⁸, at each occurrence, are independently selected from H, C₁₋₆ alkyl, C₁₋₆ haloalkyl, aryl, C₃₋₈ cycloalkyl, heteroaryl, and heterocycloalkyl, wherein the aryl, C₃₋₈ cycloalkyl, heteroaryl, and heterocycloalkyl moiety are optionally substituted with 1, 2, or 3 substituents independently selected from halo, CN, OH, C₁₋₆ alkyl, C₁₋₆-haloalkyl, C₁₋₆ alkoxy, C₁₋₆ haloalkoxy, amino, C₁₋₆ alkylamino, C₁₋₆ dialkylamino, C(O)OH, C(O) C₁₋₆ alkyl. C(O)NH₂, C(O)NH(C₁₋₆ alkyl), or C(O)N(C₁₋₆ alkyl)₂;

R⁹, R¹⁰, and R¹¹, at each occurrence, are independently selected from H, C₁₋₆ alkyl, C₁₋₆ haloalkyl, heteroaryl-C₁₋₆-alkyl-, heterocycloalkyl-C₁₋₆-alkyl-, R¹³R¹⁴N—C₁₋₆-alkyl-, aryl, C₃₋₈ cycloalkyl, heteroaryl, and heterocycloalkyl, wherein the aryl, C₃₋₈ cycloalkyl, heteroaryl, and heterocycloalkyl, alone or as part of another moiety, are optionally substituted with 1, 2, or 3 substituents independently selected from halo, CN, OH, C₁₋₆ alkyl, C₁₋₆-haloalkyl, C₁₋₆ alkoxy, C₁₋₆ haloalkoxy, amino, C₁₋₆ alkylamino, C₁₋₆ dialkylamino, C(O)OH, C(O)C₁₋₆ alkyl, C(O)NH₂, C(O)NH(C₁₋₆ alkyl), or C(O)N(C₁₋₆ alkyl)₂;

R¹², at each occurrence, is independently selected from the group consisting of halo, C₁₋₆ alkyl, C₁₋₆ haloalkyl, amino-C₁₋₆-alkyl-, C₁₋₆ alkylamino-C₁₋₆ alkyl-, C₁₋₆ dialkylamino-C₁₋₆ alkyl-, hydroxy-C₁₋₆-alkyl-, C₁₋₆ alkyl-C₁₋₆ alkoxy, aryl, C₃₋₈ cycloalkyl, heteroaryl, heterocycloalkyl, aryl-(C₁₋₆ alkyl)-, C₃₋₈ cycloalkyl-(C₁₋₆ alkyl)-, heteroaryl-(C₁₋₆ alkyl)-, heterocycloalkyl-(C₁₋₆ alkyl)-, CN, NO₂, OR^{g}, SR^{g}, C(O)R^{g}, C(O)NR^{h}Rⁱ, C(O)OR^{g}, OC(O)R^{g}, OC(O)NR^{h}Rⁱ, NR^{h}Rⁱ, NR^{h}C(O)R^{g}, S(O)R^{g}, S(O)NR^{h}Rⁱ, S(O)₂R^{g}, NR^{h}S(O)₂R^{g}, and S(O)₂NR^{h}Rⁱ, wherein the aryl, C₃₋₈ cycloalkyl, heteroaryl, and heterocycloalkyl, alone or as part of another moiety, are optionally substituted with one, two or three substituents independently selected from halo and C₁₋₆ alkyl;

R¹³ and R¹⁴, at each occurrence, are independently selected from the group consisting of H, C₁₋₆ alkyl, aryl, C₃₋₈ cycloalkyl, heteroaryl, and heterocycloalkyl, wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃—X cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{a}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{b} and R^{c}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{d}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{e} and R^{f}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{g}, at each occurrence, is independently selected from the group consisting of H, C₁₋₆ alkyl, aryl, C₃₋₈ cycloalkyl, heteroaryl, and heterocycloalkyl: wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl. C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{h} and Rⁱ, at each occurrence, are independently selected from the group consisting of H, C₁₋₆ alkyl, aryl., C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

n is 0, 1, 2, or 3; and

p is 0, 1, 2, or 3;

or a pharmaceutically acceptable salt or solvate thereof.

In one embodiment of formula (I), G¹ is

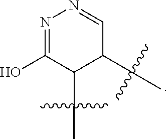 [SMILES: *C1C=NN=C(O)C1*]

In another embodiment of formula (I), G¹ is

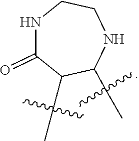 [SMILES: *C1NCCNC(=O)C1*]

In another embodiment of formula (I), G¹ is

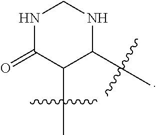 [SMILES: *C1NCNC(=O)C1*]

In one embodiment of formula (I), X is N; and Y is CH. In another embodiment of formula (I), X is CH; and Y is N. In another embodiment of formula (I), X is N; and Y is N.

In one embodiment of formula (I), G¹ is

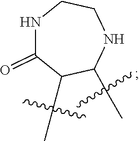 [SMILES: *C1NCCNC(=O)C1*]

X is CH; and Y is N. In another embodiment of formula (I), G¹ is

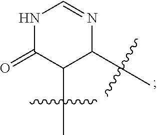 [SMILES: *C1N=CNC(=O)C1*]

### X is CH; and Y is N.

In another embodiment of formula (I), G¹ is

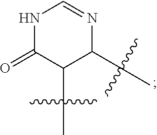 [SMILES: *C1N=CNC(=O)C1*]

X is N; and Y is N. In another embodiment of formula (I), G¹ is

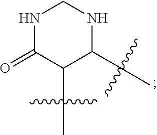 [SMILES: *C1NCNC(=O)C1*]

### X is N; and Y is CH.

In one embodiment of formula (I), Z is C₁₋₆ alkylene. In another embodiment of formula (I), Z is —CH₂—, —CH₂CH₂—, —CH₂CH₂CH₂—, or —CH₂CH₂CH₂CH₂—. In another embodiment of formula (I), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—, —C(CH₃)₂CH₂—, —CH₂CH₂C(CH₃)₂—, —CH₂C(CH₃)₂CH₂—, or —C(CH₃)₂CH₂CH₂—. In another embodiment of formula (I), Z is CH(CH₂CH₃)—, —CH₂CH(CH₂CH₃)—, —CH(CH₂CH₃)CH₂—, —CH(CH₂CH₃)CH₂CH₂—., —CH₂CH(CH₂CH₃)CH₂—, —CH₂CH₂CH(CH₂CH₃)—, —C(CH₂CH₃)₂—, —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (I), Z is —CH₂—, —CH₂CH₂—, —CH(CH₃)—, or —C(CH₃)₂—. In yet another embodiment of formula (I), Z is —CH₂—.

In another embodiment of formula (I), Z is C₂₋₆ alkenylene. In yet another embodiment of formula (I), Z is —CH═CH—, —CH₂CH₂═CH—, —CH═CHCH₂—, —CH₂—CH═CH—CH₂—, —CH═CH—CH₂CH₂—, or —CH₂CH₂—CH═CH—. In another embodiment of formula (I), Z is —CH(═CH₂)—, —CH₂CH(═CH₂)—, —CH(═CH₂)CH₂—, or —CH(═CHCH₃)—. In yet another embodiment of formula (I), Z is —CH═CH— or —CH(═CH₂)—.

In one embodiment of formula (I), Z is a bond.

In another embodiment of formula (I), Z is NR^{S}, wherein R⁵ is H or C₁₋₆ alkyl.

In one embodiment of formula (I), A is phenyl, naphthyl, indenyl or C₃₋₈ cycloalkyl. In yet another embodiment of formula (I), A is phenyl.

In another embodiment of formula (I), A is a 5-7 membered heterocycloalkyl or heterocycloalkenyl. In another embodiment of formula (I), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (I), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (I), A is a 5-7 membered heteroaryl. In another embodiment of formula (I), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (I), A is optionally substituted with —(R¹)ₙ, wherein n is 0, 1, 2, or 3. In one embodiment of formula (I), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁶, SR⁶, C(O)R⁶, C(O)NR⁷R⁸, C(O)OR⁶, OC(O)R⁶, OC(O)NR⁷R⁸, NR⁷R⁸, NR⁷C(O)R⁶, S(O)R⁶, S(O)NR⁷R⁸, S(O)₂R⁶, NR⁷S(O)₂R⁶, and S(O)₂NR⁷R⁸; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (I), A is phenyl, n is 2, and R¹, at each occurrence, is halo.

In one embodiment of formula (I), B is phenyl. In another embodiment of formula (I), B is phenyl and is unsubstituted with R². In another embodiment, the phenyl is substituted with one or two R², and R² is halo, C₁₋₆-alkyl, C₁₋₆ haloalkyl, or OR⁶.

In one embodiment of formula (I), B is phenyl, wherein the phenyl is substituted with R³, and R³ is heterocycloalkyl, wherein the heterocycloalkyl is optionally substituted with one, two, or three R¹²; wherein R¹² is halo, C₁₋₆-alkyl, or C₁₋₆-haloalkyl. In yet another embodiment, phenyl is substituted with heterocycloalkyl, and heterocycloalkyl is selected from the group consisting of azetidinyl, pyrrolidinyl, piperidinyl, morpholinyl, piperazinyl, azepanyl, diazepanyl, and hexahydropyrrolo[1,2-a]pyrazin-2(1H)yl.

In one embodiment of formula (I), B is

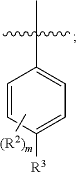

wherein R² and R³ are as defined above and m is 0, 1, or 2. In another embodiment of formula (I), m is 0. In another embodiment of formula (I), m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (I), m is 1 and R² is selected from the group consisting of halo, and C₁₋₄ alkoxy. In another embodiment of formula (I), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁹, C(O)R⁹, C(O)NR¹⁰R¹¹, C(O)OR⁹, OC(O)R⁹, OC(O)NR¹⁰R¹¹, NR¹⁰R¹¹, NR¹⁰C(O)R⁹, S(O)R⁹, S(O)NR¹⁰R¹¹, S(O)₂R⁹, NR¹⁰S(O)₂R⁹, and S(O)₂NR¹⁰R¹¹, wherein the C₃₋₈ cycloalkyl, aryl, heterocycloalkyl, and heteroaryl, alone or part of another moiety, are optionally substituted with one, two, or three R¹², wherein R¹² is defined above. In yet another embodiment of formula (I), B is phenyl, and R³ is heterocycloalkyl. In yet another embodiment of formula (I), R³ is heterocycloalkyl. In yet another embodiment of formula (I), R³ is heterocycloalkyl, which is optionally substituted with one R¹², and R¹² is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{g}, SR^{g}, C(O)R^{g}, C(O)NR^{h}Rⁱ, C(O)OR^{g}, OC(O)R^{g}, OC(O)NR^{h}Rⁱ, NR^{h}Rⁱ, NR^{h}C(O)Rⁱ, S(O)R^{g}, S(O)NR^{h}Rⁱ, S(O)₂R^{g}, NR^{h}S(O)₂R^{g}, and S(O)₂NR^{h}Rⁱ, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{g}, R^{h}, and Rⁱ are as defined above.

In another embodiment of formula (I), B is

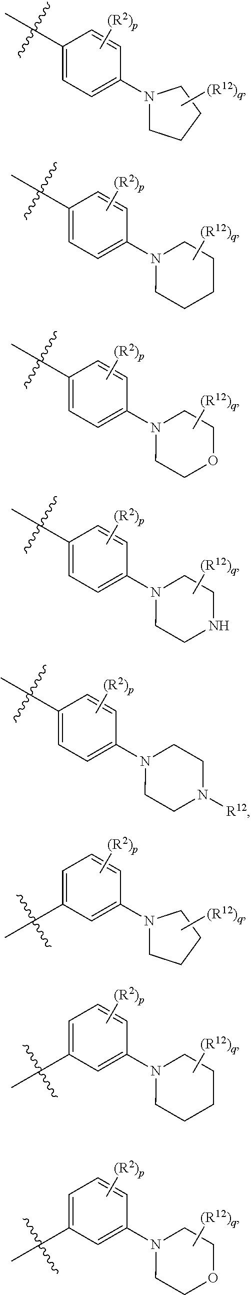

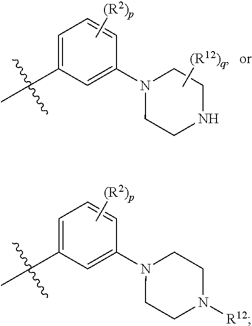

wherein R² is halo, C₁₋₆ alkyl, C₁₋₆ haloalkyl, or OR⁶: p is 0 or 1; R¹² is C₁₋₆-alkyl, C₁₋₆-haloalkyl, OR^{g}, C(O)R^{g}, C(O)NR^{h}Rⁱ, C(O)OR^{g}, NR^{h}Rⁱ, NR^{h}C(O)R^{g}, S(O)₂R^{g}, or S(O)₂NR^{h}Rⁱ; and q is 0 or 1.

In one embodiment of formula (I), B is

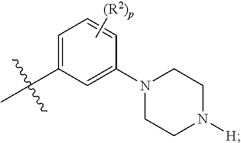

R² is halo, C₁₋₆-alkyl, C₁₋₆ haloalkyl, or OR⁶; and p is 0, 1, or 2.

In one embodiment of formula (I), B is a 4-8 membered monocyclic heterocyclyl. In another embodiment, B is a 4-8 membered heterocycloalkyl or heterocycloalkenyl. In another embodiment. B is a 5-7 membered heteroaryl. In yet another embodiment of formula (I), B is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (I), B is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl. In one embodiment, B is unsubstituted. In another embodiment. B is substituted with one, two, or three R⁴, and R⁴ is halo, C₁₋₆-alkyl, C₁₋₆-haloalkyl. OR^{d}, C(O)R^{d}, C(O)OR^{d}, NR^{e}R^{f}, or S(O)₂R^{d},

In one embodiment of formula (I), B is a 7-11 membered bicyclic heterocyclyl. In another embodiment, B is a 7-11 membered bicyclic heterocycloalkyl or bicyclic heterocyloalkenyl. In another embodiment, B is a 7-11 membered bicyclic heteroaryl. In yet another embodiment, B is 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl, dihydroisoindolyl, dihydroquinazolinyl, 3,4-dihydro-4-oxo-quinazolinyl, benzisothiazolyl, benzisoxazolyl, benzodiazinyl, benzofurazanyl, benzothiopyranyl, benzotriazolyl, benzpyrazolyl, 1,3-benzodioxolyl, dihydrobenzofuryl, dihydrobenzothienyl, dihydrobenzothiopyranyl, dihydrobenzothiopyranyl sulfone, dihydrobenzopyranyl, dihydrobenzoxazinyl, 3-oxo-3,4-dihydro-1,4-benzoxazinyl, indolinyl, indazolyl, isochromanyl, isoindolinyl, naphthyridinyl, phthalazinyl, piperonyl, purinyl, pyridopyridyl, pyrrolotriazinyl, quinazolinyl, tetrahydroquinolinyl, thienofuryl, thienopyridyl, 3H-imidazo[4,5-c]pyridinyl, or thienothienyl. In one embodiment of formula (I), B is unsubstituted. In another embodiment of formula (I). B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

In one embodiment of formula (I), B is 10-15 membered tricyclic heterocyclyl. In another embodiment, B is a 10-15 membered tricyclic heterocycloalkyl or tricyclic heterocyloalkenyl. In another embodiment, B is a 10-15 membered tricyclic heteroaryl. In one embodiment of formula (I), B is unsubstituted. In another embodiment of formula (I), B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

### Embodiments of Formula (II)

In one embodiment, the present invention is directed, in part, to a class of compounds having a structure of Formula (II),

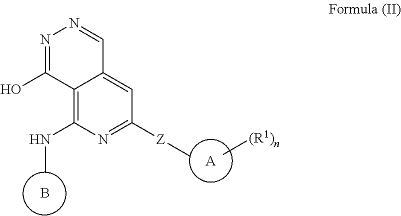

wherein R¹, A, B, Z, and n are as described in formula (II).

In one embodiment of formula (II), Z is C₁₋₆ alkylene. In another embodiment of formula (II), Z is —CH₂—, —CH₂CH₂—, —CH₂CH₂CH₂—, or —CH₂CH₂CH₂CH₂—. In another embodiment of formula (II), Z is —CH(CH₃)—, —CH₂CH(CH₃)—, —CH(CH₃)CH₂—, —CH(CH₃)CH₂CH₂—, —CH₂CH(CH₃)CH₂— —CH₂CH₂CH(CH₃)—, —C(CH₃)₂—, —CH₂C(CH₃)₂—, —C(CH₃)₂CH₂—, —CH₂CH₂C(CH₃)₂—, —CH₂C(CH₃)₂CH₂—, or —C(CH₃)₂CH₂CH₂—. In another embodiment of formula (II), Z is CH(CH₂CH₃)—, —CH₂CH(CH₂CH₃)—, —CH(CH₂CH₃)CH₂—, —CH(CH₂CH₃)CH₂CH₂—, —CH₂CH(CH₂CH₃)CH₂—, —CH₂CH₂CH(CH₂CH₃)—, —C(CH₂CH₃)₂—, —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (II), Z is —CH₂—, —CH₂CH₂—, —CH(CH₃)—, or —C(CH₃)₂—. In yet another embodiment of formula (II), Z is —CH₂—.

In another embodiment of formula (II), Z is C₂₋₆ alkenylene. In yet another embodiment of formula (II), Z is —CH═CH—, —CH₂CH₂═CH—, —CH═CHCH₂—, —CH₂—CH═CH—CH₂—, —CH═CH—CH₂CH₂—, or —CH₂CH₂—CH═CH—. In another embodiment of formula (II), Z is —CH(═CH₂)—, —CH₂CH(═CH₂)—, —CH(═CH₂)CH₂—, or —CH(═CHCH₃)—. In yet another embodiment of formula (II), Z is —CH═CH— or —CH(═CH₂)—.

In one embodiment of formula (II), Z is a bond.

In another embodiment of formula (II), Z is NR⁵, wherein R⁵ is H or C₁₋₆ alkyl.

In one embodiment of formula (II), A is phenyl, naphthyl, indenyl or C₃₋₈ cycloalkyl. In yet another embodiment of formula (II), A is phenyl.

In another embodiment of formula (II), A is a 5-7 membered heterocycloalkyl or heterocycloalkenyl. In another embodiment of formula (II), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (II), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (II), A is a 5-7 membered heteroaryl. In another embodiment of formula (II), A is pyridyl, pyrazyl, pyridinyl, pyrimidinyl, pyridazinyl, 1,3,5-, 1,2,4- or 1,2,3-triazinyl, imidazyl furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (II), A is optionally substituted with —(R¹)ₙ, wherein n is 0, 1, 2, or 3. In one embodiment of formula (II). R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁶, SR⁶, C(O)R⁶, C(O)NR⁷R⁸, C(O)OR⁶, OC(O)R⁶, OC(O)NR⁷R⁸, NR⁷R⁸, NR⁷C(O)R⁶, S(O)R⁶, S(O)NR⁷R⁸, S(O)₂R⁶, NR⁷S(O)₂R⁶, and S(O)₂NR⁷R⁸; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (II), A is phenyl, n is 2, and R¹, at each occurrence, is halo.

In one embodiment of formula (II), B is phenyl. In another embodiment of formula (II), B is phenyl and is unsubstituted with R². In another embodiment, the phenyl is substituted with one or two R², and R² is halo, C₁₋₆-alkyl, C₁₋₆ haloalkyl, or OR⁶.

In one embodiment of formula (II), B is phenyl, wherein the phenyl is substituted with R³, and R³ is heterocycloalkyl, wherein the heterocycloalkyl is optionally substituted with one, two, or three R¹², wherein R¹² is halo, C₁₋₆-alkyl, or C₁₋₆-haloalkyl. In yet another embodiment, phenyl is substituted with heterocycloalkyl, and heterocycloalkyl is selected from the group consisting of azetidinyl, pyrrolidinyl, piperidinyl, morpholinyl, piperazinyl, azepanyl, diazepanyl, and hexahydropyrrolo[1,2-a]pyrazin-2(1H)yl.

In one embodiment of formula (II), B is

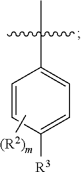

wherein R² and R³ are as defined above and m is 0.1, or 2. In another embodiment of formula (II), m is 0. In another embodiment of formula (II), m is 1., and R², at each occurrence, is independently selected from the group consisting of halo, CN, OH, C₁₋₄ alkyl. C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (II), m is 1 and R² is selected from the group consisting of halo, and C₁₋₄ alkoxy. In another embodiment of formula (II), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁹, C(O)R⁹, C(O)NR¹⁰R¹¹, C(O)OR⁹, OC(O)R⁹, OC(O)NR¹⁰R¹¹, NR¹⁰R¹¹, NR¹⁰C(O)R⁹, S(O)R⁹, S(O)NR¹⁰R¹¹, S(O)₂R⁹, NR¹⁰S(O)₂R⁹, and S(O)₂NR¹⁰R¹¹, wherein the C₃₋₈ cycloalkyl, aryl, heterocycloalkyl, and heteroaryl, alone or part of another moiety, are optionally substituted with one, two, or three R¹², wherein R¹² is defined above. In yet another embodiment of formula (II), B is phenyl, and R³ is heterocycloalkyl. In yet another embodiment of formula (II), R³ is heterocycloalkyl. In yet another embodiment of formula (II), R³ is heterocycloalkyl, which is optionally substituted with one R¹², and R¹² is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR¹, SR^{g}, C(O)R^{g}, C(O)NR^{h}Rⁱ, C(O)OR^{g}, OC(O)R^{g}, OC(O)NR^{h}Rⁱ, NR^{h}Rⁱ, NR^{h}C(O)Rⁱ, S(O)R^{g}, S(O)NR^{h}Rⁱ. S(O)₂R^{g}, NR^{h}S(O)₂R^{g}, and S(O)₂NR^{h}Rⁱ, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{g}, R^{h}, and Rⁱ are as defined above.

In another embodiment of formula (II), B is

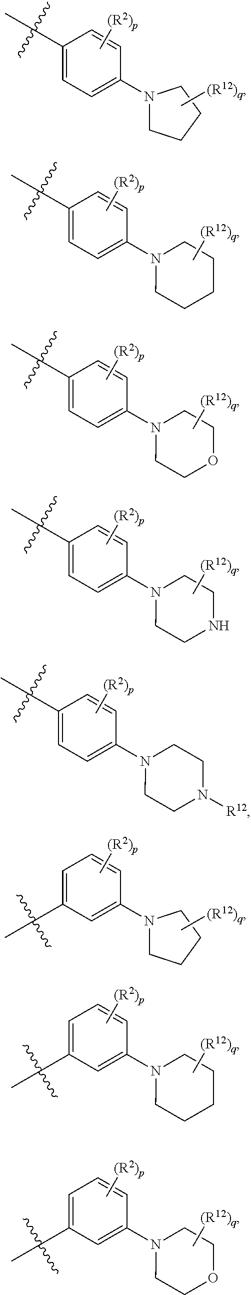

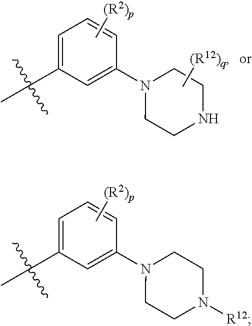

wherein R² is halo, C₁₋₆-alkyl, C₁₋₆ haloalkyl, or OR⁶; p is 0 or 1; R¹² is C₁₋₆-alkyl, C₁₋₆-haloalkyl. OR^{g}, C(O)R^{g}, C(O)NR^{h}Rⁱ, C(O)OR^{g}, NR^{h}Rⁱ, NR^{h}C(O)R^{g}, S(O)₂R^{g}, or S(O)₂NR^{h}Rⁱ; and q is 0 or 1.

In one embodiment of formula (II), B is

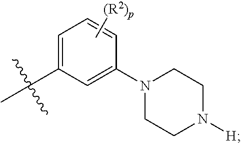

R² is halo, C₁₋₆-alkyl, C₁₋₆ haloalkyl, or OR⁶; and p is 0, 1, or 2.

In one embodiment of formula (II), B is a 4-8 membered monocyclic heterocyclyl. In another embodiment, B is a 4-8 membered heterocycloalkyl or heterocycloalkenyl. In another embodiment, B is a 5-7 membered heteroaryl. In yet another embodiment of formula (I), B is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (II), B is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl. In one embodiment, B is unsubstituted. In another embodiment, B is substituted with one, two, or three R⁴, and R⁴ is halo, C₁₋₆-alkyl, C₁₋₆-haloalkyl, OR^{d}. C(O)R^{d}, C(O)OR^{d}, NR^{e}R^{f}, or S(O)₂R^{d}.

In one embodiment of formula (II), B is a 7-11 membered bicyclic heterocyclyl. In another embodiment, B is a 7-11 membered bicyclic heterocycloalkyl or bicyclic heterocyloalkenyl. In another embodiment, B is a 7-11 membered bicyclic heteroaryl. In yet another embodiment, B is 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl, dihydroisoindolyl, dihydroquinazolinyl, 3,4-dihydro-4-oxo-quinazolinvyl, benzisothiazolyl, benzisoxazolyl, benzodiazinyl, benzofurazanyl, benzothiopyranyl, benzotriazolyl, benzpyrazolyl, 1,3-benzodioxolyl, dihydrobenzofuryl, dihydrobenzothienyl, dihydrobenzothiopyranyl, dihydrobenzothiopyranyl sulfone, dihydrobenzopyranyl, dihydrobenzoxazinyl, 3-oxo-3,4-dihydro-1,4-benzoxazinyl, indolinyl, indazolyl, isochromanyl, isoindolinyl, naphthyridinyl, phthalazinyl, piperonyl, purinyl, pyridopyridyl, pyrrolotriazinyl, quinazolinyl, tetrahydroquinolinyl, thienofuryl, thienopyridyl, 3H-imidazo[4,5-c]pyridinyl, or thienothienyl. In one embodiment of formula (II), B is unsubstituted. In another embodiment of formula (II), B is substituted with one, two, or three R⁴, and R⁴ is halo, C₁₋₆-alkyl, C₁₋₆-haloalkyl. OR^{d}, C(O)R^{d}, C(O)OR^{d}, NR^{e}R^{f}, or S(O)₂R^{d}.

In one embodiment of formula (II), B is 10-15 membered tricyclic heterocyclyl. In another embodiment, B is a 10-15 membered tricyclic heterocycloalkyl or tricyclic heterocyloalkenyl. In another embodiment, B is a 10-15 membered tricyclic heteroaryl. In one embodiment of formula (II), B is unsubstituted. In another embodiment of formula (II), B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

### Embodiments of Formula (III)

In one embodiment, the present invention is directed, in part, to a class of compounds having a structure of Formula (III).

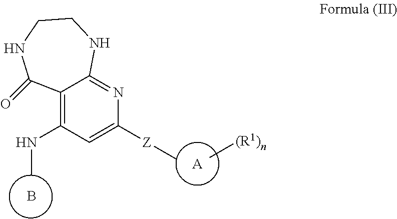

wherein R¹, A, B, Z, and n are as described in formula (I).

In one embodiment of formula (III), Z is C₁₋₆ alkylene. In another embodiment of formula (III), Z is —CH₂—, —CH₂CH₂—, —CH₂CH₂CH₂—, or —CH₂CH₂CH₂CH₂—. In another embodiment of formula (III), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—, —C(CH₃)₂CH₂—., —CH₂CH₂C(CH₃)₂—, —CH₂C(CH₃)₂CH₂—, or —C(CH₃)₂CH₂CH₂—. In another embodiment of formula (III), Z is CH(CH₂CH₃)—, —CH₂CH(CH₂CH₃)—, —CH(CH₂CH₃)CH₂—, —CH(CH₂CH₃)CH₂CH₂—, —CH₂CH(CH₂CH₃)CH₂—, —CH₂CH₂CH(CH₂CH₃)—, —C(CH₂CH₃)₂—. —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (III), Z is —CH₂—, —CH₂CH₂—, —CH(CH₃)—, or —C(CH₃)₂—. In yet another embodiment of formula (III), Z is —CH₂—.

In another embodiment of formula (III), Z is C₂₋₆ alkenylene. In yet another embodiment of formula (III), Z is —CH═CH—, —CH₂CH₂═CH—, —CH═CHCH₂—, —CH₂—CH═CH—CH₂—, —CH═CH—CH₂CH₂—, or —CH₂CH₂—CH═CH—. In another embodiment of formula (III), Z is —CH(═CH₂)—, —CH₂CH(═CH₂)—, —CH(═CH₂)CH₂—, or —CH(═CHCH₃)—. In yet another embodiment of formula (III), Z is —CH═CH— or —CH(═CH₂)—.

In one embodiment of formula (III), Z is a bond.

In another embodiment of formula (III), Z is NR⁵, wherein R⁵ is H or C₁₋₆ alkyl.

In one embodiment of formula (III), A is phenyl, naphthyl, indenyl or C₃₋₈ cycloalkyl. In yet another embodiment of formula (III), A is phenyl.

In another embodiment of formula (III), A is a 5-7 membered heterocycloalkyl or heterocycloalkenyl. In another embodiment of formula (II), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (III), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (III), A is a 5-7 membered heteroaryl. In another embodiment of formula (III), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (III), A is optionally substituted with —(R¹)ₙ, wherein n is 0, 1, 2, or 3. In one embodiment of formula (III), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁶, SR⁶, C(O)R⁶, C(O)NR⁷R⁸, C(O)OR⁶, OC(O)R⁶, OC(O)NR⁷R⁸, NR⁷R⁸, NR⁷C(O)R⁶, S(O)R⁶, S(O)NR⁷R⁸, S(O)₂R⁶, NR⁷S(O)₂R⁶, and S(O)₂NR⁷R⁸; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (III), A is phenyl, n is 2, and R¹, at each occurrence, is halo.

In one embodiment of formula (III), B is phenyl. In another embodiment of formula (III), B is phenyl and is unsubstituted with R². In another embodiment, the phenyl is substituted with one or two R², and R² is halo, C₁₋₆-alkyl, C₁₋₆ haloalkyl, or OR⁶.

In one embodiment of formula (III), B is phenyl, wherein the phenyl is substituted with R³, and R³ is heterocycloalkyl, wherein the heterocycloalkyl is optionally substituted with one, two, or three R¹²; wherein R¹² is halo, C₁₋₆-alkyl, or C₁₋₁₆-haloalkyl. In yet another embodiment, phenyl is substituted with heterocycloalkyl, and heterocycloalkyl is selected from the group consisting of azetidinyl, pyrrolidinyl, piperidinyl, morpholinyl, piperazinyl, azepanyl, diazepanyl, and hexahydropyrrolo[1,2-a]pyrazin-2(1H)yl.

In one embodiment of formula (III), B is

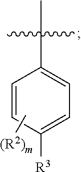

wherein R² and R³ are as defined above and m is 0.1, or 2. In another embodiment of formula (III), m is 0. In another embodiment of formula (III), m is 1, and R², at each occurrence, is independently selected from the group consisting of halo, CN, OH, C₁₋₄ alkyl. C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (III), m is 1 and R² is selected from the group consisting of halo, and C₁₋₄ alkoxy. In another embodiment of formula (III), R³ is selected from the group consisting aryl, C₃₋₈ cycloalkyl, heteroaryl, heterocycloalkyl, aryl-C₁₋₆-alky-, C₃₋₈ cycloalkyl-C₁₋₆-alkyl-, heteroaryl-C₁₋₆-alkyl-, heterocycloalkyl-C₁₋₆-alkyl-, OR⁹, C(O)R⁹, C(O)NR¹⁰R¹¹, C(O)OR⁹, OC(O)R⁹, OC(O)NR¹⁰R¹¹, NR¹⁰R¹¹, NR¹⁰C(O)R⁹, S(O)R⁹, S(O)NR¹⁰R¹¹, S(O)₂R⁹, NR¹⁰S(O)₂R⁹, and S(O)₂NR¹⁰R¹¹, wherein the C₃₋₈ cycloalkyl, aryl, heterocycloalkyl, and heteroaryl, alone or part of another moiety, are optionally substituted with one, two, or three R¹², wherein R¹² is defined above. In yet another embodiment of formula (III), B is phenyl, and R³ is heterocycloalkyl. In yet another embodiment of formula (III), R³ is heterocycloalkyl. In yet another embodiment of formula (III), R³ is heterocycloalkyl, which is optionally substituted with one R¹², and R¹² is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₁₂ alkyl)-, CN, NO₂, OR^{g}, SR^{g}, C(O)R^{g}. C(O)NR^{h}Rⁱ, C(O)OR^{g}, OC(O)R^{g}, OC(O)NR^{h}Rⁱ, NR^{h}Rⁱ, NR^{h}C(O)Rⁱ, S(O)R^{g}, S(O)NR^{h}Rⁱ, S(O)₂R^{g}, NR^{h}S(O)₂R^{g}, and S(O)₂NR^{h}Rⁱ, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{g}, R^{h}, and Rⁱ are as defined above.

In another embodiment of formula (III), B is

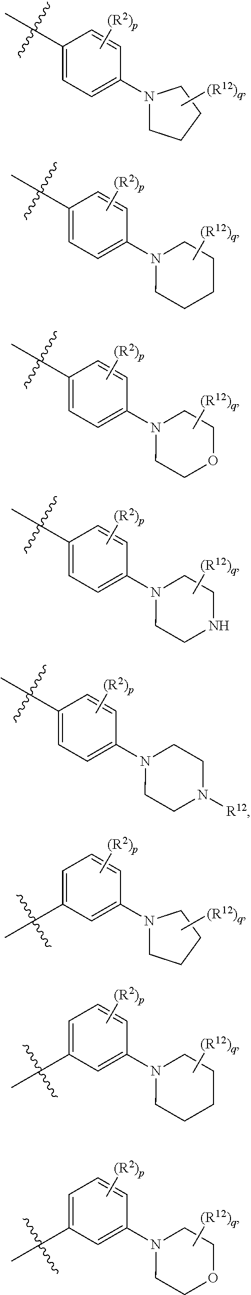

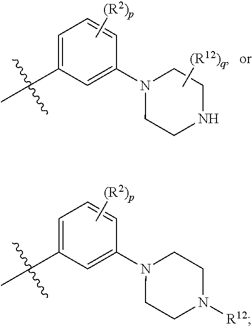

wherein R² is halo, C₁₋₆-alkyl, C₁₋₆ haloalkyl, or OR⁶; p is 0 or 1; R¹² is C₁₋₆-alkyl, C₁₋₆-haloalkyl, OR^{g}, C(O)R^{g}, C(O)NR^{h}Rⁱ, C(O)OR^{g}, NR^{h}Rⁱ, NR^{h}C(O)R^{g}, S(O)₂R^{g}, or S(O)₂NR^{h}Rⁱ; and q is 0 or 1.

In one embodiment of formula (III), B is

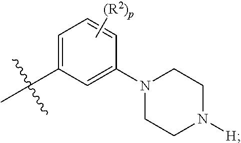

R² is halo, C₁₋₆-alkyl, C₁₋₆ haloalkyl, or OR⁶; and p is 0, 1, or 2.

In one embodiment of formula (III), B is a 4-8 membered monocyclic heterocyclyl. In another embodiment, B is a 4-8 membered heterocycloalkyl or heterocycloalkenyl. In another embodiment, B is a 5-7 membered heteroaryl. In yet another embodiment of formula (III), B is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (III), B is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl. In one embodiment, B is unsubstituted. In another embodiment, B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

In one embodiment of formula (III), B is a 7-11 membered bicyclic heterocyclyl. In another embodiment, B is a 7-11 membered bicyclic heterocycloalkyl or bicyclic heterocyloalkenyl. In another embodiment. B is a 7-11 membered bicyclic heteroaryl. In yet another embodiment, B is 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl., isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl, dihydroisoindolyl, dihydroquinazolinyl, 3,4-dihydro-4-oxo-quinazolinyl, benzisothiazolyl, benzisoxazolyl, benzodiazinyl, benzofurazanyl, benzothiopyranyl, benzotriazolyl, benzpyrazolyl, 1,3-benzodioxolyl, dihydrobenzofuryl, dihydrobenzothienyl, dihydrobenzothiopyranyl, dihydrobenzothiopyranyl sulfone, dihydrobenzopyranyl, dihydrobenzoxazinyl, 3-oxo-3,4-dihydro-1,4-benzoxazinyl, indolinyl, indazolyl, isochromanyl, isoindolinyl, naphthyridinyl, phthalazinyl, piperonyl, purinyl, pyridopyridyl, pyrrolotriazinyl, quinazolinyl, tetrahydroquinolinyl, thienofuryl, thienopyridyl, 3H-imidazo[4,5-c]pyridinyl, or thienothienyl. In one embodiment of formula (III), B is unsubstituted. In another embodiment of formula (III). B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

In one embodiment of formula (III), B is 10-15 membered tricyclic heterocyclyl. In another embodiment, B is a 10-15 membered tricyclic heterocycloalkyl or tricyclic heterocyloalkenyl. In another embodiment. B is a 10-15 membered tricyclic heteroaryl. In one embodiment of formula (III), B is unsubstituted. In another embodiment of formula (III), B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

### Embodiments of Formula (IV)

In one embodiment, the present invention is directed, in part, to a class of compounds having a structure of Formula (IV),

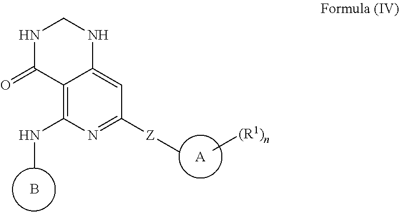

wherein R¹, A, B, Z, and n are as described in formula (I).

In one embodiment of formula (IV), Z is C₁₋₆ alkylene. In another embodiment of formula (IV), Z is —CH₂—, —CH₂CH₂—, —CH₂CH₂CH₂—, or —CH₂CH₂CH₂CH₂—. In another embodiment of formula (IV), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—., —C(CH₃)₂CH₂—, —CH₂CH₂C(CH₃)₂—, —CH₂C(CH₃)₂CH₂—, or —C(CH₃)₂CH₂CH₂—. In another embodiment of formula (IV). Z is CH(CH₂CH₃)—, —CH₂CH(CH₂CH₃)—, —CH(CH₂CH₃)CH₂—. —CH(CH₂CH₃)CH₂CH₂—, —CH₂CH(CH₂CH₃)CH₂—, —CH₂CH₂CH(CH₂CH₃)—, —C(CH₂CH₃)₂—. —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (IV), Z is —CH₂—, —CH₂CH₂—, —CH(CH₃)—, or —C(CH₃)₂—. In yet another embodiment of formula (IV), Z is —CH₂—.

In another embodiment of formula (IV), Z is C₂₋₆ alkenylene. In yet another embodiment of formula (IV), Z is —CH═CH—, —CH₂CH₂═CH—, —CH═CHCH₂—, —CH₂—CH═CH—CH₂—, —CH═CH—CH₂CH₂—, or —CH₂CH₂—CH═CH—. In another embodiment of formula (IV), Z is —CH(═CH₂)—, —CH₂CH(═CH₂)—, —CH(═CH₂)CH₂—, or —CH(═CHCH₃)—. In yet another embodiment of formula (IV), Z is —CH═CH— or —CH(═CH₂)—.

In one embodiment of formula (IV), Z is a bond.

In another embodiment of formula (IV), Z is NR⁵, wherein R⁵ is H or C₁₋₆ alkyl.

In one embodiment of formula (IV), A is phenyl, naphthyl, indenyl or C₃₋₈ cycloalkyl. In yet another embodiment of formula (IV), A is phenyl.

In another embodiment of formula (IV), A is a 5-7 membered heterocycloalkyl or heterocycloalkenyl. In another embodiment of formula (IV), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (IV), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (IV), A is a 5-7 membered heteroaryl. In another embodiment of formula (IV), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (IV), A is optionally substituted with —(R¹)ₙ, wherein n is 0, 1, 2, or 3. In one embodiment of formula (IV), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁶, SR⁶, C(O)R⁶, C(O)NR⁷R⁸, C(O)OR⁶, OC(O)R⁶, OC(O)NR⁷R⁸, NR⁷R⁸, NR⁷C(O)R⁶, S(O)R⁶, S(O)NR⁷R⁸, S(O)₂R⁶, NR⁷S(O)₂R⁶, and S(O)₂NR⁷R⁸; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (IV), A is phenyl, n is 2, and R¹, at each occurrence, is halo.

In one embodiment of formula (IV), B is phenyl. In another embodiment of formula (IV), B is phenyl and is unsubstituted with R². In another embodiment, the phenyl is substituted with one or two R², and R² is halo, C₁₋₆-alkyl, C₁₋₆ haloalkyl, or OR⁶.

In one embodiment of formula (IV), B is phenyl, wherein the phenyl is substituted with R³, and R³ is heterocycloalkyl, wherein the heterocycloalkyl is optionally substituted with one, two, or three R¹²; wherein R¹² is halo, C₁₋₆-alkyl, or C₁₋₆-haloalkyl. In yet another embodiment, phenyl is substituted with heterocycloalkyl, and heterocycloalkyl is selected from the group consisting of azetidinyl, pyrrolidinyl, piperidinyl, morpholinyl, piperazinyl, azepanyl, diazepanyl, and hexahydropyrrolo[1,2-a]pyrazin-2(1H)yl.

In one embodiment of formula (IV). B is

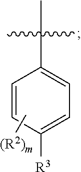

wherein R² and R³ are as defined above and m is 0, 1, or 2. In another embodiment of formula (IV), m is 0. In another embodiment of formula (IV), m is 1, and R², at each occurrence, is independently selected from the group consisting of halo. CN. OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (IV), m is 1 and R² is selected from the group consisting of halo, and C₁₋₄ alkoxy. In another embodiment of formula (IV), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁹, C(O)R⁹, C(O)NR¹⁰R¹¹, C(O)OR⁹, OC(O)R⁹, OC(O)NR¹⁰R¹¹, NR¹⁰R¹¹, NR¹⁰C(O)R⁹, S(O)R⁹, S(O)NR¹⁰R¹¹, S(O)₂R⁹, NR¹⁰S(O)₂R⁹, and S(O)₂NR¹⁰R¹¹, wherein the C₃₋₈ cycloalkyl, aryl, heterocycloalkyl, and heteroaryl, alone or part of another moiety, are optionally substituted with one, two, or three R¹², wherein R¹² is defined above. In yet another embodiment of formula (IV), B is phenyl, and R¹ is heterocycloalkyl. In yet another embodiment of formula (IV), R³ is heterocycloalkyl. In yet another embodiment of formula (IV), R³ is heterocycloalkyl, which is optionally substituted with one R¹², and R¹² is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{g}, SR^{g}, C(O)R^{g}, C(O)NR^{h}Rⁱ, C(O)OR^{g}, OC(O)R^{g}, OC(O)NR^{h}Rⁱ, NR^{h}Rⁱ, NR^{h}C(O)Rⁱ, S(O)R^{g}, S(O)NR^{h}Rⁱ, S(O)₂R^{g}, NR^{h}S(O)₂R^{g}, and S(O)₂NR^{h}Rⁱ, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{g}, R^{h}, and Rⁱ are as defined above.

In another embodiment of formula (IV), B is

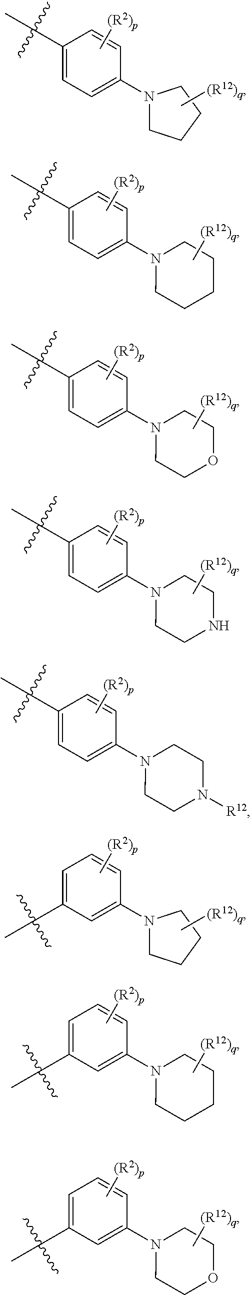

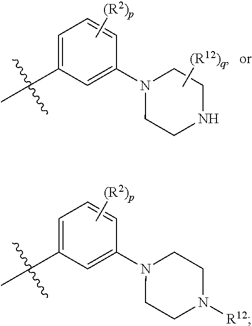

wherein R² is halo, C₁₋₆-alkyl, C₁₋₆ haloalkyl, or OR⁶; p is 0 or 1; R¹² is C₁₋₆-alkyl, C₁₋₆-haloalkyl, ORE, C(O)R^{g}, C(O)NR^{h}Rⁱ, C(O)OR^{g}, NR^{h}Rⁱ, NR^{h}C(O)R^{g}, S(O)₂R^{g}, or S(O)₂NR^{h}Rⁱ; and q is 0 or 1.

In one embodiment of formula (IV), B is

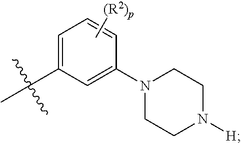

R² is halo, C₁₋₆-alkyl, C₁₋₆ haloalkyl, or OR⁶; and p is 0, 1, or 2.

In one embodiment of formula (IV), B is a 4-8 membered monocyclic heterocyclyl.

In another embodiment, B is a 4-8 membered heterocycloalkyl or heterocycloalkenyl. In another embodiment, B is a 5-7 membered heteroaryl. In yet another embodiment of formula (IV), B is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (IV), B is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl. In one embodiment, B is unsubstituted. In another embodiment, B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d},

In one embodiment of formula (IV). B is a 7-11 membered bicyclic heterocyclyl. In another embodiment, B is a 7-11 membered bicyclic heterocycloalkyl or bicyclic heterocyloalkenyl. In another embodiment, B is a 7-11 membered bicyclic heteroaryl. In yet another embodiment, B is 2,3-dihydro-2-oxo-1H-indolyl, benzothiazolyl benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl, dihydroisoindolyl, dihydroquinazolinyl, 3,4-dihydro-4-oxo-quinazolinyl, benzisothiazolyl, benzisoxazolyl, benzodiazinyl, benzofurazanyl, benzothiopyranyl, benzotriazolyl, benzpyrazolyl, 1,3-benzodioxolyl, dihydrobenzofuryl, dihydrobenzothienyl, dihydrobenzothiopyranyl, dihydrobenzothiopyranyl sulfone, dihydrobenzopyranyl, dihydrobenzoxazinyl, 3-oxo-3,4-dihydro-1,4-benzoxazinyl, indolinyl, indazolyl, isochromanyl, isoindolinyl, naphthyridinyl, phthalazinyl, piperonyl, purinyl, pyridopyridyl, pyrrolotriazinyl, quinazolinyl, tetrahydroquinolinyl, thienofuryl, thienopyridyl, 3H-imidazo[4,5-c]pyridinyl, or thienothienyl. In one embodiment of formula (I). B is unsubstituted. In another embodiment of formula (IV), B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

In one embodiment of formula (IV), B is 10-15 membered tricyclic heterocyclyl. In another embodiment, B is a 10-15 membered tricyclic heterocycloalkyl or tricyclic heterocyloalkenyl. In another embodiment, B is a 10-15 membered tricyclic heteroaryl. In one embodiment of formula (IV), B is unsubstituted. In another embodiment of formula (IV), B is substituted with one, two, or three R⁴, and R⁴ is halo, C₁₋₆-alkyl, C₁₋₆-haloalkyl, OR^{d}, C(O)R^{d}, C(O)OR^{d}, NR^{e}R^{f}, or S(O)₂R^{d}.

Specific embodiments contemplated as part of the invention include, but are not limited to, compounds of formula (I), for example;
7-(2,6-dichlorobenzyl)-5-{[2-methoxy-4-(piperazin-1-yl)phenyl]amino}-2,3-dihydropyrido[4,3-d]pyrimidin-4(1H)-one;
7-(2,6-dichlorobenzyl)-5-{[2-methoxy-4-(piperazin-1-yl)phenyl]amino}pyrido[3,4-d]pyridazin-4-ol;
8-(2,6-dichlorobenzyl)-6-{[2-methoxy-4-(piperazin-1-yl)phenyl]amino}-1,2,3,4-tetrahydro-5H-pyrido[2,3-e][1,4]diazepin-5-one;
8-(2,6-dichlorobenzyl)-6-{[4-(piperazin-1-yl)phenyl]amino}-1,2,3,4-tetrahydro-5H-pyrido[2,3-e][1,4]diazepin-5-one;
7-(2,6-dichlorobenzyl)-5-{[4-(piperazin-1-yl)phenyl]amino}pyrido[3,4-d]pyridazin-4-ol;
8-(2,6-dichlorobenzyl)-6-{[4-(piperazin-1-yl)phenyl]amino}-1,2,3,4-tetrahydro-5H-pyrido[4,3-e][1,4]diazepin-5-one;
7-(2,6-dichlorobenzyl)-5-{[3-methyl-4-(piperazin-1-yl)phenyl]amino}pyrido[3,4-d]pyridazin-4(3H)-one;
5-{[4-(4-cyclohexylpiperazin-1-yl)-2-methoxyphenyl]amino}-7-(2,6-dichlorobenzyl)pyrido[3,4-d]pyridazin-4(3H)-one;
[4-(4-{[7-(2,6-dichlorobenzyl)-4-hydroxypyrido[3,4-d]pyridazin-5-yl]amino}-3-methoxyphenyl)piperazin-1-yl](phenyl)methanone;
7-[(2,6-dichlorophenyl)amino]-5-{[2-methoxy-4-(piperazin-1-yl)phenyl]amino}pyrido[3,4-d]pyridazin-4-ol;
7-(2,6-dichlorobenzyl)-5-({2-methoxy-4-[4-(propan-2-ylsulfonyl)piperazin-1-yl]phenyl}amino)pyrido[3,4-d]pyridazin-4-ol;
1-[4-(4-{[7-(2,6-dichlorobenzyl)-4-hydroxypyrido[3,4-d]pyridazin-5-yl]amino}-3-methoxyphenyl)piperazin-1-yl]-2-(dimethylamino)ethanone;
5-{[3-chloro-4-(piperazin-1-yl)phenyl]amino}-7-(2,6-dichlorobenzyl)pyrido[3,4-d]pyridazin-4(3H)-one;
7-(2,6-dichlorobenzyl)-5-{[5-(piperazin-1-yl)pyridin-2-yl]amino}pyrido[3,4-d]pyridazin-4(3H)-one;
7-(2,6-dichlorobenzyl)-5-({2-methoxy-4-[4-(1-methylpiperidin-4-yl)piperazin-1-yl]phenyl}amino)pyrido[3,4-d]pyridazin-4(3H)-one;
7-(2,6-dichlorobenzyl)-5-({2-methoxy-4-[4-(3-methylbutyl)piperazin-1-yl]phenyl}amino)pyrido[3,4-d]pyridazin-4(3H)-one;
7-(2,6-dichlorobenzyl)-5-({2-methoxy-4-[2-(pyrrolidin-1-yl)ethyl]phenyl}amino)pyrido[3,4-d]pyridazin-4(3H)-one;
7-(2,6-dichlorobenzyl)-5-({2-methoxy-4-[2-oxo-2-(pyrrolidin-1-yl)ethyl]phenyl}amino)pyrido[3,4-d]pyridazin-4(3H)-one;
7-(2,6-dichlorobenzyl)-5-({2-methoxy-4-[4-(3-methylbutanoyl)piperazin-1-yl]phenyl}amino)pyrido[3,4-d]pyridazin-4(3H)-one;
7-(2,6-dichlorobenzyl)-5-{[2,5-difluoro-4-(piperazin-1-yl)phenyl]amino}pyrido[3,4-d]pyridazin-4(3H)-one;
7-(2,6-dichlorobenzyl)-5-{[2,6-dimethyl-4-(piperazin-1-yl)phenyl]amino}pyrido[3,4-d]pyridazin-4-ol;
5-{[2-methoxy-4-(piperazin-1-yl)phenyl]amino}-7-{[2-(propan-2-ylsulfonyl)phenyl]amino}pyrido[3,4-d]pyridazin-4(3H)-one;
5-{[2-methoxy-4-(piperazin-1-yl)phenyl]-amino}-7-{[(4R)-4-(propan-2-yl)-4,5-dihydro-1,3-oxazol-2-yl]amino}pyrido[3,4-d]pyridazin-4-ol,
7-[(2-chlorophenyl)amino]-5-{[2-methoxy-4-(piperazin-1-yl)phenyl]amino}pyrido[3,4-d]pyridazin-4(3H)-one;
7-[(5-chloro-2-fluorophenyl)amino]-5-{[2-methoxy-4-(piperazin-1-yl)phenyl]amino}pyrido[3,4-d]pyridazin-4(3H)-one;
7-(2,6-dichlorobenzyl)-5-{[2-fluoro-4-(piperazin-1-yl)phenyl]amino}pyrido[3,4-d]pyridazin-4(3H)-one;
7-[(2-chloro-5-fluorophenyl)amino]-5-{[2-methoxy-4-(piperazin-1-yl)phenyl]amino}pyrido[3,4-d]pyridazin-4(3H)-one;
7-[(2,5-dichlorophenyl)amino]-5-{[2-methoxy-4-(piperazin-1-yl)phenyl]amino}pyrido[3,4-d]pyridazin-4(3H)-one;
7-[(2-chloro-6-fluorophenyl)amino]-5-{[2-methoxy-4-(piperazin-1-yl)phenyl]amino}pyrido[3,4-d]pyridazin-4(3H)-one;
7-(2,6-dichlorobenzyl)-5-{[2-methoxy-4-(4-methylpiperazin-1-yl)phenyl]amino}pyrido[3,4-d]pyridazin-4(3H)-one;
7-[(3,5-dichlorophenyl)amino]-5-{[2-methoxy-4-(piperazin-1-yl)phenyl]amino}pyrido[3,4-d]pyridazin-4(3H)-one;
7-[(2,6-difluorophenyl)amino]-5-{[2-methoxy-4-(piperazin-1-yl)phenyl]amino}pyrido[3,4-d]pyridazin-4(3H)-one;
7-[(3,5-dichloropyridin-4-yl)amino]-5-{[2-methoxy-4-(piperazin-1-yl)phenyl]amino}pyrido[3,4-d]pyridazin-4(3H)-one;
7-[(2,4-dichlorophenyl)amino]-5-{[2-methoxy-4-(piperazin-1-yl)phenyl]amino}pyrido[3,4-d]pyridazin-4-ol;
7-[(2,6-dichloro-4-fluorophenyl)amino]-5-{[2-methoxy-4-(piperazin-1-yl)phenyl]amino}pyrido[3,4-d]pyridazin-4-ol;
7-[(2,3-difluorophenyl)amino]-5-{[2-methoxy-4-(piperazin-1-yl)phenyl]amino}pyrido[3,4-d]pyridazin-4(3H)-one;
7-[(furan-2-ylmethyl)amino]-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-(piperidin-1-yl)pyrido[3,4-d]pyridazin-4(3H)-one;
7-(benzylamino)-5-{[2-methoxy-4-(piperazin-1-yl)phenyl]amino}pyrido[3,4-d]pyridazin-4(3H)-one;
5-{[2-methoxy-4-(piperazin-1-yl)phenyl]amino}-7-phenylpyrido[3,4-d]pyridazin-4(3H)-one;
7-(2,6-dichlorobenzyl)-5-{[2,6-difluoro-4-(piperazin-1-yl)phenyl]amino}pyrido[3,4-d]pyridazin-4(3H)-one;
5-{[2-methoxy-4-(piperazin-1-yl)phenyl]amino}-7-(pyridin-2-ylamino)pyrido[3,4-d]pyridazin-4(3H)-one;
5-{[2-methoxy-4-(piperazin-1-yl)phenyl]amino}-7-(pyridin-3-ylamino)pyrido[3,4-d]pyridazin-4(3H)-one;
7-(cyclohexylamino)-5-{[2-methoxy-4-(piperazin-1-yl)phenyl]amino}pyrido[3,4-d]pyridazin-4(3H)-one;
5-{[2-methoxy-4-(piperazin-1-yl)phenyl]amino}-7-{[2-(pyridin-2-yl)ethyl]amino}pyrido[3,4-d]pyridazin-4-ol;
7-[(2-fluorophenyl)amino]-5-{[2-methoxy-4-(piperazin-1-yl)phenyl]amino}pyrido[3,4-d]pyridazin-4(3H)-one;
7-[(2,6-dimethylphenyl)amino]-5-{[2-methoxy-4-(piperazin-1-yl)phenyl]amino}pyrido[3,4-d]pyridazin-4(3H)-one;
7-[(3-chloropyridin-2-yl)amino]-5-{[2-methoxy-4-(piperazin-1-yl)phenyl]amino}pyrido[3,4-d]pyridazin-4(3H)-one:
7-[(2,3-dichlorophenyl)amino]-5-{[2-methoxy-4-(piperazin-1-yl)phenyl]amino}pyrido[3,4-d]pyridazin-4(3H)-one;
5-{[2-methoxy-4-(piperazin-1-yl)phenyl]amino}-7-[(2,4,6-trifluorophenyl)amino]pyrido[3,4-d]pyridazin-4(3H)-one;
5-{[2-methoxy-4-(piperazin-1-yl)phenyl]amino}-7-[(2,3,4-trifluorophenyl)amino]pyrido[3,4-d]pyridazin-4(3H)-one;
7-[(3-chloro-2-fluorophenyl)amino]-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-(tetrahydro-2H-pyran-4-ylamino)pyrido[3,4-d]pyridazin-4-ol;
5-{[2-methoxy-4-(piperazin-1-yl)phenyl]amino}-7-(1,3-thiazol-2-ylamino)pyrido[3,4-d]pyridazin-4(3H)-one;
5-{[2-chloro-4-(piperazin-1-yl)phenyl]amino}-7-(2,6-dichlorobenzyl)pyrido[3,4-d]pyridazin-4(3H)-one;
5-{[2-methoxy-4-(piperazin-1-yl)phenyl]amino}-7-[(1-methylpiperidin-4-yl)amino]pyrido[3,4-d]pyridazin-4(3H)-one;
7-(2,6-dichlorobenzyl)-5-{[3-fluoro-2-methoxy-4-(piperazin-1-yl)phenyl]amino}pyrido[3,4-d]pyridazin-4-ol;
7-[(3-chloropyridin-4-yl)amino]-5-{[2-methoxy-4-(piperazin-1-yl)phenyl]amino}pyrido[3,4-d]pyridazin-4(3H)-one;
5-{[2-bromo-4-(piperazin-1-yl)phenyl]amino}-7-(2,6-dichlorobenzyl)pyrido[3,4-d]pyridazin-4(3H)-one;
7-(2-chlorophenoxy)-5-({[2-methoxy-4-(piperazin-1-yl)phenyl]amino}pyrido[3,4-d]pyridazin-4(3H)-one;
7-(2,6-dichlorophenoxy)-5-{[2-methoxy-4-(piperazin-1-yl)phenyl]amino}pyrido[3,4-d]pyridazin-4(3H)-one;
7-(2,6-dichlorobenzyl)-5-{[4-(piperazin-1-yl)-2-(trifluoromethoxy)phenyl]amino}pyrido[3,4-d]pyridazin-4-ol;
7-[(2,3-dichloro-6-fluorophenyl)amino]-5-{[2-methoxy-4-(piperazin-1-yl)iphenyl]amino}pyrido[3,4-d]pyridazin-4(3H)-one;
7-(2,6-dichlorobenzyl)-5-{[4-(hexahydropyrrolo[1,2-a]pyrazin-2(1H)-yl)-2-methoxyphenyl]amino}pyrido[3,4-d]pyridazin-4(3H)-one;
7-[(2,3-dichloro-4-fluorophenyl)amino]-5-{[2-methoxy-4-(piperazin-1-yl)phenyl]amino}pyrido[3,4-d]pyridazin-4-ol;
2-[(2,6-dichlorophenyl)amino]-4-{[2-methoxy-4-(piperazin-1-yl)phenyl]amino}pyrido[2,3-d]pyridazin-5(6H)-one;
7-(2,6-dichlorobenzyl)-5-{[2,3-dimethyl-4-(piperazin-1-v)phenyl]amino}pyrido[3,4-d]pyridazin-4(3H)-one;
2-(2,6-dichlorobenzyl)-4-{[2-methoxy-4-(piperazin-1-yl)phenyl]amino}pyrido[2,3-d]pyridazin-5(6H)-one;
7-[(2-chloro-4,6-difluorophenyl)amino]-5-{[2-methoxy-4-(piperazin-1-yl)phenyl]amino}pyrido[3,4-d]pyridazin-4-ol;
7-(2,6-dichlorobenzyl)-5-{[2-methoxy-5-methyl-4-(piperazin-1-yl)phenyl]amino}pyrido[3,4-d]pyridazin-4(3H)-one;
7-(2,6-dichlorobenzyl)-5-{[2-fluoro-5-methyl-4-(piperazin-1-yl)phenyl]amino}pyrido[3,4-d]pyridazin-4(3H)-one;
6-(2,6-dichlorobenzyl)-8-[(6-methoxy-1,2,3,4-tetrahydroisoquinolin-7-yl)amino]phthalazin-1(2H)-one;
7-(2,6-difluorophenoxy)-5-{[2-methoxy-4-(piperazin-1-yl)phenyl]amino}pyrido[3,4-d]pyridazin-4(3H)-one;
7-(2-fluorophenoxy)-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-[(2,3,4-trichlorophenyl)amino]pyrido[3,4-d]pyridazin-4(3H)-one;
7-(2,3-difluorophenoxy)-5-{[2-methoxy-4-(piperazin-1-yl)phenyl]amino}pyrido[3,4-d]pyridazin-4-ol;
7-(2,3-dichlorophenoxy)-5-{[2-methoxy-4-(piperazin-1-yl)phenyl]amino}pyrido[3,4-d]pyridazin-4(3H)-one;
7-(2,6-dichloro-4-fluorophenoxy)-5-{[2-methoxy-4-(piperazin-1-yl)phenyl]amino}pyrido[3,4-d]pyridazin-4(3H)-one;
7-[(3-chloropyridin-4-yl)oxy]-5-{[2-methoxy-4-(piperazin-1-yl)phenyl]amino}pyrido[3,4-d]pyridazin-4(3H)-one;
7-(2,3-dimethylphenoxy)-5-{[2-methoxy-4-(piperazin-1-yl)phenyl]amino}pyrido[3,4-d]pyridazin-4-ol;
7-[(3-fluoropyridin-4-yl)oxy]-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-(2,3,4-trichlorophenoxy)pyrido[3,4-d]pyridazin-4-ol;
7-(2,4-dichlorophenoxy)-5-{[2-methoxy-4-(piperazin-1-yl)phenyl]amino}pyrido[3,4-d]pyridazin-4(3H)-one;
7-[(trans-4-hydroxycyclohexyl)amino]-5-{[2-methoxy-4-(piperazin-1-yl)phenyl]amino}pyrido[3,4-d]pyridazin-4-ol;
7-(cyclopentyloxy)-5-{[2-methoxy-4-(piperazin-1-yl)phenyl]amino}pyrido[3,4-d]pyridazin-4-ol;
7-(2,6-dichlorobenzyl)-5-[(7-methoxy-1,2,3,4-tetrahydroisoquinolin-6-yl)amino]pyrido[3,4-d]pyridazin-4(3H)-one;
2-(2-chlorophenoxy)-4-{[2-methoxy-4-(piperazin-1-yl)phenyl]amino}pyrido[2,3-d]pyridazin-5(6H)-one;
5-{[2-chloro-4-(pyrrolidin-1-ylmethyl)phenyl]amino}-7-(4-fluoropiperidin-1-yl)pyrido[3,4-d]pyridazin-4-ol;
2-(2-chlorobenzyl)-4-[(6-methoxy-1,2,3,4-tetrahydroisoquinolin-7-yl)amino]pyrido[2,3-d]pyridazin-5-ol;
2-(2-chlorobenzyl)-4-{[2-methoxy-4-(piperazin-1-ylmethyl)phenyl]amino}pyrido[2,3-d]pyridazin-5(6H)-one;
5-{[2-chloro-4-(pyrrolidin-1-ylmethyl)phenyl]amino}-7-[(cyclopropylmethyl)amino]pyrido[3,4-d]pyridazin-4-ol;
2-(2-chlorobenzyl)-4-{[2-methoxy-4-(morpholin-4-ylmethyl)phenyl]amino)}pyrido[2,3-d]pyridazin-5-ol;
7-(2-chlorophenoxy)-5-{[2-ethoxy-4-(pyrrolidin-1-ylmethyl)phenyl]amino}pyrido[3,4-d]pyridazin-4(3H)-one;
5-{[2-chloro-4-(pyrrolidin-1-ylmethyl)phenyl]amino}-7-(4-hydroxypiperidin-1-yl)pyrido[3,4-d]pyridazin-4(3H)-one;
5-{[2-chloro-4-(pyrrolidin-1-ylmethyl)phenyl]amino}-7-(3-hydroxyazetidin-1-yl)pyrido[3,4-d]pyridazin-4(3H)-one;
7-(2-chlorophenoxy)-5-{[2-methoxy-4-(piperidin-4-yl)phenyl]amino}pyrido[3,4-d]pyridazin-4-ol;
7-[(cyclopropylmethyl)amino]-5-{[2-methoxy-4-(piperidin-4-yl)phenyl]amino}pyrido[3,4-d]pyridazin-4-ol;
2-(2-chlorobenzyl)-4-{[2-methoxy-4-(piperidin-4-yl)phenyl]amino}pyrido[2,3-d]pyridazin-5(6H)-one;
2-(2,6-dichlorobenzyl)-4-{[2-methoxy-4-(piperidin-4-yl)phenyl]amino}pyrido[2,3-d]pyridazin-5(6H)-one;
4-{[2-methoxy-4-(piperidin-4-yl)phenyl]amino}-2-[2-(morpholin-4-yl)ethyl]pyrido[2,3-d]pyridazin-5(6H)-one;
7-(2-chlorophenoxy)-5-{[5-(piperazin-1-yl)pyridin-2-yl]amino}pyrido[3,4-d]pyridazin-4(3H)-one;
2-(2-cyclopropylethyl)-4-{[2-methoxy-4-(piperidin-4-yl)phenyl]amino}pyrido[2,3-d]pyridazin-5(6H)-one;
(4-{[7-(2-chlorophenoxy)-4-hydroxypyrido[3,4-d]pyridazin-5-yl]amino}-3-methoxyphenyl)(pyrrolidin-1-yl)methanone;
7-(2-chlorophenoxy)-5-{[2-ethoxy-4-(piperidin-4-yl)phenyl]amino}pyrido[3,4-d]pyridazin-4(3H)-one;
2-(2-fluorobenzyl)-4-{[2-methoxy-4-(piperidin-4-yl)phenyl]amino}pyrido[2,3-d]pyridazin-5(6H)-one;
2-(2,3-dichlorobenzyl)-4-{[2-methoxy-4-(piperidin-4-yl)phenyl]amino}pyrido[2,3-d]pyridazin-5(6H)-one;
2-(2,6-difluorobenzyl)-4-{[2-methoxy-4-(piperidin-4-yl)phenyl]amino}pyrido[2,3-d]pyridazin-5(6H)-one;
2-(2,5-difluorobenzyl)-4-{[2-methoxy-4-(piperidin-4-yl)phenyl]amino}pyrido[2,3-d]pyridazin-5(6H)-one;
2-(2,3-difluorobenzyl)-4-{[2-methoxy-4-(piperidin-4-yl)phenyl]amino}pyrido[2,3-d]pyridazin-5(6H)-one;
2-(2-chloro-6-fluorobenzyl)-4-{[2-methoxy-4-(piperidin-4-yl)phenyl]amino}pyrido[2,3-d]pyridazin-5(6H)-one;
5-[(2-methoxyphenyl)amino]-7-{[4-(piperazin-1-yl)phenyl]amino}pyrido[3,4-d]pyridazin-4-ol;
7-(2-chlorophenoxy)-5-{[2-ethoxy-4-(piperazin-1-yl)phenyl]amino}pyrido[3,4-d]pyridazin-4-ol;
2-[(5-{[2-methoxy-4-(piperazin-1-yl)phenyl]amino}-4-oxo-3,4-dihydropyrido[3,4-d]pyridazin-7-yl)oxy]benzonitrile;
2-(2-chlorobenzyl)-4-({2-methoxy-4-[(4-methylpiperazin-1-yl)methyl]phenyl}amino)pyrido[2,3-d]pyridazin-5(6H)-one;
7-(2-chlorophenoxy)-5-{[2-(difluoromethoxy)-4-(piperazin-1-yl)phenyl]amino}pyrido[3,4-d]pyridazin-4(3H)-one;
7-(2-chlorophenoxy)-5-{[5-(piperidin-4-yl)pyridin-2-yl]amino}pyrido[3,4-d]pyridazin-4(3H)-one;
7-(2-chlorophenoxy)-5-{[2-methoxy-4-(piperazin-1-yl)phenyl]amino}-1-methylpyrido[3,4-d]pyridazin-4-ol, and
2-{[7-(2-chlorophenoxy)-4-hydroxypyrido[3,4-d]pyridazin-5-yl]amino}-5-(piperazin-1-yl)benzonitrile.

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers of the compounds thereof.

Compounds of this invention may also contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers.

Additional geometric isomers may exist in the present compounds. For example, the invention contemplates the various geometric isomers and mixtures thereof resulting from the disposition of substituents around a cycloalkyl group or a heterocycle group. Substituents around a cycloalkyl or a heterocycle are designated as being of cis or trans configuration.

Compounds of this invention may also exist as tautomers or equilibrium mixtures thereof wherein a proton of a compound shifts from one atom to another. Examples of tautomers include, but are not limited to, keto-enol, phenol-keto, oxime-nitroso, nitro-aci, imine-enamine and the like. Tautomeric forms are intended to be encompassed by the scope of this invention, even though only one tautomeric form may be depicted.

This invention also is directed, in part, to all salts of the compounds of formula (I). A salt of a compound may be advantageous due to one or more of the salt's properties, such as, for example, enhanced pharmaceutical stability in differing temperatures and humidities, or a desirable solubility in water or other solvents. Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. Pharmaceutically acceptable salts include salts commonly used to form alkali metal salts and to form addition salts of free acids or free bases. In general, these salts typically may be prepared by conventional means by reacting, for example, the appropriate acid or base with a compound of the invention.

Pharmaceutically acceptable acid addition salts of the compounds of formula (I) can be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, oxalate, palmoate, pectinate, 2-naphthalesulfonate, 3-phenylpropionate, picrate, pivalate, thiocyanate, tosylate, and undecanoate.

Pharmaceutically acceptable base addition salts of the compounds of formula (I) include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

Compounds of formula (I) (and salts thereof) with any level of purity (including pure and substantially pure) are within the scope of Applicants' invention. The term “substantially pure” in reference to a compound/salt/isomer, means that the preparation/composition containing the compound/salt/isomer contains more than about 85% by weight of the compound/salt/isomer, preferably more than about 90% by weight of the compound/salt/isomer, preferably more than about 95% by weight of the compound/salt/isomer, preferably more than about 97% by weight of the compound/salt/isomer, and preferably more than about 99% by weight of the compound/salt/isomer.

### Preparation of Compounds

Compounds of this invention may be made by synthetic chemical processes, examples of which are shown herein. It is meant to be understood that the order of the steps in the processes may be varied, that reagents, solvents and reaction conditions may be substituted for those specifically mentioned, and that vulnerable moieties may be protected and deprotected, as necessary.

Protecting groups for C(O)OH moieties include, but are not limited to acetoxymethyl, allyl, benzoylmethyl, benzyl, benzyloxymethyl, tert-butyl, tert-butyldiphenylsilyl, diphenylmethyl, cyclobutyl, cyclohexyl, cyclopentyl, cyclopropyl, diphenylmethylsilyl, ethyl, para-methoxybenzyl, methoxymethyl, methoxyethoxymethyl, methyl, methylthiomethyl, naphthyl, para-nitrobenzyl, phenyl, n-propyl, 2,2,2-trichloroethyl, triethylsilyl, 2-(trimethylsilyl)ethyl, 2-(trimethylsilyl)ethoxymethyl, triphenylmethyl and the like.

Protecting groups for C(O) and C(O)H moieties include, but are not limited to, 1,3-dioxylketal, diethylketal, dimethylketal, 1,3-dithianylketal, O-methyloxime, O-phenyloxime and the like.

Protecting groups for NH moieties include, but are not limited to, acetyl, alanyl, benzoyl, benzyl (phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, formyl, methanesulfonyl, methoxyacetyl, 4-methoxybenzyloxycarbonyl, para-methoxybenzyl, methoxycarbonyl, methyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

### Schemes

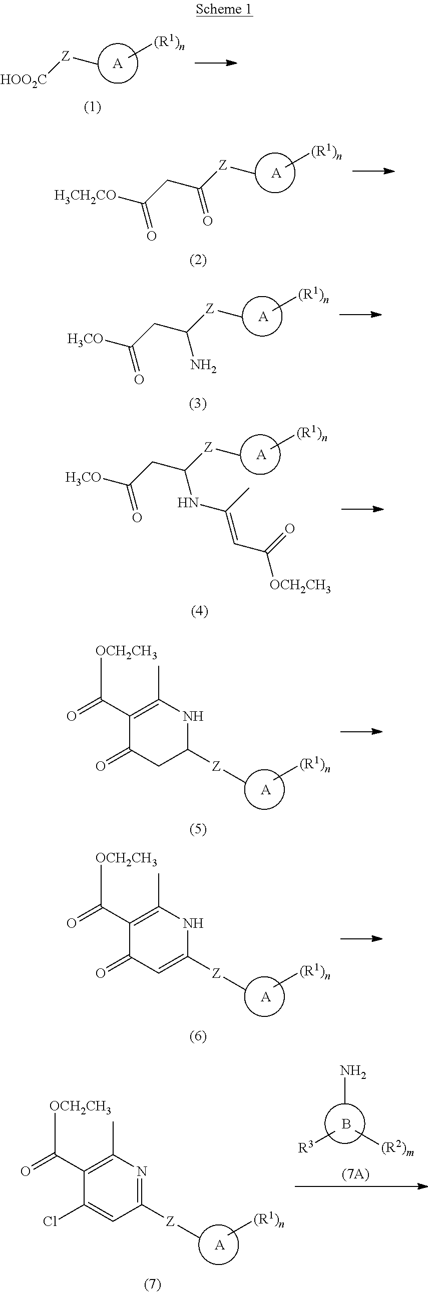

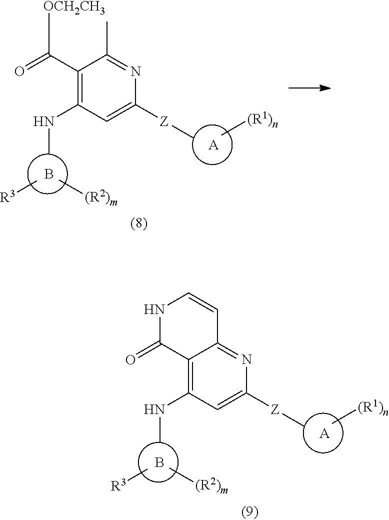

As shown in Scheme 1, compounds of formula (I), wherein A, Z, R¹ and n are as described herein, can be reacted at room temperature with N,N-carbonyldiimidazole in a solvent such as, but not limited to, dry tetrahydrofuran, followed by the addition magnesium chloride and ethyl potassium malonate at elevated temperature, to provide compounds of formula (2). Compounds of formula (3) can be prepared from compounds of formula (2) by reacting the latter with ammonium acetate, magnesium sulfate, and sodium cyanoborohydride. The reaction is typically performed in a solvent such as, but not limited to, methanol at elevated temperatures. Compounds of formula (4) can be prepared by reacting compounds of formula (3) with acetic acid, ethyl acetoacetate, and magnesium sulfate. The reaction is typically performed at elevated temperature, in a solvent such as but not limited to toluene. Compounds of formula (4) can be reacted with a base such as, but not limited to, potassium t-butoxide at ambient temperature in a solvent such as but not limited to tetrahydrofuran, to provide compounds of formula (5). Compounds of formula (6) can be prepared by reacting compounds of formula (5) with 2,3-dichloro-5,6-dicyano-1,4-benzoquinone. A solvent such as but not limited to tetrahydrofuran is typically employed. Compounds of formula (6) can be reacted with phosphorus oxychloride to provide compounds of formula (7). The reaction is typically performed at elevated temperature. Compounds of formula (8) can be prepared from compounds of formula (7) by reacting the latter with compounds of formula (7A), wherein B, R², R³, and m are as described herein, in the presence of p-toluenesulfonic acid. The reaction is typically performed at elevated temperature in a solvent such as but not limited to n-butanol. Compounds of formula (9), which are representative of the compounds of this invention, can be prepared by reacting compounds of formula (8) with sodium hydride, followed by 1,3,5-triazine at elevated temperature. The reaction is typically performed in a solvent such as but not limited to N,N-dimethylformamide.

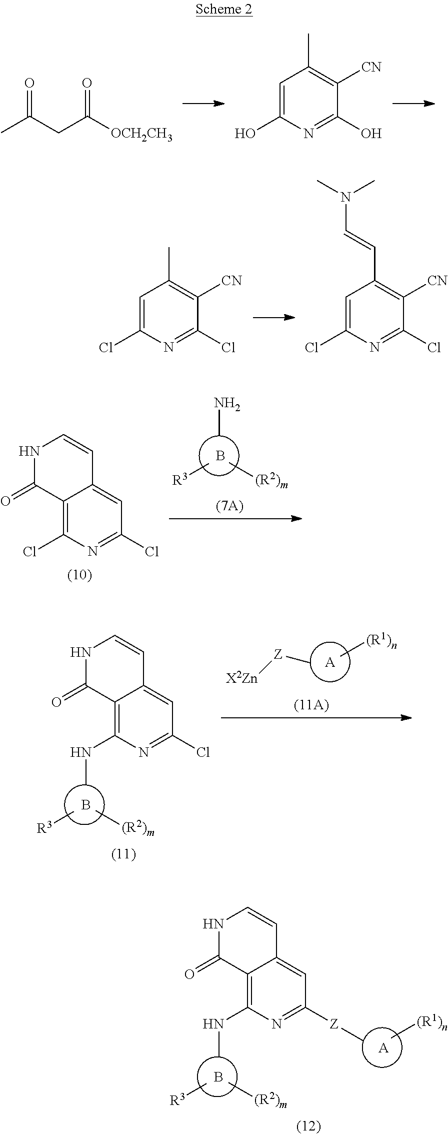

Potassium hydroxide can be added to a solution of 2-cyanoacetamide and ethyl 3-oxobutanoate in a solvent such as but not limited to methanol to provide 2,6-dihydroxy-4-methylnicotinonitrile, as shown in Scheme 2. The reaction is typically performed at elevated temperature. 2,6-Dihydroxy-4-methylnicotinonitrile and phosphorus oxychloride can heated in a sealed tube without an additional solvent to provide 2,6-dichloro-4-methylnicotinonitrile. N,N-Dimethylformamide dimethyl acetal can be added to a solution of 2,6-dichloro-4-methylnicotinonitrile in a solvent such as but not limited to N,N-dimethylformamide to provide (E)-2,6-dichloro-4-(2-(dimethylamino)vinyl)nicotinonitrile. The reaction is typically performed at an elevated temperature. Concentrated hydrochloric acid and (E)-2,6-dichloro-4-(2-(dimethylamino)vinyl)nicotinonitrile can be heated in a sealed tube to provide 6,8-dichloro-2,7-naphthyridin-1(2H)-one (10). Compounds of formula (11) can be prepared from compounds of formula (10) by reacting the latter with compounds of formula (7A), wherein B, R², R³, and m are as described herein, in a solvent such as but not limited to N-methylpyrrolidone. The reaction is typically performed at elevated temperature and may be performed in a microwave oven. Compounds of formula (12), which are representative of the compounds of this invention, can be prepared by reacting compounds of formula (11) with an organozinc compound of formula (11A), wherein Z, A, R¹, and n are as described herein and X² is a halide. The reaction typically involves the use of heat and a nickel or palladium catalyst such as but not limited to bis(triphenylphosphine)palladium(II) dichloride in a solvent such as but not limited to N-methylpyrrolidone, tetrahydrofuran, or mixtures thereof.

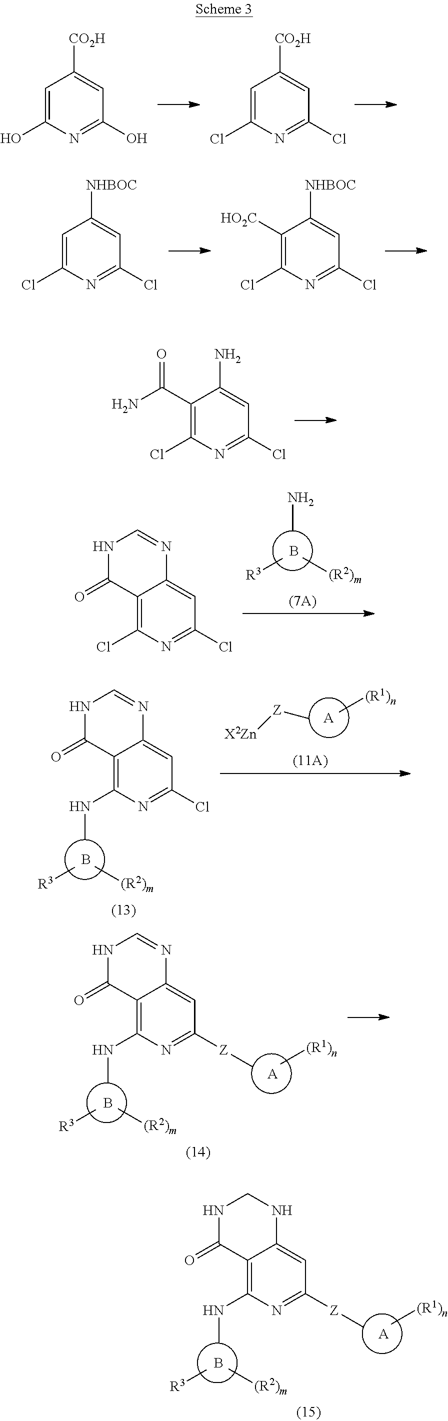

As shown in Scheme 3, 2,6-dihydroxypyridine-4-carboxylic acid and phosphoryl trichloride can be heated in a sealed tube to provide 2(6-dichloropyridine-4-carboxylic acid. Diphenylphosphoryl azide and a base such as but not limited to N,N-diisopropylethylamine in tert-butanol (200 mL) can be added to 2,6-dichloropyridine-4-carboxylic acid to provide tert-butyl 2,6-dichloropyridin-4-ylcarbamate. The reaction typically requires the use of heat. Carboxylation of tert-butyl 2,6-dichloropyridin-4-ylcarbamate to provide 4-(tert-butoxycarbonylamino)-2,6-dichloronicotinic acid can be performed by bubbling dry carbon dioxide gas through a solution of tert-butyl 2,6-dichloropyridin-4-ylcarbamate and N,N,N′,N′-tetramethylethylenediamine treated with n-butyl lithium. The n-butyl lithium is typically added at low temperature to a mixture of tert-butyl 2,6-dichloropyridin-4-ylcarbamate and N,N,N′,N′-tetramethylethylenediamine in a solvent such as but not limited to tetrathydrofuran, before adding the carbon dioxide and warming to room temperature. A solution of 4-(tert-Butoxycarbonylamino)-2,6-dichloronicotinic acid and 1,1′-carbonyldiimidazole in solvent such as but not limited to N,N-dimethylformamide can be stirred at elevated temperature before the addition of ammonia gas at reduced temperature to provide 4-amino-2,6-dichloropyridine-3-carboxamide. Triethyl orthoformate can be reacted with 4-amino-2,6-dichloropyridine-3-carboxamide to provide 5,7-dichloropyrido[4,3-d]pyrimidin-4(3H)-one. The reaction typically involves the use of heat and a solvent such as but not limited to N,N-dimethylformamide. 5,7-Dichloropyrido[4,3-d]pyrimidin-4(3H)-one can be reacted with a compound of formula (7A), wherein B, R², R³, and m are as described herein, in the presence of a base such as but not limited to triethylamine, to provide compounds of formula (13). The reaction is typically performed at elevated temperature in a solvent such as but not limited to 1,4-dioxane. Compounds of formula (14), which are representative of the compounds of this invention, can be prepared by reacting compounds of formula (13) with an organozinc compound of formula (11A), wherein Z, A, R¹, and n are as described herein and X² is a halide. The reaction typically involves the use of heat and a nickel or palladium catalyst such as but not limited to bis(triphenylphosphine)palladium(II) dichloride in a solvent such as but not limited to N-methylpyrrolidone, tetrahydrofuran, or mixtures thereof. Compounds of formula (15), which are also representative of the compounds of this invention, can be prepared by reacting compounds of formula (14) with a reducing agent such as but not limited to sodium borohydride or sodium cyanoborohydride. The reaction is typically performed at elevated temperature, in a solvent such as but not limited to tetrahydrofuran, methanol, and the like, or mixtures thereof.

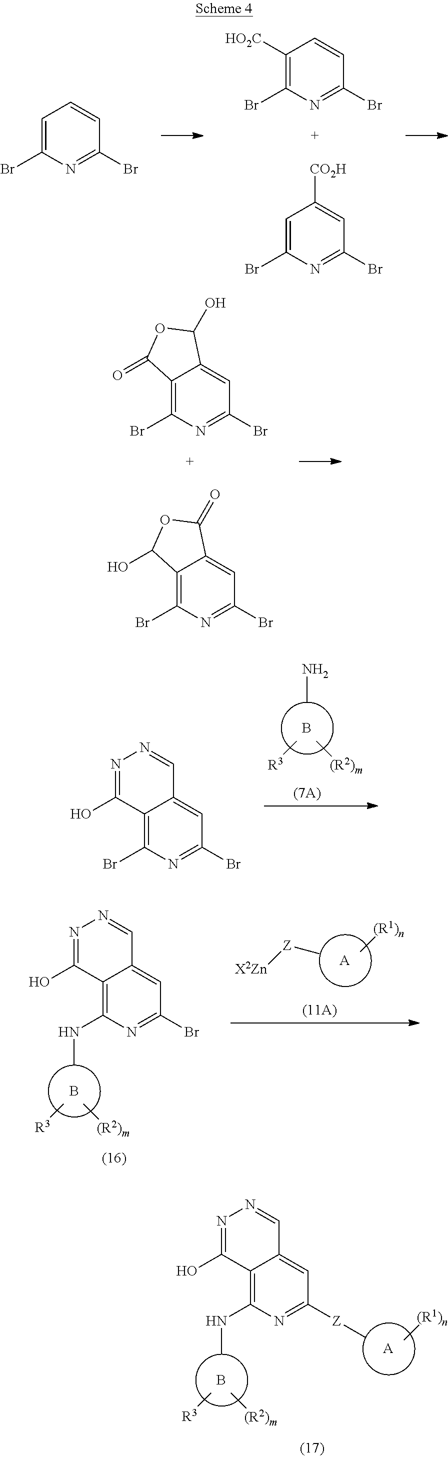

Carboxylation of 2,6-dibromopyridine to provide 2,6-dibromonicotinic acid and 2,6-dibromoisonicotinic acid can be performed by bubbling dry carbon dioxide gas to a solution of 2,6-dibromopyridine and diisopropylamine treated with n-butyl lithium. The n-butyl lithium is typically added at low temperature to a mixture of 2,6-dibromopyridine and diisopropylamine in a solvent such as but not limited to tetrathydrofuran, before adding the carbon dioxide gas and warming to room temperature. A mixture of 2,6-dibromonicotinic acid and 2,6-dibromoisonicotinic acid can be added at low temperature to a solution of a base, such as, but not limited to, 2,2,6,6-tetramethylpiperidine, in a solvent, such as, but not limited to, tetrahydrofuran, and n-butyllithium to provide 4,6-dibromo-1-hydroxyfuro[3,4-c]pyridin-3(1H)-one and 4,6-dibromo-3-hydroxyfuro[3,4-c]pyridin-1(3H)-one. The reaction is typically stirred at low temperature for several hours before the addition of N,N-dimethylformamide. A mixture of 4,6-dibromo-1-hydroxyfuro[3,4-c]pyridin-3(1H)-one and 4,6-dibromo-3-hydroxyfuro[3,4-c]pyridin-1(3H)-one can be reacted with hydrazine hydrochloride in the presence of a base such as but not limited to triethylamine to provide, after purification, 5,7-dibromopyrido[3,4-d]pyridazin-4-ol. The reaction typically requires an elevated temperature and maybe performed in a solvent such as but not limited to isopropanol. 5,7-Dibromopyrido[3,4-d]pyridazin-4-ol can be reacted with a compound of formula (7A), wherein B, R², R³, and m are as described herein, in the presence of a base such as but not limited to diisopropylethylamine or triethylamine, to provide compounds of formula (16). The reaction is typically performed at elevated temperature in a solvent such as but not limited to 1,4-dioxane. Compounds of formula (17), which are representative of the compounds of this invention, can be prepared by reacting compounds of formula (16) with an organozinc compound of formula (11A), wherein Z, A, R¹, and n are as described herein and X² is a halide. The reaction typically involves the use of heat, copper(I) iodide, and a nickel or palladium catalyst such as but not limited to 1,1′-bis(diphenylphosphino)ferrocene-palladium(II) dichloride in a solvent such as but not limited to N-methylpyrrolidone, tetrahydrofuran, or mixtures thereof.

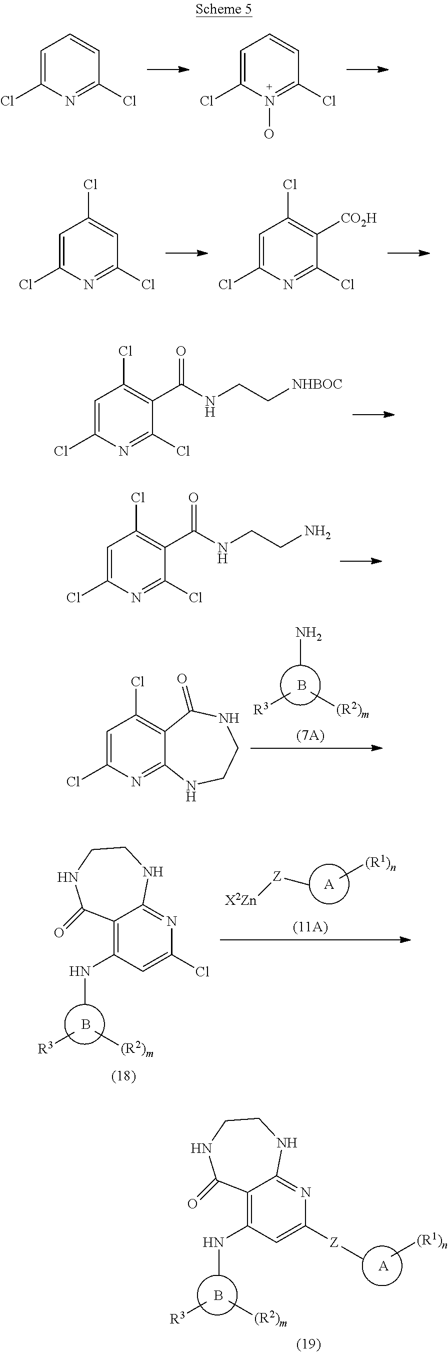

As shown in Scheme 5, 2,6-dichloropyridine 1-oxide can be prepared by reacting a solution of 2,6-dichloropyridine, 30% hydrogen peroxide, and an acid such as but not limited to trifluoroacetic acid at elevated temperature. 2,6-Dichloropyridine 1-oxide can be reacted with phosphorus oxychloride at elevated temperature to provide 2,4,6-trichloropyridine. Carboxylation of 2,4,6-trichloropyridine to provide 2,4,6-trichloronicotinic acid can be performed by adding solid carbon dioxide (dry ice) to a solution of 2,4,6-trichloropyridine and diisopropylamine treated with n-butyl lithium. The n-butyl lithium is typically added at low temperature to a mixture of 2,4,6-trichloropyridine and diisopropylamine in a solvent such as but not limited to tetrathydrofuran, before adding the carbon dioxide gas and warming to room temperature. tert-Butyl 2-(2,4,6-trichloronicotinamido)ethylcarbamate can be prepared from 2,4,6-trichloronicotinic acid by reacting the latter first with oxalyl chloride at low temperature in a solvent such as but not limited to dichloromethane, N,N-dimethylformamide, or mixtures thereof. The resulting crude acid chloride can be reacted with tert-butyl 2-aminoethylcarbamate in the presence of a base such as but not limited to triethylamine at low temperature in a solvent such as but not limited to dichloromethane to provide tert-butyl 2-(2,4,6-trichloronicotinamido)ethylcarbamate. tert-Butyl 2-(2,4,6-trichloronicotinamido)ethylcarbamate can be treated with an acid such as but not limited to trifluoroacetic acid in a solvent such as but not limited to dichloromethane, to provide N-(2-aminoethyl)-2,4,6-trichloronicotinamide. 6,8-Dichloro-1,2,3,4-tetrahydropyrido[2,3-e][1,4]diazepin-5-one can be prepared by reacting N-(2-aminoethyl)-2,4,6-trichloronicotinamide with cesium fluoride in the presence of a base such as but not limited to triethylamine. The reaction typically requires the use of heat and is performed in a solvent such as but not limited to N,N-dimethylformamide. 6,8-Dichloro-1,2,3,4-tetrahydropyrido[2,3-e][1,4]diazepin-5-one can be reacted with a compound of formula (7A), wherein B, R², R³, and m are as described herein, in the presence of a base such as but not limited to diisopropylethylamine or triethylamine, to provide compounds of formula (18). The reaction is typically performed at elevated temperature in a solvent such as but not limited to 1,4-dioxane. Compounds of formula (19), which are representative of the compounds of this invention, can be prepared by reacting compounds of formula (18) with an organozinc compound of formula (11A), wherein Z, A, R¹, and n are as described herein and X² is a halide. The reaction typically involves the use of heat, and a nickel or palladium catalyst such as but not limited to tetrakis(triphenylphosphine)palladium in a solvent such as but not limited to N-methylpyrrolidone, tetrahydrofuran, or mixtures thereof. Additionally, the reaction may be performed in a microwave oven.

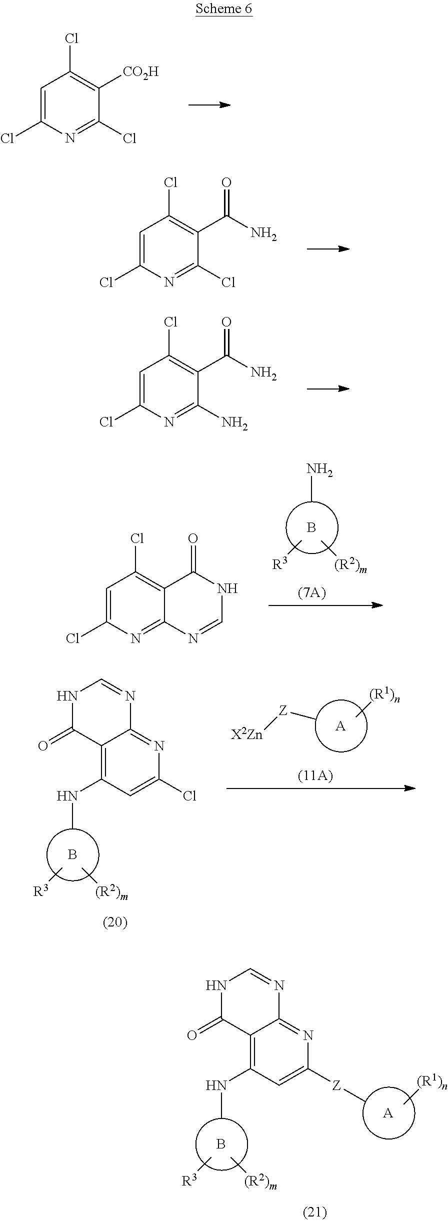

2,4,6-Trichloronicotinic acid, which can be prepared as described in Scheme 5, can be treated at ambient temperature with oxalyl chloride in a solvent such as but not limited to dichloromethane, N,N-dimethylformamide, or mixtures thereof. Ammonia gas can be bubbled through a solution of the crude acid chloride in a solvent such as but not limited to tetrahydrofuran to provide 2,4,6-trichloronicotinamide. 2-Amino-4,6-dichloronicotinamide can be prepared by reacting 2,4,6-trichloronicotinamide with ammonia. The reaction is typically performed at elevated temperature in a solvent such as but not limited to 1,4-dioxane. 2-Amino-4,6-dichloronicotinamide can be reacted with triethyl orthoformate at elevated temperature to provide 5,7-dichloropyrido[2,3-d]pyrimidin-4(3H)-one. 5,7-Dichloropyrido[2,3-d]pyrimidin-4(3H)-one can be reacted with a compound of formula (7A), wherein B, R², R³, and m are as described herein, in the presence of a base such as but not limited to diisopropylethylamine or triethylamine, to provide compounds of formula (20). The reaction is typically performed at elevated temperature in a solvent such as but not limited to 1,4-dioxane. Compounds of formula (21), which are representative of the compounds of this invention, can be prepared by reacting compounds of formula (20) with an organozinc compound of formula (11A), wherein Z, A, R¹, and n are as described herein and X² is a halide. The reaction typically involves the use of heat, and a nickel or palladium catalyst such as but not limited to tetrakis(triphenylphosphine)palladium in a solvent such as but not limited to N-methylpyrrolidone, tetrahydrofuran, or mixtures thereof.

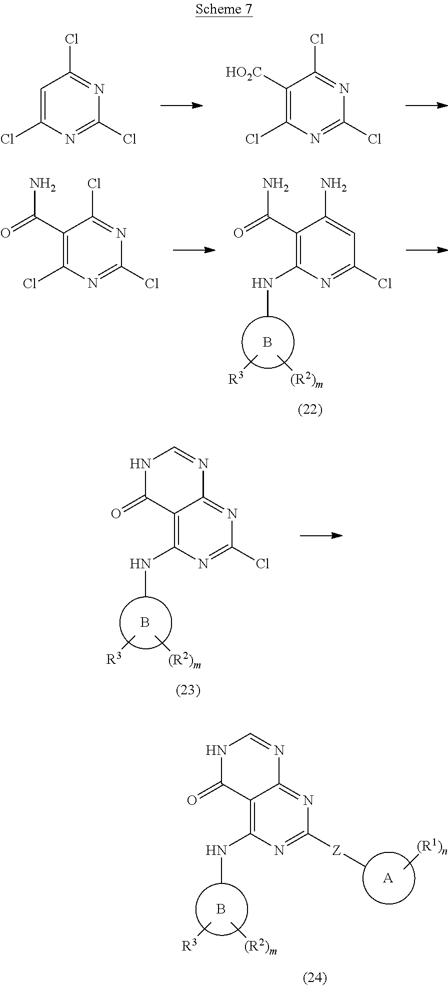

Carboxylation of 2,4,6-trichloropyrimidine to provide 2,4,6-trichloropyrimidine-5-carboxylic acid can be performed by adding solid carbon dioxide (dry ice) to a solution of 2,4,6-trichloropyridine and diisopropylamine treated with n-butyl lithium. The n-butyl lithium is typically added at low temperature to a mixture of 2,4,6-trichloropyridine and diisopropylamine in a solvent such as but not limited to tetrahydrofuran, before adding the carbon dioxide gas and warming to room temperature. 4-Amino-2,6-dichloropyrimidine-5-carboxamide can be prepared from 2,4,6-trichloropyrimidine-5-carboxylic acid by reacting the latter first with oxalyl chloride at low temperature in a solvent such as but not limited to dichloromethane, N,N-dimethylformamide, or mixtures thereof. The resulting crude acid chloride can be reacted with ammonium hydroxide at low temperature in a solvent such as but not limited to tetrahydrofuran to provide 4-amino-2,6-dichloropyrimidine-5-carboxamide. 4-Amino-2,6-dichloropyrimidine-5-carboxamide can be reacted with a compound of formula (7A), wherein B, R², R³, and m are as described herein, in the presence of a base such as but not limited to diisopropylethylamine or triethylamine, to provide compounds of formula (22). The reaction is typically performed at elevated temperature in a solvent such as but not limited to 1,4-dioxane. Triethyl orthoformate can be reacted with compounds of formula (22) to provide compounds of formula (23). The reaction typically involves the use of heat and may employ a solvent such as but not limited to N,N-dimethylformamide. Compounds of formula (24), which are representative of the compounds of this invention, can be prepared by reacting compounds of formula (23) with an organozinc compound of formula (11A), wherein Z, A, R¹, and n are as described herein and X² is a halide. The reaction typically involves the use of heat, and a nickel or palladium catalyst such as but not limited to bis(triphenylphosphine)palladium(II) dichloride in a solvent such as but not limited to N-methylpyrrolidone, tetrahydrofuran, or mixtures thereof.

### Compositions

In another aspect, the present invention provides pharmaceutical compositions for modulating kinase activity in a humans and animals that will typically contain a compound of formula (I) and a pharmaceutically acceptable carrier.

Compounds having formula (I) may be administered, for example, bucally, ophthalmically, orally, osmotically, parenterally (intramuscularly, intraperintoneally intrasternally, intravenously, subcutaneously), rectally, topically, transdermally, vaginally and intraarterially as well as by intraarticular injection, infusion, and placement in the body, such as, for example, the vasculature.

Compounds having formula (I) may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising a compound having formula (I) to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising a compound having formula (I) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I) to be administered osmotically include, but are not limited to, chlorofluorohydrocarbons, ethanol, water, mixtures thereof and the like. Excipients for preparation of compositions comprising a compound having formula (I) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P, or isotonic sodium chloride solution, water, mixtures thereof and the like. Excipients for preparation of compositions comprising a compound having formula (I) to be administered rectally or vaginally include, but are not limited to, cocoa butter, polyethylene glycol, wax, mixtures thereof and the like.

The pharmaceutical composition and the method of the present invention may further comprise other therapeutically active compounds as noted herein which are usually applied in the treatment of the above-mentioned pathological conditions.

### Methods of Use

In another aspect, the present invention provides methods of using a compound or composition of the invention to treat or prevent a disease or condition involving mediation, overexpression or disregulation of kinases in a mammal. In particular, compounds of this invention are expected to have utility in treatment of diseases or conditions during which protein kinases such as any or all CDC-7 family members are expressed.

In one group of embodiments, diseases and conditions of humans or other animals that can be treated with inhibitors of kinases, include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia. Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor.

The methods of the present invention typically involve administering to a subject in need of therapeutic treatment an effective amount of a compound of formula (I). Therapeutically effective amounts of a compound having formula (I) depend on recipient of treatment, disease treated and severity thereof, composition comprising it, time of administration, route of administration, duration of treatment, potency, rate of clearance and whether or not another drug is co-administered. The amount of a compound having formula (I) used to make a composition to be administered daily to a patient in a single dose or in divided doses is from about 0.03 to about 200 mg/kg body weight. Single dose compositions contain these amounts or a combination of submultiples thereof.

### Combination Therapy

The present invention further provides methods of using a compound or composition of the invention in combination with one or more additional active agents.

Compounds having Formula (I) are expected to be useful when used with alkylating agents, angiogenesis inhibitors, antibodies, antimetabolites, antimitotics, antiproliferatives, antivirals, aurora kinase inhibitors, apoptosis promoters (for example, Bcl-xL, Bcl-w and Bfl-1) inhibitors, activators of death receptor pathway, Bcr-Abl kinase inhibitors, BiTE (Bi-Specific T cell Engager) antibodies, antibody drug conjugates, biologic response modifiers, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNA's, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteosome inhibitors, purine analogs, pyrimidine analogs, receptor tyrosine kinase inhibitors, etinoids/deltoids plant alkaloids, small inhibitory ribonucleic acids (siRNAs), topoisomerase inhibitors, ubiquitin ligase inhibitors, and the like, and in combination with one or more of these agents.

BiTE antibodies are bi-specific antibodies that direct T-cells to attack cancer cells by simultaneously binding the two cells. The T-cell then attacks the target cancer cell. Examples of BiTE antibodies include adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (V. R. Sutton, D. L. Vaux and J. A. Trapani, J. of Immunology 1997, 158 (12), 5783).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides. 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy chain DVD polypeptides and two light chain DVD polypeptides are referred to as DVD Ig's. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site. Multispecific DVDs include DVD binding proteins that bind DLL4 and VEGF, or C-met and EFGR or ErbB3 and EGFR.

Alkylating agents include altretamine. AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, rofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (pemetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Antivirals include ritonavir, hydroxychloroquine and the like.

Aurora kinase inhibitors include ABT-348, AZD-1152, MLN-8054, VX-680, Aurora A-specific kinase inhibitors, Aurora B-specific kinase inhibitors and pan-Aurora kinase inhibitors and the like.

Bcl-2 protein inhibitors include AT-101 ((−)gossypol). GENASENSE® (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide (ABT-263), GX-070 (obatoclax) and the like.

Bcr-Abl kinase inhibitors include DASATINIB® (BMS-354825), GLEEVEC®(imatinib) and the like.

CDK inhibitors include AZD-5438, BMI-1040. BMS-032, BMS-387, CVT-2584, flavopyridol, GPC-286199, MCS-5A. PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614. VIOXX® (rofecoxib) and the like.

EGFR inhibitors include ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her21gG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FC1, PU-3, radicicol, SNX-2112, STA-9090 VER49009 and the like.

Inhibitors of inhibitors of apoptosis proteins include HGS1029, GDC-0145, GDC-0152, LCL-161, LBW-242 and the like.

Antibody drug conjugates include anti-CD22-MC-MMAF, anti-CD22-MC-MMAE, anti-CD22-MCC-DM1. CR-011-vcMMAE. PSMA-ADC, MEDI-547, SGN-19 Am SGN-35. SGN-75 and the like

Activators of death receptor pathway include TRAIL, antibodies or other agents that target TRAIL or death receptors (e.g., DR4 and DR5) such as Apomab, conatumumab, ETR2-ST01, GDC0145, (lexatumumab), HGS-1029, LBY-135, PRO-1762 and trastuzumab.

Kinesin inhibitors include Eg5 inhibitors such as AZD4877, ARRY-520; CENPE inhibitors such as GSK923295A and the like.

JAK-2 inhibitors include CEP-701 (lesaurtinib), XL019 and INCB018424 and the like.

MEK inhibitors include ARRY-142886, ARRY-438162 PD-325901. PD-98059 and the like.

mTOR inhibitors include AP-23573, CCI-779, everolimus, RAD-001, rapamycin, temsirolimus, ATP-competitive TORC1/TORC2 inhibitors, including PI-103, PP242, PP30, Torin 1 and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN®(naproxen), VOLTAREN® (diclofenac), INDOCIN® (indomethacin). CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin, picoplatin and the like.

Polo-like kinase inhibitors include BI-2536 and the like.

Phosphoinositide-3 kinase (PI3K) inhibitors include wortmannin, LY294002, XL-147, CAL-120, ONC-21, AEZS-127. ETP-45658. PX-866. GDC-0941. BGT226. BEZ235. XL765 and the like.

Thrombospondin analogs include ABT-510, ABT-567. ABT-898, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY43-9006), pazopanib (GW-786034), vatalanib (PTK-787, ZK-222584), SUTENT® (sunitinib, SU-11248), VEGF trap, ZACTIMA™ (vandetanib, ZD-6474), GA101, ofatumumab, ABT-806 (mAb-806), ErbB3 specific antibodies. BSG2 specific antibodies, DLL4 specific antibodies and C-met specific antibodies, and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin. ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGFIR-specific antibodies, lintuzumab, PANOREX® (edrecolomab), RENCAREX® (WX G250), RITUXAN® (rituximab), ticilimumab, trastuzimab, CD₂₀ antibodies types I and II and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL® (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin). TARGRETIN® (bexarotene), LGD-1550 and the like.

PARP inhibitors include ABT-888 (veliparib), olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132. NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b) or interferon gamma-n1, combinations thereof and the like. Other agents include ALFAFERONE®, (IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim). OncoVAC-CL, OVAREX® (oregovomab), pemtumomab (Y-muHMFGI). PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth or differentiation of tissue cells to direct them to have anti-tumor activity and include krestin, lentinan, sizofuran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone. XRP-9881 (larotaxel), vinflunine. ZK-EPO (synthetic epothilone) and the like.

Ubiquitin ligase inhibitors include MDM2 inhibitors, such as nutlins, NEDD8 inhibitors such as MLN4924 and the like.

Compounds of this invention can also be used as radiosensitizers that enhance the efficacy of radiotherapy. Examples of radiotherapy include external beam radiotherapy, teletherapy, brachytherapy and sealed, unsealed source radiotherapy and the like.

Additionally, compounds having Formula (I) may be combined with other chemotherapeutic agents such as ABRAXAN E™ (ABI-007). ABT-100 (farnesyl transferase inhibitor), ADVEXIN® (Ad5CMV-p53 vaccine). ALTOCOR® or MEVACOR® (lovastatin). AMPLIGEN® (poly 1:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin): O: Vincristine (ONCOVIN®); P: prednisone), CYPAT™ (cyproterone acetate), combrestatin A4P, DAB(389)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EP0906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine). GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paclitaxel, PANDIMEX™ (aglycone saponins from ginseng comprising 20(S)protopanaxadiol (aPPD) and 20(S)protopanaxatriol (aPPT)), panitumumab. PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α). TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YON DELIS® (trabectedin), ZD-6126, ZINECARD® (dexrazoxane), ZOMETA® (zolendronic acid), zorubicin and the like.

### EXAMPLES

### Example 1

### 7-(2,6-dichlorobenzyl)-5-{[2-methoxy-4-(piperazin-1-yl)phenyl]amino}-2,3-dihydropyrido[4,3-d]pyrimidin-4(1H)-one

### Example 1A

### 2,6-dichloropyridine-4-carboxylic acid

A mixture of 2,6-dihydroxypyridine-4-carboxylic acid (15.1 g, 100 mmol) and phosphoryl trichloride (45 ml) was heated for 6 hours at 160-165° C. in a 200 mL sealed tube. After cooling to ambient temperature, the mixture was poured into crushed ice (300 g) and stirred for 1 hours. The mixture was extracted with ethyl acetate (5×60 mL) and the combined organic layers were dried over anhydrous sodium sulfate, filtered and concentrated in vacuo to give the of crude product which was recrystallized from 40 mL of 2/1 ethyl acetate/petroleum ether to afford the title compound. ¹H NMR (DMSO-d₆) δ ppm 7.89 (s, 2H). MS: 192 (M+1).

### Example 1B

### tert-butyl 2,6-dichloropyridin-4-ylcarbamate

To a solution of the product of EXAMPLE 1A (18.0 g, 93.7 mmol) in anhydrous tert-butanol (200 mL) was added diphenylphosphoryl azide (27.1 g, 98 mmol) and N,N-diisopropylethylamine (24.2 g, 187.5 mmol) and the mixture was refluxed under nitrogen for 6 hours. The mixture was concentrated in vacuo and the residue was dissolved in ethyl acetate, washed with ammonium chloride solution and dried over sodium sulfate. Filtration, concentration of the filtrate, and purification by flash chromatography on silica gel using 10/1 petroleum ether/ethyl acetate afforded the title compound. ¹H NMR (DMSO-d₆) δ ppm 10.33 (s, 1H), 7.49 (s, 2H), 1.48 (s, 9H).

### Example 1C

### 4-(tert-butoxycarbonylamino)-2,6-dichloronicotinic acid

N,N,N′,N′-tetramethylethylenediamine (1.7 g, 14.7 mmol) was added to a solution of EXAMPLE 1B (1.84 g, 7.0 mmol) in anhydrous tetrahydrofuran (35 mL). The mixture was degassed and recharged with nitrogen 4 times and cooled to −60° C. n-Butyl lithium (6.4 mL, 16.1 mmol) was added dropwise and the mixture stirred at −60° C. for 2 hours. Dry carbon dioxide gas was bubbled into this solution and the mixture stirred overnight. The mixture was quenched with water and the solvent removed in vacuo. The residue was diluted with water and washed with 2/1 petroleum ether/ethyl acetate (2×20 mL). The aqueous phase was acidified to pH=2 with concentrated hydrochloric acid and the mixture extracted with ethyl acetate. The combined organic layers were dried with sodium sulfate, filtered and concentrated in vacuo to give the title compound. ¹H NMR (DMSO-d₆) δ ppm 9.83 (s, 1H), 7.93 (s, 1H), 1.47 (s, 9H).

### Example 1D

### 4-amino-2,6-dichloropyridine-3-carboxamide

To a solution of the product of EXAMPLE 1C (11.86 g, 38.6 mmol) of N,N-dimethylformamide (120 mL) was added 1,1′-carbonyldiimidazole (6.89 g, 42.5 mmol) and the mixture was stirred at 60° C. for 2 hours and then was cooled to 0-5° C. Ammonia gas was bubbled into the solution and the mixture was stirred overnight. The mixture was poured into 800 mL water and was extracted with ethyl acetate. The organic layers were dried over anhydrous sodium sulfate, filtered and concentrated in vacuo. The residue was purified by flash chromatography on silica gel (200-300 mesh) using 50/1 dichloromethane/methanol to afford the title compound. ¹H NMR (DMSO-d₆) δ ppm 7.97 (brs., 1H), 7.71 (brs., 1H), 6.61 (s, 1H), 6.59 (brs., 2H).

### Example 1E

### 5,7-dichloropyrido[4,3-d]pyrimidin-4(3H)-one

A solution of the product of EXAMPLE 1D (2.65 g, 13 mmol) in 15 mL of triethyl orthoformate was refluxed under nitrogen for 6 hours. After cooling to ambient temperature, the solid was filtered and washed with 1/1 petroleum ether/ethyl acetate (5 mL) to give the title compound. ¹H NMR (DMSO-d₆) δ ppm 12.84 (br s, 1H), 8.33 (s, 1H), 7.72 (s, 1H).

### Example 1F

### tert-butyl 4-(3-methoxy-4-nitrophenyl)piperazine-1-carboxylate

A suspension of 4-fluoro-2-methoxy-1-nitrobenzene (15 g, 87 mmol), tert-butyl piperazine-1-carboxylate (19.59 g, 105.2 mmol) and potassium carbonate (24 g, 174 mmol) in N,N-dimethylformamide (150 mL) was heated at 80° C. for 8 hours. After cooling to ambient temperature, the mixture was poured in water (500 mL). The precipitate was filtered and washed with ethanol to give the title compound. MS: 338 (M+H⁺).

### Example 1G

### tert-butyl 4-(4-amino-3-methoxyphenyl)piperazine-1-carboxylate

A suspension of EXAMPLE 13A (6.3 g, 18.7 mmol) and Raney nickel (2.0 g) in 300 mL methanol was stirred under hydrogen at ambient temperature for 5 hours. The mixture was filtered through diatomaceous earth and the filtrate was concentrated. The residue was purified by flash chromatography on silica gel (200-300 mesh) eluting with a gradient of 2/1 to 1/1 petroleum/ethyl acetate to give the title compound.

### Example 1H

### 5-(2-methoxy-4-(4-methylpiperazin-1-yl)phenylamino)-7-chloropyrido[4,3-d]pyrimidin-4(3H)-one

A solution of the product of EXAMPLE 1E (300 mg, 1.4 mmol) EXAMPLE 1G (473 mg, 1.54 mmol) and triethylamine (421 mg, 4.17 mmol) in 1,4-dioxane (30 mL) was stirred at 105° C. under nitrogen for 12 hours. The solvent was removed under vacuum and the residue was washed with sodium bicarbonate solution and ethanol. The crude product was recrystallized from 1,4-dioxane to give the title compound. ¹H NMR (DMSO-d₆) δ ppm 11.36 (brs, 1H), 8.34 (d, J=8.9 Hz, 1H), 8.25 (s, 1H), 6.79 (s, 1H), 6.72 (d, J=2.1 Hz, 1H), 6.57 (dd, J=2.1, 8.9 Hz, 1H), 3.89 (s, 3H), 3.51-3.44 (m, 4H), 3.14-3.08 (m, 4H), 1.43 (s, 9H).

### Example 1I

### tert-butyl 4-(4-(7-(2,6-dichlorobenzyl)-3,4-dihydro-4-oxopyrido[4,3-d]pyrimidin-5-ylamino)-3-methoxyphenyl)piperazine-1-carboxylate

2,6-Dichlorobenzyl zinc bromide solution in tetrahydrofuran (1 N, 2.3 mL, 2.3 mmol) was added to a solution of bis(triphenylphosphine)palladium(II) chloride (26.7 mg) and EXAMPLE 1H (142 mg, 0.29 mmol) in anhydrous tetrahydrofuran (5 mL) and the mixture was stirred under nitrogen at 65° C. for 20 hours. After cooling to ambient temperature, the mixture was diluted with water and extracted with ethyl acetate. The combined organic layers were dried over anhydrous sodium sulfate, filtered, and concentrated in vacuo. The residue was purified by flash chromatography on silica gel (200-300 mesh) using 50/1 dichloromethane/methanol to afford the crude product, which was further purified by recrystallization from methanol to give the title compound. MS: 611 (M+H⁺).

### Example 1J

### tert-butyl 4-(4-(7-(2,6-dichlorobenzyl)-1,2,3,4-tetrahydro-4-oxopyrido[4,3-d]pyrimidin-5-ylamino)-3-methoxyphenyl)piperazine-1-carboxylate

A solution of sodium cyanoborohydride (130 mg, 2.1 mmol), acetic acid (0.5 mL) and EXAMPLE 11 (130 mg, 0.21 mmol) in 1/1 tetrahydrofuran/methanol (30 mL) was stirred under nitrogen at 60° C. for 4 hours. After cooling to ambient temperature, the solvents were removed under vacuum. The residue was dissolved in water (5 mL) and 1N aqueous hydrochloric acid was added. After stirring for 30 minutes, sodium carbonate solution was added until the mixture reached pH=9.0. The mixture was extracted with ethyl acetate and the solvent was removed under vacuum. The residue was purified by flash chromatography on silica gel (200-300 mesh) 50/1 dichloromethane/methanol to give the title compound. MS: 613 (M+H⁺).

### Example 1K

### 7-(2,6-dichlorobenzyl)-5-{[2-methoxy-4-(piperazin-1-yl)phenyl]amino}-2,3-dihydropyrido[4,3-d]pyrimidin-4(1H)-one

Hydrochloric acid (4 mL) was added to EXAMPLE 1J (120 mg, 0.2 mmol) in 1,4-dioxane (15 mL) and methanol (5 mL) and the mixture stirred at ambient temperature for 4 hours. The solvents were removed in vacuo and the residue was dissolved in water, and brought to pH=11 with sodium hydroxide. The mixture was extracted with ethyl acetate and the organic layers were dried with sodium sulfate, filtered and concentrated in vacuo to give the crude product, which was recrystallized from methanol to afford the title compound. ¹H NMR (CD₃OD) δ ppm 8.11 (d, J=8.9 Hz, 1H), 7.45 (s, 1H), 7.43 (s, 1H), 7.33-7.25 (m, 1H), 6.60 (d, J=2.5 Hz, 1H), 6.32 (dd, J=2.5, 8.9 Hz, 1H), 5.80 (s, 1H), 4.57 (s, 2H), 4.27 (s, 2H), 3.88 (s, 3H), 3.09-3.02 (m, 4H), 3.02-2.95 (m, 4H). MS: 513 (M+1).

### Example 2

### 7-(2,6-dichlorobenzyl)-5-{[2-methoxy-4-(piperazin-1-yl)phenyl]amino}pyrido[3,4-d]pyridazin-4-ol

### Example 2A

### 2,6-dibromonicotinic acid and 2,6-dibromoisonicotinic acid

To a solution of diisopropylamine (7.5 g, 74.3 mmol) in tetrahydrofuran (50 mL) at −78° C. was added n-butyllithium (27.8 mL, 2.5 M solution in hexane, 69.6 mmol). The mixture was stirred at −78° C. for 30 minutes and a solution of 2,6-dibromopyridine (16.0 g, 67.6 mmol) in tetrahydrofuran (50 mL) was added over a period of 40 minutes. The mixture was stirred at −78° C. for 3 hours. Dry carbon dioxide was bubbled into the reaction mixture and the mixture was stirred at ambient temperature overnight. The solvent was removed under reduced pressure and the residue was dissolved in a mixture of ethyl acetate (50 mL) and 10% aqueous sodium hydroxide (100 mL). The aqueous phase was made acidic with concentrated hydrochloric acid and extracted with ethyl acetate (3×150 mL). The organic layers were dried over sodium sulfate, filtered and concentrated to afford the title compound, which was used in the next step without further purification. MS: 282 (M+1).

### Example 2B

### 4,6-dibromo-1-hydroxyfuro[3,4-c]pyridin-3(1H)-one and 4,6-dibromo-3-hydroxyfuro[3,4-c]pyridin-1(3H)-one

To a solution of 2,2,6,6-tetramethylpiperidine (9.1 g, 64.2 mmol) in dry tetrahydrofuran (114 mL) at 0° C., was added n-butyllithium (25.7 mL, 2.5 M solution in hexane, 64.2 mmol) over 15 minutes. The mixture was cooled to −78° C., and a solution of EXAMPLE 2A (6.0 g, 21.4 mmol) in tetrahydrofuran (28 mL) was added. After stirring at −78° C. for 1.5 hours, N,N-dimethylformamide (11.4 mL) was added and the mixture stirred at ambient temperature for 15 hours. The mixture was poured into water, acidified with 1M aqueous hydrochloric acid, and the aqueous phase was extracted with ethyl acetate (3×100 mL). The combined organic phase was dried over sodium sulfate, filtered, and concentrated to give the title mixture, which was used in the next step without further purification.

### Example 2C

### 5,7-dibromopyrido[3,4-d]pyridazin-4-ol

To a solution of EXAMPLE 2B (6.6 g, 21.4 mmol) and hydrazine hydrochloride (2.2 g, 21.4 mmol) in isopropanol (50 mL) was added triethylamine (6.5 g, 64.1 mmol) and the mixture heated at 90° C. for 1.5 hours. The solution was concentrated and absorbed on silica gel. Flash chromatography on silica gel (200-300 mesh) eluting with 75/25 to 55/45 petroleum ether/ethyl acetate afforded the title compound. ¹H NMR (DMSO-d₆) δ ppm 13.09 (s, 1H), 8.32 (s, 1H), 8.21 (s, 1H). MS: 306 (M+1).

### Example 2D

### tert-butyl 4-(4-(7-bromo-4-hydroxypyrido[3,4-d]pyridazin-5-ylamino)-3-methoxyphenyl)piperazine-1-carboxylate

A solution of the product of EXAMPLE 2C (180 mg, 0.6 mmol), tert-butyl 4-(4-amino-3-methoxyphenyl)piperazine-1-carboxylate (181 mg, 0.6 mmol), and N,N-diisopropylethylamine (76 mg, 0.6 mmol) in 1,4-dioxane (3 mL) was heated to 130° C. in a sealed tube for 30 hours. The mixture was concentrated in vacuo to give the crude product, which was used in the next step without further purification.

### Example 2E

### tert-butyl 4-(4-(7-(2,6-dichlorobenzyl)-4-hydroxypyrido[3,4-d]pyridazin-5-ylamino)-3-methoxyphenyl)piperazine-1-carboxylate

EXAMPLE 2D (crude, 0.3 mmol), copper(I) iodide (13 mg, 0.07 mmol), and 1,1′-bis(diphenylphosphino)ferrocene-palladium(II) dichloride (24 mg, 0.03 mmol) in tetrahydrofuran (2 mL) were stirred for 2 minutes under nitrogen. 2,6-Dichlorobenzyl zinc bromide (4.8 mL, 0.5 M solution in N,N-dimethylformamide, 2.4 mmol) was added and the mixture stirred at 70° C. overnight. The mixture was diluted with brine (100 mL) and was extracted with ethyl acetate (3×100 mL). The organic layers were dried over sodium sulfate, filtered, and concentrated and the residue was purified by flash chromatography on silica gel (200-300 mesh) eluting with 1:1 petroleum ether/ethyl acetate to give the title compound. MS: 611 (M+1).

### Example 2F

### 7-(2,6-dichlorobenzyl)-5-{[2-methoxy-4-(piperazin-1-yl)phenyl]amino}pyrido[3,4-d]pyridazin-4-ol

EXAMPLE 2E (103 mg, 0.17 mmol) was dissolved in a mixture of methanol (7 mL) and concentrated hydrochloric acid (3 mL). The mixture was concentrated under reduced pressure and was neutralized with saturated aqueous sodium bicarbonate solution. The aqueous phase was extracted with ethyl acetate (3×100 mL) and the combined organic layers were dried over sodium sulfate, filtered, and concentrated. The residue was purified by preparative HPLC (acetonitrile/water containing 0.1% trifluoroacetic acid) to give the title compound as a mono-trifluoroacetate salt. ¹H NMR (DMSO-d₆) δ ppm 12.87 (s, 1H, exchangeable with D₂O), 11.47 (s, 1H, exchangeable with D₂O), 8.75 (brs, 2H, exchangeable with D₂O), 8.23 (s, 1H), 8.01 (d, J=8.7 Hz, 1H), 7.59-7.56 (m, 2H), 7.42 (t, J=8.7 Hz, 1H), 6.85 (s, 1H), 6.68 (s, 1H), 6.23 (d, J=8.7 Hz, 1H), 4.47 (s, 2H), 3.87 (s, 3H), 3.30 (brs, 8H). MS: 511 (M+1).

### Example 3

### 8-(2,6-dichlorobenzyl)-6-{[2-methoxy-4-(piperazin-1-yl)phenyl]amino}-1,2,3,4-tetrahydro-5H-pyrido[2,3-e][1,4]diazepin-5-one

### Example 3A

### 2,6-dichloropyridine 1-oxide

A solution of 2,6-dichloropyridine (4.0 g, 27.0 mmol), 30% hydrogen peroxide (5.2 g, 46.0 mmol) and trifluoroacetic acid (40.0 g) was stirred at 100° C. for 6 hours. The mixture was diluted with water (50 mL) and extracted with ethyl acetate (3×50 mL). The organic layers were separated, washed with aqueous sodium bicarbonate and water, and concentrated under vacuum to give the title compound, which was used in the next step without further purification.

### Example 3B

### 2,4,6-trichloropyridine

A solution of the crude product of EXAMPLE 3A (3.8 g) in phosphorus oxychloride was stirred at 100° C. for 6 hours. The mixture was concentrated, quenched with crushed ice and adjusted to pH 8-9 with sodium carbonate. The residue was extracted with ethyl acetate (3×5(mL) and the organic layers were combined and concentrated under vacuum. The residue was purified by flash chromatography on silica gel (200-300 mesh) eluting with 80/1 petroleum ether/ethyl acetate to give the title compound. ¹H NMR (CDCl₃) δ ppm 7.31 (s, 2H).

### Example 3C

### 2,4,6-trichloronicotinic acid

A mixture of diisopropylamine (2.54 g, 22.1 mmol), n-butyl lithium (1.6 M in hexane, 15.7 mL, 25.1 mmol) and tetrahydrofuran (100 mL) was stirred for 30 minutes at −78° C. A solution of EXAMPLE 3B (2.0 g, 11.0 mmol) in tetrahydrofuran (8 mL) was added dropwise over a period of 30 minutes, followed by stirring for 1 hour. The mixture was poured into dry ice and stirred for 1 hour at room temperature. The mixture was acidified with 10% aqueous hydrochloric acid (20 mL), diluted with aqueous saturated sodium chloride and extracted with ethyl acetate. The organic layer was washed, dried over anhydrous sodium sulfate, filtered, and concentrated under vacuum. The solvent was removed under vacuum to give the crude title compound which was used in the next step without further purification.

### Example 3D

### tert-butyl 2-(2,4,6-trichloronicotinamido)ethylcarbamate

A solution of the product of EXAMPLE 3C (6.0 g, 26.5 mmol) in dichloromethane (150 mL) was treated at 0° C. with 2 drops of N,N-dimethylformamide. Oxalyl chloride (6.73 g, 53 mmol) was added dropwise over 30 minutes and stirring was continued for 2 hours. The solution was concentrated and dried under vacuum to give the crude acid chloride. A solution of the acid chloride (4.5 g, 18.4 mmol) in 60 mL of dry dichloromethane was added dropwise over 1 hour to a solution of tert-butyl 2-aminoethylcarbamate (5.9 g, 36.8 mmol) and triethylamine (3.7 g, 36.8 mmol) in 40 mL of dry dichloromethane at 0° C. and stirring was continued for 2 hours. The mixture was concentrated under vacuum and the residue was purified by flash chromatography on silica gel (200-300 mesh) eluting with 100/1 dichloromethane/methanol to give the title compound. MS: 390 (M+Na⁺).

### Example 3E

### N-(2-aminoethyl)-2,4,6-trichloronicotinamide

To a solution of EXAMPLE 3D (1.13 g, 3.07 mmol) in 100 mL of dichloromethane was added 20 mL of trifluoroacetic acid and the mixture stirred at room temperature over night. The solvent was removed under vacuum to give the crude product which was used in the next step without further purification. MS: 268 (M+H⁺).

### Example 3F

### 6,8-dichloro-1,2,3,4-tetrahydropyrido[2,3-e][1,4]diazepin-5-one

A suspension of EXAMPLE 3E (823 mg, 3.07 mmol), cesium fluoride (2.3 g, 15.35 mmol) and triethylamine (1.0 mL) in 300 mL of N,N-dimethylformamide was heated at 75° C. under nitrogen for 12 hours. The mixture was concentrated under vacuum and the residue was purified by flash chromatography on silica gel (200-300 mesh) eluting with 30/1 dichloromethane/methanol to give the title compound. ¹H NMR (DMSO-d₆) δ ppm 8.40 (t, 1H), 7.33 (t, 1H), 6.85 (s, 1H), 3.39-3.37 (m, 2H), 2.90 (s, 1H), 2.74 (s, 1H). MS: 232 (M+H⁺).

### Example 3G

### tert-butyl 4-(4-(8-chloro-5-oxo-2,3,4,5-tetrahydro-1H-pyrido[2,3-e][1,4]diazepin-6-ylamino)-3-methoxyphenyl)piperazine-1-carboxylate

A solution of N,N-diisopropylethylamine (0.5 mL), EXAMPLE 3F (200 mg, 0.86 mmol), tert-butyl 4-(4-amino-3-methoxyphenyl)piperazine-1-carboxylate (318 mg, 1.03 mmol) in 1,4-dioxane (4 mL) was heated in a 15 mL sealed tube at 120° C. for 2 days. The mixture was concentrated under vacuum and the residue purified by flash chromatography on silica gel (200-300 mesh) eluting with 30/1 dichloromethane/methanol to give the title compound. MS: 503 (M+H⁺).

### Example 3H

### 8-(2,6-dichlorobenzyl)-6-{[2-methoxy-4-(piperazin-1-yl)phenyl]amino}-1,2,3,4-tetrahydro-5H-pyrido[2,3-e][1,4]diazepin-5-one

A suspension of EXAMPLE 3G (100 mg, 0.2 mmol), 4 mL (2,6-dichlorobenzyl)zinc(II) bromide (0.5 M in tetrahydrofuran, 2.0 mmol) and tetrakis(triphenylphosphine)palladium (23 mg, 0.02 mmol) in dry tetrahydrofuran (6 mL) was heated in a microwave for 1 hour at 120° C. The solution was concentrated under vacuum and the residue purified by preparative HPLC to give the title compound. ¹H-NMR (CD₃OD) δ ppm 7.42 (m, 1H), 7.39 (s, 1H), 7.31-7.26 (m, 1H), 6.92 (d, J=8.7 Hz, 1H), 6.59 (d, J=2.4 Hz, 1H), 6.50 (dd, J=8.7 Hz, J=2.4 Hz, 1H), 5.36 (s, 1H), 4.24 (s, 1H), 3.71 (s, 3H), 3.67-3.69 (m, 2H), 3.54-3.56 (m, 2H), 3.41 (m, 8H). MS: 264 (M/2+H⁺).

### Example 4

### 8-(2,6-dichlorobenzyl)-6-{[4-(piperazin-1-yl)phenyl]amino}-1,2,3,4-tetrahydro-5H-pyrido[2,3-e][1,4]diazepin-5-one

### Example 4A

### 2,6-dichloropyridine 1-oxide

A solution of 2,6-dichloropyridine (4.0 g, 27.0 mmol), 30% hydrogen peroxide (5.2 g, 46.0 mmol) and trifluoroacetic acid (40.0 g) was stirred at 100° C. for 6 hours. The mixture was diluted with water (50 mL) and extracted with ethyl acetate (3×50 mL). The combined organic layers were washed with aqueous sodium bicarbonate and water and concentrated under vacuum to give the title compound, which was used in the next step without further purification.

### Example 4B

### 2,4,6-trichloropyridine

A solution of EXAMPLE 4A (3.8 g, crude) in phosphorus oxychloride was stirred at 100° C. for 6 hours. The mixture was concentrated, quenched with crushed ice and adjusted to pH 8-9 with sodium carbonate. The residue was extracted with ethyl acetate (3×50 mL) and the combined organic layers concentrated under vacuum. The residue was purified by flash chromatography on silica gel (200-300 mesh) eluting with 80/1 petroleum ether/ethylacetate to give the title compound. ¹H NMR (CDCl₃,) δ ppm 7.31 (s, 2H).

### Example 4C

### 2,4,6-trichloronicotinic acid

A solution of diisopropylamine (2.54 g, 22.1 mmol) and n-butyl lithium (1.6 M in hexane, 15.7 mL, 25.1 mmol) in tetrahydrofuran (100 mL) was stirred for 30 minutes at −78° C. A solution of the product of EXAMPLE 4B (2.0 g, 11.0 mmol) in tetrahydrofuran (8 mL) was added dropwise over 30 minutes, followed by stirring for 1 hour. The mixture was poured into dry ice and stirred for 1 hour at room temperature. The mixture was acidified with 10% aqueous hydrochloric acid (20 mL), diluted with an aqueous saturated sodium chloride solution and extracted with ethyl acetate. The organic layer was washed, dried over anhydrous sodium sulfate, filtered, and concentrated under vacuum. The solvent was removed under vacuum to give the crude title compound which was used in the next step without further purification.

### Example 4D

### tert-butyl 2-(2,4,6-trichloronicotinamido)ethylcarbamate

A solution of EXAMPLE 4C (6.0 g, 26.5 mmol) in dichloromethane (150 mL) was treated at 0° C. with 2 drops of N,N-dimethylformamide. Oxalyl chloride (6.73 g, 53 mmol) was added dropwise over 30 minutes and stirring was continued for 2 hours. The solution was concentrated and dried under vacuum to give the crude acid chloride. A solution of the acid chloride (4.5 g, 18.4 mmol) in 60 mL dry dichloromethane was added dropwise to a solution of tert-butyl 2-aminoethylcarbamate (5.9 g, 36.8 mmol) and triethylamine (3.7 g, 36.8 mmol) in 40 mL of dry dichloromethane at 0° C. over 1 hour and stirring was continued for 2 hours. The mixture was concentrated under vacuum and the residue was purified by flash chromatography on silica gel (200-300 mesh) eluting with 100/1 dichloromethane/methanol to give the title compound. MS: 390 (M+Na⁺).

### Example 4E

### N-(2-aminoethyl)-2,4,6-trichloronicotinamide

A solution of the product of EXAMPLE 4D (1.13 g, 3.07 mmol) in 100 mL of dichloromethane was treated with 20 mL of trifluoroacetic acid and the mixture was stirred at room temperature over night. The solvent was removed under vacuum to give the crude product which was used in the next step without further purification. MS: 268 (M+H⁺).

### Example 4F

### 6,8-dichloro-1,2,3,4-tetrahydropyrido[2,3-e][1,4]diazepin-5-one

A suspension of EXAMPLE 4E (823 mg, 3.07 mmol), cesium fluoride (2.3 g, 15.35 mmol) and 1 mL of triethylamine in 300 mL N,N-dimethylformamide was heated to 75° C. under nitrogen overnight. The mixture was concentrated under vacuum and the residue was purified by flash chromatography on silica gel (200-300 mesh) eluting with 30/1 dichloromethane/methanol to give the title compound. ¹H-NMR (DMSO-d₆): δ ppm 8.40 (t, 1H), 7.33 (t, 1H), 6.85 (s, 1H), 3.39-3.37 (m, 2H), 2.90 (s, 1H), 2.74 (s, 1H). MS: 232 (M+H⁺).

### Example 4G

### tert-butyl 4-(4-(8-chloro-5-oxo-2,3,4,5-tetrahydro-1H-pyrido[2,3-e][1,4]diazepin-6-yl amino)phenyl)piperazine-1-carboxylate

A solution of EXAMPLE 4F (200 mg, 0.86 mmol), tert-butyl 4-(4-aminophenyl)piperazine-1-carboxylate (285 mg, 1.03 mmol) and diisopropylethylamine (0.5 mL) in 1,4-dioxane (4 mL) was heated in a sealed tube at 120° C. for 2 days. The mixture was concentrated under vacuum and the residue purified by flash chromatography on silica gel (200-300 mesh) eluting with 30/1 dichloromethane/methanol to give the title compound. MS: 473 (M+H⁺).

### Example 4H

### 8-(2,6-dichlorobenzyl)-6-{[4-(piperazin-1-yl)phenyl]amino}-1,2,3,4-tetrahydro-5H-pyrido[2,3-e][1,4]diazepin-5-one

A suspension of the product of EXAMPLE 4G (110 mg, 0.23 mmol), (2,6-dichlorobenzyl)zinc(II) bromide (4.6 mL, 2.3 mmol, 0.5 M in tetrahydrofuran) and tetrakis(triphenylphosphine)palladium (27 mg, 0.023 mmol) in 5 mL dry tetrahydrofuran was heated to 120° C. by microwave for 1 hour. The crude product was purified by preparative HPLC (acetonitrile/water containing 0.1% trifluoroacetic acid) to give the title compound. ¹H-NMR (DMSO-d₆): δ ppm 11.60 (br, 1H), 8.76 (br, 2H), 8.65 (br, 1H), 7.55-7.47 (m, 2H), 7.37-7.31 (m, 1H), 6.92 (s, 4H), 5.44 (s, 1H), 4.16 (s, 1H), 3.41-3.60 (m, 4H), 3.31-3.23 (m, 8H). MS: 249 (M/2+H⁺).

### Example 5

### 7-(2,6-dichlorobenzyl)-5-{[4-(piperazin-1-yl)phenyl]amino}pyrido[3,4-d]pyridazin-4-ol

### Example 5A

### tert-butyl 4-(4-(7-bromo-4-hydroxypyrido[3,4-d]pyridazin-5-ylamino)phenyl)piperazine-1-carboxylate

The title compound was obtained following the procedure of EXAMPLE 2D substituting tert-butyl 4-(4-aminophenyl)piperazine-1-carboxylate for tert-butyl 4-(4-amino-3-methoxyphenyl)piperazine-1-carboxylate. Purification by flash chromatography on silica gel (200-300 mesh) eluting with 50/1 dichloromethane/methanol gave the title compound. MS: 501 (M+H⁺).

### Example 5B

### tert-butyl 4-(4-(7-(2,6-dichlorobenzyl)-4-hydroxypyrido[3,4-d]pyridazin-5-ylamino)phenyl)piperazine-1-carboxylate

The title compound was obtained following the procedure of EXAMPLE 2E substituting EXAMPLE 5A for EXAMPLE 2D. The residue was purified by flash chromatography on silica gel (200-300 mesh) eluting with 3/1 petroleum ether/ethyl acetate to give the title compound. MS: 581 (M+H⁺).

### Example 5C

### 7-(2,6-dichlorobenzyl)-5-{[4-(piperazin-1-yl)phenyl]amino}pyrido[3,4-d]pyridazin-4-ol

The title compound was obtained following the procedure of EXAMPLE 2F substituting EXAMPLE 5B for EXAMPLE 2E. Purification by preparative HPLC (acetonitrile/water containing 0.1% trifluoroacetic acid) gave the title compound as the mono-trifluoroacetic acid salt. ¹H NMR (DMSO-d₆): δ ppm 12.99 (s, 1H), 11.26 (s, 1H), 8.70 (s, 2H), 8.26 (s, 1H), 7.59 (s, 1H), 7.57 (s, 1H), 7.47-7.38 (m, 3H), 6.90-6.77 (m, 3H), 4.47 (s, 2H), 3.27 (s, 8H). MS: 481 (M+H⁺).

### Example 6

### 8-(2,6-dichlorobenzyl)-6-{[4-(piperazin-1-yl)phenyl]amino}-1,2,3,4-tetrahydro-5H-pyrido[4,3-e][1,4]diazepin-5-one

### Example 6A

### tert-butyl 2-(2,4,6-trichloronicotinamido)ethylcarbamate

A solution of 2,4,6-trichloronicotinic acid (6.0 g, 26.5 mmol) in dichloromethane (150 mL) at 0° C. was treated with 2 drops of N,N-dimethyl formamide. Oxalyl chloride (6.73 g, 53 mmol) was added dropwise within 30 minutes and stirring was continued for 2 hours. The solution was concentrated and dried under vacuum to give crude 2,4,6-trichloronicotinoyl chloride. A solution of the crude acid chloride (4.5 g, 18.4 mmol) in dichloromethane (60 mL) was added dropwise to a solution of tert-butyl 2-aminoethyl carbamate (5.9 g, 36.8 mmol) and triethylamine (3.7 g, 36.8 mmol) in dichloromethane (40 mL) at 0° C. over 1 hour and stirring was continued for 2 hours. The mixture was concentrated and the residue was purified by flash chromatography on silica gel (200-300 mesh) eluting with 100/1 dichloromethane/methanol to give the title compound. MS: 390 (M+Na⁺).

### Example 6B

### N-(2-aminoethyl)-2,4,6-trichloronicotinamide

A solution of EXAMPLE 6A (1.13 g, 3.07 mmol) in dichloromethane (100 mL) was treated with trifluoroacetic acid (20 mL) and the mixture was stirred at room temperature overnight. Concentration provided the crude title compound which was used in the next step without further purification. MS: 268 (M+H⁺).

### Example 6C

### 6,8-dichloro-1,2,3,4-tetrahydropyrido[4,3-e][1,4]diazepin-5-one

A suspension of EXAMPLE 6B (823 mg, 3.07 mmol), cesium fluoride (2.3 g, 15.35 mmol) and triethylamine (1 mL) in N,N-dimethylformamide (300 mL) was heated at 75° C. under nitrogen overnight. The mixture was concentrated and the residue was purified by flash chromatography on silica gel (200-300 mesh) eluting with 30/1 dichloromethane/methanol to give the title compound. ¹H NMR (DMSO-d₆, 300 MHz): δ 8.33 (t, 1H), 7.46 (t, 1H), 6.67 (s, 1H), 3.29-3.38 (m, 4H). MS: 232 (M+H⁺).

### Example 6D

### tert-butyl 4-(4-(8-chloro-5-oxo-2,3,4,5-tetrahydro-1H-pyrido[4,3-e][1,4]diazepin-6-ylamino)phenyl)piperazine-1-carboxylate

A solution of EXAMPLE 6C (170 mg, 0.73 mmol), tert-butyl 4-(4-aminophenyl)piperazine-1-carboxylate (244 mg, 0.88 mmol) and N,N-diisopropylethylamine (0.5 mL) in 1,4-dioxane (4 mL) was heated in a sealed tube at 125° C. for 3 days. The mixture was concentrated and the residue was purified by flash chromatography on silica gel (200-300 mesh) eluting with 20/1 dichloromethane/methanol to give the title compound. MS: 473 (M+H⁺).

### Example 6E

### 8-(2,6-dichlorobenzyl)-6-(4-(piperazin-1-yl)phenylamino)-1,2,3,4-tetrahydropyrido[4,3-e][1,4]diazepin-5-one

A suspension of EXAMPLE 6D (260 mg, 0.34 mmol), 0.5M (2,6-dichloro benzyl)zinc(II) bromide in tetrahydrofuran (6.8 mL, 3.4 mmol,) and tetrakis(triphenylphosphine)palladium (79 mg, 0.07 mmol) in tetrahydrofuran (10 mL) was heated at 120° C. in a Biotage Microwave Synthesizer for 1 hour. The crude title compound was purified by preparative HPLC eluting with a gradient of 12 to 90% acetonitrile/water (containing 0.1% trifluoroacetic acid) to give the title compound. ¹H NMR (CD₃OD, 300 MHz): δ ppm 7.50 (d, 1H), 7.48 (s, 1H), 7.37 (m, 1H), 7.27-7.30 (m, 2H), 7.16-7.19 (m, 2H), 5.66 (s, 1H), 4.21 (s, 2H), 3.39-3.54 (m, 12H). MS: 249 (M/2+H⁺).

### Example 7

### 7-(2,6-dichlorobenzyl)-5-{[3-methyl-4-(piperazin-1-yl)phenyl]amino}pyrido[3,4-d]pyridazin-4(3H)-one

### Example 7A

### tert-butyl 4-(2-methyl-4-nitrophenyl)piperazine-1-carboxylate

A mixture of 1-bromo-2-methyl-4-nitrobenzene (5.16 g, 24 mmol), tert-butyl piperazine-1-carboxylate (4.46 g, 24 mmol), 2-dicyclohexylphosphino-2′,4′,6′-triisopropylbiphenyl (1.15 g, 2.4 mmol), cesium carbonate (15.65 g, 48 mmol) and tris(dibenzylideneacetone)dipalladium (2.21 g, 2.4 mmol) in 1,4-dioxane (120 mL) was heated at 100° C. for 16 hours. The mixture was filtered and the filtrate was concentrated to give the crude product which was purified by flash chromatography on silica gel (200-300 mesh) eluting with 20/1 petroleum ether/ethyl acetate to give the title compound. MS: 322 (M+H⁺).

### Example 7B

### tert-buty 14-(4-amino-2-methylphenyl)piperazine-1-carboxylate

A suspension of EXAMPLE 7A (3.10 g, 9.7 mmol) and 10% palladium on carbon (310 mg) in methanol (200 mL) was stirred under hydrogen at ambient temperature for 5 hours. The catalyst was filtered off and the filtrate was concentrated. Purification by flash chromatography on silica gel (200-300 mesh) eluting with 8/1 petroleum ether/ethyl acetate gave the title compound. MS: 292 (M+H⁺).

### Example 7C

### 2,6-dichloroisonicotinic acid

A mixture of 2,6-dihydroxyisonicotinic acid (10 g, 64.5 mmol) and phosphoryl trichloride (30 mL) was heated in a sealed tube at 140° C. for 6 hours. After cooling to room temperature, the mixture was concentrated and the residue was poured into ice-water and vigorously stirred for 15 minutes. The suspension was filtered and the solid was washed with cooled water and dried in vacuo to afford the title compound. MS: 192 (M+He).

### Example 7D

### ethyl 2,6-dichloroisonicotinate

To a solution of EXAMPLE 7C (5 g, 26.2 mmol) in ethanol (50 mL) was added dropwise thionyl chloride (5.6 mL, 78.6 mmol) in an ice-water bath and the mixture was stirred at room temperature for 5 hours. After concentration, the residue was purified by flash chromatography eluting with a gradient of 1/20 to 1/10 ethyl acetate/petroleum ether to provide the title compound. MS: 220 (M+H⁺).

### Example 7E

### (2,6-dichloropyridin-4-yl)methanol

To a solution of EXAMPLE 7D (3.1 g, 14.2 mmol) in ethanol (100 mL) at 0° C. was added sodium triacetoxyborohydride (2.7 g, 71.1 mmol) in portions and the mixture was heated at 80° C. for 3 hours. The mixture was concentrated and the residue was diluted with water (20 mL). 1N Hydrochloric acid was added to quench excess sodium triacetoxyborohydride and then the mixture was neutralized with saturated aqueous sodium carbonate. The mixture was extracted with dichloromethane (3×20 mL) and the combined organic phase was washed with brine, dried over anhydrous sodium sulfate, filtered and concentrated. The residue was recrystallized from methanol to give the title compound. MS: 178 (M+H⁺).

### Example 7F

### 2,6-dichloroisonicotinaldehyde

To a solution of EXAMPLE 7E (3.2 g, 18.1 mmol) in dichloromethane (100 mL) was added Dess-Martin periodinane (9.2 g, 21.7 mmol) in portions and the mixture was stirred at room temperature for 30 minutes. The mixture was filtered and the filtrate was concentrated. The residue was purified by flash chromatography eluting with 1/20 ethyl acetate/petroleum ether to afford the title compound. MS: 176 (M+H⁺).

### Example 7G

### 2,6-dichloro-4-(dimethoxymethyl)pyridine

To a solution of EXAMPLE 7F (2 g, 11.4 mmol) in methanol (10 mL) and toluene (10 mL) was added methyl orthoformate (2.4 g, 22.9 mmol) and p-toluenesulfonic acid (98 mg, 0.57 mmol) and the mixture was heated at reflux for 2 hours. After concentration, the residue was diluted with dichloromethane and was washed with saturated aqueous sodium carbonate and brine. The organic phase was dried over anhydrous sodium sulfate, filtered and concentrated to give the title compound.

### Example 7H

### methyl 2,6-dichloro-4-(dimethoxymethyl)nicotinate

To a solution of diisopropylamine (9.5 mL, 67.9 mmol) in tetrahydrofuran (50 mL) at −78° C. was added 2.5M n-butyl lithium in hexane (27 mL, 67.9 mmol) and the mixture was stirred at −78° C. for 30 minutes. A solution of EXAMPLE 7G (5 g, 22.6 mmol) in tetrahydrofuran (20 mL) was added dropwise and the mixture was stirred at −78° C. for 3 hours. A solution of methyl carbonochloridate (6 mL, 67.9 mmol) in tetrahydrofuran (20 mL) was added and the mixture was warmed to room temperature over 2 hours. The mixture was carefully quenched by addition of water and the mixture was extracted with dichloromethane (3×100 mL). The combined organic phase was washed with brine, dried over anhydrous sodium sulfate, filtered and concentrated. The residue was purified by flash chromatography eluting with 1/20 ethyl acetate/petroleum ether to afford the title compound. MS: 280 (M+H⁺).

### Example 7I

### methyl 2,6-dichloro-4-formylnicotinate

To a solution of EXAMPLE 7H (0.5 g, 1.8 mmol) in dichloromethane (5 mL) was added trifluoroacetic acid (2 mL) and the mixture was stirred at room temperature overnight. The mixture was concentrated to give the title compound which was used in the next step without further purification. MS: 234 (M+H⁺).

### Example 7J

### (E)-methyl 4-((2-(tert-butoxy carbonyl)hydrazono)methyl)-2,6-dichloronicotinate

To a solution of EXAMPLE 71 (2.3 g, 9.9 mmol) in 1,4-dioxane (30 mL) was added tert-butyl hydrazine carboxylate (1.33 g, 10.1 mmol) and the mixture was stirred at room temperature overnight. The mixture was concentrated to provide the title compound which was used in the next step without further purification. MS: 348 (M+H⁺).

### Example 7K

### 5,7-dichloropyrido[3,4-d]pyridazin-4(3H)-one

To a solution of EXAMPLE 7J (2.9 g, 8.4 mmol) in dichloromethane (50 mL) was added trifluoroacetic acid (10 mL) and the mixture was stirred at room temperature for 2 hours. The mixture was concentrated and the residue was washed with 1/10 ethyl acetate/petroleum ether to provide the title compound. MS: 216 (M+H⁺).

### Example 7L

### tert-butyl 4-(4-(7-chloro-4-oxo-3,4-dihydropyrido[3,4-d]pyridazin-5-ylamino)-2-methylphenyl)piperazine-1-carboxylate

A solution of EXAMPLE 7K (100 mg, 0.465 mmol), EXAMPLE 7B, (135 mg, 0.465 mmol), and N,N-diisopropylethylamine (2 mL) in dioxane (30 mL) was stirred at 120° C. for 16 hours. The mixture was cooled to ambient temperature and concentrated. The residue was diluted with water (50 mL) and extracted with ethyl acetate (3×100 mL). The combined organic extracts were dried over anhydrous sodium sulfate, filtered and purified by flash chromatography on silica gel (200-300 mesh) eluting with a gradient of 50/1 to 10/1 dichloromethane/methanol to give the title compound. MS: 471 (M+H⁺).

### Example 7M

### tert-butyl 4-(4-(7-(2,6-dichlorobenzyl)-4-oxo-3,4-dihydropyrido[3,4-d]pyridazin-5-ylamino)-2-methylphenyl)piperazine-1-carboxylate

A mixture of EXAMPLE 7L (150 mg, 0.32 mmol), 1M (2,6-dichlorobenzyl)zinc(II) bromide in tetrahydrofuran (15.0 mL, 15.0 mmol), tetrakis(triphenylphosphine)palladium (173 mg, 0.15 mmol) in tetrahydrofuran (10 mL) was heated in a Biotage Microwave Synthesizer at 120° C. for 1 hour. After cooling to ambient temperature, the mixture was filtered and purified by flash chromatography on silica gel (200-300 mesh) eluting with a gradient of 50/1 to 10/1 dichloromethane/methanol to provide the title compound. MS: 595 (M+H⁺).

### Example 7N

### 7-(2,6-dichlorobenzyl)-5-{[3-methyl-4-(piperazin-1-yl)phenyl]amino}pyrido[3,4-d]pyridazin-4(3H)-one

The title compound was obtained following the procedure described in EXAMPLE 20F, using EXAMPLE 7M in place of EXAMPLE 20E. ¹H NMR (DMSO-d₆, 300 MHz): δ 13.02 (s, 1H), 11.34 (s, 1H), 8.65 (brs, 2H), 8.27 (s, 1H), 7.57-7.36 (m, 5H), 6.88-6.84 (m, 2H), 4.47 (s, 2H), 3.25 (brs, 4H), 2.98 (brs, 4H), 2.22 (s, 3H). MS: 495 (M+H⁺).

### Example 8

### 5-{[4-(4-cyclohexylpiperazin-1-yl)-2-methoxyphenyl]amino}-7-(2,6-dichlorobenzyl)pyrido[3,4-d]pyridazin-4(3H)-one

To a solution of EXAMPLE 2F (70 mg, 0.137 mmol) in methanol (10 mL) was added acetic acid (0.1 mL) and cyclohexanone (16 mg, 0.164 mmol). After stirring at 60° C. for 2 hours, the mixture was cooled to ambient temperature and sodium cyanoborohydride (173 mg, 2.74 mmol) was added. The mixture was stirred for 16 hours, poured into water (50 mL) and extracted with ethyl acetate (2×50 mL). The combined organic layers were dried over anhydrous sodium sulfate, filtered, concentrated and purified by preparative HPLC eluting with a gradient of 10/90 to 30/20 acetonitrile/water (containing 0.1% trifluoroacetic acid) to give the title compound. ¹H NMR (CD₃OD, 300 MHz): δ 8.17 (d, J=9.0 Hz, 1H), 8.11 (s, 1H), 7.49 (d, J=7.8 Hz, 2H), 7.34 (t, J=7.5 Hz, 1H), 6.79 (s, 1H), 6.69 (s, 1H), 6.34 (d, J=9.0 Hz, 1H), 4.58 (s, 2H), 3.96 (s, 3H), 3.95-3.63 (m, 4H), 3.08-3.03 (m, 3H), 2.25-2.21 (m, 2H), 2.04-1.44 (m, 10H). MS: 593.2 (M+H⁺).

### Example 9

### [4-(4-{[7-(2,6-dichlorobenzyl)-4-hydroxypyrido[3,4-d]pyridazin-5-yl]amino}-3-methoxyphenyl)piperazin-1-yl](phenyl)methanone

To a solution of EXAMPLE 2F (51.1 mg, 0.100 mmol) in N,N-dimethylformamide (5 mL) was added N,N-diisopropylethylamine (38.7 mg, 0.300 mmol) and the solution was stirred at ambient temperature for 5 minutes. The mixture was cooled to 0° C. and a solution of benzoyl chloride (20.9 mg, 0.150 mmol) in N,N-dimethylformamide (1 mL) was added dropwise and the mixture was stirred at ambient temperature for 15 hours. The mixture was diluted with ethyl acetate (50 mL) and washed with brine (50 mL). The organic layer was dried over anhydrous sodium sulfate, concentrated, filtered, and purified by flash chromatography on silica gel (200-300 mesh) eluting with 2/3 petroleum ether/ethyl acetate to give the title compound. ¹H NMR (DMSO-d₆, 300 MHz): δ 12.84 (s, 1H), 11.45 (s, 1H), 8.21 (s, 1H), 7.96-7.93 (m, 1H), 7.58-7.43 (m, 8H), 6.85 (s, 1H), 6.66 (s, 1H), 6.22-6.19 (m, 1H), 4.47 (s, 2H), 3.86 (s, 3H), 3.61 (m, 4H), 3.14 (m, 4H). MS: 615 (M+H⁺).

### Example 10

### 7-[(2,6-dichlorophenyl)amino]-5-{[2-methoxy-4-(piperazin-1-yl)phenyl]amino}pyrido[3,4-d]pyridazin-4-ol

### Example 10A

### tert-buty 14-(4-(7-(2,6-dichlorophenylamino)-4-oxo-3,4-dihydropyrido[3,4-d]pyridazin-5-ylamino)-3-methoxyphenyl)piperazine-1-carboxylate

A mixture of EXAMPLE 24A (0.1 g, 0.21 mmol), 2,6-dichlorobenzenamine (33 mg, 0.21 mmol) and potassium tert-butoxide (47 mg, 0.42 mmol) was degassed with nitrogen three times. Tris[dibenzylideneacetone]dipalladium (4 mg) and 2-dicyclohexylphosphino-2′,4′,6′-triisopropylbiphenyl (8 mg) were added and the mixture was stirred at 120° C. overnight. The mixture was concentrated and the residue was purified by flash chromatography on silica gel eluting with 100/1 dichloromethane/methanol to give the title compound.

### Example 10B

### 7-[(2,6-dichlorophenyl)amino]-5-{[2-methoxy-4-(piperazin-1-yl)phenyl]amino}pyrido[3,4-d]pyridazin-4-ol

EXAMPLE 10A (80 mg, 0.13 mmol) was dissolved in a mixture of methanol (7 mL) and concentrated hydrochloric acid (3 mL). The mixture was concentrated under reduced pressure and was neutralized with saturated aqueous sodium bicarbonate solution. The aqueous phase was extracted with ethyl acetate (3×100 mL) and the combined organic layers were dried over sodium sulfate, filtered, and concentrated. The residue was purified by preparative HPLC (acetonitrile/water containing 0.1% trifluoroacetic acid) to give the title compound as a mono-trifluoroacetate salt. ¹H NMR (DMSO-d₆, 300 MHz): δ 12.24 (s, 1H), 11.41 (s, 1H), 9.34 (s, 1H), 8.68 (s, 2H), 8.01 (s, 1H), 7.62 (d, J=8.7 Hz, 3H), 7.41 (d, J=7.8 Hz, 1H), 7.42 (d, J=7.8 Hz, 1H), 6.61 (s, 1H), 6.67 (s, 1H), 6.12 (dd, J=1.8 Hz, 8.7 Hz, 1H), 3.82 (s, 3H), 3.23 (s, 8H). MS: 514 (M+H⁺).

### Example 11

### 7-(2,6-dichlorobenzyl)-5-({2-methoxy-4-[4-(propan-2-ylsulfonyl)piperazin-1-yl]phenyl}amino)pyrido[3,4-d]pyridazin-4-ol

To a solution of EXAMPLE 2F (51.1 mg, 0.100 mmol) in N,N-dimethylformamide (5 mL) at 0° C. was added N,N-diisopropylethylamine (38.7 mg, 0.300 mmol). After 5 minutes, a solution of propane-2-sulfonyl chloride (21.4 mg, 0.150 mmol) in N,N-dimethylformamide (1 mL) was added dropwise and the mixture was stirred at 0° C. for 15 hours. The mixture was diluted with ethyl acetate (50 mL) and washed with brine (50 mL). The organic layer was dried over anhydrous sodium sulfate, concentrated, filtered, and purified by flash chromatography on silica gel (200-300 mesh) eluting with 2/3 petroleum ether/ethyl acetate to give the title compound. ¹H NMR (DMSO-d₆, 300 MHz): δ 12.86 (s, 1H), 11.47 (s, 1H), 8.23 (s, 1H), 7.98 (d, J=8.7 Hz, 1H), 7.60 (d, J=7.8 Hz, 2H), 7.45 (t, J=8.1 Hz, 1H), 6.87 (s, 1H), 6.68 (s, 1H), 6.24 (d, J=8.7 Hz, 1H), 4.49 (s, 2H), 3.87 (s, 3H), 3.47-3.38 (m, 5H), 3.16 (m, 4H), 1.29 (d, J=6.9 Hz, 6H). MS: 617 (M+H⁺).

### Example 12

### 1-[4-(4-{[7-(2,6-dichlorobenzyl)-4-hydroxypyrido[3,4-d]pyridazin-5-yl]amino}-3-methoxyphenyl)piperazin-1-yl]-2-(dimethylamino)ethanone

To a solution of 2-(dimethylamino)acetic acid (12 mg, 0.11 mmol) in N,N-dimethylformamide (2 mL) was added ethyldiisopropylamine (0.05 mL, 0.2 mmol) and O-(7-azabenzotriazol-1-yl)-N,N,N′,N′-tetramethyluroniumhexafluoro phosphate (46 mg, 0.12 mmol), and the mixture was stirred at room temperature for 15 minutes. The product of EXAMPLE 2F (50 mg, 0.1 mmol) was added and the mixture was stirred at room temperature overnight. The mixture was diluted with water and extracted with ethyl acetate (3×5 mL). The combined organic phase was washed with brine, dried over anhydrous sodium sulfate, filtered and concentrated. The residue was washed with 1/10 ethyl acetate/petroleum ether to afford the title compound. ¹H NMR (DMSO-d₆, 300 MHz): δ 12.85 (s, 1H), 11.47 (s, 1H), 8.53 (s, 1H), 8.23 (s, 1H), 8.02 (d, J=9 Hz, 1H), 7.59 (d, J=8.4 Hz, 2H), 7.46-7.41 (m, 1H), 6.86 (s, 1H), 6.70 (s, 1H), 6.24 (d, J=9 Hz, 1H), 4.49 (s, 2H), 4.29 (s, 2H), 3.93 (s, 3H), 3.68-3.71 (m, 2H), 3.53-3.56 (m, 2H), 3.17 (d, J=13.5 Hz, 4H), 2.75 (s, 6H). MS: 598 (M+H⁺).

### Example 13

### 5-{[3-chloro-4-(piperazin-1-yl)phenyl]amino}-7-(2,6-dichlorobenzyl)pyrido[3,4-d]pyridazin-4(3H)-one

### Example 13A

### tert-butyl 4-(2-chloro-4-nitrophenyl)piperazine-1-carboxylate

A mixture of 1-bromo-2-chloro-4-nitrobenzene (1 g, 4.2 mmol), tert-butyl piperazine-1-carboxylate (0.86 g, 4.6 mmol), potassium carbonate (878 mg, 6.4 mmol) and tetrabutyl ammonium bromide (137 mg, 0.42 mmol) in dimethylsulfoxide (20 mL) was heated at 125° C. for 3 hours. After cooling to ambient temperature, the mixture was diluted with water and extracted with ethyl acetate. The organic layer was washed with brine, dried over anhydrous sodium sulfate, filtered, and concentrated. The residue was purified by flash chromatography on silica gel eluting with 5/1 petroleum ether/ethyl acetate to give the title compound. MS: 342 (M+H⁺)

### Example 13B

### tert-butyl 4-(4-amino-2-chlorophenyl)piperazine-1-carboxylate

To a solution of EXAMPLE 13A (1.5 g, 4.4 mmol) in 1:1 tetrahydrofuran/methanol (80 mL) at ambient temperature was slowly added zinc power (1.43 g, 22 mmol) and acetic acid (5 mL). The mixture was stirred for 1 hour, followed by addition of a saturated aqueous sodium hydrogen carbonate solution. The mixture was stirred for 1 hour and filtered, followed by extraction with ethyl acetate. The organic layer was washed with brine, dried over anhydrous sodium sulfate, filtered, and concentrated. The residue was purified by flash chromatography on silica gel eluting with 5/1 petroleum ether/ethyl acetate to give title compound. MS: 312 (M+H⁺).

### Example 13C

### tert-butyl 4-(2-chloro-4-(7-chloro-4-oxo-3,4-dihydropyrido[3,4-d]pyridazin-5-ylamino)phenyl)piperazine-1-carboxylate

The title compound was obtained following the procedure described in EXAMPLE 20D, using EXAMPLE 13B in place of EXAMPLE 20C. MS: 491 (M+H⁺).

### Example 13D

### tert-butyl 4-(4-(7-(2,6-dichlorobenzyl)-4-oxo-3,4-dihydropyrido[3,4-d]pyridazin-5-ylamino)-2-chlorophenyl)piperazine-1-carboxylate

The title compound was obtained following the procedure described in EXAMPLE 20E, using EXAMPLE 13C in place of EXAMPLE 20D. MS: 615 (M+H⁺).

### Example 13E

### 5-{[3-chloro-4-(piperazin-1-yl)phenyl]amino}-7-(2,6-dichlorobenzyl)pyrido[3,4-d]pyridazin-4(3H)-one

The title compound was obtained following the procedure described in EXAMPLE 20F, using EXAMPLE 13D in place of EXAMPLE 20E. ¹H NMR (DMSO-d₆, 300 MHz): δ 13.11 (s, 1H), 11.49 (s, 1H), 8.78 (brs, 2H), 8.33 (s, 1H), 7.92 (s, 1H), 7.59-7.38 (m, 4H), 7.08-6.99 (m, 2H), 4.50 (s, 2H), 3.28 (brs, 4H), 3.14 (brs, 4H). MS: 515 (M+H⁺).

### Example 14

### 7-(2,6-dichlorobenzyl)-5-{[5-(piperazin-1-yl)pyridin-2-yl]amino}pyrido[3,4-d]pyridazin-4(3H)-one

### Example 14A

### tert-butyl 4-(6-(7-chloro-4-oxo-3,4-dihydropyrido[3,4-d]pyridazin-5-ylamino)pyridin-3-yl)piperazine-1-carboxylate

The title compound was obtained following the procedure described in EXAMPLE 20D, using EXAMPLE 103C in place of EXAMPLE 20C. MS: 458 (M+H⁺).

### Example 14B

### tert-butyl 4-(6-(7-(2,6-dichlorobenzyl)-4-oxo-3,4-dihydropyrido[3,4-d]pyridazin-5-ylamino)pyridin-3-yl)piperazine-1-carboxylate

The title compound was obtained following the procedure described in EXAMPLE 20E, using EXAMPLE 14A in place of EXAMPLE 20D. MS: 582 (M+H⁺).

### Example 14C

### 7-(2,6-dichlorobenzyl)-5-{[5-(piperazin-1-yl)pyridin-2-yl]amino}pyrido[3,4-d]pyridazin-4(3H)-one

The title compound was obtained following the procedure described in EXAMPLE 20F, using EXAMPLE 14B in place of EXAMPLE 20E. ¹H NMR (CD₃OD, 300 MHz): δ 8.29 (s, 1H), 8.00-7.82 (m, 3H), 7.59-7.39 (m, 3H), 7.18 (s, 1H), 4.77 (s, 2H), 3.53-3.45 (m, 8H). MS: 515 (M+H⁺).

### Example 15

### 7-(2,6-dichlorobenzyl)-5-({2-methoxy-4-[4-(1-methylpiperidin-4-yl)piperazin-1-yl]phenyl}amino)pyrido[3,4-d]pyridazin-4(3H)-one

To a solution of EXAMPLE 2F (60 mg, 0.12 mmol) in methanol (10 mL) was added acetic acid (0.05 mL) and 1-methylpiperidin-4-one (135 mg, 1.2 mmol) and the mixture was stirred at 60° C. for 3 hours. After cooling to ambient temperature, sodium cyanoborohydride (151 mg, 2.4 mmol) was added and the mixture was stirred overnight. The mixture was poured into water (50 mL) and extracted with ethyl acetate (3×50 mL). The organic layer was dried over anhydrous sodium sulfate, filtered, concentrated and purified by preparative HPLC eluting with a gradient of 10/90 to 30/20 acetonitrile/water (containing 0.1% trifluoroacetic acid) to give the title compound as a solid trifluoroacetate salt. ¹H NMR (CD₃OD, 300 MHz): δ 8.20 (d, J=8.4 Hz, 1H), 8.13 (s, 1H), 7.51 (d, J=7.8 Hz, 2H), 7.37 (t, J=7.5 Hz, 1H), 6.82 (s, 1H), 6.73 (s, 1H), 6.38 (brs, 1H), 4.80 (s, 2H), 3.98 (s, 3H), 3.80-3.76 (m, 2H), 3.58 (brs, 8H), 3.35-3.20 (m, 3H), 2.98 (s, 3H), 2.54-2.20 (m, 2H), 2.15-2.13 (m, 2H). MS: 608.3 (M+H⁺).

### Example 16

### 7-(2,6-dichlorobenzyl)-5-({2-methoxy-4-[4-(3-methylbutyl)piperazin-1-yl]phenyl}amino)pyrido[3,4-d]pyridazin-4(3H)-one

To a solution of EXAMPLE 2F (0.1 g, 0.2 mmol) in methanol (3 mL) was added 3-methylbutanal (20 mg, 1.2 mmol), sodium cyanoborohydride (250 mg, 4 mmol) and a drop of acetic acid and the mixture was stirred at room temperature overnight. The mixture was carefully quenched with saturated aqueous ammonium chloride and extracted with ethyl acetate (3×10 mL). The combined organic phase was washed with brine, dried over anhydrous sodium sulfate, filtered and concentrated. The residue was purified by preparative HPLC using a gradient of 10/90 to 80/20 acetronitrile in water (containing 0.1% trifluoroacetic acid) to give the title compound. ¹H NMR (DMSO-d₆, 300 MHz): δ 12.90 (s, 1H), 11.50 (s, 1H), 8.41 (s, 1H), 8.11 (s, 1H), 8.04 (d, J=8.7 Hz, 1H), 7.60 (d, J=7.8 Hz, 2H), 7.46-7.41 (m, 1H), 6.86 (s, 1H), 6.71 (s, 1H), 6.25 (d, J=8.4 Hz, 1H), 4.49 (s, 2H), 3.90 (s, 3H), 3.84 (d, J=13.2 Hz, 2H), 3.62 (d, J=11.4 Hz, 2H), 3.19-3.12 (m, 4H), 2.98-2.91 (m, 2H), 1.68-1.59 (m, 3H), 0.96 (d, J=6 Hz, 6H). MS: 583 (M+H⁺).

### Example 17

### 7-(2,6-dichlorobenzyl)-5-({2-methoxy-4-[2-(pyrrolidin-1-yl)ethyl]phenyl}amino)pyrido[3,4-d]pyridazin-4(3H)-one

### Example 17A

### diethyl 2-(3-methoxy-4-nitrophenyl)malonate

To a suspension of sodium hydride (3.51 g, 88 mmol) in dry N,N-dimethylformamide (40 mL) at 0° C. was added diethyl malonate (10.3 g, 64.3 mmol) and the mixture was stirred at room temperature for 30 minutes. 4-Fluoro-2-methoxy-1-nitrobenzene (10 g, 58.5 mmol) was added and the mixture was stirred at 90° C. overnight. The mixture was diluted with water and extracted with ethyl acetate (3×20 mL). The combined organic phase was washed with brine, dried over anhydrous sodium sulfate, filtered and concentrated. The residue was purified by flash chromatography on silica gel eluting with a gradient of 1/20 to 1/10 ethyl acetate/petroleum ether to afford the title compound.

### Example 17B

### 2-(3-methoxy-4-nitrophenyl)acetic acid

To a solution of EXAMPLE 17A (0.3 g, 0.96 mmol) in ethanol (2 mL) was added 2N aqueous sodium hydroxide (2 mL) and the mixture was stirred at room temperature for 12 hours. The mixture was concentrated and the residue was diluted with water and extracted with ethyl acetate. The aqueous phase was acidified with concentrated hydrochloric acid to pH 2-3 and extracted with ethyl acetate (3×5 mL). The combined organic phase was washed with brine, dried over anhydrous sodium sulfate, filtered and concentrated and the crude material was used in the next step without further purification.

### Example 17C

### 2-(3-methoxy-4-nitrophenyl)-1-(pyrrolidin-1-yl)ethanone

A solution of EXAMPLE 17B (1 g, 4.74 mmol) in thionyl dichloride (20 mL) was stirred at reflux for 3 hours and was concentrated. The residue was diluted with dichloromethane (20 mL) and was added to a solution of pyrrolidine (0.63 mL, 7.1 mmol) and diisopropyl ethylamine (1.7 mL, 9.5 mmol). The mixture was stirred at room temperature overnight and was washed with 1N aqueous hydrochloridic acid (20 mL) and brine. The organic phase was dried over anhydrous sodium sulfate, filtered and concentrated and the residue was purified by flash chromatography on silica gel eluting with 1/100 methanol/dichloromethane to give the title compound.

### Example 17D

### 2-(4-amino-3-methoxyphenyl)-1-(pyrrolidin-1-yl)ethanone

To a suspension of Raney Nickel (0.5 g) in methanol (10 mL) was added EXAMPLE 17C (0.9 g, 3.4 mmol). The mixture was degassed with hydrogen three times and stirred at room temperature under hydrogen overnight. The mixture was filtered through a pad of diatomaceous earth and was washed with methanol. The filtrate was concentrated to give the title compound.

### Example 17E

### 2-methoxy-4-(2-(pyrrolidin-1-yl)ethyl)benzenamine

To a solution of EXAMPLE 17D (0.1, 0.43 mmol) in tetrahydrofuran (10 mL) was added lithium aluminium tetrahydride (49 mg, 1.28 mmol) and the mixture was stirred at room temperature for 2 hours. The mixture was quenched carefully by addition of water and 15% aqueous sodium hydroxide. The suspension was filtered and washed with ethyl acetate. The filtrate was dried over anhydrous sodium sulfate, filtered and concentrated to give the title compound.

### Example 17F

### 7-chloro-5-(2-methoxy-4-(2-(pyrrolidin-1-yl)ethyl)phenylamino)pyrido[3,4-d]pyridazin-4(3H)-one

The title compound was obtained following the procedure described in EXAMPLE 20D, using EXAMPLE 17E in place of EXAMPLE 20C.

### Example 17G

### 7-(2,6-dichlorobenzyl)-5-({2-methoxy-4-[2-(pyrrolidin-1-yl)ethyl]phenyl}amino)pyrido[3,4-d]pyridazin-4(3H)-one

The title compound was obtained following the procedure described in EXAMPLE 2E, using EXAMPLE 17F in place of EXAMPLE 2D. ¹H NMR (DMSO-d₆, 300 MHz): δ 12.95 (s, 1H), 11.66 (s, 1H), 8.28 (s, 1H), 8.10 (d, J=7.8 Hz, 1H), 7.60 (d, J=8.1 Hz, 2H), 7.47-7.44 (m, 1H), 6.97 (d, J=4.8 Hz, 2H), 6.58 (d, J=7.8 Hz, 1H), 4.52 (s, 2H), 3.90 (s, 3H), 3.01-3.30 (m, 5H), 2.80-2.91 (m, 3H), 1.81-1.93 (m, 4H). MS: 526 (M+H⁺).

### Example 18

### 7-(2,6-dichlorobenzyl)-5-({2-methoxy-4-[2-oxo-2-(pyrrolidin-1-yl)ethyl]phenyl}amino)pyrido[3,4-d]pyridazin-4(3H)-one

### Example 18A

### diethyl 2-(3-methoxy-4-nitrophenyl)malonate

To a solution of diethyl malonate (10.3 g, 64.3 mmol) in dry N,N-dimethylformamide (40 mL) at 0° C. was added 60% sodium hydride in mineral oil (3.51 g, 88 mmol) in portions over 30 minutes. A solution of 4-fluoro-2-methoxy-1-nitrobenzene (10 g, 58.5 mmol) in N,N-dimethylformamide (10 mL) was added dropwise and the mixture was stirred at 90° C. overnight. The mixture was diluted with water and exacted with ethyl acetate (3×20 mL). The combined organic phase was washed with brine, dried over anhydrous sodium sulfate, filtered and concentrated. The residue was purified by flash chromatography on silica gel eluting with 1/20 ethyl acetate/petroleum ether to provide the title compound.

### Example 18B

### 2-(3-methoxy-4-nitrophenyl)acetic acid

To a solution of EXAMPLE 18A (0.3 g, 0.96 mmol) in ethanol (2 mL) was added 2N sodium hydroxide (2 mL) and the mixture was stirred at room temperature for 12 hours. The mixture was concentrated, diluted with water and washed with ethyl acetate. The aqueous phase was acidified with concentrated hydrochloridic acid to pH 2-3 and extracted with ethyl acetate (3×5 mL). The combined organic phase was washed with brine, dried over anhydrous sodium sulfate, filtered and concentrated and the residue used in the next step without further purification.

### Example 18C

### 2-(3-methoxy-4-nitrophenyl)-1-(pyrrolidin-1-yl)ethanone

A solution of EXAMPLE 18B (1 g, 4.74 mmol) in thionyl dichloride (20 mL) was stirred at reflux for 3 hours and the mixture was concentrated. The residue was dissolved in dichloromethane (20 mL) and N,N-diisopropylethylamine (1.7 mL, 9.5 mmol) and pyrrolidine (0.63 mL, 7.1 mmol) was added. The mixture was stirred at room temperature overnight. The mixture was washed with 1N aqueous hydrochloric acid and brine, dried over anhydrous sodium sulfate, filtered and concentrated. The residue was purified by flash chromatography on silica gel eluting with 1/100 methanol/dichloromethane to provide the title compound.

### Example 18D

### 2-(4-amino-3-methoxyphenyl)-1-(pyrrolidin-1-yl)ethanone

To a solution of EXAMPLE 18C (0.9 g, 3.4 mmol) in methanol (10 mL) was added Raney Nickel (0.5 g). The mixture was degassed with hydrogen and stirred at room temperature under hydrogen overnight. The mixture was filtered through a pad of diatomaceous earth and the filtrate was concentrated to afford the title compound.

### Example 18E

### 7-chloro-5-(2-methoxy-4-(2-oxo-2-(pyrrolidin-1-yl)ethyl)phenylamino)pyrido[3,4-d]pyridazin-4(3H)-one

The title compound was obtained following the procedure described in EXAMPLE 20D, using EXAMPLE 18D in place of EXAMPLE-20C.

### Example 18F

### 7-(2,6-dichlorobenzyl)-5-({2-methoxy-4-[2-oxo-2-(pyrrolidin-1-yl)ethyl]phenyl}amino)pyrido[3,4-d]pyridazin-4(3H)-one

The title compound was obtained following the procedure described in EXAMPLE 2E, using EXAMPLE 18E in place of EXAMPLE 2D. ¹H NMR (DMSO-d₆, 300 MHz): δ 12.93 (s, 1H), 11.63 (s, 1H), 8.27 (s, 1H), 8.04 (d, J=8.1 Hz, 1H), 7.58 (d, J=8.1 Hz, 2H), 7.44-7.39 (m, 1H), 6.97 (s, 2H), 6.88 (s, 1H), 6.54-6.51 (m, 1H), 4.51 (s, 2H), 3.86 (s, 3H), 3.57 (s, 2H), 3.48 (t, 2H), 3.32 (t, 2H), 1.92-1.77 (m, 4H). MS: 539 (M+H⁺).

### Example 19

### 7-(2,6-dichlorobenzyl)-5-({2-methoxy-4-[4-(3-methylbutanoyl)piperazin-1-yl]phenyl}amino)pyrido[3,4-d]pyridazin-4(3H)-one

To a solution of EXAMPLE 2F (51 mg, 0.1 mmol) in N,N-dimethylformamide (3 mL) at 0° C. was added 3-methylbutanoyl chloride (13.5 mg, 0.11 mmol) and the solution was stirred at 0° C. for 1 hour. The reaction was quenched with saturated sodium bicarbonate solution (10 mL) and extracted with ethyl acetate (2×50 mL). The combined organic phase was dried, concentrated and purified by flash chromatography on silica gel (200-300 mesh) eluting with 100:1 dichloromethane/ethanol to give the title compound. ¹H NMR (DMSO-d₆, 300 MHz): δ 8.24 (s, 1H), 7.97 (d, 1H), 7.62-7.59 (m, 2H), 7.48-7.42 (m, 1H), 7.29 (s, 1H), 7.12 (s, 1H), 6.95 (s, 1H), 6.87 (s, 1H), 6.67 (s, 1H), 6.21 (d, 1H), 4.49 (s, 2H), 3.88 (s, 3H), 3.64 (m, 4H), 3.07 (m, 4H), 2.29-2.26 (m, 2H), 2.06-2.04 (m, 1H), 0.96-0.94 (m, 6H). MS: 595 (M+H⁺).

### Example 20

### 7-(2,6-dichlorobenzyl)-5-{[2,5-difluoro-4-(piperazin-1-yl)phenyl]amino}pyrido[3,4-d]pyridazin-4(3H)-one

### Example 20A

### N,N-dibenzyl-4-bromo-2,5-difluoroaniline

To a mixture of 4-bromo-2,5-difluoroaniline (8 g, 39 mmol) and potassium carbonate (16 g, 116 mmol) in acetonitrile (200 mL) was added (bromomethyl)benzene (14.5 g, 85 mmol). After refluxing for 20 hours, the mixture was filtered and the filtrate was concentrated. The residue was purified by flash chromatography on silica gel (200-300 mesh) eluting with 5/1 petroleum ether/ethyl acetate to give the title compound. MS: 388 (M+H⁺).

### Example 20B

### tert-butyl 4-(4-(dibenzylamino)-2,5-difluorophenyl)piperazine-1-carboxylate

A mixture of EXAMPLE 20A (5.12 g, 13.2 mmol), tert-butyl piperazine-1-carboxylate (2.95 g, 15.8 mmol), palladium acetate (149 mg, 0.66 mmol), 2,2′-bis(diphenylphosphosino)-1,1′-binaphthyl (616 mg, 0.99 mmol) and cesium carbonate (8.61 g, 26.4 mmol) in toluene (100 mL) was heated at 100° C. under nitrogen for 16 hours. After cooling to room temperature, the mixture was concentrated and the residue was purified by flash chromatography on silica gel (200-300 mesh) eluting with 5/1 petroleum ether/ethyl acetate to give the title compound.

### Example 20C

### tert-butyl 4-(4-amino-2,5-difluorophenyl)piperazine-1-carboxylate

To a solution of EXAMPLE 20B (4 g, 8 mmol) in methanol (100 mL) was added 10% palladium on carbon (400 mg). The mixture was stirred at room temperature under hydrogen for 16 hours. The catalyst was filtered off and the filtrate was concentrated to give the title compound. MS: 314 (M+H⁺).

### Example 20D

### tert-butyl 4-(4-(7-chloro-4-oxo-3,4-dihydropyrido[3,4-d]pyridazin-5-ylamino)-2,5-difluorophenyl) piperazine-1-carboxylate

A solution of EXAMPLE 7K (70 mg, 0.33 mmol), EXAMPLE 20C (100 mg, 0.38 mmol) and N,N-diisopropylethylamine (0.1 mL, 0.57 mmol) in dioxane (5 mL) was stirred at 120° C. for 15 hours. After cooling to ambient temperature, the mixture was concentrated and was purified by flash chromatography on silica gel (200-300 mesh) eluting with 30/1 dichloromethane/methanol to give the title compound. MS: 493 (M+H⁺).

### Example 20E

### tert-butyl 4-(4-(7-(2,6-dichlorobenzyl)-4-oxo-3,4-dihydropyrido[3,4-d]pyridazin-5-ylamino)-2,5-difluorophenyl)piperazine-1-carboxylate

A solution of EXAMPLE 20D (70 mg, 0.14 mmol), a solution of 0.5M (2,6-dichloro benzyl) zinc(II) bromide in tetrahydrofuran (2.8 mL, 1.4 mmol), tris(dibenzylideneacetone) dipalladium (4 mg, 0.005 mmol), 2-(dicyclohexylphosphino)-2′,4′,6′-triisopropyl-1,1′-biphenyl (8 mg, 0.02 mmol) and potassium 2-methylpropan-2-olate (50 mg, 0.5 mmol) in 2-methylpropan-2-ol (2 mL) was degassed with nitrogen twice and was stirred in a sealed tube at 120° C. for 15 hours. After cooling to ambient temperature, the mixture was concentrated and was purified by flash chromatography on silica gel eluting with 100:1 dichloromethane/ethanol to give the title compound. MS: 617 (M+H⁺).

### Example 20F

### 7-(2,6-dichlorobenzyl)-5-{[2,5-difluoro-4-(piperazin-1-yl)phenyl]amino}pyrido[3,4-d]pyridazin-4(3H)-one

To a solution of EXAMPLE 20E (100 mg, 0.16 mmol) in dichloromethane (10 mL) was added 2,2,2-trifluoroacetic acid (2 mL) dropwise. After stirring at ambient temperature for 12 hours, the mixture was concentrated and was purified by preparative HPLC using a gradient of 10/90 to 80/20 acetronitrile in water (containing 0.1% trifluoroacetic acid) to give the title compound. ¹H NMR (DMSO-d₆, 300 MHz): δ13.13 (s, 1H), 11.66 (s, 1H), 8.72 (brs, 2H), 8.37 (s, 1H), 8.16-8.09 (m, 1H), 7.56-7.54 (m, 2H), 7.42-7.37 (m, 1H), 7.18-7.13 (m, 2H), 4.54 (s, 2H), 3.28-3.18 (m, 8H). MS: 517 (M+H⁺).

### Example 21

### 7-(2,6-dichlorobenzyl)-5-{[2,6-dimethyl-4-(piperazin-1-yl)phenyl]amino}pyrido[3,4-d]pyridazin-4-ol

### Example 21A

### tert-butyl 4-(3,5-dimethyl-4-nitrophenyl)piperazine-1-carboxylate

A suspension of 5-fluoro-1,3-dimethyl-2-nitrobenzene (3.5 g, 20.49 mmol), tert-butyl piperazine-1-carboxylate (4.62 g, 24.82 mmol) and potassium carbonate (4.29 g, 31.04 mmol) in N,N-dimethylformamide (50 mL) was heated at 110° C. for 15 hours. After cooling to ambient temperature, water (50 mL) was added and the mixture was extracted with dichloromethane. The combined organic layers were dried over anhydrous sodium sulfate, filtered and concentrated. The residue was recrystallized from ethanol to give the title compound.

### Example 21B

### tert-buty 14-(4-amino-3,5-dimethylphenyl)piperazine-1-carboxylate

A mixture of EXAMPLE 21A and Raney-Nickel in methanol (10 mL) was stirred under hydrogen at ambient temperature for 15 hours. The solution was filtered through diatomaceous earth and the filtrate was concentrated. The residue was purified by flash chromatography on silica gel (200-300 mesh) eluting with a gradient of 3/1 to 2/1 petroleum ether/ethyl acetate to give the title compound.

### Example 21C

### tert-butyl 4-(4-(7-chloro-4-oxo-3,4-dihydropyrido[3,4-d]pyridazin-5-ylamino)-3,5-dimethylphenyl)piperazine-1-carboxylate

The title compound was obtained following the procedure described in EXAMPLE 20D, using EXAMPLE 21B in place of EXAMPLE 20C. MS: 485 (M+H⁴).

### Example 21D

### tert-butyl 4-(4-(7-(2,6-dichlorobenzyl)-4-oxo-3,4-dihydropyrido[3,4-d]pyridazin-5-ylamino)-3,5-dimethylphenyl)piperazine-1-carboxylate

The title compound was obtained following the procedure described in EXAMPLE 20E, using EXAMPLE 21C in place of EXAMPLE 20D. MS: 609 (M+H⁺).

### Example 21E

### 7-(2,6-dichlorobenzyl)-5-{[2,6-dimethyl-4-(piperazin-1-yl)phenyl]amino}pyrido[3,4-d]pyridazin-4-ol

The title compound was obtained following the procedure described in EXAMPLE 20F, using EXAMPLE 21D in place of EXAMPLE 20E. ¹H NMR (CD₃OD, 300 MHz): δ 8.03 (s, 1H), 7.32 (d, J=1.8 Hz, 1H), 7.29 (s, 1H), 7.21 (m, 1H), 6.71 (s, 1H), 6.48 (s, 1H), 4.29 (s, 2H), 3.41 (m, 8H), 2.05 (s, 6H). MS: 509 (M+H⁺).

### Example 22

### 5-{[2-methoxy-4-(piperazin-1-yl)phenyl]amino}-7-{[2-(propan-2-ylsulfonyl)phenyl]amino}pyrido[3,4-d]pyridazin-4(3H)-one

### Example 22A

### tert-butyl 4-(4-(7-(2-(isopropylsulfonyl)phenylamino)-4-oxo-3,4-dihydropyrido[3,4-d]pyridazin-5-ylamino)-3-methoxyphenyl)piperazine-1-carboxylate

To a solution of EXAMPLE 24A (153 mg, 0.25 mmol) in tert-butanol (5 mL) was added 2-(isopropylsulfonyl)benzenamine (50 mg, 0.25 mmol), potassium tert-butoxide (84 mg, 0.75 mmol), 2-dicyclohexylphosphino-2′,4′,6′-triisopropylbiphenyl (12 mg, 0.025 mmol) and tris(dibenzylideneacetone)dipalladium(0) (23 mg, 0.025 mmol) and the mixture was heated at 120° C. in a Biotage Microwave Synthesizer for 1 hour. After cooling to ambient temperature, the mixture was concentrated and the residue was purified by flash chromatography on silica gel eluting with 50/1 dichloromethane/methanol to give the title compound. MS: 650 (M+H⁺).

### Example 22B

### 5-{[2-methoxy-4-(piperazin-1-yl)phenyl]amino}-7-{[2-(propan-2-ylsulfonyl)phenyl]amino}pyrido[3,4-d]pyridazin-4(3H)-one

To a solution of EXAMPLE 22A (90 mg, 0.14 mmol) in dichloromethane (20 mL) was added 2,2,2-trifluoroacetic acid (4 mL) and the mixture was stirred at ambient temperature for 3 hours. After concentration, the residue was purified by preparative HPLC eluting with a gradient of 10/90 to 30/20 acetonitrile/water (containing 0.1% trifluoroacetic acid) to give the title compound as a solid trifluoroacetate salt. ¹H NMR (DMSO-d₆, 300 MHz): δ 12.46 (s, 1H), 11.46 (s, 1H), 9.08 (s, 1H), 8.70 (br, 2H), 8.11 (s, 1H), 8.02-7.76 (m, 3H), 7.45 (t, J=8.7 Hz, 1H), 6.71 (s, 1H), 6.46 (s, 1H), 6.24 (d, J=8.7 Hz, 1H), 3.88 (s, 3H), 3.43-3.30 (m, 9H), 1.12 (d, J=6.6 Hz, 6H). MS: 550.1 (M+H⁺).

### Example 23

### 5-{[2-methoxy-4-(piperazin-1-yl)phenyl]amino}-7-{[(4R)-4-(propan-2-yl)-4,5-dihydro-1,3-oxazol-2-yl]amino}pyrido[3,4-d]pyridazin-4-ol

### Example 23A

### (R)-tert-butyl 4-(4-(7-(4-isopropyl-4,5-dihydrooxazol-2-ylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 10A, using (R)-4-isopropyl-4,5-dihydrooxazol-2-amine in place of 2,6-dichlorobenzenamine.

### Example 23B

### 5-{[2-methoxy-4-(piperazin-1-yl)phenyl]amino}-7-{[(4R)-4-(propan-2-yl)-4,5-dihydro-1,3-oxazol-2-yl]amino}pyrido[3,4-d]pyridazin-4-ol

The title compound was obtained following the procedure described in EXAMPLE 2F, using EXAMPLE 23A in place of EXAMPLE 2E. ¹H NMR (DMSO-d₆, 300 MHz): δ 11.11 (s, 1H), 8.27 (s, 1H), 7.82 (d, J=8.7 Hz, 1H), 6.77 (s, 1H), 6.63-6.58 (m, 2H), 4.90-4.84 (m, 1H), 4.71-4.66 (m, 1H), 4.17-4.10 (m, 1H), 3.92 (s, 3H), 3.39-3.36 (m, 4H), 3.27-3.24 (m, 4H), 1.77-1.72 (m, 1H), 0.72 (dd, J=6.6 Hz, 16.8 Hz, 6H). MS: 479 (M+H⁺).

### Example 24

### 7-[(2-chlorophenyl)amino]-5-{[2-methoxy-4-(piperazin-1-yl)phenyl]amino}pyrido[3,4-d]pyridazin-4(3H)-one

### Example 24A

### tert-butyl 4-(4-(7-chloro-4-oxo-3,4-dihydropyrido[3,4-d]pyridazin-5-ylamino)-3-methoxyphenyl)piperazine-1-carboxylate

A solution of EXAMPLE 7K (70 mg, 0.33 mmol), EXAMPLE 1G (100 mg, 0.34 mmol) and N,N-diisopropylethylamine (0.1 mL, 0.57 mmol) in dioxane (5 mL) was stirred at 120° C. for 15 hours. After cooling to ambient temperature, the mixture was concentrated and was purified by flash chromatography on silica gel (200-300 mesh) eluting with 20/1 dichloromethane/methanol to give the title compound. MS: 487 (M+H⁺).

### Example 24B

### tert-butyl 4-(4-(7-(2-chlorophenylamino)-4-oxo-3,4-dihydropyrido[3,4-d]pyridazin-5-ylamino)-3-methoxyphenyl)piperazine-1-carboxylate

A mixture of EXAMPLE 24A (100 mg, 0.21 mmol), 2-chlorobenzenamine (40 mg, 0.31 mmol), tris(dibenzylideneacetone)dipalladium (4 mg, 0.005 mmol), 2-(dicyclohexylphosphino)-2′,4′,6′-triisopropyl-1,1′-biphenyl (8 mg, 0.02 mmol) and potassium 2-methylpropan-2-olate (50 mg, 0.5 mmol) in 2-methylpropan-2-ol (2 mL) was degassed with nitrogen twice and was stirred in a sealed tube at 120° C. for 15 hours. After cooling to ambient temperature, the mixture was concentrated and purified by flash chromatography on silica gel eluting with 100:1 dichloromethane/ethanol to provide the title compound. MS: 578 (M+H⁺).

### Example 24C

### 7-[(2-chlorophenyl)amino]-5-{[2-methoxy-4-(piperazin-1-yl)phenyl]amino}pyrido[3,4-d]pyridazin-4(3H)-one

To a solution of EXAMPLE 24B (160 mg, 0.27 mmol) in dichloromethane (10 mL) was added 2,2,2-trifluoroacetic acid (2 mL) dropwise. The solution was stirred at ambient temperature for 12 hours, concentrated and purified by preparative HPLC using a gradient of 10/90 to 80/20 acetronitrile in water (containing 0.1% trifluoroacetic acid) to give the title compound. ¹H NMR (DMSO-d₆, 300 MHz): δ 12.31 (s, 1H), 11.50 (s, 1H), 9.19 (s, 1H), 8.70 (brs, 2H), 8.15-8.05 (m, 2H), 7.71-7.45 (m, 2H), 7.42-7.27 (m, 2H), 6.71 (s, 1H), 6.24-6.20 (m, 2H), 4.01 (s, 3H), 2.76 (brs, 8H). MS: 478 (M+H⁺).

### Example 25

### 7-[(5-chloro-2-fluorophenyl)amino]-5-{[2-methoxy-4-(piperazin-1-yl)phenyl]amino}pyrido[3,4-d]pyridazin-4(3H)-one

### Example 25A

### tert-butyl 4-(4-(7-(5-chloro-2-fluorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 24B, using 5-chloro-2-fluoroaniline in place of 2-chlorobenzenamine. MS: 596 (M+H⁺).

### Example 25B

### 7-[(5-chloro-2-fluorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 24C, using EXAMPLE 25A in place of EXAMPLE 24B. ¹H NMR (DMSO-d₆, 300 MHz): δ 12.41 (s, 1H), 11.49 (s, 1H), 8.42 (s, 1H), 8.71 (brs, 2H), 8.14-8.08 (m, 2H), 8.01-7.87 (m, 1H), 7.39-7.35 (m, 1H), 7.22-7.18 (m, 1H), 6.75 (s, 1H), 6.42-6.38 (m, 2H), 3.90 (s, 2H), 8.32 (brs, 8H). MS: 496 (M+H⁺).

### Example 26

### 7-(2,6-dichlorobenzyl)-5-{[2-fluoro-4-(piperazin-1-yl)phenyl]amino}pyrido[3,4-d]pyridazin-4(3H)-one

### Example 26A

### tert-butyl 4-(3-fluoro-4-nitrophenyl)piperazine-1-carboxylate

A mixture of 4-bromo-2-fluoro-1-nitrobenzene (5 g, 23 mmol), tert-butyl piperazine-1-carboxylate (4.24 g, 23 mmol), palladium diacetate (0.51 g, 2.3 mmol), (±)-2,2′-bis(diphenylphosphino)-1,1′-binaphthalene (2.13 g, 3.4 mmol) and cesium carbonate (14.8 g, 45 mmol) in toluene (120 mL) was heated under nitrogen at 60° C. for 20 hours. After cooling to ambient temperature, the mixture was concentrated and the residue was diluted with dichloromethane (300 mL) and washed with water. The combined organic phase was washed with brine, dried over sodium sulfate, filtered and concentrated. The residue was purified by flash chromatography on silica gel (200-300 mesh) eluting with 3/1 petroleum ether/ethyl acetate to give the title compound. MS: 348 (M+Na⁺).

### Example 26B

### tert-butyl 4-(4-amino-3-fluorophenyl)piperazine-1-carboxylate

A mixture of EXAMPLE 26A (1.6 g, 4.9 mmol), zinc dust (3.2 g, 49 mmol) and acetic acid (5.4 mL) in 1/1 tetrahydrofuran/methanol (100 mL) was stirred at ambient temperature for 1 hour. The mixture was filtered and the filtrate was diluted with water and adjusted to pH 9. The mixture was extracted with ethyl acetate and the combined organic phase was washed with brine, dried over sodium sulfate, filtered and concentrated. The residue was purified by flash chromatography on silica gel (200-300 mesh) eluting with 99/1 dichloromethane/methanol to give the title compound. MS: 296 (M+H⁺).

### Example 26C

### tert-butyl 4-(4-(7-chloro-4-hydroxypyrido[3,4-d]pyridazin-5-ylamino)-3-fluorophenyl)piperazine-1-carboxylate

A solution of EXAMPLE 7K (70 mg, 0.33 mmol), EXAMPLE 26B (100 mg, 0.34 mmol) and N,N-diisopropylethylamine (0.1 mL, 0.57 mmol) in dioxane (15 mL) was stirred at 120° C. for 15 hours. After cooling to ambient temperature, the mixture was concentrated and was purified by flash chromatography on silica gel eluting with 100/1 dichloromethane/methanol. MS: 475 (M+H⁺).

### Example 26D

### tert-butyl 4-(4-(7-(2,6-dichlorobenzyl)-4-hydroxypyrido[3,4-d]pyridazin-5-ylamino)-3-fluorophenyl)piperazine-1-carboxylate

To a solution of EXAMPLE 26C (159 mg, 0.33 mmol) in tetrahydrofuran (2 mL) was added tetrakis(triphenylphosphine) palladium(0) (5 mg) and 0.6M (2,6-dichlorobenzyl)zinc(II) bromide in tetrahydrofuran (8 mL, 4.8 mmol) and the mixture was degassed with nitrogen twice and was stirred at 65° C. for 15 hours. After cooling to ambient temperature, the reaction was quenched with saturated ammonium chloride solution (10 mL) and the mixture was extracted with ethyl acetate (3×25 mL). The combined organic phase was washed with saturated brine solution, dried over anhydrous magnesium sulfate, filtered and concentrated. The residue was purified by flash chromatography on silica gel (200-300 mesh) eluting with 100/1 dichloromethane/methanol to give the title compound. MS: 599 (M+H⁺).

### Example 26E

### 7-(2,6-dichlorobenzyl)-5-{[2-fluoro-4-(piperazin-1-yl)phenyl]amino}pyrido[3,4-d]pyridazin-4(3H)-one

To a solution of EXAMPLE 26D (160 mg, 0.27 mmol) in dichloromethane (10 mL) was added 2,2,2-trifluoroacetic acid (2 mL) dropwise. The solution was stirred at ambient temperature for 12 hours, and was concentrated and purified by flash chromatography on silica gel (200-300 mesh) eluting with 100/1 dichloromethane/ethanol to give the title compound. ¹H NMR (DMSO-d₆, 300 MHz): δ 13.00 (s, 1H), 11.41 (s, 1H), 8.74 (brs, 2H), 8.32 (s, 1H), 8.06-8.00 (m, 1H), 7.63-7.60 (m, 2H), 7.48-7.43 (m, 1H), 7.03-6.97 (m, 2H), 6.52 (d, J=9.0 Hz 1H), 4.51 (s, 2H), 3.36-3.28 (m, 8H).

### Example 27

### 7-[(2-chloro-5-fluorophenyl)amino]-5-{[2-methoxy-4-(piperazin-1-yl)phenyl]amino}pyrido[3,4-d]pyridazin-4(3H)-one

### Example 27A

### tert-butyl 4-(4-(7-(2-chloro-5-fluorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 24B, using 2-chloro-5-fluoroaniline in place of 2-chlorobenzenamine. MS: 596 (M+H⁺).

### Example 27B

### 7-[(2-chloro-5-fluorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 24C, using EXAMPLE 27A in place of EXAMPLE 24B. ¹H NMR (DMSO-d₆, 300 MHz): δ 12.41 (s, 1H), 11.45 (s, 1H), 9.20 (s, 1H), 8.74 (brs, 2H), 8.11-8.08 (m, 2H), 7.78-7.75 (m, 1H), 7.48-7.43 (m, 2H), 7.63-7.58 (m, 1H), 7.12-7.05 (m, 1H), 6.73 (s, 1H), 6.42 (s, 1H), 6.31-6.28 (m, 1H), 3.89 (s, 3H), 3.31 (brs, 8H). MS: 496 (M+H⁺).

### Example 28

### 7-[(2,5-dichlorophenyl)amino]-5-{[2-methoxy-4-(piperazin-1-yl)phenyl]amino}pyrido[3,4-d]pyridazin-4(3H)-one

### Example 28A

### tert-butyl 4-(4-(7-(2,5-dichlorophenylamino)-4-oxo-3,4-dihydropyrido[3,4-d]pyridazin-5-ylamino)-3-methoxyphenyl)piperazine-1-carboxylate

A mixture of EXAMPLE 24A (100 mg, 0.21 mmol), 2,5-dichloroaniline (33 mg, 0.21 mmol), tris(dibenzylideneacetone)dipalladium (19 mg, 0.02 mmol), dimethylbisdiphenyl phosphinoxanthene (12 mg, 0.02 mmol), potassium tert-butoxide (71 mg, 0.63 mmol) and tert-butanol (2 mL) was heated at 120° C. under nitrogen for 48 hours. The mixture was concentrated and the residue was dissolved in dichloromethane, washed with saturated aqueous sodium bicarbonate and brine, dried over sodium sulfate, filtered and concentrated. The residue was purified by flash chromatography on silica gel (200-300 mesh) eluting with 98/2 dichloromethane/methanol to give the title compound. MS: 611 (M+H⁺).

### Example 28B

### 7-[(2,5-dichlorophenyl)amino]-5-{[2-methoxy-4-(piperazin-1-yl)phenyl]amino}pyrido[3,4-d]pyridazin-4(3H)-one

To a solution of EXAMPLE 28A (50 mg, 0.08 mmol) in dichloromethane (4 mL) was added trifluoroacetic acid (1 mL) dropwise. After stirring at ambient temperature for 4 hours, the mixture was concentrated and the residue was purified by preparative HPLC using a gradient of 10/90 to 90/10 acetronitrile in water (containing 0.1% trifluoroacetic acid) to give the title compound. ¹H NMR (DMSO-d₆, 300 MHz): δ 12.39 (s, 1H), 11.53 (s, 1H), 9.24 (s, 1H), 8.75-8.07 (m, 2H), 7.90 (d, J=2.4 Hz, 1H), 7.61 (d, J=8.7 Hz, 1H), 7.29 (dd, J=8.4 Hz, 2.4 Hz, 1H), 6.73 (d, J=2.4 Hz, 1H), 6.38 (s, 1H), 6.31 (dd, J=9.0 Hz, 2.4 Hz, 1H), 3.89 (s, 3H), 3.29 (s, 8H). MS: 512 (M+H⁺).

### Example 29

### 7-[(2-chloro-6-fluorophenyl)amino]-5-{[2-methoxy-4-(piperazin-1-yl)phenyl]amino}pyrido[3,4-d]pyridazin-4(3H)-one

### Example 29A

### tert-butyl 4-(4-(7-(2-chloro-6-fluorophenylamino)-4-oxo-3,4-dihydropyrido[3,4-d]pyridazin-5-ylamino)-3-methoxyphenyl)piperazine-1-carboxylate

A mixture of EXAMPLE 24A (370 mg, 0.76 mmol), 2-chloro-6-fluoroaniline (121 mg, 0.84 mmol), tris(dibenzylideneacetone)dipalladium (70 mg, 0.08 mmol), 2-dicyclohexylphosphino-2′,4′,6′-triisopropylbiphenyl (44 mg, 0.1 mmol), potassium tert-butoxide (257 mg, 2.28 mmol) and tert-butanol (5 mL) was heated under nitrogen at 120° C. for 48 hours. The mixture was concentrated and the residue was dissolved in dichloromethane, washed with saturated aqueous sodium bicarbonate and brine, dried over sodium sulfate, filtered and concentrated. The residue was purified by flash chromatography on silica gel (200-300 mesh) eluting with 98/2 dichloromethane/methanol to give the title compound. MS: 596 (M+H⁺).

### Example 29B

### 7-[(2-chloro-6-fluorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 28B, using EXAMPLE 29A in place of EXAMPLE 28A. ¹H NMR (CD₃OD. 300 MHz): δ 8.02-7.95 (m, 2H), 7.45-7.37 (m, 2H), 7.29-7.24 (m, 1H), 6.70 (brs, 2H), 6.15 (brs, 2H), 3.96 (brs, 3H), 3.40-3.38 (m, 8H). MS: 496 (M+Hi).

### Example 30

### 7-(2,6-dichlorobenzyl)-5-{[2-methoxy-4-(4-methylpiperazin-1-yl)phenyl]amino}pyrido[3,4-d]pyridazin-4(3H)-one

### Example 30A

### 2-methoxy-4-(4-methylpiperazin-1-yl)aniline

The title compound was obtained following the procedure described in EXAMPLE 1F-G, using 1-methylpiperazine in place of tert-butyl piperazine-1-carboxylate.

### Example 30B

### 7-chloro-5-(2-methoxy-4-(4-methylpiperazin-1-yl)phenylamino)pyrido[3,4-d]pyridazin-4(3H)-one

To a solution of EXAMPLE 7K (120 mg, 0.56 mmol) in dioxane (10 mL) was added EXAMPLE 30A (136 mg, 0.61 mmol) and N,N-diisopropylethylamine (724 mg, 5.6 mmol) and the mixture was stirred at 120° C. in a sealed tube for 16 hours. The mixture was cooled to ambient temperature, poured into water (50 mL) and extracted with ethyl acetate (3×50 mL). The organic layer was dried over anhydrous sodium sulfate, filtered, concentrated and purified by flash chromatography on silica gel (200-300 mesh) eluting with 50/1 dichlomethane/methanol to give the title compound. MS: 401 (M+H⁺).

### Example 30C

### 7-(2,6-dichlorobenzyl)-5-{[2-methoxy-4-(4-methylpiperazin-1-yl)phenyl]amino}pyrido[3,4-d]pyridazin-4(3H)-one

To a solution of EXAMPLE 30B (200 mg, 0.5 mmol) in tetrahydrofuran (10 mL) was added 0.6M (2,6-dichlorobenzyl)zinc(II) bromide in tetrahydrofuran (8 mL, 5 mmol) and tetrakis(triphenylphosphine)palladium (21 mg, 0.05 mmol) and the mixture was stirred at 70° C. under nitrogen for 16 hours. The mixture was cooled to ambient temperature, poured into water (50 mL) and extracted with ethyl acetate (3×50 mL). The organic layer was dried over anhydrous sodium sulfate, filtered, concentrated and purified by preparative HPLC eluting with a gradient of 10/90 to 30/20 acetonitrile/water (containing 0.1% trifluoroacetic acid) to give the title compound as a solid trifluoroacetate salt. ¹H NMR (DMSO-d₆, 300 MHz): δ 12.88 (s, 1H), 11.47 (s, 1H), 9.70 (br, 1H), 8.23 (s, 1H), 8.01 (d, J=9.0 Hz, 1H), 7.58 (d, J=8.4 Hz, 2H), 7.42 (t, J=8.4 Hz, 1H), 6.85 (s, 1H), 6.69 (s, 1H), 6.24 (d, J=9.0 Hz, 1H), 4.46 (s, 2H), 3.87 (s, 3H), 3.83-3.79 (m, 2H), 3.56-3.52 (m, 2H), 3.19-3.16 (m, 2H), 2.95-2.88 (m, 5H). MS: 525.1 (M+H⁺).

### Example 31

### 7-[(3,5-dichlorophenyl)amino]-5-{[2-methoxy-4-(piperazin-1-yl)phenyl]amino}pyrido[3,4-d]pyridazin-4(3H)-one

### Example 31A

### tert-butyl 4-(4-(7-(3,5-dichlorophenylamino)-4-oxo-3,4-dihydropyrido[3,4-d]pyridazin-5-ylamino)-3-methoxyphenyl)piperazine-1-carboxylate

A mixture of EXAMPLE 24A (120 mg, 0.25 mmol), 3,5-dichloro benzenamine (80 mg, 0.5 mmol), tris(dibenzyldeneacetone)dipalladium(0) (23 mg, 0.025 mmol), 2-dicyclohexylphosphino-2′,4′,6′-triisopropylbiphenyl (12 mg, 0.025 mmol), potassium tert-butoxide (47 g, 0.42 mmol) and tert-butanol (6.0 mL) was degassed with nitrogen for 5 minutes and heated at 120° C. under nitrogen for 16 hours. After cooling to ambient temperature, the mixture was concentrated and the residue was purified by flash chromatography on silica gel (200-300 mesh) eluting with a gradient of 100/1 to 80/1 dichloromethane/methanol to provide the title compound. MS: 612 (M+H⁺).

### Example 31B

### 7-[(3,5-dichlorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 20F, using EXAMPLE 31A in place of EXAMPLE 20E. ¹H NMR (DMSO-d₆, 300 MHz): δ 12.45 (s, 1H), 11.37 (s, 1H), 9.85 (s, 1H), 8.72 (brs, 2H), 8.14 (s, 1H), 8.07 (d, J=8.7 Hz, 1H), 7.67 (d, J=1.8 Hz, 2H), 7.15 (d, J=1.8 Hz, 1H), 6.79 (s, 1H), 6.56 (d, J=8.7 Hz, 1H), 6.26 (s, 1H), 3.90 (s, 3H), 3.35 (brs, 4H), 3.30 (brs, 4H). MS: 515 (M+H⁺).

### Example 32

### 7-[(2,6-difluorophenyl)amino]-5-{[2-methoxy-4-(piperazin-1-yl)phenyl]amino}pyrido[3,4-d]pyridazin-4(3H)-one

### Example 32A

### tert-butyl 4-(4-(7-(2,6-difluorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 31A, using 2,6-difluorobenzenamine in place of 3,5-dichlorobenzenamine. MS: 580 (M+H⁺).

### Example 32B

### 7-[(2,6-difluorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 20F, using EXAMPLE 32A in place of EXAMPLE 20E. ¹H NMR (DMSO-d₆, 300 MHz): δ 12.32 (s, 1H), 11.51 (s, 1H), 9.18 (s, 1H), 8.71 (brs, 2H), 8.07-7.90 (m, 2H), 7.31-7.26 (m, 3H), 6.67 (d, J=2.4 Hz, 1H), 6.20-6.15 (m, 2H), 3.87 (s, 3H), 3.28 (brs, 8H). MS: 480 (M+H⁺).

### Example 33

### 7-[(3,5-dichloropyridin-4-yl)amino]-5-{[2-methoxy-4-(piperazin-1-yl)phenyl]amino}pyrido[3,4-d]pyridazin-4(3H)-one

### Example 33A

### tert-butyl 4-(4-(7-(3,5-dichloropyridin-4-ylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 31A, using 3,5-dichloropyridin-4-amine in place of 3,5-dichlorobenzenamine. MS: 613 (M+H⁺).

### Example 33B

### 7-[(3,5-dichloropyridin-4-yl)amino]-5-{[2-methoxy-4-(piperazin-1-yl)phenyl]amino}pyrido[3,4-d]pyridazin-4(3H)-one

The title compound was obtained following the procedure described in EXAMPLE 20F, using EXAMPLE 33A in place of EXAMPLE 20E. ¹H NMR (DMSO-d₆, 300 MHz): δ 12.44 (s, 1H), 11.50 (s, 1H), 9.79 (s, 1H), 8.77 (brs, 4H), 8.13 (s, 1H), 7.68 (d, J=8.7 Hz, 1H), 6.68 (brs, 1H), 6.34 (s, 1H), 6.04 (d, J=8.7 Hz, 1H), 3.87 (s, 3H), 3.27 (brs, 8H). MS: 513 (M+H⁺).

### Example 34

### 7-[(2,4-dichlorophenyl)amino]-5-{[2-methoxy-4-(piperazin-1-yl)phenyl]amino}pyrido[3,4-d]pyridazin-4-ol

### Example 34A

### tert-butyl 14-(4-(7-(2,4-dichlorophenylamino)-4-oxo-3,4-dihydropyrido[3,4-d]pyridazin-5-ylamino)-3-methoxyphenyl)piperazine-1-carboxylate

A suspension of EXAMPLE 24A (100 mg, 0.21 mmol), 2,4-dichlorobenzenamine (33 mg, 0.21 mmol), tris(dibenzylideneacetone)dipalladium (19 mg, 0.021 mmol), 2-dicyclohexylphosphino-2′,4′,6′-triisopropylbiphenyl (10 mg, 0.021 mmol) and potassium 2-methylpropan-2-olate (71 mg, 0.63 mmol) in 2-methylpropan-2-ol (3 mL) was degassed with nitrogen 6 times and was heated in a sealed tube at 120° C. for 2 days. After cooling to ambient temperature, the mixture was concentrated and the residue was diluted with dichloromethane (25 mL) and water (25 mL). The aqueous phase was separated and extracted with dichloromethane (2×25 mL). The combined organic layer was washed with brine, dried over sodium sulfate, filtered, and concentrated. The residue was purified by flash chromatography on silica gel (200-300 mesh) eluting with a gradient of 75/1 to 70/1 dichloromethane/methanol to give the title compound. MS: 612 (M+H⁺).

### Example 34B

### 7-[(2,4-dichlorophenyl)amino]-5-{[2-methoxy-4-(piperazin-1-yl)phenyl]amino}pyrido[3,4-d]pyridazin-4-ol

To a solution of EXAMPLE 34A (55 mg, 0.107 mmol) in dichloromethane (6 mL) was added trifluoroacetic acid (3 mL) and the mixture was stirred at ambient temperature for 3 hours. The mixture was concentrated and the residue was purified by preparative HPLC using a gradient of 10/90 to 80/20 acetronitrile in water (containing 0.1% trifluoroacetic acid) to provide the title compound. ¹H NMR (DMSO-d₆, 300 MHz): δ 12.28 (s, 1H), 11.35 (s, 1H), 9.20 (s, 1H), 8.68 (brs, 2H), 8.01 (s, 1H), 7.92 (d, J=9 Hz, 1H), 7.71 (d, J=2.1 Hz, 1H), 7.63 (d, J=8.4 Hz, 1H), 7.42 (J=8.7 Hz, 2.4 Hz, 1H), 6.67 (d, J=2.7 Hz, 1H), 6.21 (s, 1H), 6.15 (dd, J=8.4 Hz, 1.8 Hz, 1H), 3.83 (s, 3H), 3.26 (m, 8H). MS: 512 (M+H⁺).

### Example 35

### 7-[(2,6-dichloro-4-fluorophenyl)amino]-5-{[2-methoxy-4-(piperazin-1-yl)phenyl]amino}pyrido[3,4-d]pyridazin-4-ol

### Example 35A

### tert-butyl 4-(4-(7-(2,6-dichloro-4-fluorophenylamino)-4-oxo-3,4-dihydropyrido[4,3-d]pyridazin-5-ylamino)-3-methoxyphenyl)piperazine-1-carboxylate

The title compound was obtained following the procedure described in EXAMPLE 34A, using 2,6-dichloro-4-fluoroaniline in place of 2,4-dichlorobenzenamine. MS: 630 (M+H⁺).

### Example 35B

### 7-[(2,6-dichloro-4-fluorophenyl)amino]-5-{[2-methoxy-4-(piperazin-1-yl)phenyl]amino}pyrido[3,4-d]pyridazin-4-ol

The title compound was obtained following the procedure described in EXAMPLE 34B, using EXAMPLE 35A in place of EXAMPLE 34A. ¹H NMR (CD₃OD, 300 MHz): δ 8.12 (d, J=8.4 Hz, 1H), 7.90 (d, J=6.3 Hz, 1H), 7.69 (m, 1H), 7.43 (d, J=8.4 Hz, 1H), 7.35 (dd, J=8.4 Hz, 2.7 Hz, 1H), 7.12 (m, 1H), 6.68 (d, J=8.7 Hz, 1H), 6.30 (s, 1H), 6.14 (s, 1H), 3.92 (s, 3H), 3.315 (m, 8H). MS: 529.4 (M+H⁺).

### Example 36

### 7-[(2-methoxyethyl)amino]-5-{[2-methoxy-4-(piperazin-1-yl)phenyl]amino}pyrido[3,4-d]pyridazin-4-ol

### Example 36A

### tert-butyl 4-(3-methoxy-4-(7-(2-methoxyethylamino)-4-oxo-3,4-dihydropyrido[3,4-d]pyridazin-5-ylamino)phenyl)piperazine-1-carboxylate

The title compound was obtained following the procedure described in EXAMPLE 34A, using 2-methoxyethanamine in place of 2,4-dichlorobenzenamine. MS: 526.6 (M+H⁺).

### Example 36B

### 7-[(2-methoxyethyl)amino]-5-{[2-methoxy-4-(piperazin-1-yl)phenyl]amino}pyrido[3,4-d]pyridazin-4-ol

The title compound was obtained following the procedure described in EXAMPLE 34B, using EXAMPLE 36A in place of EXAMPLE 34A. ¹H NMR (DMSO-d₆, 300 MHz): δ 12.04 (s, 1H), 11.50 (s, 1H), 8.57 (brs, 2H), 8.51 (d, J=8.7 Hz, 1H), 7.86 (s, 1H), 7.47 (brs, 1H), 0.72 (d, J=2.7, 1H), 6.49 (dd, J=6 Hz, 2.4 Hz, 1H), 5.90 (s, 1H), 3.87 (s, 3H), 3.51 (s, 3H), 3.30 (m, 5H), 3.24 (m, 7H). MS: 426.6 (M+H⁺).

### Example 37

### 7-[(2,3-difluorophenyl)amino]-5-{[2-methoxy-4-(piperazin-1-yl)phenyl]amino}pyrido[3,4-d]pyridazin-4(3H)-one

### Example 37A

### tert-butyl 4-(4-(7-(2,3-difluorophenylamino)-4-oxo-3,4-dihydropyrido[3,4-d]pyridazin-5-ylamino)-3-methoxyphenyl)piperazine-1-carboxylate

A suspension of EXAMPLE 24A (600 mg, 1.2 mmol), 2,3-difluorobenzenamine (191 mg, 1.5 mmol), tris(dibenzylidene acetone) dipalladium (110 mg, 0.12 mmol), 2-dicyclohexylphosphino-2′,4′,6′-triisopropylbiphenyl (114 mg, 0.24 mmol) and potassium tert-butoxide (276 mg, 2.5 mmol) in tert-butanol (50 mL) was heated in sealed tube at 120° C. under nitrogen for 16 hours. After concentration, the residue was purified by flash chromatography on silica gel (200-300 mesh) 50/1 eluting with dichloromethane/methanol to give the title compound. MS: 580 (M+He).

### Example 37B

### 7-[(2,3-difluorophenyl)amino]-5-{[2-methoxy-4-(piperazin-1-yl)phenyl]amino}pyrido[3,4-d]pyridazin-4(3H)-one

To a solution of EXAMPLE 37A (380 mg, 0.66 mmol) in dichloromethane (30 mL) was added trifluoroacetic acid (6 mL) and the mixture was stirred at ambient temperature for 2 hours. The mixture was concentrated and the residue was washed with 1/10 dichloromethane/hexane to give the title compound. ¹H NMR (DMSO-d₆, 300 MHz): δ 12.35 (s, 1H), 11.42 (s, 1H), 9.43 (s, 1H), 8.70 (brs, 2H), 8.07 (m, 2H), 7.50 (m, 1H), 7.19 (m, 2H), 6.70 (d, 1H), 6.25 (m, 2H), 3.90 (s, 3H), 3.28 (m, 8H). MS: 480 (M+H⁺).

### Example 38

### 7-[(furan-2-ylmethyl)amino]-5-{[2-methoxy-4-(piperazin-1-yl)phenyl]amino}pyrido[3,4-d]pyridazin-4-ol

### Example 38A

### tert-butyl 4-(4-(7-(furan-2-ylmethylamino)-4-oxo-3,4-dihydropyrido[3,4-d]pyridazin-5-ylamino)-3-methoxyphenyl)piperazine-1-carboxylate

A mixture of EXAMPLE 24A (52.6 mg, 0.109 mmol) and furan-3-ylmethanamine (21.1 mg, 0.218 mmol) in 1,4-dioxane (4 mL) was heated in a sealed tube at 120° C. for 15 hours. After cooling to ambient temperature, the mixture was concentrated and the residue was purified by flash chromatography on silica gel (20-300 mesh) eluting with 50/1 dichloromethane/methanol to give the title compound. MS: 548 (M+H⁺).

### Example 38B

### 7-[(furan-2-ylmethyl)amino]-5-{[2-methoxy-4-(piperazin-1-yl)phenyl]amino}pyrido[3,4-d]pyridazin-4-ol

The title compound was obtained following the procedure described in EXAMPLE 34B, using EXAMPLE 38A in place of EXAMPLE 34A. ¹H NMR (DMSO-d₆, 300 MHz): δ 12.07 (s, 1H), 11.47 (s, 1H), 8.74 (brs, 2H), 8.48 (d, J=2.1 Hz, 1H), 7.89 (m, 2H), 7.60 (m, 1H), 6.71 (d, J=1.8 Hz, 1H), (dd, J=5.7 Hz, 1.8 Hz, 1H), 6.39 (m, 1H), 6.27 (m, 1H), 5.95 (s, 1H), 4.54 (d, J=4.8 Hz, 2H), 3.87 (s, 3H), 3.32 (m, 4H), 3.25 (m, 4H). MS: 448 (M+H⁺).

### Example 39

### 5-{[2-methoxy-4-(piperazin-1-yl)phenyl]amino}-7-(piperidin-1-yl)pyrido[3,4-d]pyridazin-4(3H)-one

### Example 39A

### tert-butyl 4-(3-methoxy-4-(4-oxo-7-(piperidin-1-yl)-3,4-dihydropyrido[3,4-d]pyridazin-5-ylamino)phenyl)piperazine-1-carboxylate

A solution of EXAMPLE 24A (100 mg, 0.21 mmol) and piperidine (0.1 mL, 1 mmol) in dioxane (10 mL) was stirred at 120° C. for 13 hours. After cooling to ambient temperature, the mixture was concentrated to give the title compound which was used in the next step without further purification. MS: 536 (M+H⁺).

### Example 39B

### 5-{[2-methoxy-4-(piperazin-1-yl)phenyl]amino}-7-(piperidin-1-yl)pyrido[3,4-d]pyridazin-4(3H)-one

The title compound was obtained following the procedure described in EXAMPLE 26E, using EXAMPLE 39A in place of EXAMPLE 26D. ¹H NMR (DMSO-d₆, 300 MHz): δ 12.17 (s, 1H), 11.36 (s, 1H), 8.73 (brs, 2H), 8.35 (d, J=9.0 Hz, 1H), 8.14-8.08 (m, 2H), 8.01-7.87 (m, 1H), 7.39-7.35 (m, 1H), 7.22-7.18 (m, 1H), 6.75 (s, 1H), 6.42-6.38 (m, 2H), 3.90 (s, 3H), 3.68 (brs, 4H), 3.32 (brs, 8H), 1.68-1.59 (m, 6H). MS: 436 (M+H⁺).

### Example 40

### 7-(benzylamino)-5-{[2-methoxy-4-(piperazin-1-yl)phenyl]amino}pyrido[3,4-d]pyridazin-4(3H)-one

### Example 40A

### tert-butyl 4-(4-(7-(benzylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 24B, using benzylamine in place of 2-chlorobenzenamine. MS: 558 (M+H⁺).

### Example 40B

### 7-(benzylamino)-5-{[2-methoxy-4-(piperazin-1-yl)phenyl]amino}pyrido[3,4-d]pyridazin-4(3H)-one

The title compound was obtained following the procedure described in EXAMPLE 26E, using EXAMPLE 40A in place of EXAMPLE 26D. ¹H NMR (DMSO-d₆, 300 MHz): δ 12.07 (s, 1H), 11.46 (s, 1H), 8.69 (brs, 2H), 8.35 (brs, 1H), 7.98-7.91 (m, 2H), 7.38-7.26 (m, 5H), 6.71 (s, 1H), 6.43-6.40 (m, 1H), 5.98 (s, 1H), 4.59 (brs, 1H), 3.88 (s, 3H), 3.31-3.27 (m, 8H). MS: 458 (M+H⁺).

### Example 41

### 5-{[2-methoxy-4-(piperazin-1-yl)phenyl]amino}-7-phenylpyrido[3,4-d]pyridazin-4(3H)-one

### Example 41A

### tert-butyl tert-butyl 4-(3-methoxy-4-(4-oxo-7-phenyl-3,4-dihydropyrido[3,4-d]pyridazin-5-ylamino)phenyl)piperazine-1-carboxylate

To a solution of EXAMPLE 24A (120 mg, 0.25 mmol) in dioxane (8 mL) and water (2 mL) was added phenylboronic acid (36 mg, 0.30 mmol), 1,1′-bis(diphenylphosphino)ferrocene]dichloropalladium(II) (18 mg, 0.025 mmol) and potassium carbonate (102 mg, 0.71 mmol) and the mixture was stirred at 100° C. for 3 hours. After cooling to ambient temperature, the mixture was extracted with ethyl acetate (3×50 mL). The combined organic phase was dried over anhydrous sodium sulfate, filtered, concentrated and purified by flash chromatography on silica gel (200-300 mesh) 50/1 eluting with dichlomethane/methanol to give the title compound. MS: 529 (M+H⁺).

### Example 41B

### 5-{[2-methoxy-4-(piperazin-1-yl)phenyl]amino}-7-phenylpyrido[3,4-d]pyridazin-4(3H)-one

To a solution of EXAMPLE 41A (120 mg, 0.23 mmol) in dichloromethane (10 mL) was added 2,2,2-trifluoroacetic acid (4 mL) and the mixture was stirred at ambient temperature for 3 hours. After concentration, the residue was purified by preparative HPLC eluting with a gradient of 10/90 to 30/20 acetonitrile/water (containing 0.1% trifluoroacetic acid) to give the title compound as a solid trifluoroacetate salt. ¹H NMR (DMSO-d₆, 300 MHz): δ 12.9 (s, 1H), 11.5 (s, 1H), 8.72-8.69 (m, 3H), 8.30 (s, 1H), 8.14 (d, J=6.6 Hz, 2H), 7.60-7.55 (m, 4H), 6.79 (d, J=2.1 Hz, 1H), 6.67 (dd, J=9.0 Hz, 2.1 Hz, 1H), 3.93 (s, 3H), 3.37 (brs, 4H), 3.27 (brs, 4H). MS: 429.2 (M+H⁺).

### Example 42

### 7-(2,6-dichlorobenzyl)-5-{[2,6-difluoro-4-(piperazin-1-yl)phenyl]amino}pyrido[3,4-d]pyridazin-4(3H)-one

### Example 42A

### 4-bromo-2,6-difluorobenzenamine

2,6-Difluorobenzeneamine (6.0 g, 45 mmol) was dissolved in acetic acid (20 mL) and bromine (2.4 mL, 50 mmol) was added and the mixture was stirred at ambient temperature for 15 minutes. After concentration, the residue was treated with aqueous sodium carbonate and extracted with ethyl acetate. The organic extract was dried over sodium sulfate, filtered, and concentrated. The residue was purified by flash chromatography on silica gel (200-300 mesh) eluting with 25/1 petroleum ether/ethyl acetate to give the title compound. MS: 208 (M+H⁺).

### Example 42B

### N,N-dibenzyl-4-bromo-2,6-difluorobenzenamine

A mixture of EXAMPLE 42A (1.1 g, 5.3 mmol), benzyl bromide (949 mg, 0.66 ml) and potassium bicarbonate (1.46 mg, 10.6 mmol) in N,N-dimethylformamide (3 mL) was stirred at ambient temperature until TLC indicated no starting material remained. Ethyl acetate was added, and the mixture was washed with water and brine and dried over anhydrous sodium sulfate. After filtration and concentration of the filtrate, the residue was purified by flash chromatography on silica gel (200-300 mesh) eluting with 10/1 petroleum ether/dichoromethane to give the title compound.

### Example 42C

### tert-butyl 4-(4-(dibenzylamino)-3,5-difluorophenyl)piperazine-1-carboxylate

A mixture of EXAMPLE 42B (490 mg, 1.3 mmol), tert-butyl piperazine-1-carboxylate (258 mg, 1.4 mmol), palladium diacetate (14 mg, 0.06 mmol), (±)-2,2′-bis(diphenylphosphino)-1,1′-binaphthalene (59 mg, 0.09 mmol) and cesium carbonate (848 mg, 2.6 mmol) in 1,4-dioxane (20 mL) was heated at reflux for 16 hours. The mixture was filtered through diatomaceous earth and the filtrate was concentrated. The residue was purified by flash chromatography on silica gel (200-300 mesh) eluting with 8/1 petroleum ether/ethyl acetate to give the title compound.

### Example 42D

### tert-butyl 4-(4-amino-3,5-difluorophenyl)piperazine-1-carboxylate

A mixture of EXAMPLE 42C (560 mg, 1.1 mmol) and 10% palladium on carbon in methanol (15 mL) was stirred at ambient temperature under hydrogen until TLC indicated no starting material remained. The mixture was filtered through diatomaceous earth and the filtrate was concentrated. The residue was purified by flash chromatography on silica gel (200-300 mesh) eluting with 4/1 petroleum ether/ethyl acetate to give the title compound. MS: 314 (M+H⁺).

### Example 42E

### tert-butyl 4-(4-(7-chloro-4-oxo-3,4-dihydropyrido[3,4-d]pyridazin-5-ylamino)-3,5-difluorophenyl)piperazine-1-carboxylate

The title compound was obtained following the procedure described in EXAMPLE 26C, using EXAMPLE 42D in place of EXAMPLE 26B. MS: 493 (M+H⁺).

### Example 42F

### tert-butyl 4-(4-(7-(2,6-dichlorobenzyl)-4-oxo-3,4-dihydropyrido[3,4-d]pyridazin-5-ylamino)-3,5-difluorophenyl)piperazine-1-carboxylate

The title compound was obtained following the procedure described in EXAMPLE 26D, using EXAMPLE 42E in place of EXAMPLE 26C. MS: 617 (M+H⁺).

### Example 42G

### 7-(2,6-dichlorobenzyl)-5-{[2,6-difluoro-4-(piperazin-1-yl)phenyl]amino}pyrido[3,4-d]pyridazin-4(3H)-one

The title compound was obtained following the procedure described in EXAMPLE 26E, using EXAMPLE 42F in place of EXAMPLE 26D. ¹H NMR (DMSO-d₆, 300 MHz): δ 12.97 (s, 1H), 9.98 (s, 1H), 8.78 (brs, 2H), 8.25 (s, 1H), 7.43-7.34 (m, 3H), 6.74-6.68 (m, 3H), 4.25 (s, 2H), 3.44-3.27 (m, 8H). MS: 517 (M+H⁺).

### Example 43

### 5-{[2-methoxy-4-(piperazin-1-yl)phenyl]amino}-7-(pyridin-2-ylamino)pyrido[3,4-d]pyridazin-4(3H)-one

### Example 43A

### tert-butyl 4-(3-methoxy-4-(4-oxo-7-(pyridin-2-ylamino)-3,4-dihydropyrido[3,4-d]pyridazin-5-ylamino)phenyl)piperazine-1-carboxylate

The title compound was obtained following the procedure described in EXAMPLE 31A, using 2-aminopyridine in place of 3,5-dichloro benzenamine. MS: 545 (M+H⁺).

### Example 43B

### 5-{[2-methoxy-4-(piperazin-1-yl)phenyl]amino}-7-(pyridin-2-ylamino)pyrido[3,4-d]pyridazin-4(3H)-one

The title compound was obtained following the procedure described in EXAMPLE 20F, using EXAMPLE 43A in place of EXAMPLE 20E. ¹H NMR (DMSO-d₆, 300 MHz,): δ 12.46 (s, 1H), 11.37 (s, 1H), 10.07 (brs, 1H), 8.70 (brs, 2H), 8.45-8.28 (m, 2H), 8.11 (s, 1H), 7.72-7.58 (m, 2H), 7.18 (s, 1H), 7.03-6.98 (m, 1H), 6.76 (s, 1H), 6.57 (d, J=11.1 Hz, 1H), 3.89 (s, 3H), 3.35 (brs, 4H), 3.28 (brs, 4H). MS: 445 (M+H⁺).

### Example 44

### 5-{[2-methoxy-4-(piperazin-1-yl)phenyl]amino}-7-(pyridin-3-ylamino)pyrido[3,4-d]pyridazin-4(3H)-one

### Example 44A

### tert-butyl 4-(3-methoxy-4-(4-oxo-7-(pyridin-3-ylamino)-3,4-dihydropyrido[3,4-d]pyridazin-5-ylamino)phenyl)piperazine-1-carboxylate

The title compound was obtained following the procedure described in EXAMPLE 31A, using 3-aminopyridine in place of 3,5-dichloro benzenamine. MS: 545 (M+H⁺).

### Example 44B

### 5-{[2-methoxy-4-(piperazin-1-yl)phenyl]amino}-7-(pyridin-3-ylamino)pyrido[3,4-d]pyridazin-4(3H)-one

The title compound was obtained following the procedure described in EXAMPLE 2020F. using EXAMPLE 44A in place of EXAMPLE 20E. ¹H NMR (DMSO-d₆, 300 MHz,): δ 12.39 (s, 1H), 11.31 (s, 1H), 9.84 (s, 1H), 8.76-8.72 (m, 3H), 8.30-8.04 (m, 4H), 7.45 (brs, 1H), 6.74 (d, J=2.1 Hz, 1H), 6.49-6.46 (m, 1H), 6.28 (s, 1H), 3.87 (s, 3H), 3.35 (brs, 4H), 3.28 (brs, 4H). MS: 445 (M+H⁺).

### Example 45

### 7-{[2-(dimethylamino)ethyl]amino}-5-{[2-methoxy-4-(piperazin-1-yl)phenyl]amino}pyrido[3,4-d]pyridazin-4(3H)-one

### Example 45A

### tert-butyl 4-(4-(7-(2-(dimethylamino)ethylamino)-4-oxo-3,4-dihydropyrido[3,4-d]pyridazin-5-ylamino)-3-methoxyphenyl)piperazine-1-carboxylate

A solution of EXAMPLE 24A (120 mg, 0.25 mmol), N¹,N¹-dimethylethane-1,2-diamine (66 mg, 0.75 mmol) and N,N-diisopropylethylamine (0.5 mL) in 1,4-dioxane (10 mL) was heated in a sealed tube at 130° C. for 48 hours. After concentration, the residue was purified by flash chromatography on silica gel (200-300 mesh) eluting with 5/1 dichloromethane/methanol to give the title compound. MS: 539 (M+H⁺).

### Example 45B

### 7-{[2-(dimethylamino)ethyl]amino}-5-{[2-methoxy-4-(piperazin-1-yl)phenyl]amino}pyrido[3,4-d]pyridazin-4(3H)-one

The title compound was obtained following the procedure described in EXAMPLE 37B, using EXAMPLE 45A in place of EXAMPLE 37A. ¹H NMR (DMSO-d, 300 MHz): δ 12.19 (s, 1H), 11.49 (s, 1H), 9.61 (brs, 1H), 8.81 (brs, 2H), 8.42 (d, J=9 Hz, 1H), 7.97 (s, 1H), 7.51 (brs, 1H), 6.76 (d, J=2.1 Hz, 1H), 6.57 (dd, J=9 Hz, 2.1 Hz, 1H), 5.97 (s, 1H), 3.90 (s, 3H), 3.70 (m, 2H), 3.31 (m, 10H), 2.84 (s, 3H), 2.82 (s, 3H). MS: 439 (M+H⁺).

### Example 46

### 7-(cyclohexylamino)-5-{[2-methoxy-4-(piperazin-1-yl)phenyl]amino}pyrido[3,4-d]pyridazin-4(3H)-one

### Example 46A

### tert-butyl 4-(4-(7-(cyclohexylamino)-4-oxo-34-dihydropyrido[3,4-d]pyridazin-5-ylamino)-3-methoxyphenyl)piperazine-1-carboxylate

A solution of EXAMPLE 24A (120 mg, 0.25 mmol), cyclohexanamine (74 mg, 0.75 mmol) and N,N-diisopropylethylamine (0.5 mL) in 1,4-dioxane (10 mL) was heated in a sealed tube at 130° C. for 48 hours. The mixture was concentrated to provide the crude title compound which was used in the next reaction without further purification. MS: 550 (M+H⁺).

### Example 46B

### 7-(cyclohexylamino)-5-{[2-methoxy-4-(piperazin-1-yl)phenyl]amino}pyrido[3,4-d]pyridazin-4(3H)-one

The title compound was obtained as following the procedure described in EXAMPLE 37B, using EXAMPLE 46A in place of EXAMPLE 37A. ¹H NMR (DMSO-d₆, 300 MHz,): δ 12.00 (s, 1H), 11.50 (s, 1H), 8.70 (brs, 2H), 8.62 (d, J=9 Hz, 1H), 7.87 (s, 1H), 7.34 (brs, 1H), 6.75 (d, 1H), 6.52 (d, J=9 Hz, 1H), 5.88 (s, 1H), 3.91 (s, 3H), 3.78 (m, 1H), 3.31 (m, 8H), 1.21-2.04 (m, 10H). MS: 449 (M+H⁺).

### Example 47

### 5-{[2-methoxy-4-(piperazin-1-yl)phenyl]amino}-7-{[2-(pyridin-2-yl)ethyl]amino}pyrido[3,4-d]pyridazin-4-ol

### Example 47A

### tert-butyl 4-(3-methoxy-4-(4-oxo-7-(2-(pyridin-2-yl)ethylamino)-3,4-dihydropyrido[3,4-d]pyridazin-5-ylamino)phenyl)piperazine-1-carboxylate

The title compound was obtained following the procedure described in EXAMPLE 39A, using 2-(pyridin-2-yl)ethanamine in place of piperidine. MS: 573 (M+H⁺).

### Example 47B

### 5-{[2-methoxy-4-(piperazin-1-yl)phenyl]amino}-7-{[2-(pyridin-2-yl)ethyl]amino}pyrido[3,4-d]pyridazin-4-ol

The title compound was obtained following the procedure described in EXAMPLE 26E, using EXAMPLE 47A in place of EXAMPLE 26D. ¹H NMR (DMSO-d₆, 300 MHz): δ 12.08 (s, 1H), 11.41 (s, 1H), 8.78-8.54 (m, 4H), 8.06 (br, 1H), 7.91 (s, 1H), 7.57 (br, 2H), 6.76-6.75 (m, 1H), 6.48-6.44 (m, 1H), 5.90 (s, 1H), 3.91 (s, 3H), 3.78 (br, 2H), 3.32 (b, 8H), 3.21-3.17 (m, 2H). MS: 473 (M+H⁺).

### Example 48

### 7-[(2-fluorophenyl)amino]-5-{[2-methoxy-4-(piperazin-1-yl)phenyl]amino}pyrido[3,4-d]pyridazin-4(3H)-one

### Example 48A

### tert-butyl 4-(4-(7-(2-fluorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 10A, using 2-fluoroaniline in place of 2,6-dichlorobenzenamine.

### Example 48B

### 7-[(2-fluorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 2F, using EXAMPLE 48A in place of EXAMPLE 2E. ¹H NMR (DMSO-d, 300 MHz): δ 12.30 (s, 1H), 11.45 (s, 1H), 9.28 (s, 1H), 8.78 (s, 2H), 8.16 (d, J=9 Hz, 1H), 7.80 (s, 1H), 7.72-7.69 (m, 1H), 7.38-7.30 (m, 1H), 7.23-7.20 (m, 2H), 6.70 (s, 1H), 6.30-6.24 (m, 2H), 3.88 (s, 3H), 3.34-3.25 (m, 8H). MS: 462 (M+H⁺).

### Example 49

### 7-[(2,6-dimethylphenyl)amino]-5-{[2-methoxy-4-(piperazin-1-yl)phenyl]amino}pyrido[3,4-d]pyridazin-4(3H)-one

### Example 49A

### tert-butyl 4-(4-(7-(2,6-dimethylphenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 31A, using 2,6-dimethylaniline in place of 3,5-dichloro benzenamine. MS: 572 (M+H⁺).

### Example 49B

### 7-[(2,6-dimethylphenyl)amino]-5-{[2-methoxy-4-(piperazin-1-yl)phenyl]amino}pyrido[3,4-d]pyridazin-4(3H)-one

The title compound was obtained following the procedure described in EXAMPLE 20F, using EXAMPLE 49A in place of EXAMPLE 20E. ¹H NMR (DMSO-d₆, 300 MHz): δ 12.15 (s, 1H), 11.48 (s, 1H), 8.85-8.68 (m, 4H), 7.98 (s, 1H), 7.21 (brs, 4H), 6.67 (s, 1H), 3.87 (s, 3H), 3.38 (brs, 8H), 2.19 (s, 6H). MS: 472 (M+H⁺).

### Example 50

### 7-[(3-chloropyridin-2-yl)amino]-5-{[2-methoxy-4-(piperazin-1-yl)phenyl]amino}pyrido[3,4-d]pyridazin-4(3H)-one

### Example 50A

### tert-butyl 4-(4-(7-(3-chloropyridin-2-ylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 37A, using 3-chloropyridin-2-amine in place of 2,3-difluorobenzenamine. MS: 579 (M+H⁺).

### Example 50B

### 7-[(3-chloropyridin-2-yl)amino]-5-{[2-methoxy-4-(piperazin-1-yl)phenyl]amino}pyrido[3,4-d]pyridazin-4(3H)-one

The title compound was obtained following the procedure described in EXAMPLE 37B, using EXAMPLE 50A in place of EXAMPLE 37A. ¹H NMR (DMSO-d₆, 300 MHz): δ 12.55 (s, 1H), 11.55 (s, 1H), 9.04 (s, 1H), 8.70 (br, 2H), 8.48 (d, J=9 Hz, 1H), 8.42 (m, 1H), 8.16 (s, 1H), 8.05 (d, J=9 Hz, 1H), 7.23 (m, 1H), 7.13 (s, 1H), 6.73 (s, 1H), 6.43 (m, 1H), 3.91 (s, 3H), 3.34 (m, 8H). MS: 479 (M+H⁺).

### Example 51

### 7-[(2,3-dichlorophenyl)amino]-5-{[2-methoxy-4-(piperazin-1-yl)phenyl]amino}pyrido[3,4-d]pyridazin-4(3H)-one

### Example 51A

### tert-butyl 4-(4-(7-(2,3-dichlorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 37A, using 2,3-dichloroaniline in place of 2,3-difluorobenzenamine. MS: 612 (M+H⁺).

### Example 51B

### 7-[(2,3-dichlorophenyl)amino]-5-{[2-methoxy-4-(piperazin-1-yl)phenyl]amino}pyrido[3,4-d]pyridazin-4(3H)-one

The title compound was obtained following the procedure described in EXAMPLE 37B, using EXAMPLE 51A in place of EXAMPLE 37A. ¹H NMR (DMSO-d₆, 300 MHz): δ 12.35 (s, 1H), 11.48 (s, 1H), 9.34 (s, 1H), 8.70 (br, 2H), 8.06 (s, 1H), 8.02 (d, J=8.7 Hz, 1H), 7.66 (d, J=7.8 Hz, 1H), 7.54 (d, J=7.8 Hz, 1H), 7.42 (t. J=7.8 Hz, 1H), 6.70 (d, J=2.4 Hz, 1H), 6.29 (s, 1H), 6.23 (dd, J=8.7 Hz, 1H), 3.88 (s, 3H), 3.29 (m, 8H). MS: 512 (M+H⁺).

### Example 52

### 5-{[2-methoxy-4-(piperazin-1-yl)phenyl]amino}-7-[(2,4,6-trifluorophenyl)amino]pyrido[3,4-d]pyridazin-4(3H)-one

### Example 52A

### tert-butyl 4-(3-methoxy-4-(4-oxo-7-(2,4,6-trifluorophenylamino)-3,4-dihydropyrido-[3,4-d]pyridazin-5-ylamino)phenyl)piperazine-1-carboxylate

The title compound was obtained following the procedure described in EXAMPLE 24B, using in 2,4,6-trifluooaniline place of 2-chlorobenzenamine. MS: 598 (M+H⁺).

### Example 52B

### 5-{[2-methoxy-4-(piperazin-1-yl)phenyl]amino}-7-[(2,4,6-trifluorophenyl)amino]pyrido[3,4-d]pyridazin-4(3H)-one

The title compound was obtained following the procedure described in EXAMPLE 28B, using EXAMPLE 52A in place of EXAMPLE 28A. ¹H NMR (DMSO-d₆, 300 MHz): δ 8.00-7.97 (d, J=8.7 Hz, 1H), 7.92 (s, 1H), 7.05-6.99 (m, 2H), 6.67 (d, J=2.7 Hz, 1H), 6.21 (dd, J=9.0 Hz, 3.0 Hz, 1H), 6.13 (s, 1H), 3.92 (s, 3H), 3.36-3.34 (m, 8H). MS: 498 (M+H⁺).

### Example 53

### 5-{[2-methoxy-4-(piperazin-1-yl)phenyl]amino}-7-[(2,3,4-trifluorophenyl)amino]pyrido[3,4-d]pyridazin-4(3H)-one

### Example 53A

### tert-butyl 4-(3-methoxy-4-(4-oxo-7-(2,3,4-trifluorophenyl)amino)-3,4-dihydro pyrido[3,4-d]pyridazin-5-ylamino)phenyl)piperazine-1-carboxylate

The title compound was obtained following the procedure described in EXAMPLE 10A, using 2,3,4-trifluoroaniline in place of 2,6-dichlorobenzenamine.

### Example 53B

### 5-{[2-methoxy-4-(piperazin-1-yl)phenyl]amino}-7-[(2,3,4-trifluorophenyl)amino]pyrido[3,4-d]pyridazin-4(3H)-one

The title compound was obtained following the procedure described in 2F, using EXAMPLE 53A in place of EXAMPLE 2E. ¹H NMR (DMSO-d₆, 300 MHz): δ 12.36 (s, 1H), 11.42 (s, 1H), 9.41 (s, 1H), 8.66 (s, 2H), 8.07 (s, 1H), 7.98 (d, J=9 Hz, 1H), 7.46-7.30 (m, 2H), 6.72 (s, 1H), 6.25 (s, 2H), 3.89 (s, 3H), 3.39 (s, 8H). MS: 498 (M+H⁺).

### Example 54

### 7-[(3-chloro-2-fluorophenyl)amino]-5-{[2-methoxy-4-(piperazin-1-yl)phenyl]amino}pyrido[3,4-d]pyridazin-4-ol

### Example 54A

### tert-butyl 4-(4-(7-(3-chloro-2-fluorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 24B, using 2-fluoro-3-chloroaniline in place of 2-chlorobenzenamine. MS: 596 (M+H⁺).

### Example 54B

### 7-[(3-chloro-2-fluorophenyl)amino]-5-{[2-methoxy-4-(piperazin-1-yl)phenyl]amino}pyrido[3,4-d]pyridazin-4-ol

The title compound was obtained following the procedure described in EXAMPLE 26E, using EXAMPLE 54A in place of EXAMPLE 26D. ¹H NMR (DMSO-d₆, 300 MHz): δ 12.24 (s, 1H), 11.41 (s, 1H), 9.28 (s, 1H), 8.65 (brs, 2H), 8.05-8.03 (m, 2H), 7.69-7.64 (m, 1H), 7.38 (br, 1H), 7.24-7.18 (m, 1H), 6.71 (s, 1H), 6.30 (brs, 2H), 3.89 (s, 3H), 3.33 (brs, 8H). MS: 496 (M+H⁺).

### Example 55

### 5-{[2-methoxy-4-(piperazin-1-yl)phenyl]amino}-7-(tetrahydro-2H-pyran-4-ylamino)pyrido[3,4-d]pyridazin-4-ol

### Example 55A

### tert-butyl 4-(3-methoxy-4-(4-oxo-7-(tetrahydro-2H-pyran-4-ylamino)-3,4-dihydropyrido[3,4-d]pyridazin-5-ylamino)phenyl)piperazine-1-carboxylate

The title compound was obtained following the procedure described in EXAMPLE 34A, using tetrahydro-2H-pyran-4-amine in place of 2,4-dichlorobenzenamine. MS: 552 (M+H⁺).

### Example 55B

### 5-{[2-methoxy-4-(piperazin-1-yl)phenyl]amino}-7-(tetrahydro-2H-pyran-4-ylamino)pyrido[3,4-d]pyridazin-4-ol

The title compound was obtained following the procedure described in EXAMPLE 34B, using EXAMPLE 55A in place of EXAMPLE 34A. ¹H NMR (CD₃OD, 300 MHz): δ 8.63 (d, J=9 Hz, 1H), 7.82 (s, 1H), 6.77 (d, J=2.4 Hz, 1H), 6.63 (m, 1H), 5.91 (s, 1H), 3.68 (m, 6H), 3.61 (t, 2H), 3.43 (m, 8H), 2.10 (m, 2H), 2.06 (m, 2H). MS: 452 (M+H⁺).

### Example 56

### 5-{[2-methoxy-4-(piperazin-1-yl)phenyl]amino}-7-(1,3-thiazol-2-ylamino)pyrido[3,4-d]pyridazin-4(3H)-one

### Example 56A

### tert-butyl4-(3-methoxy-4-(4-oxo-7-(thiazol-2-ylamino)-3,4-dihydropyrido[3,4-d]pyridazin-5-ylamino)phenyl)piperazine-1-carboxylate

A mixture of EXAMPLE 24A (150 mg, 0.31 mmol), 2-aminothiazole (37 mg, 0.37 mmol), dimethylbisdiphenylphosphinoxanthene (36 mg, 0.62 mmol), tris(dibenzylideneacetone) dipalladium (28 mg, 0.03 mmol) and potassium tert-butoxide (69 mg, 0.68 mmol) in tert-butoxide (20 ml) was heated at 120° C. for 14 hours under nitrogen. After cooling to ambient temperature, the mixture was filtered and purified by flash chromatography on silica gel (200-300 mesh) eluting with 5/1 petroleum ether/ethyl acetate to provide the title compound. MS: 551 (M+H⁺).

### Example 56B

### 5-{[2-methoxy-4-(piperazin-1-yl)phenyl]amino}-7-(1,3-thiazol-2-ylamino)pyrido[3,4-d]pyridazin-4(3H)-one

To a solution of EXAMPLE 56A (80 mg, 0.15 mmol) in dichloromethane (20 ml) was added 2,2,2-trifluoroacetic acid (6 mL) and the mixture was stirred at ambient temperature for 6 hours. The mixture was concentrated and the residue purified by preparative HPLC using a gradient of 10/90 to 80/20 acetronitrile in water (containing 0.1% trifluoroacetic acid) to give the title compound. ¹H NMR (DMSO-d₆, 300 MHz): δ 12.52 (s, 1H), 11.48 (s, 1H), 11.06 (s, 1H), 8.72 (br, 1H), 8.13 (s, 1H), 7.93-7.90 (d, J=8.4 Hz, 1H), 7.41-7.40 (d, J=3.3 Hz, 1H), 6.96-6.95 (d, J=3.3 Hz, 1H), 6.77 (s, 1H), 6.63-6.60 (m, 2H), 3.84 (s, 3H), 3.40-3.31 (m, 8H). MS: 451 (M+H⁺)

### Example 57

### 5-{[2-chloro-4-(piperazin-1-yl)phenyl]amino}-7-(2,6-dichlorobenzyl)pyrido[3,4-d]pyridazin-4(3H)-one

### Example 57A

### N,N-bis(tert-butoxycarbonyl)-4-bromo-2-chloroaniline

A mixture of 4-bromo-2-chloroaniline (10 g, 48.4 mmol), di-tert-butyl dicarbonate (12.68 g, 58.12 mmol) and potassium carbonate (20.07 g, 145 mmol) in dimethylacetamide (300 mL) was stirred at ambient temperature for 24 hours. The mixture was poured into water and extracted with ethyl acetate (3×100 mL). The combined organic layers were dried over sodium sulfate, filtered, and concentrated and the residue was purified by flash chromatography on silica gel (200-300 mesh) eluting with 10/1 petroleum ether/ethyl acetate to give the title compound. MS: 428 (M+Na⁺).

### Example 57B

### tert-butyl 4-(4-(bis(tert-butoxy carbonyl)amino)-3-chlorophenyl)piperazine-1-carboxylate

A mixture of EXAMPLE 57A (280 g, 0.9 mmol), tert-butyl piperazine-1-carboxylate (205 mg, 1.09 mmol), palladium acetate (21 mg, 0.092 mmol), (±)-2,2′-bis(diphenylphosphino)-1,1′-binaphthalene (86 mg, 0.14 mmol) and cesium carbonate (900 mg, 2.76 mmol) in toluene (10 mL) was heated under nitrogen at 100° C. for 16 hours. After cooling to ambient temperature, the mixture was concentrated. The residue was diluted with dichloromethane (300 mL), washed with water and brine, dried over sodium sulfate, filtered and concentrated. The residue was purified by flash chromatography on silica gel (200-300 mesh) eluting with 5/1 petroleum ether/ethyl acetate to give the title compound.

### Example 57C

### 2-chloro-4-(piperazin-1-yl)aniline

To a solution of EXAMPLE 57B (320 mg, 0.63 mmol) in dichloromethane (4 mL) was added trifluoroacetic acid (1 mL) dropwise and the mixture was stirred at ambient temperature for 4 hours. The mixture was concentrated and the crude title compound was used in the next step without further purification. MS: 212 (M+H⁺).

### Example 57D

tert-butyl 4-(4-amino-3-chlorophenyl)piperazine-1-carboxylate

A mixture of EXAMPLE 57C (133 mg, 0.63 mmol), di-tert-butyl dicarbonate (173 mg, 0.79 mmol) and potassium carbonate (546 mg, 3.95 mmol) in dimethylacetamide (5 mL) was stirred at ambient temperature for 1 hour. The mixture was poured into water and extracted with ethyl acetate (3×50 mL). The combined organic layers were dried over sodium sulfate, filtered, and concentrated and the residue was purified by flash chromatography on silica gel (200-300 mesh) eluting with 3/1 petroleum ether/ethyl acetate to give the title compound. MS: 312 (M+H⁺).

### Example 57E

### tert-butyl 4-(3-chloro-4-(7-chloro-4-oxo-3,4-dihydropyrido[3,4-d]pyridazin-5-ylamino)phenyl)piperazine-1-carboxylate

A mixture of EXAMPLE 57D (95 mg, 0.3 mmol), EXAMPLE 7K (66 mg, 0.3 mmol) and N,N-diisopropylethylamine (79 mg, 0.6 mmol) in 1,4-dioxane (2 mL) was heated in a sealed tube at 120° C. for 18 hours. The mixture was concentrated and the residue was purified by flash chromatography on silica gel (200-300 mesh) eluting with 3/1 petroleum ether/ethyl acetate to give the title compound. MS: 491 (M+H⁺).

### Example 57F

### tert-butyl 4-(3-chloro-4-(7-(2,6-dichlorobenzyl)-4-oxo-3,4-dihydropyrido[3,4-d]pyridazin-5-ylamino)phenyl)piperazine-1-carboxylate

To a mixture of EXAMPLE 57E (147 mg, 0.3 mmol) and tetrakis(triphenylphosphine) palladium (40 mg, 0.03 mmol) was added 0.5M 2,6-dichlorobenzyl zinc bromide in tetrahydrofuran (4 mL, 2 mmol) under nitrogen and the mixture was heated at 60° C. for 16 hours. After cooling to ambient temperature, the mixture was diluted with dichloromethane (50 mL) and washed with saturated aqueous sodium bicarbonate. The organic phase was separated and the aqueous phase was extracted with dichloromethane (2×20 mL). The combined organic phase was washed with brine, dried over sodium sulfate, filtered and concentrated. The residue was purified by flash chromatography on silica gel (200-300 mesh) eluting with 98/2 dichloromethane/methanol to give the title compound. MS: 615 (M+H⁺).

### Example 57G

### 5-{[2-chloro-4-(piperazin-1-yl)phenyl]amino}-7-(2,6-dichlorobenzyl)pyrido[3,4-d]pyridazin-4(3H)-one

The title compound was obtained following the procedure described in EXAMPLE 28B, using EXAMPLE 57F in place of EXAMPLE 28A. ¹H NMR (DMSO-d₆, 300 MHz): δ 13.02 (s, 1H), 11.58 (s, 1H), 8.80 (br, 2H), 8.31 (s, 1H), 8.16 (d, J=9.3 Hz, 1H), 7.61 (d, J=8.1 Hz, 2H), 7.47-7.41 (m, 1H), 7.13 (d, J=2.4 Hz, 1H), 6.99 (s, 1H), 6.70 (dd, J=9.0 Hz, 2.4 Hz, 1H), 4.50 (s, 3H), 3.36-3.28 (m, 8H). MS: 515 (M+H⁺).

### Example 58

### 5-{[2-methoxy-4-(piperazin-1-yl)phenyl]amino}-7-[(1-methylpiperdin-4-yl)amino]pyrido[3,4-d]pyridazin-4(3H)-one

### Example 58A

### tert-butyl 4-(3-methoxy-4-(7-(1-methylpiperidin-4-ylamino)-4-oxo-3,4-dihydropyrido[3,4-d]pyridazin-5-ylamino)phenyl)piperazine-1-carboxylate

The title compound was obtained following the procedure described in EXAMPLE 37A, using 1-methylpiperidin-4-amine in place of 2,3-difluorobenzenamine. MS: 565 (M+H⁺).

### Example 58B

### 5-{[2-methoxy-4-(piperazin-1-yl)phenyl]amino}-7-[(1-methylpiperidin-4-yl)amino]pyrido[3,4-d]pyridazin-4(3H)-one

The title compound was obtained following the procedure described in EXAMPLE 37B, using EXAMPLE 58A in place of EXAMPLE 37A. ¹H NMR (DMSO-d₆, 300 MHz): δ 12.12 (s, 1H), 11.55 (s, 1H), 9.47 (br, 1H), 8.79 (br, 2H), 8.53 (m, 1H), 7.92 (s, 1H), 7.52 (br, 1H), 6.77 (d, 1H), 6.65 (d, J=9 Hz, 1H), 5.92 (s, 1H), 3.91 (s, 3H), 3.57 (d, 2H), 3.32 (m, 8H), 3.12 (q, 2H), 2.85 (m, 3H), 2.28 (d, 2H), 2.06 (m, 1H), 1.70 (q, 2H). MS: 465 (M+H⁺).

### Example 59

### 7-(2,6-dichlorobenzyl)-5-{[3-fluoro-2-methoxy-4-(piperazin-1-yl)phenyl]amino}pyrido[3,4-d]pyridazin-4-ol

### Example 59A

### 3-fluoro-2-methoxy-4-(piperazin-1-yl)aniline

The title compound was obtained following the procedure described in EXAMPLE 1F and 1G using 1,2-difluoro-3-methoxy-4-nitrobenzene in place of 4-fluoro-2-methoxy-1-nitrobenzene.

### Example 59B

### tert-butyl 4-(4-(7-chloro-4-oxo-3,4-dihydropyrido[3,4-d]pyridazin-5-ylamino)-2-fluoro-3-methoxyphenyl)piperazine-1-carboxylate

The title compound was obtained following the procedure described in EXAMPLE 24A, using EXAMPLE 59A in place of EXAMPLE 1G. MS: 505 (M+H⁺).

### Example 59C

### tert-butyl 4-(4-(7-(2,6-dichlorobenzyl)-4-oxo-3,4-dihydropyrido[3,4-d]pyridazin-5-ylamino)-2-fluoro-3-methoxyphenyl)piperazine-1-carboxylate

The title compound was obtained following the procedure described in EXAMPLE 26D, using EXAMPLE 59B in place of EXAMPLE 26C. MS: 630 (M+H⁺).

### Example 59D

### 7-(2,6-dichlorobenzyl)-5-{[3-fluoro-2-methoxy-4-(piperazin-1-yl)phenyl]amino}pyrido[3,4-d]pyridazin-4-ol

The title compound was obtained following the procedure described in EXAMPLE 34B, using EXAMPLE 59C in place of EXAMPLE 34A. ¹H NMR (DMSO-d₆, 300 MHz): δ 13.03 (s, 1H), 11.79 (s, 1H), 7.89 (br, 2H), 8.31 (s, 1H), 7.78 (d, J=9.3 Hz, 1H), 7.62 (d, J=8.1 Hz, 1H), 7.45 (m, 1H), 6.98 (s, 1H), 6.52 (m, 1H), 4.53 (s, 2H), 3.95 (s, 3H), 3.30 (m, 4H), 3.19 (m, 4H). MS: 529.1 (M+H⁺).

### Example 60

### 7-[(3-chloropyridin-4-yl)amino]-5-{[2-methoxy-4-(piperazin-1-yl)phenyl]amino}pyrido[3,4-d]pyridazin-4(3H)-one

### Example 60A

### tert-butyl 4-(4-(7-(3-chloropyridin-4-ylamino)-4-oxo-3,4-dihydropyrido[3,4-d]pyridazin-5-ylamino)-3-methoxyphenyl)piperazine-1-carboxylate

A mixture of EXAMPLE 24A (160 mg, 0.33 mmol), 3-chloropyridin-4-amine (65 mg, 0.45 mmol), tris(dibenzylideneacetone)dipalladium (30 mg, 0.03 mmol), dimethylbisdiphenyl phosphinoxanthene (20 mg, 0.03 mmol), potassium tert-butoxide (112 mg, 1 mmol) and tert-butanol (2 mL) was bubbled with nitrogen and heated at 100° C. for 18 hours. The mixture was concentrated and the residue was dissolved in dichloromethane, washed with saturated aqueous sodium bicarbonate and brine, dried over sodium sulfate, filtered and concentrated. The residue was purified by flash chromatography on silica gel (200-300 mesh) eluting with 98/2 dichloromethane/methanol to give the title compound. MS: 579 (M+H⁺).

### Example 60B

### 7-[(3-chloropyridin-4-yl)amino]-5-{[2-methoxy-4-(piperazin-1-yl)phenyl]amino}pyrido[3,4-d]pyridazin-4(3H)-one

To a solution of EXAMPLE 60A (100 mg, 0.16 mmol) in dichloromethane (10 mL) was added 2,2,2-trifluoroacetic acid (2 mL) dropwise. The solution was stirred at ambient temperature for 12 hours, concentrated and purified by preparative HPLC using a gradient of 10/90 to 80/20 acetronitrile in water (containing 0.1% trifluoroacetic acid) to give the title compound. ¹H NMR (DMSO-d₆, 300 MHz): δ 12.62 (s, 1H), 11.34 (s, 1H), 8.46 (s, 1H), 8.73 (br, 2H), 8.64 (s, 1H), 8.33-8.31 (m, 1H), 8.16-8.13 (m, 2H), 8.04-8.01 (m, 2H), 6.76-6.74 (m, 2H), 6.48-6.45 (m, 1H), 3.89 (s, 3H), 3.36-3.29 (m, 8H). MS: 479 (M+

### Example 61

### 5-{[2-bromo-4-(piperazin-1-yl)phenyl]amino}-7-(2,6-dichlorobenzyl)pyrido[3,4-d]pyridazin-4(3H)-one

### Example 61A

### 3-fluoro-2-methoxy-4-(piperazin-1-yl)aniline

The title compound was obtained following the procedure described in EXAMPLE 1F and 1G using 2-bromo-4-fluoro-1-nitrobenzene in place of 4-fluoro-2-methoxy-1-nitrobenzene.

### Example 61B

### tert-butyl 4-(3-bromo-4-(7-chloro-4-oxo-3,4-dihydropyrido[3,4-d]pyridazin-5-ylamino)phenyl)piperazine-1-carboxylate

The title compound was obtained following the procedure described in EXAMPLE 24A, using EXAMPLE 61A in place of EXAMPLE 1G.

### Example 61C

### tert-butyl 4-(3-bromo-4-(7-(2,6-dichlorobenzyl)-4-oxo-3,4-dihydropyrido[3,4-d]pyridazin-5-ylamino)phenyl)piperazine-1-carboxylate

The title compound was obtained following the procedure described in EXAMPLE 2E, using EXAMPLE 61B in place of EXAMPLE 2D.

### Example 61D

### 5-{[2-bromo-4-(piperazin-1-yl)phenyl]amino}-7-(2,6-dichlorobenzyl)pyrido[3,4-d]pyridazin-4(3H)-one

The title compound was obtained following the procedure described in EXAMPLE 2F, using EXAMPLE 61C in place of EXAMPLE 2E. ¹H NMR (DMSO-d₆, 300 MHz): δ 12.96 (s, 1H), 11.40 (s, 1H), 8.69 (s, 1H), 8.26 (s, 1H), 8.05 (d, J=9 Hz, 1H), 7.56 (d, J=7.8 Hz, 2H), 7.40 (m, 1H), 7.22 (s, 1H), 6.93 (s, 1H), 6.70 (dd, J=2.7 Hz, 9 Hz, 1H), 4.5 (s, 2H), 3.31-3.20 (m, 8H). MS: 561 (M+H⁺).

### Example 62

### 7-(2-chlorophenoxy)-5-{[2-methoxy-4-(piperazin-1-yl)phenyl]amino}pyrido[3,4-d]pyridazin-4(3H)-one

### Example 62A

### tert-butyl 4-(4-(7-(2-chlorophenoxy)-4-oxo-3,4-dihydropyrido[3,4-d]pyridazin-5-ylamino)-3-methoxyphenyl)piperazine-1-carboxylate

A mixture of EXAMPLE 24A (0.1 g, 0.21 mmol), 2-chlorophenol (53 mg, 0.42 mmol), cuprous iodide (4 mg, 0.02 mmol), 2-(dimethylamino)acetic acid 6 mg, 0.06 mmol) and cesium carbonate (0.14 g, 0.42 mmol) in 1,4-dioxane (2 mL) was heated in a sealed tube at 120° C. overnight. After concentration, the residue was purified by flash chromatography on silica gel eluting with 1/100 methanol/dichloromethane to give the title compound.

### Example 62B

### 7-(2-chlorophenoxy)-5-{[2-methoxy-4-(piperazin-1-yl)phenyl]amino}pyrido[3,4-d]pyridazin-4(3H)-one

The title compound was obtained following the procedure described in EXAMPLE 2F, using EXAMPLE 62A in place of EXAMPLE 2E. ¹H NMR (DMSO-d₆, 300 MHz): δ 12.74 (s, 1H), 11.54 (s, 1H), 8.56 (s, 2H), 8.21 (s, 1H), 7.67 (d, J=8.1 Hz, 1H), 7.50-7.41 (m, 4H), 6.64-6.60 (m, 2H), 5.95 (d, J=9 Hz, 1H), 3.84 (s, 3H), 3.24 (s, 8H). MS: 480(M+H⁺).

### Example 63

### 7-(2,6-dichlorophenoxy)-5-{[2-methoxy-4-(piperazin-1-yl)phenyl]amino}pyrido[3,4-d]pyridazin-4(3H)-one

### Example 63A

### tert-butyl 4-(4-(7-(2,6-dichlorophenoxy)-4-oxo-3,4-dihydropyrido[3,4-d]pyridazin-5-ylamino)-3-methoxyphenyl)piperazine-1-carboxylate

The title compound was obtained following the procedure described in EXAMPLE 76A, using 2,6-dichlorophenol in place of 2-fluorophenol.

### Example 63B

### 7-(2,6-dichlorophenoxy)-5-{[2-methoxy-4-(piperazin-1-yl)phenyl]amino}pyrido[3,4-d]pyridazin-4(3H)-one

The title compound was obtained following the procedure described in EXAMPLE 2F. using EXAMPLE 63A in place of EXAMPLE 2E. ¹H NMR (DMSO-d₆, 300 MHz): δ 12.78 (s, 1H), 11.55 (s, 1H), 8.63 (s, 2H), 8.23 (s, 1H), 7.69 (d, J=8.1 Hz, 2H), 7.45 (t, J=8.4 Hz, 1H), 7.29 (d, J=9 Hz, 1H), 6.71 (s, 1H), 6.64 (s, 1H), 5.95 (d, J=9 Hz, 1H), 3.84 (s, 3H), 3.25 (s, 8H). MS: 514 (M+H⁺).

### Example 64

### 7-(2,6-dichlorobenzyl)-5-{[4-(piperazin-1-yl)-2-(trifluoromethoxy)phenyl]amino}pyrido[3,4-d]pyridazin-4-ol

### Example 64A

### N,N-dibenzyl-4-bromo-2-(trifluoromethoxy)aniline

To a mixture of 4-bromo-2-(trifluoromethoxy)aniline (5 g, 19.53 mmol) and potassium carbonate (8.09 g, 58.89 mmol) in acetonitrile (200 mL) was added (bromomethyl)benzene (6.96 mL, 58.59 mmol) and the mixture was refluxed for 20 hours. The mixture was filtered and the filtrate was concentrated. The residue was purified by flash chromatography on silica gel (200-300 mesh) eluting with 5/1 petroleum ether/ethyl acetate to give the title compound. MS: 436 (M+H⁺).

### Example 64B

### tert-butyl 4-(4-(dibenzylamino)-3-(trifluoromethoxy)phenyl)piperazine-1-carboxylate

A mixture of EXAMPLE 64A (7.92 g, 18.21 mmol), tert-butyl piperazine-1-carboxylate (3.73 g, 20 mmol), palladium diacetate (205 mg, 0.91 mmol), (±)-2,2′-bis(diphenylphosphino)-1,1′-binaphthalene (850 mg, 1.36 mmol) and cesium carbonate (11.88 g, 36 mmol) in toluene (200 mL) was heated under nitrogen at 100° C. for 20 hours. After cooling to ambient temperature, the mixture was concentrated and the residue was diluted with dichloromethane (300 mL), washed with water and brine, dried over sodium sulfate, filtered and concentrated. The residue was purified by flash chromatography on silica gel (200-300 mesh) eluting with 10/1 petroleum ether/ethyl acetate to give the title compound. MS: 542 (M+H⁺).

### Example 64C

### tert-butyl 4-(4-amino-3-(trifluoromethoxy)phenyl)piperazine-1-carboxylate

To a solution of EXAMPLE 64B (8.98 g, 16.63 mmol) in methanol (100 mL) was added 10% palladium on carbon (900 mg) and the mixture was stirred at ambient temperature under hydrogen for 5 hours. The catalyst was filtered off and the filtrate was concentrated to give the title compound. MS: 362 (M+H⁺).

### Example 64D

### tert-butyl 4-(4-(7-chloro-4-oxo-3,4-dihydropyrido[3,4-d]pyridazin-5-ylamino)-3-(trifluoromethoxy)phenyl)piperazine-1-carboxylate

The title compound was obtained following the procedure described in EXAMPLE 57E, using EXAMPLE 64C in place of EXAMPLE 57D. MS: 541 (M+H⁺).

### Example 64E

### tert-butyl 4-(4-(7-(2,6-dichlorobenzyl)-4-oxo-3,4-dihydropyrido[3,4-d]pyridazin-5-ylamino)-3-(trifluoromethoxy)phenyl)piperazine-1-carboxylate

The title compound was obtained following the procedure described in EXAMPLE 57F, using EXAMPLE 64D in place of EXAMPLE 57E. MS: 665 (M+H⁺).

### Example 64F

### 7-(2,6-dichlorobenzyl)-5-{[4-(piperazin-1-yl)-2-(trifluoromethoxy)phenyl]amino}pyrido[3,4-d]pyridazin-4-ol

The title compound was obtained following the procedure described in EXAMPLE 28B, using EXAMPLE 64E in place of EXAMPLE 28A. ¹H NMR (DMSO-d₆, 300 MHz): δ 13.09 (s, 1H), 11.74 (s, 1H), 8.77 (br, 2H), 8.31 (s, 1H), 8.21 (d, J=9.0 Hz, 1H), 7.62 (d, J=7.8 Hz, 2H), 7.48-7.42 (m, 1H), 7.03-7.00 (m, 2H), 6.73 (dd, J=9.0 Hz, 2.4 Hz, 1H), 4.52 (s, 2H), 3.33-3.19 (m, 8H). MS: 565 (M+H⁺).

### Example 65

### 7-[(2,3-dichloro-6-fluorophenyl)amino]-5-{[2-methoxy-4-(piperazin-1-yl)iphenyl]amino}pyrido[3,4-d]pyridazin-4(3H)-one

### Example 65A

### tert-butyl 4-(4-((7-((2,3-dichloro-6-fluorophenyl)amino)-4-oxo-3,4-dihydropyrido[3,4-d]pyridazin-5-yl)amino)-3-methoxyphenyl)piperazine-1-carboxylate

To a solution of EXAMPLE 24A (1.05 g, 2.16 mmol), 2,3-dichloro-6-fluoroaniline (0.76 g, 4.4 mmol) and tert-butanol (10 mL) was added tris(dibenzyldeneacetone)dipalladium (0) (0.2 g, 10 mmol %), 2-dicyclohexylphosphino-2′,4′,6′-triisopropylbiphenyl (0.1 g, 10 mmol %) and potassium tert-butoxide (0.6 g, 5.3 mmol). The mixture was degassed twice and heated in a sealed tube under nitrogen at 120° C. for 20 hours. After concentration, the residue was purified by flash column eluting with 3/1 petroleum ether/ethyl acetate to give the title compound. MS: 630 (M+H⁺).

### Example 65B

### 7-[(2,3-dichloro-6-fluorophenyl)amino]-5-{[2-methoxy-4-(piperazin-1-yl)phenyl]amino}pyrido[3,4-d]pyridazin-4(3H)-one

To a solution of EXAMPLE 65A (80 mg, 0.127 mmol) in dichloromethane (15 mL) was added trifluoroacetic acid (3 mL) and the mixture was stirred at room temperature overnight. Concentration provided the title compound. ¹H NMR (DMSO-d₆, 300 MHz): δ 12.03 (s, 1H), 11.42 (s, 1H), 9.38 (s, 1H), 8.70 (s, 2H), 8.04 (s, 1H), 7.72-7.68 (m, 2H), 7.51-7.45 (m, 1H), 6.64 (d, J=2.7 Hz, 1H), 6.20 (s, 1H), 6.08 (d, J=8.7 Hz, 1H), 4.02 (s, 3H), 3.25 (s, 8H). MS: 531 (M+H⁺).

### Example 66

### 7-(2,6-dichlorobenzyl)-5-{[4-(hexahydropyrrolo[1,2-a]pyrazin-2(1H)-yl)-2-methoxyphenyl]amino}pyrido[3,4-d]pyridazin-4(3H)-one

### Example 66A

### 2-(3-methoxy-4-nitrophenyl)octahydropyrrolo[1,2-a]pyrazine

A mixture of 4-fluoro-2-methoxy-1-nitrobenzene (342 mg, 2 mmol), octahydropyrrolo[1,2-a]pyrazine (252 mg, 2 mmol) and potassium carbonate (552 mg, 4 mmol) in dimethylacetamide (10 mL) was heated at 100° C. for 16 hours. After cooling to ambient temperature, the mixture was poured into water and extracted with ethyl acetate (3×50 mL). The combined organic phase was washed with brine, dried over sodium sulfate, filtered and concentrated. The residue was purified by flash chromatography on silica gel (200-300 mesh) eluting with 98/2 dichloromethane/methanol to give the title compound. MS: 278 (M+H⁺).

### Example 66B

### 4-(hexahydropyrrolo[1,2-a]pyrazin-2(1H)-yl)-2-methoxyaniline

To a suspension of EXAMPLE 66A (1.58 g, 5.68 mmol) in methanol (100 mL) was added Raney-Ni (158 mg) and the mixture was stirred at ambient temperature under hydrogen for 4 hours. The catalyst was filtered off and the filtrate was concentrated to give the title compound, which was used in the next step without further purification. MS: 248 (M+H⁺).

### Example 66C

### 7-chloro-5-((4-(hexahydropyrrolo[1,2-a]pyrazin-2(1H)-yl)-2-methoxyphenyl)amino)pyrido[3,4-d]pyridazin-4(3H)-one

To mixture of EXAMPLE 7K (150 mg, 0.7 mmol) and EXAMPLE 66B (173 mg, 0.7 mmol) in 1,4-dioxane (7 mL) was added N-ethyl-N-isopropylpropan-2-amine (270 mg, 2.1 mmol) and the mixture was heated in a sealed tube at 100° C. for 15 hours. After concentration, the residue was purified by flash chromatography on silica gel eluting with 3/1 petroleum ether/ethyl acetate to give the title compound. MS: 427 (M+H⁺).

### Example 66D

### 7-(2,6-dichlorobenzyl)-5-{[4-(hexahydropyrrolo[1,2-a]pyrazin-2(1H)-yl)-2-methoxyphenyl]amino}pyrido[3,4-d]pyridazin-4(3H)-one

Zinc powder (2.06 g, 31.5 mmol) in tetrahydrofuran (2 mL) was heated under nitrogen to 65° C., followed by addition of 1,2-dibromoethane (131 mg, 1.2 mmol) and chlorotrimethylsilane (226 mg, 1.2 mmol). After stirring for 0.5 hour, a solution of 2-(bromomethyl)-1,3-dichlorobenzene (7.2 g, 30 mmol) in tetrahydrofuran (28 mL) was added over 20 minutes and the mixture was stirred for 2 hours and then allowed to cool to room temperature to provide 30 mL of 1N (2,6-dichlorobenzyl)zinc(II) bromide solution. To a solution of EXAMPLE 66C (230 mg, 0.54 mmol) in tetrahydrofuran (20 mL) under nitrogen was added tetrakis(triphenylphospine) palladium (30 mg, 0.027 mmol). The mixture was degassed twice with nitrogen and 1N (2,6-dichlorobenzyl)zinc(II) bromide (5.4 mL, 5.4 mmol) was added and the mixture was stirred at 60° C. for overnight. After concentration, the residue was purified by flash chromatography on silica gel eluting with 3/1 petroleum ether/ethyl acetate to give the title compound. ¹H NMR (DMSO, 300 MHz): δ 12.59 (s, 1H), 11.34 (s, 1H), 8.15 (s, 1H), 8.00 (d, J=9.0 Hz, 1H), 7.55 (d, J=7.8 Hz, 1H), 7.43-7.38 (m, 1H), 6.79 (s, 1H), 6.64 (s, 1H), 6.27 (dd, J=9.90 Hz, 2.4 Hz, 1H), 4.49 (s, 2H), 3.93 (s, 3H), 3.73-3.55 (m, 2H), 3.14-3.10 (m, 2H), 4.49 (t, 1H), 3.51-3.24 (m, 4H), 1.91-1.80 (m, 3H), 1.53-1.50 (m, 1H). MS: 553 (M+H⁺).

### Example 67

### 7-[(2,3-dichloro-4-fluorophenyl)amino]-5-{[2-methoxy-4-(piperazin-1-yl)phenyl]amino}pyrido[3,4-d]pyridazin-4-ol

### Example 67A

### tert-butyl 4-(4-(7-(2,3-dichloro-4-fluorophenylamino)-4-oxo-3,4-dihydropyrido[3,4-d]pyridazin-5-ylamino)-3-methoxyphenyl)piperazine-1-carboxylate

A mixture of EXAMPLE 24A (162 mg, 0.33 mmol), 2,3-dichloro-4-fluorobenzenamine (90 mg, 0.50 mmol), tris(dibenzylideneacetone)dipalladium (31 mg, 0.03 mmol), 4,5-bis(diphenylphosphino)-9,9-dimethylxanthenexanthene (20 mg, 0.03 mmol) and potassium 2-methylpropan-2-olate (112 mg, 1 mmol) in 2-methylpropan-2-ol (2 mL) was degassed with nitrogen twice and stirred in a sealed tube at 130° C. for 15 hours. After cooling to ambient temperature, the solution was concentrated and the residue was purified by flash chromatography on silica gel eluting with 100/1 dichloromethane/methanol to give the title compound. MS: 630 (M+H⁺).

### Example 67B

### 7-[(2,3-dichloro-4-fluorophenyl)amino]-5-{[2-methoxy-4-(piperazin-1-yl)phenyl]amino}pyrido[3,4-d]pyridazin-4-ol

The title compound was obtained following the procedure described in EXAMPLE 26E, using EXAMPLE 67A in place of EXAMPLE 26D. ¹H NMR (DMSO-d₆, 300 MHz): δ 12.34 (s, 1H), 11.44 (s, 1H), 9.97 (s, 1H), 8.71 (br, 2H), 8.06 (s, 1H), 7.93-7.90 (m, 1H), 7.68-7.51 (m, 2H), 6.70 (s, 1H), 6.23-6.16 (m, 2H), 3.67 (s, 3H), 3.35 (br, 8H). MS: 530 (M+H⁺).

### Example 68

### 2-[(2,6-dichlorophenyl)amino]-4-{[2-methoxy-4-(piperazin-1-yl)phenyl]amino}pyrido[2,3-d]pyridazin-5(6H)-one

### Example 68A

### ethyl 4,6-dihydroxy-2-methylnicotinate

A mixture of 2,4,6-trichlorophenol (175 g, 886 mmol), malonic acid (57.6 g, 554 mmol) and phosphorus oxychloride (160 mL, 1.77 mol) was heated at 100° C. for 3 hours. After cooling to ambient temperature and concentration, the residue was poured into ice-water and filtered. The solid was washed with water and dried under vacuum. The solid was suspended in bromobenzene (400 mL) and ethyl 3-aminocrotonate (62.9 g, 487 mmol) and the mixture was heated at 155° C. for 1.5 hours. The mixture was concentrated and washed with 2/1 petroleum ether/ethyl acetate to give the title compound. MS: 198 (M+H⁺).

### Example 68B

### ethyl 4,6-dichloro-2-methylnicotinate

A mixture of EXAMPLE 68A (87.3 g, 443 mmol) and phosphorus oxychloride (300 mL) was refluxed at 140° C. for 2.5 hours. After cooling to ambient temperature and concentration, the residue was poured into ice-water and extracted with ethyl acetate (2×300 mL). The organic phase was dried over sodium sulfate, concentrated and purified by flash chromatography on silica gel (200-300 mesh) eluting with a gradient of 100/1 to 20/1 petroleum ether/ethyl acetate to give the title compound. MS: 234 (M+H⁺).

### Example 68C

### ethyl 2-(bromomethyl)-4,6-dichloronicotinate

To a solution of EXAMPLE 68B (30.0 g, 128 mmol) in tetrachloromethane (500 mL) was added N-bromosuccinimide (68.4 g, 384 mmol) and benzoyl peroxide (9.29 g, 38.4 mmol) and the mixture was heated at 85° C. for 1 day. The mixture was cooled to ambient temperature and filtered and the filtrate was concentrated. The residue was dissolved in ethyl acetate (400 mL), washed with brine (400 mL), dried over anhydrous sodium sulfate, filtered, concentrated and purified by flash chromatography on silica gel (200-300 mesh) eluting with 80/1 petroleum ether/ethyl acetate to give the title compound. MS: 314 (M+H⁺).

### Example 68D

### ethyl 4,6-dichloro-2-formylnicotinate

To a suspension of 4 Å molecular sieves (30 g) in acetonitrile (300 mL) was added 1-methylpiperidine 1-oxide (20.6 g, 176 mmol) and the mixture was stirred at ambient temperature for 10 minutes. A solution of EXAMPLE 68C (25.0 g, 79.9 mmol) in acetonitrile (100 mL) was added and the mixture was stirred at ambient temperature for 1 hour. The mixture was filtered and the filtrate was concentrated and purified by flash chromatography on silica gel (200-300 mesh) eluting with a gradient of 100/1 to 80/1 petroleum ether/ethyl acetate to give the title compound. MS: 248 (M+H⁺).

### Example 68E

### ethyl 2-((2-(tert-butoxy carbonyl)hydrazono)methyl)-4,6-dichloronicotinate

To a solution of EXAMPLE 68D (11.7 g, 47.2 mmol) in dioxane (200 mL) was added tert-butyl hydrazinecarboxylate (7.47 g, 56.6 mmol) and the mixture was stirred at ambient temperature for 15 hours. After concentration, the residue was purified by flash chromatography on silica gel (200-300 mesh) eluting with a gradient of 30/1 to 4/1 petroleum ether/ethyl acetate to give the title compound. MS: 362 (M+He).

### Example 68F

### 2,4-dichloropyrido[2,3-d]pyridazin-5(6H)-one

To a solution of EXAMPLE 68E (10.6 g, 29.3 mmol) in dichloromethane (120 mL) was added trifluoroacetic acid (20 mL) and the mixture was stirred at ambient temperature for 5 hours. After concentration, the residue was washed with 1/1 petroleum ether/ethyl acetate. The filtrate was concentrated and purified by flash chromatography on silica gel (200-300 mesh) eluting with 10/1 petroleum ether/ethyl acetate to give the title compound. MS: 216 (M+H⁺).

### Example 68G

### tert-butyl 4-(4-((2-chloro-5-oxo-5,6-dihydropyrido[2,3-d]pyridazin-4-yl)amino)-3-methoxyphenyl)piperazine-1-carboxylate

A mixture of EXAMPLE 68F (0.350 g, 1.62 mmol), EXAMPLE 1G (0.596 g, 1.94 mmol), N,N-diisopropylethylamine (0.418 g, 3.24 mmol) and 1,4-dioxane (8 mL) was heated in a sealed tube at 120° C. for 3 hours. After cooling to ambient temperature, the mixture was concentrated. The residue was washed with 20/1 petroleum ether/ethyl acetate and purified by flash chromatography on silica gel (200-300 mesh) eluting with 50/1 dichloromethane/methanol to give the title compound. MS: 487 (M+H⁺).

### Example 68H

### tert-butyl 4-(4-((2-((2,6-dichlorophenyl)amino)-5-oxo-5,6-dihydropyrido[2,3-d]pyridazin-4-yl)amino)-3-methoxyphenyl)piperazine-1-carboxylate

A mixture of EXAMPLE 68G (60.0 mg, 0.123 mmol), 2,6-dichloroaniline (24.0 mg, 0.148 mmol), tris(dibenzylideneacetone)dipalladium (11.3 mg, 0.0123 mmol), 2-dicyclohexylphosphino-2′,4′,6′-triisopropylbiphenyl (11.7 mg, 0.0246 mmol), potassium tert-butoxide (27.6 mg, 0.246 mmol) and n-butanol (3 mL) was degassed with nitrogen and heated in a sealed tube at 110° C. for 15 hours. After cooling to ambient temperature, the mixture was concentrated and the residue was dissolved in dichloromethane, filtered and purified by preparative TLC using 2/3 petroleum ether/ethyl acetate to give the title compound. MS: 612 (M+H⁺).

### Example 68I

### 2-[(2,6-dichlorophenyl)amino]-4-{[2-methoxy-4-(piperazin-1-yl)phenyl]amino}pyrido[2,3-d]pyridazin-5(6H)-one

To a solution of EXAMPLE 68H (29.4 mg, 0.048 mmol) in dichloromethane (10 mL) was added trifluoroacetic acid (4 mL) and the mixture was stirred at ambient temperature for 2 hours. After concentration, the residue was washed with 20/1 petroleum ether/dichloromethane to give the title compound as a solid trifluoroacetate salt. ¹H NMR (DMSO-d₆, 300 MHz,): δ 12.57 (s, 1H), 10.40 (s, 1H), 9.10 (s, 1H), 8.77 (s, 2H), 7.79 (s, 1H), 7.56 (d, J=8.4 Hz, 2H), 7.37-7.26 (m, 2H), 6.78 (s, 1H), 6.61 (d, J=8.7 Hz, 1H), 6.06 (s, 1H), 3.83 (s, 3H), 3.42-3.38 (m, 4H), 3.28 (br, 4H). MS: 512 (M+H⁺).

### Example 69

### 7-(2,6-dichlorobenzyl)-5-{[2,3-dimethyl-4-(piperazin-1-yl)phenyl]amino}pyrido[3,4-d]pyridazin-4(3H)-one

### Example 69A

### N,N-dibenzyl-4-bromo-2,3-dimethylaniline

The title compound was obtained following the procedure described in EXAMPLE 64A, using 4-bromo-2,3-dimethylaniline in place of 4-bromo-2-(trifluoromethoxy)aniline. MS: 380 (M+H⁺).

### Example 69B

### tert-butyl 4-(4-(dibenzylamino)-2,3-dimethylphenyl)piperazine-1-carboxylate

The title compound was obtained following the procedure described in EXAMPLE 64B, using EXAMPLE 69A in place of EXAMPLE 64A. MS: 486 (M+H⁺).

### Example 69C

### tert-butyl 4-(4-amino-2,3-dimethylphenyl)piperazine-1-carboxylate

The title compound was obtained following the procedure described in EXAMPLE 64C, using EXAMPLE 69B in place of EXAMPLE 64B. MS: 306 (M+H⁺).

### Example 69D

### tert-butyl 4-(4-(7-chloro-4-oxo-34-dihydropyrido[3,4-d]pyridazin-5-ylamino)-2,3-dimethylphenyl)piperazine-1-carboxylate

The title compound was obtained following the procedure described in EXAMPLE 57E, using EXAMPLE 69C in place of EXAMPLE 57D. MS: 485 (M+H⁺).

### Example 69E

### tert-butyl 4-(4-(7-(2,6-dichlorobenzyl)-4-oxo-3,4-dihydropyrido[3,4-d]pyridazin-5-ylamino-2,3-dimethylphenyl)piperazine-1-carboxylate

The title compound was obtained following the procedure described in EXAMPLE 57F, using EXAMPLE 69D in place of EXAMPLE 57E. MS: 609 (M+H⁺).

### Example 69F

### 7-(2,6-dichlorobenzyl)-5-{[2,3-dimethyl-4-(piperazin-1-yl)phenyl]amino}pyrido[3,4-d]pyridazin-4(3H)-one

The title compound was obtained following the procedure described in EXAMPLE 28B, using EXAMPLE 69E in place of EXAMPLE 28A. ¹H NMR (DMSO-d₆, 300 MHz): δ 12.99 (s, 1H), 11.11 (s, 1H), 8.77 (brs, 2H), 8.27 (s, 1H), 7.79 (d, J=8.7 Hz, 1H), 7.54 (d. J=7.8 Hz, 2H), 7.39 (dd, J=8.7 Hz, 7.2 Hz, 1H), 6.82 (s, 1H), 6.73 (d, J=8.7 Hz, 1H), 4.42 (s, 2H), 3.30 (br, 4H), 3.01 (brs, 4H), 2.24 (s, 3H), 2.18 (s, 3H). MS: 509 (M+H⁺).

### Example 70

### 2-(2,6-dichlorobenzyl)-4-{[2-methoxy-4-(piperazin-1-yl)phenyl]amino}pyrido[2,3-d]pyridazin-5(6H)-one

### Example 70A

### tert-butyl 4-(4-((2-(2,6-dichlorobenzyl)-5-oxo-5,6-dihydropyrido[2,3-d]pyridazin-4-yl)amino)-3-methoxyphenyl)piperazine-1-carboxylate

A mixture of EXAMPLE 68G (50.0 mg, 0.103 mmol), 0.5M (2,6-dichlorobenzyl)zinc(II) bromide in tetrahydrofuran (2.1 mL, 1.05 mmol), tetrakis(triphenylphosphine)palladium (11.9 mg, 0.0103 mmol) and tetrahydrofuran (3 mL) was degassed with nitrogen and heated in a sealed tube at 80° C. for 15 hours. After cooling to ambient temperature, the reaction was quenched with saturated aqueous ammonium chloride (10 mL). The mixture was extracted with tetrahydrofuran (3×20 mL) and the organic phase was dried over anhydrous sodium sulfate, concentrated and purified by preparative TLC using 3/4 petroleum ether/ethyl acetate to give the title compound. MS: 611 (M+H⁺).

### Example 70B

### 2-(2,6-dichlorobenzyl)-4-{[2-methoxy-4-(piperazin-1-yl)phenyl]amino}pyrido[2,3-d]pyridazin-5(6H)-one

To a solution of EXAMPLE 70A (30.9 mg, 0.0505 mmol) in dichloromethane (6 mL) was added trifluoroacetic acid (3 mL) and the mixture was stirred at ambient temperature for 2 hours. After concentration, the residue was washed with 10/1 petroleum ether/dichloromethane to give the title compound as a solid trifluoroacetate salt. ¹H NMR (DMSO-d₆, 300 MHz): δ 12.86 (s, 1H), 10.59 (s, 1H), 8.74 (s, 2H), 8.10 (s, 1H), 7.51 (d, J=8.1 Hz, 2H), 7.38-7.33 (m, 1H), 7.15 (d, J=8.7 Hz, 1H), 6.70 (s, 1H), 6.55 (d, J=8.4 Hz, 1H), 6.34 (s, 1H), 4.37 (s, 2H), 3.75 (s, 3H), 3.42-3.38 (m, 4H), 3.28 (m, 4H). MS: 511 (M+H⁺).

### Example 71

### 7-[(2-chloro-4,6-difluorophenyl)amino]-5-{[2-methoxy-4-(piperazin-1-yl)phenyl]amino}pyrido[3,4-d]pyridazin-4-ol

### Example 71A

### tert-butyl 4-(4-(7-(2-chloro-4,6-difluorophenylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 67A, using 2-chloro-4,6-difluoroaniline in place of 2,3-dichloro-4-fluorobenzenamine. MS: 614(M+H⁺).

### Example 71B

### 7-[(2-chloro-4,6-difluorophenyl)amino]-5-{[2-methoxy-4-(piperazin-1-yl)phenyl]amino}pyrido[3,4-d]pyridazin-4-ol

The title compound was obtained following the procedure described in EXAMPLE 26E, using EXAMPLE 71A in place of EXAMPLE 26D. ¹H NMR (DMSO-d₆, 300 MHz): δ 12.32 (s, 1H), 11.45 (s, 1H), 9.19 (s, 1H), 8.75 (brs, 2H), 8.06 (s, 1H), 7.80 (brs, 1H), 7.60-7.53 (m, 2H), 6.69 (s, 1H), 6.18-6.10 (m, 2H), 3.87 (s, 3H), 3.35 (brs, 8H). MS: 514(M+H⁺).

### Example 72

### 7-(2,6-dichlorobenzyl)-5-{[2-methoxy-5-methyl-4-(piperazin-1-yl)phenyl]amino}pyrido[3,4-d]pyridazin-4(3H)-one

### Example 72A

### 4-bromo-2-methoxy-5-methylbenzenamine

To a solution of 2-methoxy-5-methylbenzenamine (5 g, 36.5 mmol) in N,N-dimethylformamide (150 mL) at ambient temperature was added dropwise N-bromobutanimide (6.5 g, 36.5 mmol). After stirring for 16 hours, the mixture was diluted with water (150 mL) and extracted with ethyl acetate (3×100 mL). The organic layers were dried over anhydrous sodium sulfate, filtered, concentrated and purified by flash chromatography on silica gel (200-300 mesh) eluting with 10/1 petroleum ether/ethyl acetate to give the title compound. MS: 216 (M+H⁺).

### Example 72B

### N,N-dibenzyl-4-bromo-2-methoxy-5-methylbenzenamine

A suspension of EXAMPLE 72A (3 g, 14 mmol), (bromo methyl)benzene (6 g, 35 mmol) and potassium carbonate (4.8 g, 35 mmol) in acetonitrile (150 mL) was heated at reflux for 20 hours. After concentration, the residue was diluted with ethyl acetate (100 mL) and washed with water (30 mL). The organic layer was dried over anhydrous sodium sulfate, filtered, concentrated and purified by flash chromatography on silica gel (200-300 mesh) eluting with 50/1 petroleum ether/ethyl acetate to give the title compound. MS: 396 (M+H⁺).

### Example 72C

### tert-butyl 4-(4-(dibenzylamino)-5-methoxy-2-methylphenyl)piperazine-1-carboxylate

A suspension of EXAMPLE 72B (2 g, 5.1 mmol), tert-butyl piperazine-1-carboxylate (1.1 g, 6.1 mmol), palladium(II) acetate (115 mg, 0.5 mmol), 2,2′-bis(diphenylphosphino)-1,1′-binaphthyl (635 mg, 1 mmol) and cesium carbonate (3.3 g, 10.1 mmol) in toluene (100 mL) was heated to reflux under nitrogen for 16 hours. After concentration, the residue was purified by flash chromatography on silica gel (200-300 mesh) eluting with 10/1 petroleum ether/ethyl acetate to give the title compound. MS: 502 (M+H⁺).

### Example 72D

### tert-butyl 4-(4-amino-5-methoxy-2-methylphenyl)piperazine-1-carboxylate

A suspension of EXAMPLE 72C (1.6 g, 3.2 mmol) and 10% palladium on carbon (160 mg) in methanol (100 mL) was stirred under hydrogen for 18 hours. The catalyst was filtered off and the filtrate was concentrated to give the title compound which was used in the next step without further purification. MS: 322 (M+H⁺).

### Example 72E

### tert-butyl 4-(4-(7-chloro-4-oxo-3,4-dihydropyrido[3,4-d]pyridazin-5-ylamino)-5-methoxy-2-methylphenyl)piperazine-1-carboxylate

A solution of EXAMPLE 7K (200 mg, 0.93 mmol), EXAMPLE 72D (360 mg, 1.12 mmol) and N,N-diisopropylethylamine (240 mg, 1.86 mmol) in 1,4-dioxane (20 mL) was heated in a sealed tube at 120° C. for 18 hours and cooled to ambient temperature. The precipitated solid was filtered and washed with hexane (20 mL) to give the title compound. MS: 501 (M+H⁺).

### Example 72F

### tert-butyl 4-(4-(7-(2,6-dichlorobenzyl)-4-oxo-3,4-dihydropyrido[3,4-d]pyridazin-5-ylamino)-5-methoxy-2-methylphenyl)piperazine-1-carboxylate

A suspension of EXAMPLE 72E (200 mg, 0.4 mmol), 0.5M (2,6-dichlorobenzyl)zinc(II) bromide in tetrahydrofuran (8 mL, 4 mmol) and tetrakis(triphenylphosphine) palladium(0) (46 mg, 0.04 mmol) in tetrahydrofuran (30 mL) was heated at 70° C. under nitrogen for 16 hours. After cooling, the mixture was diluted with saturated ammonium chloride (50 mL) and extracted with dichloromethane (3×20 mL). The organic layers were dried over anhydrous sodium sulfate, filtered, concentrated and purified by flash chromatography on silica gel (200-300 mesh) eluting with 3/1 petroleum ether/ethyl acetate to give the title compound. MS: 625 (M+H⁺).

### Example 72G

### 7-(2,6-dichlorobenzyl)-5-{[2-methoxy-5-methyl-4-(piperazin-1-yl)phenyl]amino}pyrido[3,4-d]pyridazin-4(3H)-one

A solution of EXAMPLE 72F (200 mg, 0.32 mmol) and trifluoroacetic acid (6 mL) in dichloromethane (30 mL) was stirred at ambient temperature for 3 hours. After concentration, the residue was washed with methanol (3×5 mL) to give the title compound. ¹H NMR (DMSO-d₆, 300 MHz): δ. 12.92 (s, 1H), 11.65 (s, 1H), 8.70 (s, 2H), 8.26 (s, 1H), 8.21 (s, 1H), 7.55 (d, 2H), 7.38 (t, 1H), 6.91 (s, 1H), 6.71 (s, 1H), 4.49 (s, 2H), 3.81 (s, 3H), 3.27 (m, 4H), 3.03 (m, 4H), 2.14 (s, 3H). MS: 525 (M+H⁺).

### Example 73

### 7-(2,6-dichlorobenzyl)-5-{[2-fluoro-5-methyl-4-(piperazin-1-yl)phenyl]amino}pyrido[3,4-d]pyridazin-4(3H)-one

### Example 73A

### N,N-dibenzyl-4-bromo-2-fluoro-5-methylbenzenamine

A suspension of 4-bromo-2-fluoro-5-methylbenzenamine (1 g, 4.9 mmol), benzylbromide (2.5 g, 14.7 mmol) and potassium carbonate (2 g, 14.7 mmol) in acetonitrile (80 mL) was heated in a sealed tube at 100° C. for 16 hours. After concentration, the residue was diluted with ethyl acetate (80 mL) and washed with water (20 mL). The organic layer was dried over anhydrous sodium sulfate, filtered, concentrated and purified by flash chromatography on silica gel (200-300 mesh) eluting with 50/1 petroleum ether/ethyl acetate to give the title compound. MS: 384 (M+H⁺).

### Example 73B

### tert-butyl 4-(4-(dibenzylamino)-5-methoxy-2-methylphenyl)piperazine-1-carboxylate

A suspension of EXAMPLE 73A (3.2 g, 8.4 mmol), tert-butyl piperazine-1-carboxylate (1.9 g, 10.1 mmol), palladium(II) acetate (189 mg, 0.84 mmol), 2,2′-bis(diphenylphosphino)-1,1′-binaphthyl (1.05 g, 1.7 mmol) and cesium carbonate (5.5 g, 16.8 mmol) in toluene (200 mL) was heated at reflux under nitrogen for 16 hours. After concentration, the residue was purified by flash chromatography on silica gel (200-300 mesh) eluting with 10/1 petroleum ether/ethyl acetate to give the title compound. MS: 490 (M+<f.

### Example 73C

### tert-butyl 4-(4-amino-5-fluoro-2-methylphenyl)piperazine-1-carboxylate

A suspension of EXAMPLE 73B (2 g, 4.1 mmol) and 10% palladium on carbon (200 mg) in methanol (100 mL) was stirred under hydrogen for 18 hours. The catalyst was filtered off and the filtrate was concentrated and purified by flash chromatography on silica gel (200-300 mesh) eluting with 1/1 petroleum ether/ethyl acetate to give the title compound. MS: 310 (M+H⁺).

### Example 73D

### tert-butyl 4-(4-(7-chloro-4-oxo-3,4-dihydropyrido[3,4-d]pyridazin-5-ylamino)-5-fluoro-2-methylphenyl)piperazine-1-carboxylate

A solution of EXAMPLE 7K (200 mg, 0.93 mmol), EXAMPLE 73C (346 mg, 1.12 mmol) and N,N-diisopropylethylamine (240 mg, 1.86 mmol) in 1,4-dioxane (20 mL) was heated in a sealed tube at 120° C. for 18 hours and cooled to ambient temperature. After concentration, the residue was purified by flash chromatography on silica gel (200-300 mesh) eluting with 5/1 petroleum ether/ethyl acetate to give the title compound. MS: 489 (M+H⁺).

### Example 73E

### tert-butyl 4-(4-(7-(2,6-dichlorobenzyl)-4-oxo-3,4-dihydropyrido[3,4-d]pyridazin-5-ylamino)-5-fluoro-2-methylphenyl)piperazine-1-carboxylate

A suspension of EXAMPLE 73D (180 mg, 0.4 mmol), 0.5M (2,6-dichlorobenzyl)zinc(II) bromide in tetrahydrofuran (6 mL, 3 mmol) and tetrakis(triphenylphosphine) palladium(0) (43 mg, 0.04 mmol) in tetrahydrofuran (30 mL) was heated at 70° C. under nitrogen for 16 hours. After cooling, the mixture was diluted with saturated ammonium chloride (50 mL) and extracted with dichloromethane (3×20 mL). The organic layers were dried over anhydrous sodium sulfate, filtered, concentrated and purified by flash chromatography on silica gel (200-300 mesh) eluting with 5/1 petroleum ether/ethyl acetate to give the title compound. MS: 613 (M+H⁺).

### Example 73F

### 7-(2,6-dichlorobenzyl)-5-{[2-fluoro-5-methyl-4-(piperazin-1-yl)phenyl]amino}pyrido[3,4-d]pyridazin-4(3H)-one

A solution of EXAMPLE 73E (170 mg, 0.28 mmol) and trifluoroacetic acid (5 mL) in dichloromethane (25 mL) was stirred at ambient temperature for 2 hours. After concentration, the residue was washed with methanol (3×5 mL) to give the title compound. ¹H NMR (DMSO-d₆, 300 MHz): δ 13.01 (s, 1H), 11.57 (s, 1H), 8.69 (s, 2H), 8.33 (s, 1H), 8.20 (d, 1H), 7.55 (d, 2H), 7.37 (t, 1H), 7.05 (d, 2H), 7.00 (s, 1H), 4.50 (s, 2H), 3.26 (m, 4H), 3.01 (m, 4H), 2.18 (s, 3H). MS: 513 (M+H⁺).

### Example 74

### 6-(2,6-dichlorobenzyl)-8-[(6-methoxy-1,2,3,4-tetrahydroisoquinolin-7-yl)amino]phthalazin-1(2H)-one

### Example 74A

### methyl 3-methoxyphenethylcarbamate

To a solution of methyl carbonochloridate (25 g, 265 mmol) in dichloromethane (100 mL) at 0° C. was added slowly methyl 3-methoxyphenethylcarbamate (40 g, 265 mmol) and the mixture was stirred for 0.5 hours at 0° C. and at ambient temperature for 16 hours. The mixture was poured into ice-brine (200 mL) and extracted with dichloromethane (3×100 mL). The combined organic layers were dried over anhydrous sodium sulfate, filtered and concentrated. The residue was washed with hexane (50 mL) and the filtrate was concentrated to give the crude title compound. MS: 210 (M+H⁺).

### Example 74B

### 6-methoxy-3,4-dihydroisoquinolin-1(2H)-one

To polyphosphoric acid (70 mL) at 120° C. was added slowly EXAMPLE 74A (10 g, 47.8 mmol) and the mixture was stirred at 120° C. for 1 hour. After cooling, the mixture was poured into ice-water (300 mL) and extracted with dichloromethane (2×200 mL). The combined organic layers were washed with brine (100 mL), dried over anhydrous sodium sulfate, filtered and concentrated to give the title compound. MS: 178 (M+H⁺).

### Example 74C

### 6-methoxy-1,2,3,4-tetrahydroisoquinoline

To a suspension of lithium aluminum hydride (10 g, 46 mmol) in tetrahydrofuran (100 mL) at 0° C. under nitrogen was added slowly a solution of EXAMPLE 74B (4.1 g, 23 mmol) in tetrahydrofuran (50 mL) over 0.5 hours and the mixture was heated at 70° C. for 2 hours. After cooling to 0° C., 15% sodium hydroxide (4.9 mL) was added slowly and the mixture was filtered and washed with ethyl acetate (50 mL). The filtrate was concentrated to give the crude title compound. MS: 164 (M+H⁺).

### Example 74D

### tert-butyl 6-methoxy-3,4-dihydroisoquinoline-2(1H)-carboxylate

To a solution of EXAMPLE 74C (1.88 g, 11.5 mmol) in dichloromethane (40 mL) was added triethylamine (2.3 g, 23 mmol) and di-tert-butyl dicarbonate (3 g, 13.8 mmol). After stirring for 16 hours, the mixture was poured into water (50 mL) and extracted with dichloromethane (2×100 mL). The combined organic layers were dried over anhydrous sodium sulfate, filtered, concentrated and purified by flash chromatography on silica gel eluting with 10:1 hexane:ethyl acetate to give the title compound. MS: 264 (M+H⁺).

### Example 74E

### tert-butyl 6-methoxy-7-nitro-3,4-dihydroisoquinoline-2(1H)-carboxylate

To a solution of EXAMPLE 74D (2.46 g, 9.35 mmol) in nitromethane (30 mL) at −10° C. was added acetic anhydride (5.7 g, 56.1 mmol) and concentrated nitric acid (0.88 g, 14 mmol). After stirring for 3 hours, the mixture was adjusted to pH 7 with aqueous sodium bicarbonate solution and extracted with dichloromethane (2×100 mL). The combined organic layers were dried over anhydrous sodium sulfate, filtered, concentrated and purified by flash chromatography on silica gel eluting with 5:1 hexane:ethyl acetate to give the title compound. MS: 309 (M+H⁺).

### Example 74F

### tert-butyl 7-amino-6-methoxy-3,4-dihydroisoquinoline-2(1H)-carboxylate

To a solution of EXAMPLE 74E (550 mg, 1.78 mmol) in methanol (10 mL) was added Raney Nickel (55 mg) and the mixture was stirred at ambient temperature under hydrogen for 16 hours. The mixture was filtered, concentrated and dried under vacuum to give the crude title compound. MS: 279 (M+H⁺).

### Example 74G

### tert-butyl 7-(7-chloro-4-oxo-3,4-dihydropyrido[3,4-d]pyridazin-5-ylamino)-6-methoxy-3,4-dihydroisoquinoline-2(1H)-carboxylate

To a solution of EXAMPLE 7K (200 mg, 0.93 mmol) in dioxane (10 mL) was added EXAMPLE 74F (284 mg, 1.02 mmol) and N,N-diisopropylethylamine (600 mg, 4.65 mmol), and the mixture was stirred in a sealed tube at 120° C. for 16 hours. After cooling to ambient temperature, the mixture was poured into water (50 mL) and extracted with dichloromethane (100 mL). The combined organic layers were dried over anhydrous sodium sulfate, filtered, concentrated and purified by flash chromatography on silica gel eluting with 100:1 dichlomethane/methanol to give the title compound. MS: 458 (M+W).

### Example 74H

### tert-butyl 7-(7-(2,6-dichlorobenzyl)-4-oxo-3,4-dihydropyrido[3,4-d]pyridazin-5-ylamino)-6-methoxy-3,4-dihydroisoquinoline-2(1H)-carboxylate

To a solution of EXAMPLE 74G (200 mg, 0.44 mmol) in tetrahydrofuran (10 mL) was added 0.5M (2,6-dichlorobenzyl)zinc(II) bromide in tetrahydrofuran (8.8 mL, 4.4 mmol) and tetrakis(triphenylphosphine)palladium (46 mg, 0.04 mmol). After stirring at 70° C. under nitrogen overnight, the mixture was cooled to ambient temperature, poured into water (0 mL) and extracted with ethyl acetate (3×50 mL). The combined organic layers were dried over anhydrous sodium sulfate, filtered, concentrated and purified by flash chromatography on silica gel eluting with 100:1 dichlomethane/methanol to give the title compound. MS: 582.1 (M+H⁺).

### Example 74I

### 6-(2,6-dichlorobenzyl)-8-[(6-methoxy-1,2,3,4-tetrahydroisoquinolin-7-yl)amino]phthalazin-1(2H)-one

To EXAMPLE 74H (220 mg, 0.38 mmol) in dichloromethane (10 mL) was added 2,2,2-trifluoroacetic acid (4 mL) and the mixture was stirred at ambient temperature for 3 hours. After concentration, the residue was purified by preparative HPLC eluting with a gradient of 10/90 to 30/20 acetonitrile/water (containing 0.1% trifluoroacetic acid) to give the title compound as a the trifluoroacetate salt. ¹H NMR (DMSO-d₆, 300 MHz): δ 12.99 (s, 1H), 11.86 (s, 1H), 9.03 (s, 2H), 8.30 (s, 1H), 8.21 (s, 1H), 7.60 (d, J=8.1 Hz, 2H), 7.42 (t, J=8.1 Hz, 1H), 6.99 (s, 1H), 6.92 (s, 1H), 4.53 (s, 2H), 4.06 (s, 2H), 3.90 (s, 3H), 3.41 (br, 2H), 2.96 (d, J=6.0 Hz, 2H). MS: 482.1 (M+H⁺).

### Example 75

### 7-(2,6-difluorophenoxy)-5-{[2-methoxy-4-(piperazin-1-yl)phenyl]amino}pyrido[3,4-d]pyridazin-4(3H)-one

### Example 75A

### tert-butyl 4-(4-(7-(2,6-difluorophenoxy)-4-oxo-3,4-dihydropyrido[3,4-d]pyridazin-5-ylamino)-3-methoxyphenyl)piperazine-1-carboxylate

A mixture of EXAMPLE 24A (200 mg, 0.4 mmol), 2,6-difluorophenol (107 mg, 0.82 mmol), cuprous iodide (8 mg, 0.04 mmol), N,N-dimethylglycine (13 mg, 0.12 mmol) and cesium carbonate (267 mg, 0.8 mmol) in 1,4-dioxane (4 mL) was heated at 120° C. for 20 hours. After concentration, the residue was purified by flash chromatography on silica gel (200-300 mesh) eluting with 98/2 dichloromethane/methanol to give the title compound. MS: 581 (M+H⁺).

### Example 75B

### 7-(2,6-difluorophenoxy)-5-{[2-methoxy-4-(piperazin-1-yl)phenyl]amino}pyrido[3,4-d]pyridazin-4(3H)-one

The title compound was obtained following the procedure described in EXAMPLE 28B, using EXAMPLE 75A in place of EXAMPLE 28A. ¹H NMR (DMSO-d₆, 300 MHz): δ 12.84 (s, 1H), 11.63 (s, 1H), 8.78-8.73 (br, 2H), 8.26 (s, 1H), 7.51-7.37 (m, 4H), 6.74 (s, 1H), 6.69 (d, J=2.1 Hz, 1H), 6.00 (dd, J=2.1 Hz, J=9.0 Hz, 1H), 3.88 (s, 3H), 3.28 (s, 8H). MS: 481 (M+H⁺).

### Example 76

### 7-(2-fluorophenoxy)-5-{[2-methoxy-4-(piperazin-1-yl)phenyl]amino}pyrido[3,4-d]pyridazin-4-ol

### Example 76A

### tert-butyl 4-(4-(7-(2-fluorophenoxy)-4-oxo-3,4-dihydropyrido[3,4-d]pyridazin-5-ylamino)-3-methoxyphenyl)piperazine-1-carboxylate

To a solution of EXAMPLE 24A (200 mg, 0.4 mmol) and 2-fluorophenol (100 mg, 0.8 mmol) in dioxane (5 mL) was added (S)-2-(dimethylamino)pentanedioic acid (13 mg, 0.13 mmol) and copper(I) iodide (10 mg, 0.05 mmol). The mixture was degassed with nitrogen twice and stirred at 120° C. for 15 hours. After cooling to ambient temperature, the mixture was concentrated and purified by flash chromatography on silica gel eluting with 80:1 dichloromethane:methanol to give the title compound. MS: 563 (M+H⁺).

### Example 76B

### 7-(2-fluorophenoxy)-5-{[2-methoxy-4-(piperazin-1-yl)phenyl]amino}pyrido[3,4-d]pyridazin-4-ol

The title compound was obtained following the procedure described in EXAMPLE 26E, using EXAMPLE 76A in place of EXAMPLE 26D. ¹H NMR (DMSO-d₆, 300 MHz): δ 12.78 (s, 1H), 11.61 (s, 1H), 8.70 (br, 2H), 8.25 (s, 1H), 7.52-7.37 (m, 5H), 6.68-6.64 (m, 2H), 6.02-5.99 (m, 2H), 3.88 (s, 3H), 3.35 (s, 8H). MS: 463 (M+H⁺).

### Example 77

### 5-{[2-methoxy-4-(piperazin-1-yl)phenyl]amino}-7-[(2,3,4-trichlorophenyl)amino]pyrido[3,4-d]pyridazin-4(3H)-one

### Example 77A

### tert-butyl-4-(3-methoxy-4-(4-oxo-7-(2,3,4-trichlorophenylamino)-3,4-dihydropyrido[3,4-d]pyridazin-5-ylamino)phenyl)piperazine-1-carboxylate

To a solution of EXAMPLE 24A (200 mg, 0.412 mmol) in 1,4-dioxane (20 mL) was added 2,3,4-trichlorobenzenamine (121 mg, 0.617 mmol), dimethylbisdiphenyl phosphinoxanthene (24 mg, 0.041 mmol), tris(dibenzylideneacetone)dipalladium (38 mg, 0.041 mmol) and potassium tert-butoxide (138 mg, 1.24 mmol) in tert-butoxide (20 mL). After heating at 120° C. under nitrogen for 14 hours, the mixture was cooled to ambient temperature, filtered, concentrated and purified by flash chromatography on silica gel (200-300 mesh) eluting with 3/1 petroleum ether/ethyl acetate to provide the title compound. MS: 646 (M+H⁺).

### Example 77B

### 5-{[2-methoxy-4-(piperazin-1-yl)phenyl]amino}-7-[(2,3,4-trichlorophenyl)amino]pyrido[3,4-d]pyridazin-4(3H)-one

To a solution of EXAMPLE 77A (120 mg, 0.19 mmol) in dichloromethane (20 mL) was added 2,2,2-trifluoroacetic acid (6 mL) and the mixture was stirred at ambient temperature for 6 hours. The mixture was concentrated and the residue was purified by preparative HPLC using a gradient of 10/90 to 90/10 acetronitrile in water (containing 0.1% trifluoroacetic acid) to give the title compound. ¹H NMR (DMSO-d₆, 300 MHz): δ 12.37 (s, 1H), 11.39 (s, 1H), 9.42 (s, 1H), 8.73-8.68 (brs, 2H), 8.08 (s, 1H), 7.86-7.83 (d, J=9 Hz, 1H), 7.68-7.67 (d, J=3 Hz, 1H), 6.71-6.70 (d, J=3 Hz, 1H), 6.30-6.15 (m, 2H), 3.87 (s, 2H), 3.30 (m, 8H). MS: 451 (M++).

### Example 78

### 7-(2,3-difluorophenoxy)-5-{[2-methoxy-4-(piperazin-1-yl)phenyl]amino}pyrido[3,4-d]pyridazin-4-ol

### Example 78A

### tert-butyl 4-(4-(7-(2,3-difluorophenoxy)-4-oxo-3,4-dihydropyrido[3,4-d]pyridazin-5-ylamino)-3-methoxy phenyl)piperazine-1-carboxylate

The title compound was obtained following the procedure described in EXAMPLE 76A, using 2,3-difluorophenol in place of 2-fluorophenol. MS: 581 (M+H⁺).

### Example 78B

### 7-(2,3-difluorophenoxy)-5-{[2-methoxy-4-(piperazin-1-yl)phenyl]amino}pyrido[3,4-d]pyridazin-4-ol

The title compound was obtained following the procedure described in EXAMPLE 77B, using EXAMPLE 78A in place of EXAMPLE 77A. ¹H NMR (DMSO-d₆, 300 MHz,): δ 11.52 (s, 1H), 8.21 (s, 1H), 7.40-7.28 (m, 5H), 6.37-6.61 (m, 2H), 5.95 (dd, J=9.0, 2.4 Hz, 1H), 3.84 (s, 3H), 3.05 (dd, J=20.7, 5.7 Hz, 4H), 2.84-2.81 (dd, J=20.4, 5.7 Hz, 4H). MS: 481 (M+H⁺).

### Example 79

### 7-(2,3-dichlorophenoxy)-5-{[2-methoxy-4-(piperazin-1-yl)phenyl]amino}pyrido[3,4-d]pyridazin-4(3H)-one

### Example 79A

### tert-butyl 4-(4-(7-(2,3-dichlorophenoxy)-4-oxo-3,4-dihydropyrido[3,4-d]pyridazin-5-ylamino)-3-methoxyphenyl)piperazine-1-carboxylate

A mixture of EXAMPLE 24A (200 mg, 0.41 mmol), 2,3-dichlorophenol (134 mg, 0.82 mmol), cuprous iodide (8 mg, 0.04 mmol), N,N-dimethylglycine (13 mg, 0.02 mmol) and cesium carbonate (267 mg, 0.82 mmol) in dioxane (20 mL) was degassed with nitrogen for 5 minutes and heated at 120° C. for 16 hours. After concentration, the residue was purified by flash chromatography on silica gel (200-300 mesh) eluting with a gradient of 80/1 to 20/1 dichloromethane/methanol to give the title compound. MS: 613 (M+H⁺).

### Example 79B

### 7-(2,3-dichlorophenoxy)-5-{[2-methoxy-4-(piperazin-1-yl)phenyl]amino}pyrido[3,4-d]pyridazin-4(3H)-one

To a solution of EXAMPLE 79A (230 mg, 0.38 mmol) in dichloromethane (5 mL) was added trifluoroacetic acid (2 mL). After stirring at ambient temperature for 3 hours, the mixture was concentrated and the residue was purified by preparative HPLC using a gradient of 10/90 to 90/10 acetronitrile in water (containing 0.1% trifluoroacetic acid) to give the title compound. ¹H NMR (CD₃OD, 300 MHz): δ 12.80 (s, 1H), 11.56 (s, 1H), 8.74 (s, 2H), 8.26 (s, 1H), 7.73 (dd, J=1.5, 8.1 Hz, 1H), 7.56 (t, J=8.1 Hz, 1H), 7.48 (dd, J=1.5, 8.1 Hz, 1H), 7.40 (d, J=8.7 Hz, 1H), 6.69-6.68 (m, 2H), 6.03 (dd, J=2.4, 8.7 Hz, 1H), 3.88 (s, 3H), 3.29 (brs, 8H). MS: 513 (M+H⁺).

### Example 80

### 7-(2,6-dichloro-4-fluorophenoxy)-5-{[2-methoxy-4-(piperazin-1-yl)phenyl]amino}pyrido[3,4-d]pyridazin-4(3H)-one

### Example 80A

### tert-butyl 4-(4-(7-(2,6-dichloro-4-fluorophenoxy)-4-oxo-3,4-dihydropyrido[3,4-d]pyridazin-5-ylamino)-3-methoxyphenyl)piperazine-1-carboxylate

The title compound was obtained following the procedure described in EXAMPLE 75A, using 2,6-dichloro-4-fluorophenol in place of 2,6-difluorophenol. MS: 631 (M+H⁺).

### Example 80B

### 7-(2,6-dichloro-4-fluorophenoxy)-5-{[2-methoxy-4-(piperazin-1-yl)phenyl]amino}pyrido[3,4-d]pyridazin-4(3H)-one

The title compound was obtained following the procedure described in EXAMPLE 28B, using EXAMPLE 80A in place of EXAMPLE 28A. ¹H NMR (DMSO-d₆, 300 MHz): δ 12.83 (s, 1H), 11.55 (s, 1H), 8.72 (brs, 2H), 8.27 (s, 1H), 7.83 (d, J=8.4 Hz, 1H), 7.39 (d, J=9.0 Hz, 1H), 6.75 (s, 1H), 6.71 (d, J=2.4 Hz, 1H), 6.05 (dd, J=3.2 Hz, J=9.0 Hz, 1H), 3.88 (s, 3H), 3.29 (s, 8H). MS: 531 (M+H⁺).

### Example 81

### 7-[(3-chloropyridin-4-yl)oxy]-5-{[2-methoxy-4-(piperazin-1-yl)phenyl]amino}pyrido[3,4-d]pyridazin-4(3H)-one

### Example 81A

### tert-buty 14-(4-(7-(3-chloropyridin-4-yloxy)-4-oxo-3,4-dihydropyrido[3,4-d]pyridazin-5-ylamino)-3-methoxyphenyl)piperazine-1-carboxylate

The title compound was obtained following the procedure described in EXAMPLE 75A, using 3-chloropyridin-4-ol in place of 2,6-difluorophenol. MS: 580 (M+H⁺).

### Example 81B

### 7-[(3-chloropyridin-4-yl)oxy]-5-{[2-methoxy-4-(piperazin-1-yl)phenyl]amino}pyrido[3,4-d]pyridazin-4(3H)-one

The title compound was obtained following the procedure described in EXAMPLE 28B, using EXAMPLE 81A in place of EXAMPLE 28A. ¹H NMR (DMSO-d₆, 300 MHz): δ 13.15 (s, 1H), 11.55 (s, 1H), 8.88 (s, 1H), 8.75-8.74 (brs, 2H), 8.56 (dd, J=2.1 Hz, J=7.8 Hz, 1H), 8.26-8.21 (m, 2H), 7.44 (s, 1H), 6.80 (s, 1H), 6.65 (d, J=9.0 Hz, 1H), 6.54 (d, J=7.8 Hz, 1H), 3.40 (brs, 4H), 3.29 (brs, 4H). MS: 480 (M+H⁺).

### Example 82

### 7-(2,3-dimethylphenoxy)-5-{[2-methoxy-4-(piperazin-1-yl)phenyl]amino}pyrido[3,4-d]pyridazin-4-ol

### Example 82A

### tert-butyl 4-(4-(7-(2,3-dimethylphenoxy)-4-oxo-3,4-dihydropyrido[3,4-d]pyridazin-5-ylamino)-3-methoxyphenyl)piperazine-1-carboxylate

The title compound was obtained following the procedure described in EXAMPLE 76A, using 2,3-dimethylphenol in place of 2-fluorophenol. MS: 573 (M+H⁺).

### Example 82B

### 7-(2,3-dimethylphenoxy)-5-{[2-methoxy-4-(piperazin-1-yl)phenyl]amino}pyrido[3,4-d]pyridazin-4-ol

The title compound was obtained following the procedure described in EXAMPLE 26E, using EXAMPLE 82A in place of EXAMPLE 26D. ¹H NMR (DMSO-d₆, 300 MHz): δ 12.71 (s, 1H), 11.56 (s, 1H), 8.69 (brs, 2H), 8.22 (s, 1H), 7.60-7.57 (m, 1H), 7.26-7.23 (m, 1H), 6.69-6.66 (m, 2H), 6.50 (s, 1H), 6.05-6.02 (m, 1H), 3.87 (s, 3H), 3.39 (brs, 8H), 2.30 (s, 3H), 2.03 (s, 3H). MS: 473 (M+H⁺).

### Example 83

### 3) 7-[(3-fluoropyridin-4-yl)oxy]-5-{[2-methoxy-4-(piperazin-1-yl)phenyl]amino}pyrido[3,4-d]pyridazin-4-ol

### Example 83A

### tert-butyl 4-(4-((7-((3-fluoropyridin-4-yl)oxy)-4-oxo-3,4-dihydropyrido[3,4-d]pyridazin-5-yl)amino)-3-methoxyphenyl)piperazine-1-carboxylate

A mixture of EXAMPLE 24A (200 mg, 0.41 mmol), 3-fluoropyridin-4-ol (93 mg, 0.82 mmol), copper(I) iodide (8 mg, 0.043 mmol), N,N-dimethylglycine (13 mg, 0.123 mmol) and cesium carbonate (267 mg, 0.82 mmol) in 1,4-dioxane (10 mL) was degassed twice with nitrogen and heated in a sealed tube at 120° C. for 28 hours. After concentration, the residue was purified by flash chromatography on silica gel eluting with 30/1 dichloromethane/methanol to give the title compound. MS: 564 (M+H⁺).

### Example 83B

### 7-[(3-fluoropyridin-4-yl)oxy]-5-{[2-methoxy-4-(piperazin-1-yl)phenyl]amino}pyrido[3,4-d]pyridazin-4-ol

To a solution of EXAMPLE 83A (170 mg, 0.3 mmol) in dichloromethane (20 mL) at room temperature was added trifluoroacetic acid (3 mL) and the mixture was stirred for 2 hours. The mixture was concentrated and methanol (10 mL) was added. Filtration gave the title compound. ¹H NMR (DMSO-d₆, 300 MHz): δ 13.09 (s, 1H), 11.53 (s, 1H), 8.77-8.73 (m, 3H), 8.50 (dd. J=7.8, 2.4 Hz, 1H), 8.19 (d, J=8.7 Hz, 1H), 8.19 (s, 1H), 7.35 (s, 1H), 6.75 (d, J=3.2 Hz, 1H), 6.63-6.52 (m, 2H), 3.89 (s, 3H), 3.37-3.15 (m, 8H). MS: 464 (M+H⁺).

### Example 84

### 5-{[2-methoxy-4-(piperazin-1-yl)phenyl]amino}-7-(2,3,4-trichlorophenoxy)pyrido[3,4-d]pyridazin-4-ol

### Example 84A

### tert-butyl 4-(3-methoxy-4-(4-oxo-7-(2,3,4-trichlorophenoxy)-3,4-dihydropyrido[3,4-d]pyridazin-5-ylamino)phenyl)piperazine-1-carboxylate

The title compound was obtained following the procedure described in EXAMPLE 76A, using 2,3,4-trichlorophenol in place of 2-fluorophenol. MS: 647(M+H⁺).

### Example 84B

### 5-{[2-methoxy-4-(piperazin-1-yl)phenyl]amino}-7-(2,3,4-trichlorophenoxy)pyrido[3,4-d]pyridazin-4-ol

The title compound was obtained following the procedure described in EXAMPLE 26E, using EXAMPLE 84A in place of EXAMPLE 26D. ¹H NMR (DMSO-d₆, 300 MHz): δ 12.83 (s, 1H), 11.49 (s, 1H), 8.67 (brs, 2H), 8.27 (s, 1H), 7.88-7.86 (m, 1H), 7.57-7.54 (m, 1H), 7.30-7.27 (m, 1H), 6.72 (s, 2H), 5.99-5.96 (m, 1H), 3.86 (s, 3H), 3.35 (brs, 8H). MS: 547 (M+H⁺).

### Example 85

### 7-(2,4-dichlorophenoxy)-5-{[2-methoxy-4-(piperazin-1-yl)phenyl]amino}pyrido[3,4-d]pyridazin-4(3H)-one

### Example 85A

### tert-butyl 4-(4-(7-(2,4-dichlorophenoxy)-4-oxo-3,4-dihydropyrido[3,4-d]pyridazin-5-ylamino)-3-methoxyphenyl)piperazine-1-carboxylate

The title compound was obtained following the procedure described in EXAMPLE 62A, using 2,4-dichlorophenol in place of 2-chlorophenol.

### Example 85B

### 7-(2,4-dichlorophenoxy)-5-{[2-methoxy-4-(piperazin-1-yl)phenyl]amino}pyrido[3,4-d]pyridazin-4(3H)-one

The title compound was obtained following the procedure described in EXAMPLE 2F, using EXAMPLE 85A in place of EXAMPLE 2E. ¹H NMR (DMSO-d₆, 300 MHz): δ 12.78 (s, 1H), 11.51 (s, 1H), 8.60 (s, 2H), 8.26 (s, 1H), 7.91 (s, 1H), 7.61 (s, 1H), 7.51 (d, J=9 Hz, 1H), 7.37 (d, J=9 Hz, 1H), 6.70 (m, 2H), 6.05 (d, J=9 Hz, 1H), 3.88 (s, 3H), 3.30 (s, 8H). MS: 514 (M+H⁺).

### Example 86

### 7-[(trans-4-hydroxycyclohexyl)amino]-5-{[2-methoxy-4-(piperazin-1-yl)phenyl]amino}pyrido[3,4-d]pyridazin-4-ol

### Example 86A

### (trans)-4-(tert-butyldimethylsilyloxy)cyclohexanamine

To a solution of (1r,4r)-4-aminocyclohexanol (500 mg, 4.3 mmol) and tert-butyl chlorodimethylsilane (981 mg, 6.5 mmol) in N,N-dimethylformamide (8 mL) was added imidazole (469 mg, 7.8 mmol). The mixture was stirred at ambient temperature for 12 hours, quenched with water and extracted with ethyl acetate. The organic phase was washed with brine and concentrated to yield the title compound.

### Example 86B

### tert-butyl 4-(4-(7-((trans)-4-(tert-butyldimethylsilyloxy)cyclohexylamino)-4-oxo-3,4-dihydropyrido[3,4-d]pyridazin-5-ylamino)-3-methoxyphenyl)piperazine-1-carboxylate

The title compound was obtained following the procedure described in EXAMPLE 24B, using EXAMPLE 86A in place of 2-chlorobenzenamine. MS: 680 (M+H⁺)

### Example 86C

### 7-[(trans-4-hydroxycyclohexyl)amino]-5-{[2-methoxy-4-(piperazin-1-yl)phenyl]amino}pyrido[3,4-d]pyridazin-4-ol

The title compound was obtained following the procedure described in EXAMPLE 26E, using EXAMPLE 86B in place of EXAMPLE 26D. ¹H NMR (DMSO-d₆, 300 MHz): δ 12.01 (s, 1H), 11.51 (s, 1H), 8.75-8.60 (m, 3H), 7.88 (s, 1H), 7.31 (brs, 1H), 6.76 (s, 2H), 6.51 (d, J=9 Hz, 1H), 5.87 (s, 1H), 3.91 (s, 3H), 3.85 (brs, 1H), 3.33 (brs, 8H), 2.03-1.91 (m, 4H), 1.32 (brs, 4H). MS: 466 (M+H⁺).

### Example 87

### 7-(cyclopentyloxy)-5-{[2-methoxy-4-(piperazin-1-yl)phenyl]amino}pyrido[3,4-d]pyridazin-4-ol

To a solution of cyclopentanol (71 mg, 0.84 mmol) in tetrahydrofuran (10 mL) was added 60% sodium hydride (42 mg, 1.05 mmol) and the mixture was stirred at room temperature for 30 minutes. The product of EXAMPLE 24A (0.1 g, 0.21 mmol) was added and the mixture was stirred at 150° C. for 24 hours. The mixture was quenched with water and extracted with tetrahydrofuran. The organic phase was washed with brine, dried over anhydrous sodium sulfate, filtered and concentrated. The residue was purified by preparative HPLC using a gradient of 10/90 to 90/10 acetronitrile in water (containing 0.1% trifluoroacetic acid) to give the title compound. ¹H NMR (DMSO-d₆, 300 MHz): δ 12.55 (s, 1H), 11.60 (s, 1H), 8.70 (s, 2H), 8.49 (d, J=9.0 Hz, 1H), 8.09 (s, 1H), 6.86 (s, 1H), 6.64 (d, J=9.0 Hz, 1H), 6.27 (s, 1H), 5.40 (s, 1H), 3.92 (s, 3H), 3.38 (s, 4H), 3.27 (s, 4H), 2.01-1.67 (m, 8H). MS: 438 (M+H⁺).

### Example 88

### 7-(2,6-dichlorobenzyl)-5-[(7-methoxy-1,2,3,4-tetrahydroisoquinolin-6-yl)amino]pyrido[3,4-d]pyridazin-4(3H)-one

### Example 88A

### methyl 4-methoxyphenethylcarbamate

To a solution of 2-(4-methoxyphenyl)ethanamine (20 g, 132 mmol) in dichloromethane (200 mL) was added sodium carbonate (28 g, 264 mmol) and methyl carbonochloridate (13.1 g, 139 mmol). After stirring at ambient temperature for 4 hours, the mixture was poured into water (200 mL) and extracted with dichloromethane (2×200 mL). The combined organic phase was washed with brine (100 mL), dried over anhydrous sodium sulfate, filtered, and concentrated to give the title compound. MS: 210 (M+H⁺).

### Example 88B

### 7-methoxy-3,4-dihydroisoquinolin-1(2H)-one

To polyphosphoric acid (150 mL) at 120° C. was added slowly EXAMPLE 88A (29 g, 139 mmol). After stirring for 0.5 hours, the mixture was cooled to 50° C. and poured into ice-water (300 mL). The mixture was extracted with dichloromethane (2×200 mL) and the combined organic layers were washed with brine (100 mL), dried over anhydrous sodium sulfate, filtered, and concentrated to give the title compound. MS: 178 (M+H⁺).

### Example 88C

### 7-methoxy-1,2,3,4-tetrahydroisoquinoline

To a suspension of lithium aluminum hydride (10 g, 46 mmol) in tetrahydrofuran (100 mL) at 0° C. under nitrogen was added slowly a solution of EXAMPLE 88B (4.1 g, 23 mmol) in tetrahydrofuran (50 mL) over 0.5 hour and the mixture was heated to 70° C. for 2 hours. After cooling to 0° C., 15% sodium hydroxide (4.9 mL) was added slowly and the mixture was filtered and washed with ethyl acetate (50 mL). The filtrate was concentrated to give the crude title compound. MS: 164 (M+H⁺).

### Example 88D

### tert-butyl 7-methoxy-3,4-dihydroisoquinoline-2(1H)-carboxylate

To a solution of EXAMPLE 88C (1.88 g, 11.5 mmol) in dichloromethane (40 mL) was added triethylamine (2.3 g, 23 mmol) and di-tert-butyl dicarbonate (3 g, 13.8 mmol). After stirring at ambient temperature for 16 hours, the mixture was poured into water (50 mL) and extracted with dichloromethane (2×100 mL). The combined organic layers were dried over anhydrous sodium sulfate, filtered, concentrated and purified by flash chromatography on silica gel eluting with 10:1 hexane:ethyl acetate to give the title compound. MS: 264 (M+H⁺).

### Example 88E

### tert-butyl 7-methoxy-6-nitro-3,4-dihydroisoquinoline-2(1H)-carboxylate

To a solution of EXAMPLE 88D (2.46 g, 9.35 mmol) in nitromethane (30 mL) at −10° C. was added acetic anhydride (5.7 g, 56.1 mmol) and concentrated nitric acid (0.88 g, 14 mmol). After stirring for 3 hours, the mixture was adjusted to pH 7 with aqueous sodium bicarbonate solution and extracted with dichloromethane (2×100 mL). The combined organic layers were dried over anhydrous sodium sulfate, filtered, concentrated and purified by flash chromatography on silica gel eluting with 5:1 hexane:ethyl acetate to give the title compound. MS: 309 (M+H⁺).

### Example 88F

### tert-butyl 6-amino-7-methoxy-3,4-dihydroisoquinoline-2(1H)-carboxylate

To a solution of EXAMPLE 88E (550 mg, 1.78 mmol) in methanol (10 mL) was added Raney Nickel (55 mg) and the mixture was stirred at ambient temperature under hydrogen for 16 hours. The mixture was filtered and the filtrate was concentrated to give the crude title compound. MS: 279 (M+H⁺).

### Example 88G

### tert-butyl 6-(7-chloro-4-oxo-3,4-dihydropyrido[3,4-d]pyridazin-5-ylamino)-7-methoxy-3,4-dihydroisoquinoline-2(H)-carboxylate

To a solution of EXAMPLE 7K (200 mg, 0.93 mmol) in dioxane (10 mL) was added EXAMPLE 88F (284 mg, 1.02 mmol) and N,N-diisopropylethylamine (600 mg, 4.65 mmol) and the mixture was heated in a sealed tube at 120° C. for 16 hours. After cooling to ambient temperature, the mixture was poured into water (50 mL) and extracted with dichloromethane (100 mL). The organic layer was dried over anhydrous sodium sulfate, filtered, concentrated and purified by flash chromatography on silica gel eluting with 100:1 dichlomethane/methanol to give the title compound. MS: 458 (M+H⁺).

### Example 88H

### tert-butyl 6-(7-(2,6-dichlorobenzyl)-4-oxo-3,4-dihydropyrido[3,4-d]pyridazin-5-ylamino)-7-methoxy-3,4-dihydroisoquinoline-2(1H)-carboxylate

To a solution of EXAMPLE 88G (200 mg, 0.44 mmol) in tetrahydrofuran (10 mL) was added 0.5M (2,6-dichlorobenzyl)zinc(II) bromide in tetrahydrofuran (8.8 mL, 4.4 mmol) and tetrakis(triphenylphosphine)palladium (46 mg, 0.04 mmol). After heating at 70° C. under nitrogen for 16 hours, the mixture was cooled to ambient temperature, poured into water (50 mL) and extracted with ethyl acetate (3×50 mL). The combined organic layers were dried over anhydrous sodium sulfate, filtered, concentrated and purified by flash chromatography on silica gel eluting with 100:1 dichlomethane/methanol to give the title compound. MS: 582.1 (M+H⁺).

### Example 881

### 7-(2,6-dichlorobenzyl)-5-[(7-methoxy-1,2,3,4-tetrahydroisoquinolin-6-yl)amino]pyrido[3,4-d]pyridazin-4(3H)-one

To EXAMPLE 88H (220 mg, 0.38 mmol) in dichloromethane (10 mL) was added 2,2,2-trifluoroacetic acid (4 mL) and the mixture was stirred at ambient temperature for 3 hours. After concentration, the residue was purified by preparative HPLC eluting with a gradient of 10/90 to 30/20 acetonitrile/water (containing 0.1% trifluoroacetic acid) to give the title compound. ¹H NMR (DMSO-d₆, 300 MHz): δ 12.99 (s, 1H), 11.86 (s, 1H), 9.02 (s, 2H), 8.31 (s, 1H), 8.22 (s, 1H), 7.60 (d, J=7.8 Hz, 2H), 7.41 (t. J=8.4 Hz, 1H), 7.05 (s, 1H), 6.92 (s, 1H), 4.53 (s, 2H), 4.22 (s, 2H), 3.89 (s, 3H), 3.42 (br, 2H), 2.83 (d, J=6.0 Hz, 2H). MS: 482.1 (M+H⁺).

### Example 89

### 2-(2-chlorophenoxy)-4-{[2-methoxy-4-(piperazin-1-yl)phenyl]amino}pyrido[2,3-d]pyridazin-5(6H)-one

### Example 89A

### tert-butyl 4-(4-((2-(2-chlorophenoxy)-5-oxo-5,6-dihydropyrido[2,3-d]pyridazin-4-yl)amino)-3-methoxyphenyl)piperazine-1-carboxylate

A mixture of EXAMPLE 68G (0.350 g, 0.719 mmol), 2-chlorophenol (0.139 g, 1.08 mmol), copper(I) iodide (13.7 mg, 0.0719 mmol), N,N-dimethylglycine (22.2 mg, 0.216 mmol) and cesium carbonate (0.468 g, 1.44 mmol) in dioxane (15 mL) was degassed with nitrogen and heated in a sealed tube at 120° C. for 1 day. After cooling to ambient temperature, the mixture was concentrated and purified by flash chromatography on silica gel (200-300 mesh) eluting with 120/1 dichloromethane/methanol to give the title compound. MS: 579 (M+H⁺).

### Example 89B

### 2-(2-chlorophenoxy)-4-{[2-methoxy-4-(piperazin-1-yl)phenyl]amino}pyrido[2,3-d]pyridazin-5(6H)-one

To a solution of EXAMPLE 89A (99.9 mg, 0.173 mmol) in dichloromethane (5 mL) was added trifluoroacetic acid (2 mL) and the mixture was stirred at ambient temperature for 1 hour. After concentration, the residue was purified by preparative HPLC using a gradient of 10/90 to 90/10 acetronitrile in water (containing 0.1% trifluoroacetic acid) to give the title compound. ¹H NMR (DMSO-d₆, 300 MHz): δ 12.95 (s, 1H), 10.60 (s, 1H), 7.92 (s, 1H), 7.60 (dd, J=7.8 Hz, 1.5 Hz, 1H), 7.46-7.25 (m, 4H), 6.71 (d, J=2.1 Hz, 1H), 6.58 (dd, J=8.7 Hz, 2.1 Hz, 1H), 6.20 (s, 1H), 3.84 (s, 3H), 3.18-3.14 (m, 4H), 2.93-2.89 (m, 4H). MS: 479 (M+H⁺).

### Example 90

### 5-{[2-chloro-4-(pyrrolidin-1-ylmethyl)phenyl]amino}-7-(4-fluoropiperidin-1-yl)pyrido[3,4-d]pyridazin-4-ol

### Example 90A

### 7-chloro-5-(2-chloro-4-(pyrrolidin-1-ylmethyl)phenylamino)pyrido[3,4-d]pyridazin-4(3H)-one

To a solution of EXAMPLE 7K (0.1 g, 0.47 mmol) and EXAMPLE 93B (0.1 g, 0.47 mmol) in butyl alcohol (3 mL) was added p-toluenesulfonic acid (16 mg, 0.093 mmol) and the mixture was stirred at 130° C. overnight. The mixture was concentrated and purified by flash chromatography on silica gel (200-300 mesh) eluting with eluting with 30/1 dichloromethane/methanol to give the title compound.

### Example 90B

### 5-{[2-chloro-4-(pyrrolidin-1-ylmethyl)phenyl]amino}-7-(4-fluoropiperidin-1-yl)pyrido[3,4-d]pyridazin-4-ol

A mixture of EXAMPLE 90A (50 mg, 0.13 mmol), 4-fluoropiperidine (54 mg, 0.39 mmol) and ethyl diisopropylamine (0.1 mL, 0.54 mmol) in 1,4-dioxane (5 mL) was stirred in a sealed tube at 130° C. overnight. The mixture was concentrated and the residue was purified by preparative HPLC using a gradient of 10/90 to 90/10 acetronitrile in water (containing 0.1% trifluoroacetic acid) to give the title compound. ¹H NMR (DMSO-d₆, 300 MHz): δ 12.44 (s, 1H), 11.86 (s, 1H), 10.46 (s, 2H), 8.69 (d, J=8.4 Hz, 1H), 8.04 (s, 1H), 7.88 (s, 1H), 7.57 (d, J=8.4 Hz, 1H), 6.55 (s, 1H), 5.06-4.91 (m, 1H), 4.33 (d, J=5.4 Hz, 2H), 3.85-3.63 (m, 4H), 3.34-3.36 (m, 2H), 3.19-3.16 (m, 2H), 2.05-1.81 (m, 8H). MS: 458 (M+H⁺).

### Example 91

### 2-(2-chlorobenzyl)-4-[(6-methoxy-1,2,3,4-tetrahydroisoquinolin-7-yl)amino]pyrido[2,3-d]pyridazin-5-ol

### Example 91A

### tert-butyl 7-(7-chloro-4-oxo-3,4-dihydropyrido[3,4-d]pyridazin-5-ylamino)-6-methoxy-3,4-dihydroisoquinoline-2(1H)-carboxylate

The title compound was obtained following the procedure described in EXAMPLE 24A, using EXAMPLE 74F in place of EXAMPLE 1G. MS: 458 (M+H⁺).

### Example 91B

### tert-butyl 7-(7-(2-chlorobenzyl)-4-oxo-3,4-dihydropyrido[3,4-d]pyridazin-5-ylamino)-6-methoxy-3,4-dihydroisoquinoline-2(1H)-carboxylate

The title compound was obtained following the procedure described in EXAMPLE 92F, using EXAMPLE 91A in place of EXAMPLE 92E. MS: 548 (M+H⁺).

### Example 91C

### 2-(2-chlorobenzyl)-4-[(6-methoxy-1,2,3,4-tetrahydroisoquinolin-7-yl)amino]pyrido[2,3-d]pyridazin-5-ol

The title compound was obtained following the procedure described in EXAMPLE 34B, using EXAMPLE 91B in place of EXAMPLE 34A. ¹H NMR (DMSO-d₆, 300 MHz): δ 13.28 (s, 1H), 11.33 (s, 1H), 8.50 (s, 2H), 8.37 (s, 1H), 7.55-7.52 (m, 1H), 7.43-7.35 (m, 3H), 7.23 (s, 1H), 7.03 (s, 1H), 6.73 (s, 1H), 4.31 (s, 2H), 4.15 (m, 2H), 3.78 (s, 3H), 3.40-3.38 (m, 2H), 3.06-3.02 (m, 2H).

### Example 92

### 2-(2-chlorobenzyl)-4-{[2-methoxy-4-(piperazin-1-ylmethyl)phenyl]amino}pyrido[2,3-d]pyridazin-5(6H)-one

### Example 92A

### 3-methoxy-4-nitrobenzoyl chloride

To a solution of 3-methoxy-4-nitrobenzoic acid (5.7 g, 30 mmol) in dichloromethane (100 mL) and N,N-dimethylformamide (5 mL) at 0° C. was added slowly oxalyl chloride (5.08 mL, 60 mmol). After stirring for 2 hours, the mixture was concentrated and the residue used without further purification.

### Example 92B

### tert-buty 14-(3-methoxy-4-nitrobenzoyl)piperazine-1-carboxylate

To a solution of EXAMPLE 92A (13 mmol) in dichloromethane (100 mL) at 0° C. was added slowly tert-butyl piperazine-1-carboxylate (1.06 g, 13 mmol) and triethylamine (3.6 mL). After stirring for 2 hours, water was added slowly and the mixture was extracted with dichloromethane (3×200 mL). The combined organic phase was washed with brine, dried over sodium sulfate, filtered, concentrated and purified by flash chromatography on silica gel (200-300 mesh) eluting with 100/1 dichloromethane/methanol to provide the title compound. MS: 388 (M+TH).

### Example 92C

### tert-butyl 4-(4-amino-3-methoxybenzoyl)piperazine-1-carboxylate

To a solution of EXAMPLE 92B (3 g, 11 mmol) in methanol (100 mL) was added Raney Ni (300 mg) and the mixture was stirred under hydrogen for 14 hours. The catalyst was filtered off and the filtrate was concentrated and purified by flash chromatography on silica gel (200-300 mesh) eluting with 100/1 dichloromethane/methanol to provide the title compound. MS: 336 (M+H⁺).

### Example 92D

### tert-butyl 4-(4-amino-3-methoxybenzyl)piperazine-1-carboxylate

The title compound was obtained following the procedure described in EXAMPLE 93B, using EXAMPLE 92C in place of EXAMPLE 93A.

### Example 92E

### tert-butyl 4-(4-((2-chloro-5-oxo-5,6-dihydropyrido[2,3-d]pyridazin-4-yl)amino)-3-methoxybenzyl)piperazine-1-carboxylate

A mixture of EXAMPLE 68F (0.311 g, 1.44 mmol), EXAMPLE 92D (0.419 g, 1.31 mmol), N,N-diisopropylethylamine (0.338 g, 2.62 mmol) and dioxane (10 mL) was heated in a sealed tube at 120° C. for 1 day. After cooling to ambient temperature, the mixture was concentrated and purified by flash chromatography on silica gel (200-300 mesh) eluting with 50/1 dichloromethane/methanol to give the title compound. MS: 501 (M+He).

### Example 92F

### tert-butyl 4-(4-((2-(2-chlorobenzyl)-5-oxo-5,6-dihydropyrido[2,3-d]pyridazin-4-yl)amino)-3-methoxybenzyl)piperazine-1-carboxylate

A mixture of EXAMPLE 92E (0.150 g, 0.300 mmol), 0.5M (2-chlorobenzyl)zinc(II) bromide in tetrahydrofuran (3.0 mL, 1.50 mmol), tetrakis(triphenylphosphine)palladium (17.3 mg, 0.015 mmol) and tetrahydrofuran (5 mL) was degassed with nitrogen 6 times and heated at 72° C. for 15 hours. After cooling to ambient temperature, the mixture was diluted with saturated aqueous ammonium chloride (10 mL) and extracted with dichloromethane (3×20 mL). The organic phase was dried over anhydrous sodium sulfate, filtered, concentrated and purified by flash chromatography on silica gel (200-300 mesh) eluting with 100/1 dichloromethane/methanol to give the title compound. MS: 591 (M+H⁺).

### Example 92G

### 2-(2-chlorobenzyl)-4-{[2-methoxy-4-(piperazin-1-ylmethyl)phenyl]amino}pyrido[2,3-d]pyridazin-5(6H)-one

To a solution of EXAMPLE 92F (0.140 g, 0.237 mmol) in dichloromethane (5 mL) was added trifluoroacetic acid (3 mL) and the mixture was stirred at ambient temperature for 2 hours. After concentration, the residue was purified by preparative HPLC using a gradient of 10/90 to 80/20 acetronitrile in water (containing 0.1% trifluoroacetic acid) to give the title compound. ¹H NMR (DMSO-d₆, 300 MHz): δ 12.87 (s, 1H), 10.84 (s, 1H), 8.17 (s, 1H), 7.49-7.46 (m, 1H), 7.39-7.28 (m, 5H), 7.06 (s, 1H), 6.90 (d, J=8.1 Hz, 1H), 6.70 (s, 1H), 4.20 (s, 2H), 3.79 (s, 3H), 3.53 (s, 2H), 3.36 (m, 4H), 3.00 (m, 4H). MS: 491 (M+H⁺).

### Example 93

### 5-{[2-chloro-4-(pyrrolidin-1-ylmethyl)phenyl]amino}-7-[(cyclopropylmethyl)amino]pyrido[3,4-d]pyridazin-4-ol

### Example 93A

### (4-amino-3-chlorophenyl)(pyrrolidin-1-yl)methanone

A suspension of 4-amino-3-chlorobenzoic acid (1 g, 5.8 mmol), pyrrolidine (705 mg, 8.7 mmol), 1-ethyl-(3-dimethylaminopropyl)carbodiimide hydrochloride (4.5 g, 23.2 mmol), 1-hydroxybenzotrizole (3.1 g, 23.2 mmol) and N,N-diisoproylethylamine (6 g, 46.4 mmol) in dichloromethane (100 mL) was stirred at ambient temperature for 4 hours. The mixture was washed with water (50 mL), dried over anhydrous sodium sulfate, filtered, concentrated and purified by flash chromatography on silica gel (200-300 mesh), eluting with 1/1 petroleum ether/ethyl acetate to give the title compound. MS: 225 (M+H⁴).

### Example 93B

### 2-chloro-4-(pyrrolidin-1-ylmethyl)benzenamine

To a solution of EXAMPLE 93A (1.2 g, 5.3 mmol) in tetrahydrofuran (50 mL) was added dropwise 1M borane-tetrahydrofuran complex (26.5 mL, 26.5 mmol) at 0° C. and the mixture was stirred at 0° C. for 20 minutes and heated at 70° C. for 4 hours. The reaction mixture was quenched with methanol and concentrated. The residue was dissolved in tetrahydrofuran (50 mL) and N,N,N′,N′-tetramethylethylenediamine (2 mL) and the mixture was stirred at ambient temperature for 4 hours. After concentration, the residue was purified by flash chromatography on silica gel (200-300 mesh) eluting with 1/1 petroleum ether/ethyl acetate to give the title compound. MS: 211 (M+H⁺).

### Example 93C

### 7-chloro-5-(2-chloro-4-(pyrrolidin-1-ylmethyl)phenylamino)pyrido[3,4-d]pyridazin-4(3H)-one

To a solution of EXAMPLE 7K (5 g, 23.1 mmol) and EXAMPLE 93B (0.4 g, 2.3 mmol) in butyl alcohol (100 mL) was added p-toluenesulfonic acid (0.4 g, 2.3 mmol) and the mixture was heated at 130° C. for 20 hours. After cooling to ambient temperature, the mixture was concentrated and purified by flash chromatography eluting with 80:1 dichloromethane:methanol to give the title compound. MS: 390 (M+H⁺).

### Example 93D

### 5-{[2-chloro-4-(pyrrolidin-1-ylmethyl)phenyl]amino}-7-[(cyclopropylmethyl)amino]pyrido[3,4-d]pyridazin-4-ol

To a solution of EXAMPLE 93C (50 mg, 0.13 mmol) and cyclopropylmethanamine (50 mg, 0.7 mmol) in dioxane (5 mL) was added N,N-dimethylethanamine (100 mg, 1.1 mmol) and the mixture was stirred at 100 C for 15 hours. After cooling to ambient temperature, the mixture was concentrated and purified by flash chromatography on silica get eluting with 80:1 dichloromethane/methanol to give the title compound. ¹H NMR (DMSO-d₆, 300 MHz): δ 12.25 (s, 1H), 12.00 (s, 1H), 10.40 (brs, 1H), 9.03 (brs, 1H), 8.01 (s, 1H), 7.60 (s, 2H), 7.58-7.55 (m, 1H), 6.13 (m, 1H), 4.35-4.33 (m, 2H), 3.39-3.10 (m, 6H), 2.05-1.89 (m, 4H), 1.15 (brs, 1H), 0.53-0.51 (m, 2H), 0.29-0.28 (m, 2H). MS: 425 (M+H⁺).

### Example 94

### 2-(2-chlorobenzyl)-4-{[2-methoxy-4-(morpholin-4-ylmethyl)phenyl]amino}pyrido[2,3-d]pyridazin-5-ol

### Example 94A

### (4-amino-3-methoxyphenyl)(morpholino)methanone

The title compound was obtained following the procedure as described in EXAMPLE 93A, using 4-amino-3-methoxybenzoic acid in place of 4-amino-3-chlorobenzoic acid and morpholine in place of pyrrolidine.

### Example 94B

### 2-methoxy-4-(morpholinomethyl)benzenamine

The title compound was obtained following the procedure described in EXAMPLE 93B, using EXAMPLE 94A in place of EXAMPLE 93A.

### Example 94C

### 2-chloro-4-((2-methoxy-4-(morpholinomethyl)phenyl)amino)pyrido[2,3-d]pyridazin-5(6H)-one

To a solution of EXAMPLE 68F (246 mg, 1.15 mmol). EXAMPLE 94B (280 mg, 1.26 mmol) in 1,4-dioxane (10 mL) was added N-ethyl-N-isopropylpropan-2-amine (296 mg, 2.3 mmol) and the mixture was heated in a sealed tube at 120° C. overnight. After concentration, the residue was purified by flash chromatography on silica gel eluting with 1/1 petroleum ether/ethyl acetate to give the title compound. MS: 402 (M+H⁺).

### Example 94D

### 2-(2-chlorobenzyl)-4-{[2-methoxy-4-(morpholin-4-ylmethyl)phenyl]amino}pyrido[2,3-d]pyridazin-5-ol

To a solution of EXAMPLE 94C (160 mg, 0.4 mmol) in tetrahydrofuran (20 mL) under nitrogen was added tetrakis(triphenylphosphine) palladium(0) (23 mg, 0.02 mmol), followed by 0.5N (2-chlorobenzyl)zinc(II) bromide in tetrahydrofuran (4 mL, 2 mmol) and the mixture was stirred at 70° C. overnight. After concentration, the residue was purified by flash chromatography on silica gel eluting with 1/1 petroleum ether/ethyl acetate to give the title compound. ¹H NMR (CD₃OD, 300 MHz): δ 8.14 (s, 1H), 7.42-7.23 (m, 5H), 7.13 (s, 1H), 6.95 (d, J=8.7 Hz, 1H), 6.67 (s, 1H), 4.23 (s, 2H), 3.83 (brs, 9H), 2.83 (brs, 4H). MS: 492 (M+H⁺).

### Example 95

### 7-(2-chlorophenoxy)-5-{[2-ethoxy-4-(pyrrolidin-1-ylmethyl)phenyl]amino}pyrido[3,4-d]pyridazin-4(3H)-one

### Example 95A

### (3-hydroxy-4-nitrophenyl)(pyrrolidin-1-yl)methanone

A suspension of 3-hydroxy-4-nitrobenzoic acid (5.4 g, 30 mmol), pyrrolidine (2.6 g, 3.6 mmol), triethylamine (9 g, 90 mmol) and N,N,N′,N′-tetramethyluronium hexafluorophosphate (17 g, 45 mmol) in dichloromethane (150 mL) was stirred at ambient temperature for 17 hours. The mixture was washed with water (3×50 mL), concentrated and purified by flash chromatography on silica gel eluting with 1/1 petroleum eter/ethyl acetate to give the title compound. MS: 237 (M+H⁺).

### Example 95B

### (3-ethoxy-4-nitrophenyl)(pyrrolidin-1-yl)methanone

To a mixture of EXAMPLE 95A (119 mg, 0.5 mmol) and potassium carbonate (138 mg, 1 mmol) in acetonitrile (10 mL) at 0° C. was added bromoethane (1 mL) dropwise and the mixture was stirred at 0° C. for 0.5 hour and at ambient temperature for 16 hours. The mixture was diluted with water (20 mL) and extracted with ethyl acetate (100 mL). The organic layer was concentrated and purified by flash chromatography on silica gel eluting with 1/1 petroleum ether/ethyl acetate to give the title compound. MS: 265 (M+H⁺).

### Example 95C

### (4-amino-3-ethoxyphenyl)(pyrrolidin-1-yl)methanone

A suspension of EXAMPLE 95B (1.1 g, crude) and Raney Nickel (0.4 g) was stirred under hydrogen at ambient temperature for 15 hours. The mixture was filtered and the filtrate was concentrated to give the crude title compound. MS: 235 (M+H⁺).

### Example 95D

### 2-ethoxy-4-(pyrrolidin-1-ylmethyl)benzenamine

To a solution of EXAMPLE 95C (1.05 g, 4.5 mmol) in tetrahydrofuran (50 mL) at 0° C. was added 1M borane-tetrahydrofuran complex (22 mL, 22 mmol) and the mixture was stirred at 0° C. for 3 hours and at ambient temperature for 3 hours. The mixture was quenched with methanol, concentrated and purified by flash chromatography on silica gel eluting with 3/1 petroleum ether/ethyl acetate to the title compound. MS: 221 (M+H⁺).

### Example 95E

### 7-chloro-5-(2-ethoxy-4-(pyrrolidin-1-ylmethyl)phenylamino)pyrido[3,4-d]pyridazin-4(3H) one

A solution of EXAMPLE 95D (200 mg, 0.9 mmol), EXAMPLE 7K (200 mg, 0.93 mmol) and N,N-diisopropyethylamine (2 mL) in 1,4-dioxane (30 mL) was heated in a sealed-tube at 120° C. for 1.5 hours. The mixture was concentrated and purified by flash chromatography on silica gel eluting with 20/1 dichloromethane/methane to give the title compound. MS: 400 (M+H⁺).

### Example 95F

### 7-(2-chlorophenoxy)-5-{[2-ethoxy-4-(pyrrolidin-1-ylmethyl)phenyl]amino}pyrido[3,4-d]pyridazin-4(3H)-one

A mixture of EXAMPLE 95E (150 mg, 0.38 mmol), 2-chlorophenol (65 mg, 0.5 mmol), dimethylglycine (10 mg, 0.1 mmol), copper(I) iodide (7.6 mg, 0.04 mmol) and cesium carbonate (248 mg, 0.76 mmol) in 1,4-dioxane (20 mL) was heated in a sealed-tube at 120° C. for 15 hours. The mixture was concentrated and purified by flash chromatography on silica gel eluting with 20/1 dichloromethane/methane to give the title compound. ¹H NMR (DMSO-d₆, 300 MHz): δ 12.92 (s, 1H), 11.95 (s, 1H), 10.74 (s, 1H), 8.30 (s, 1H), 7.73-7.70 (d, J=9 Hz, 1H), 7.56-7.48 (m, 4H), 7.35 (s, 1H), 4.03 (s, 2H), 6.79 (s, 1H), 6.63-6.60 (d, J=9 Hz, 1H), 4.23-4.15 (m, 4H), 3.33-3.30 (m, 2H), 3.04-3.00 (m, 2H), 2.02-1.90 (m, 4H), 1.47 (t, 3H). MS: 492 (M+H⁺).

### Example 96

### 5-{[2-chloro-4-(pyrrolidin-1-ylmethyl)phenyl]amino}-7-(4-hydroxypiperidin-1-yl)pyrido[3,4-d]pyridazin-4(3H)-one

To a solution of EXAMPLE 93C (39 mg, 0.1 mmol), piperidin-4-ol (16 mg, 0.15 mmol) in 1,4-dioxane (4 mL) was added N-ethyl-N-isopropyl propan-2-amine (25 mg, 0.2 mmol) and the mixture was heated in a sealed tube at 120° C. overnight. After concentration, the residue was purified by preparative HPLC using a gradient of 10/90 to 80/20 acetronitrile in water (containing 0.1% trifluoroacetic acid) to give the title compound. ¹H NMR (DMSO-d₆, 300 MHz) δ 12.38 (s, 1H), 11.83 (s, 1H), 10.86 (s, 1H), 6.69 (d, J=8.4 Hz, 1H), 8.01 (s, 1H), 7.84 (s, 1H), 7.59 (d, J=6.9 Hz, 1H), 6.52 (s, 1H), 4.84 (d, J=4.2 Hz, 2H), 4.09-4.04 (brs, 2H), 3.82 (brs, 1H), 3.36 (brs, 4H), 3.09 (brs, 2H), 2.01-1.79 (m, 6H), 1.46-1.43 (m, 2H). MS: 455 (M+H⁺).

### Example 97

### 5-{[2-chloro-4-(pyrrolidin-1-ylmethyl)phenyl]amino}-7-(3-hydroxyazetidin-1-yl)pyrido[3,4-d]pyridazin-4(3H)-one

To a solution of EXAMPLE 93C (50 mg, 0.13 mmol) and azetidin-3-ol (14 mg, 0.19 mmol) in 1,4-dioxane (4 mL) was added N-ethyl-N-isopropyl propan-2-amine (34 mg, 0.26 mmol) and the mixture was heated in a sealed tube at 130° C. for 30 hours. After concentration, the residue was purified by preparative HPLC using a gradient of 10/90 to 90/10 acetronitrile in water (containing 0.1% trifluoroacetic acid) to give the title compound. ¹H NMR (CD₃OD, 300 MHz): δ 11.84 (s, 1H), 9.08 (d, J=8.4 Hz, 1H), 7.89 (s, 1H), 7.63 (s, 1H), 7.43 (d, J=8.4 Hz, 1H), 5.87 (s, 1H), 4.76 (brs, 1H), 4.41-4.29 (brs, 4H), 3.96-3.92 (m, 2H), 3.33 (brs, 4H), 2.10 (brs, 6H). MS: 427 (M+H⁺).

### Example 98

### 7-(2-chlorophenoxy)-5-{[2-methoxy-4-(piperidin-4-yl)phenyl]amino}pyrido[3,4-d]pyridazin-4-ol

### Example 98A

### tert-butyl 4-(4-(7-(2-chlorophenoxy)-4-oxo-3,4-dihydropyrido[3,4-d]pyridazin-5-ylamino)-3-methoxyphenyl)piperidine-1-carboxylate

The title compound was obtained following the procedure described in EXAMPLE 62A, using EXAMPLE 99C in place of EXAMPLE 24A.

### Example 98B

### 7-(2-chlorophenoxy)-5-{[2-methoxy-4-(piperidin-4-yl)phenyl]amino}pyrido[3,4-d]pyridazin-4-ol

The title compound was obtained following the procedure described in EXAMPLE 2F, using EXAMPLE 98A in place of EXAMPLE 2E. ¹H NMR (DMSO-d₆, 300 MHz): δ 12.88 (s, 1H), 11.88 (s, 1H), 8.31 (s, 1H), 7.73 (s, 1H), 7.60-7.49 (m, 4H), 7.14 (s, 1H), 6.81 (s, 1H), 6.59 (d, J=6.0 Hz, 1H), 3.89 (s, 3H), 3.47-3.37 (m, 4H), 1.91-1.80 (m, 4H). MS: 479(M+H⁺).

### Example 99

### 7-[(cyclopropylmethyl)amino]-5-{[2-methoxy-4-(piperidin-4-yl)phenyl]amino}pyrido[3,4-d]pyridazin-4-ol

### Example 99A

### tert-buty 14-(4-amino-3-methoxyphenyl)-5,6-dihydropyridine-1(2H)-carboxylate

A mixture of 4-bromo-2-methoxybenzenamine (1.21 g, 6.0 mmol), tert-butyl 4-(3,3,4,4-tetramethylborolan-1-yl)-5,6-dihydropyridine-1(2H)-carboxylate (1.95 g, 6.3 mmol), sodium carbonate (1.91 g, 18 mmol) and 1,1′-bis(diphenylphosphino)ferrocene]dichloropalladium(II) (0.22 g, 0.3 mmol) in dioxane (25 mL) and water (5 mL) was degassed with nitrogen and heated at 90° C. for 15 hours. After cooling to ambient temperature, the mixture was filtered, concentrated and purified by flash chromatography eluting with 200:1 dichloromethane/methanol to give the title compound. MS: 305 (M+H⁺).

### Example 99B

### tert-buty 14-(4-amino-3-methoxyphenyl)piperidine-1-carboxylate

To a mixture of 10% palladium on carbon (0.1 g) in methanol (30 mL) was added EXAMPLE 99A (0.8 g, 2.6 mmol) and the mixture was stirred at ambient temperature under hydrogen for 8 hours. The mixture was filtered and concentrated to give the title compound. MS: 307 (M+H⁺).

### Example 99C

### tert-butyl 4-(4-(7-chloro-4-oxo-3,4-dihydropyrido[3,4-d]pyridazin-5-ylamino)-3-methoxyphenyl)piperidine-1-carboxylate

The title compound was obtained following the procedure described in EXAMPLE 26C, using EXAMPLE 99B in place of EXAMPLE 26B. MS: 486 (M+1).

### Example 99D

### tert-butyl 4-(4-(7-(cyclopropylmethylamino)-4-oxo-3,4-dihydropyrido[3,4-d]pyridazin-5-ylamino)-3-methoxyphenyl)piperidine-1-carboxylate

To a solution of EXAMPLE 99C (100 mg, 0.2 mmol) and cyclopropylmethanamine (50 mg, 0.7 mmol) in dioxane (6 mL) was added N,N-dimethylethanamine (100 mg, 1.1 mmol) and the mixture was stirred at 100° C. for 15 hours. After cooling to ambient temperature, the mixture was concentrated and purified by flash chromatography on silica gel eluting with 80/1 dichloromethane/methanol to give the title compound. MS: 521 (M+H⁺).

### Example 99E

### 7-[(cyclopropylmethyl)amino]-5-{[2-methoxy-4-(piperidin-4-yl)phenyl]amino}pyrido[3,4-d]pyridazin-4-ol

The title compound was obtained following the procedure described in EXAMPLE 26E, using EXAMPLE 99D in place of EXAMPLE 26D. ¹H NMR (CD₃OD, 300 MHz): δ 8.02 (s, 1H), 7.18-7.16 (s, 1H), 7.08-7.05 (m, 2H), 3.96 (s, 3H), 3.58-3.54 (m, 2H), 3.23-3.15 (m, 5H), 2.18-2.01 (m, 4H), 1.47-1.45 (m, 1H), 0.63-0.61 (m, 2H), 0.36-0.32 (m, 2H). MS: 421 (M+H⁺).

### Example 100

### 2-(2-chlorobenzyl)-4-{[2-methoxy-4-(piperidin-4-yl)phenyl]amino}pyrido[2,3-d]pyridazin-5(6H)-one

### Example 100A

### tert-butyl 4-(4-(2-(2-chlorobenzyl)-5-oxo-5,6-dihydropyrido[3,2-d]pyridazin-4-ylamino)-3-methoxyphenyl)piperidine-1-carboxylate

The title compound was obtained following the procedure described in EXAMPLE 101B, using (2-chlorobenzyl)zinc(II) bromide in place of (2,6-dichlorobenzyl)zinc(II) bromide. MS: 576 (M+H⁺).

### Example 100B

### 2-(2-chlorobenzyl)-4-{[2-methoxy-4-(piperidin-4-yl)phenyl]amino}pyrido[2,3-d]pyridazin-5(6H)-one

The title compound was obtained following the procedure described in EXAMPLE 26E, using EXAMPLE 100A in place of EXAMPLE 26D. ¹H NMR (CD₃OD, 300 MHz): δ 8.34 (s, 1H), 7.48-7.25 (m, 5H), 7.01 (s, 1H), 6.93 (d, J=7.8 Hz, 1H), 6.31 (s, 1H), 4.35 (s, 2H), 3.78 (s, 3H), 3.57-3.53 (m, 2H), 3.22-3.14 (m, 2H), 3.02-2.95 (m, 1H), 2.08-1.90 (m, 4H). MS: 476 (M+H⁺).

### Example 101

### 2-(2,6-dichlorobenzyl)-4-{[2-methoxy-4-(piperidin-4-yl)phenyl]amino}pyrido[2,3-d]pyridazin-5(6H)-one

### Example 101A

### tert-butyl 4-(4-((2-chloro-5-oxo-5,6-dihydropyrido[2,3-d]pyridazin-4-yl)amino)-3-methoxyphenyl)piperidine-1-carboxylate

A mixture of EXAMPLE 68F (0.505 g, 2.34 mmol), EXAMPLE 99B (0.651 g, 2.13 mmol), N,N-diisopropylethylamine (0.550 g, 4.26 mmol) and dioxane (20 mL) was heated in a sealed tube at 130° C. for 18 hours. After cooling to ambient temperature, the mixture was concentrated and purified by flash chromatography on silica gel (200-300 mesh) eluting with 80/1 dichloromethane/methanol to give the title compound. MS: 486 (M+H⁺).

### Example 101B

### tert-butyl 4-(4-((2-(2,6-dichlorobenzyl)-5-oxo-5,6-dihydropyrido[2,3-d]pyridazin-4-yl)amino)-3-methoxyphenyl)piperidine-1-carboxylate

A mixture of EXAMPLE 101A (0.100 g, 0.206 mmol), 0.5M (2,6-dichlorobenzyl)zinc(II) bromide in tetrahydrofuran (4.2 mL, 2.1 mmol), tetrakis(triphenylphosphine)palladium (23.8 mg, 0.0206 mmol) in tetrahydrofuran (3 mL) was degassed with nitrogen and heated in a sealed tube at 80° C. for 15 hours. After cooling to ambient temperature, the mixture was quenched with saturated aqueous ammonium chloride solution (10 mL) and extracted with dichloromethane (3×20 mL). The organic phase was dried over anhydrous sodium sulfate, filtered, concentrated and purified by flash chromatography on silica gel (200-300 mesh) eluting with 50/1 dichloromethane/methanol to give the title compound. MS: 610 (M+H⁺).

### Example 101C

### 2-(2,6-dichlorobenzyl)-4-{[2-methoxy-4-(piperidin-4-yl)phenyl]amino}pyrido[2,3-d]pyridazin-5(6H)-one

To a solution of EXAMPLE 101B (0.126 g, 0.206 mmol) in 5 mL dichloromethane was added trifluoroacetic acid (3 mL) and the mixture was stirred at ambient temperature for 1 hour. After concentration, the residue was purified by preparative HPLC using a gradient of 10/90 to 90/10 acetronitrile in water (containing 0.1% trifluoroacetic acid) to give the title compound as a solid trifluoroacetate salt. ¹H NMR (DMSO-d₆, 300 MHz): δ 12.85 (s, 1H), 10.70 (s, 1H), 8.08 (s, 1H), 7.49 (d, J=7.8 Hz, 2H), 7.36-7.30 (m, 1H), 7.18 (d, J=8.1 Hz, 1H), 6.92 (s, 1H), 6.76 (d, J=8.1 Hz, 1H), 6.45 (s, 1H), 4.37 (s, 2H), 3.75 (s, 3H), 3.10-3.05 (m, 2H), 2.67-2.59 (m, 3H), 1.74-1.48 (m, 4H). MS: 510 (M+H⁺).

### Example 102

### 4-{[2-methoxy-4-(piperidin-4-yl)phenyl]amino}-2-[2-(morpholin-4-yl)ethyl]pyrido[2,3-d]pyridazin-5(6H)-one

### Example 102A

### tert-butyl 4-(3-methoxy-4-(5-oxo-2-vinyl-5,6-dihydropyrido[3,2-d]pyridazin-4-ylamino)phenyl)piperidine-1-carboxylate

To a solution of EXAMPLE 101A (200 mg, 0.41 mmol) in dioxane (10 mL) was added tributyl(vinyl)stannane (196 mg, 0.62 mmol) and tetrakis(triphenylphosphine)palladium (47 mg, 0.041 mmol). After heating at 100° C. for 5 hours, the mixture was cooled to ambient temperature, concentrated and purified by flash chromatography on silica gel eluting with 100:1 dichloromethane:methanol to give the title compound. MS: 478 (M+H⁺).

### Example 102B

### tert-butyl 4-(3-methoxy-4-(2-(2-morpholinoethyl)-5-oxo-5,6-dihydropyrido[3,2-d]pyridazin-4-ylamino)phenyl)piperidine-1-carboxylate

To a solution of EXAMPLE 102A (120 mg, 0.25 mmol) in methanol (10 mL) were added acetic acid (300 mg, 5 mmol) and morpholine (109 mg, 1.26 mmol). After heating at 65° C. for 16 hours, the mixture was cooled to ambient temperature and concentrated to give the crude title compound. MS: 565 (M+H⁺).

### Example 102C

### 4-{[2-methoxy-4-(piperidin-4-yl)phenyl]amino}-2-[2-(morpholin-4-yl)ethyl]pyrido[2,3-d]pyridazin-5(6H)-one

To a solution of EXAMPLE 102B (0.25 mmol) in dichloromethane (12 mL) was added 2,2,2-trifluoroacetic acid (3 mL) and the mixture was stirred at ambient temperature for 5 hours. After concentration, the residue was purified by preparative HPLC eluting with a gradient of 10/90 to 30/20 acetonitrile/water (containing 0.1% trifluoroacetic acid) to afford the title compound which was converted into the hydrochloride salt with 1N aqueous hydrochloric acid. ¹H NMR (DMSO-d₆, 300 MHz): δ 13.2 (s, 1H), 11.3 (s, 1H), 9.04 (br, 2H), 8.36 (s, 1H), 7.56 (d, J=8.1 Hz, 1H), 7.09-6.94 (m, 3H), 3.92 (br, 7H), 3.53-3.34 (m, 10H), 3.03-2.93 (br, 3H), 2.00 (br, 4H). MS: 465.2 (M+H⁺).

### Example 103

### 7-(2-chlorophenoxy)-5-{[5-(piperazin-1-yl)pyridin-2-yl]amino}pyrido[3,4-d]pyridazin-4(3H)-one

### Example 103A

### 5-fluoro-2-nitropyridine

To concentrated sulfuric acid (6 mL) at 0° C. was added dropwise 30% aqueous hydrogen peroxide (2.5 mL). To the mixture was added a pre-cooled solution of 5-fluoropyridin-2-amine (1 g, 8.9 mmol) in concentrated sulfuric acid (6 mL). The mixture was stirred at ambient temperature for 16 hours, poured into ice-water and extracted with ethyl acetate. The combined organic phase was washed with brine, dried over sodium sulfate, filtered and concentrated. The residue was purified by flash chromatography on silica gel (200-300 mesh) eluting with 98/2 dichloromethane/methanol to give the title compound. MS: 143 (M+H⁺).

### Example 103B

### tert-butyl 4-(6-nitropyridin-3-yl)piperazine-1-carboxylate

A mixture of EXAMPLE 103A (3.84 g, 27 mmol), tert-butyl piperazine-1-carboxylate (6.04 g, 2.4 mmol) and triethylamine (8.20, 81 mmol) in toluene (150 mL) was heated at 100° C. for 16 hours. The mixture was concentrated and the residue was washed with petroleum ether and dried under vacuum to give the title compound. MS: 309 (M+H⁺).

### Example 103C

### tert-butyl 4-(6-aminopyridin-3-yl)piperazine-1-carboxylate

To a suspension of EXAMPLE 103B (4.5 g, 14.6 mmol) in methanol (100 mL) was added Raney-Nickel (450 mg) and the mixture was stirred at ambient temperature under hydrogen for 4 hours. The catalyst was filtered off and the filtrate was concentrated to give the title compound, which was used in the next step without further purification.

### Example 103D

### tert-butyl 4-(6-(7-chloro-4-oxo-3,4-dihydropyrido[3,4-d]pyridazin-5-ylamino)pyridin-3-yl)piperazine-1-carboxylate

The title compound was obtained following the procedure described in EXAMPLE 57E, using EXAMPLE 103C in place of EXAMPLE 57D. MS: 458 (M+H⁺).

### Example 103E

### tert-butyl 4-(6-(7-(2-chlorophenoxy)-4-oxo-3,4-dihydropyrido[3,4-d]pyridazin-5-ylamino)pyridin-3-yl)piperazine-1-carboxylate

The title compound was obtained following the procedure described in EXAMPLE 75A, using EXAMPLE 103D in place of EXAMPLE 24A and 2-chlorophenol in place of 2,6-difluorophenol. MS: 550 (M+H⁺).

### Example 103F

### 7-(2-chlorophenoxy)-5-{[5-(piperazin-1-yl)pyridin-2-yl]amino}pyrido[3,4-d]pyridazin-4(3H)-one

The title compound was obtained following the procedure described in EXAMPLE 28B, using EXAMPLE 103E in place of EXAMPLE 28A. ¹H NMR (CD₃OD. 300 MHz): δ 8.29 (s, 1H), 7.90 (dd, J=2.7 Hz, J=9.6 Hz, 1H), 7.76-7.69 (m, 2H), 7.62-7.46 (m, 4H), 6.94 (s, 1H), 3.54-3.52 (m, 4H), 3.46-3.44 (m, 4H). MS: 450 (M+H⁺).

### Example 104

### 2-(2-cyclopropylethyl)-4-{[2-methoxy-4-(piperidin-4-yl)phenyl]amino}pyrido[2,3-d]pyridazin-5(6H)-one

### Example 104A

### (E)-tert-butyl 4-(4-(2-(2-cyclopropylvinyl)-5-oxo-5,6-dihydropyrido[3,2-d]pyridazin-4-ylamino)-3-methoxyphenyl)piperidine-1-carboxylate

To a mixture EXAMPLE 101A (200 mg, 0.41 mmol) in dioxane (20 mL) and water (5 mL) were added (E)-2-(2-cyclopropylvinyl)-4,4,5,5-tetramethyl-1,3,2-dioxaborolane (160 mg, 0.82 mmol), 1,1′-bis(diphenylphosphino)ferrocene palladium(II) dichloride (30 mg, 0.041 mmol) and potassium carbonate (170 mg, 1.23 mmol). After heating at 100° C. under nitrogen for 4 hours, the mixture was cooled to ambient temperature, poured into water (50 mL) and extracted with dichloromethane (2×50 mL). The combined organic layers were dried over anhydrous sodium sulfate, filtered, concentrated and purified by flash chromatography on silica gel eluting with 100:1 dichloromethane/methanol to give the title compound. MS: 518 (M+H⁺).

### Example 104B

### tert-butyl 4-(4-(2-(2-cyclopropylethyl)-5-oxo-5,6-dihydropyrido[3,2-d]pyridazin-4-ylamino)-3-methoxyphenyl)piperidine-1-carboxylate

To a solution of EXAMPLE 104A (200 mg, 0.39 mmol) in methanol (20 mL) was added Raney Nickel (50 mg) and the mixture was stirred at ambient temperature under hydrogen for 1 hour. The mixture was filtered and the filtrate was concentrated to give the crude title compound. MS: 520 (M+H⁺).

### Example 104C

### 2-(2-cyclopropylethyl)-4-{[2-methoxy-4-(piperidin-4-yl)phenyl]amino}pyrido[2,3-d]pyridazin-5(6H)-one

To a solution of EXAMPLE 104B (202 mg, 0.39 mmol) in dichloromethane (12 mL) was added 2,2,2-trifluoroacetic acid (3 mL). After stirring at ambient temperature for 5 hours, the mixture was concentrated and the residue was purified by preparative HPLC eluting with a gradient of 10/90 to 30/20 acetonitrile/water (containing 0.1% trifluoroacetic acid) to give the title compound which was converted into the hydrochloride salt with 1N aqueous hydrochloric acid. ¹H NMR (DMSO-d₆, 300 MHz): δ 13.70 (s, 1H), 11.73 (s, 1H), 9.24 (br, 2H), 8.68 (s, 1H), 7.53 (d, J=8.1 Hz, 1H), 7.13 (s, 1H), 7.00-6.98 (m, 2H), 3.88 (s, 3H), 3.41-3.37 (m, 2H), 2.99-2.94 (m, 5H), 2.02-2.00 (m, 4H), 1.66-1.59 (m, 2H), 0.74 (t, J=6.0 Hz, 1H), 0.43-0.38 (m, 2H), 0.13-0.07 (m, 2H). MS: 420.1 (M+H⁺).

### Example 105

### (4-{[7-(2-chlorophenoxy)-4-hydroxypyrido[3,4-d]pyridazin-5-yl]amino}-3-methoxyphenyl)(pyrrolidin-1-yl)methanone

### Example 105A

### (4-amino-3-methoxyphenyl)(pyrrolidin-1-yl)methanone

To a solution of 4-amino-3-methoxybenzoic acid (167 mg, 1 mmol) in dichloromethane (20 mL) at 0° C. was added pyrrolidine (71 mg, 1 mmol), hydroxybenzotriazole monohydrate (270 mg, 2 mmol), 1-ethyl-3-(3-dimethylaminopropyl)carbodiimide (384 mg, 2 mmol) and triethylamine (0.5 mL, 3 mmol). After stirring for 2 hours, the mixture was diluted with water and extracted by dichloromethane. The organic phase was concentrated and the residue was purified by flash chromatography on silica gel (200-300 mesh) eluting with 5/1 petroleum ether/ethyl acetate to give the title compound. MS: 220 (M+H⁺).

### Example 105B

### 7-chloro-5-(2-methoxy-4-(pyrrolidine-1-carbonyl)phenylamino)pyrido[3,4-d]pyridazin-4(3H)-one

A mixture of EXAMPLE 7K (100 mg, 0.47 mmol), EXAMPLE 105A (110 mg, 0.51 mmol) and ethyl diisopropylamine (0.3 mL, 1.41 mmol) in 1,4-dioxane (10 mL) was heated in a sealed tube at 140° C. for 16 hours. The mixture was concentrated and the residue was washed with 1/10 methanol/dichloromethane to give the title compound.

### Example 105C

### (4-{[7-(2-chlorophenoxy)-4-hydroxypyrido[3,4-d]pyridazin-5-yl]amino}-3-methoxyphenyl)(pyrrolidin-1-yl)methanone

A mixture of EXAMPLE 105B (120 mg, 0.3 mmol), 2-chlorophenol (160 mg, 1.2 mmol), cuprous iodide (6 mg, 0.03 mmol), 2-(dimethylamino)acetic acid (17 mg, 0.09 mmol) and cesium carbonate (0.2 g, 0.6 mmol) in 1,4-dioxane (5 mL) was heated in a sealed tube at 120° C. overnight. After concentration, the residue was purified by preparative HPLC using a gradient of 10/90 to 90/10 acetronitrile in water (containing 0.1% trifluoroacetic acid) to give the title compound. ¹H NMR (DMSO-d₆, 300 MHz): δ 12.88 (s, 1H), 11.88 (s, 1H), 8.31 (s, 1H), 7.90 (s, 1H), 7.78-7.72 (m, 1H), 7.60-7.49 (m, 4H), 7.14 (s, 1H), 6.82 (s, 1H), 6.59 (d, J=8.1 Hz, 2H), 3.92 (s, 3H), 3.46-3.40 (m, 4H), 3.91-3.85 (m, 4H). MS: 493 (M+H⁺).

### Example 106

### 7-(2-chlorophenoxy)-5-{[2-ethoxy-4-(piperidin-4-yl)phenyl]amino}pyrido[3,4-d]pyridazin-4(3H)-one

### Example 106A

### tert-butyl 4-(4-amino-3-ethoxyphenyl)-5,6-dihydropyridine-1(2H)-carboxylate

To a solution of 4-bromo-2-ethoxyaniline (215 mg, 4.65 mmol) in 1,4-dioxane (40 mL) and water (10 mL) was added tert-butyl 4-(4,4,5,5-tetramethyl-1,3,2-dioxborolan-2-yl)-5,6-dihydropyridine-1(2H)-carboxylate (1.58 g, 5.12 mmol), 1,1′-bis(diphenylphosphino)ferrocene palladium(II) dichloride (339 mg, 0.46 mmol) and potassium carbonate (1.9 g, 3.95 mmol) and the mixture was heated at 100° C. for 14 hours. After cooling to ambient temperature, the mixture was filtered and purified by flash chromatography on silica gel eluting with 100/1 dichloromethane/methanol to provide the title compound. MS: 319 (M+HE).

### Example 106B

### tert-butyl 4-(4-amino-3-ethoxyphenyl)piperidine-1-carboxylate

To a solution of EXAMPLE 106A (360 mg, 1.13 mmol) in methanol (100 mL) was added 10% palladium on carbon (500 mg) and the mixture was stirred under hydrogen for 14 hours. The solid was filtered off and the residue was purified by flash chromatography on silica gel (200-300 mesh) eluting with 100/1 dichloromethane/methanol to provide the title compound. MS: 265 (M−56+H⁺).

### Example 106C

### tert-butyl 4-(4-(7-chloro-4-oxo-3,4-dihydropyrido[3,4-d]pyridazin-5-ylamino)-3-ethoxyphenyl)piperidine-1-carboxylate

To a solution of EXAMPLE 106B (270 mg, 0.84 mmol) in 1,4-dioxane (20 mL) was added EXAMPLE 7K (150 mg, 0.7 mmol) and N,N-diisopropylethylamine (0.25 mL, 1.4 mmol) and the mixture was heated in a sealed tube at 120° C. for 14 hours. After cooling to ambient temperature, the mixture was filtered and purified by flash chromatography on silica gel eluting with 100/1 dichloromethane/methanol to provide the title compound. MS: 500 (M+H⁺).

### Example 106D

### 7-(2-chlorophenoxy)-5-{[2-ethoxy-4-(piperidin-4-yl)phenyl]amino}pyrido[3,4-d]pyridazin-4(3H)-one

To a solution of EXAMPLE 106C (240 mg, 0.48 mmol) in 1,4-dioxane (20 mL) was added 2-chlorophenol (0.06 mL, 0.58 mmol), cuprous iodide (9 mg, 0.048 mmol), 2-(dimethylamino)acetic acid (15 mg, 0.14 mmol) and cesium carbonate (312 mg, 0.96 mmol) and the mixture was heated in a sealed tube at 100° C. for 14 hours. After cooling to ambient temperature, the mixture was filtered and purified by flash chromatography on silica gel eluting with 100/1 dichloromethane/methanol to provide the title compound. MS: 592 (M+H⁺).

### Example 107

### 2-(2-fluorobenzyl)-4-{[2-methoxy-4-(piperidin-4-yl)phenyl]amino}pyrido[2,3-d]pyridazin-5(6H)-one

### Example 107A

### tert-buty 14-(4-((2-(2-fluorobenzyl)-5-oxo-5,6-dihydropyrido[2,3-d]pyridazin-4-yl)amino)-3-methoxyphenyl)piperidine-1-carboxylate

A mixture of EXAMPLE 101A (80.0 mg, 0.165 mmol), 0.5M (2-fluorobenzyl)zinc(II) bromide in tetrahydrofuran (3.3 mL, 1.65 mmol), tetrakis(triphenylphosphine)palladium (19.1 mg, 0.0165 mmol) in tetrahydrofuran (2 mL) was degassed with nitrogen and heated in a sealed tube at 80° C. for 15 hours. After cooling to ambient temperature, the mixture was quenched with saturated aqueous ammonium chloride solution (10 mL) and extracted with dichloromethane (3×20 mL). The combined organic phase was dried over anhydrous sodium sulfate, filtered, concentrated and purified by flash chromatography on silica gel (200-300 mesh) eluting with 50/1 dichloromethane/methanol to give the title compound. MS: 560 (M+H⁺).

### Example 107B

### 2-(2-fluorobenzyl)-4-{[2-methoxy-4-(piperidin-4-yl)phenyl]amino}pyrido[2,3-d]pyridazin-5(6H)-one

To a solution of EXAMPLE 107A (50.0 mg, 0.0893 mmol) in dichloromethane (5 mL) was added trifluoroacetic acid (3 mL) and the mixture was stirred at ambient temperature for 1 hour. After concentration, the residue was purified by preparative HPLC using a gradient of 10/90 to 90/10 acetronitrile in water (containing 0.1% trifluoroacetic acid) to give the title compound. ¹H NMR (DMSO-d₆+D₂O, 300 MHz): δ 8.22 (s, 1H), 7.35-7.29 (m, 3H), 7.20-7.14 (m, 2H), 7.00 (s, 1H), 6.90-6.87 (m, 1H), 6.61 (s, 1H), 3.75 (s, 3H), 3.42-3.38 (m, 2H), 3.06-2.85 (m, 3H), 2.01-1.73 (m, 4H). MS: 460 (M+H⁺).

### Example 108

### 2-(2,3-dichlorobenzyl)-4-{[2-methoxy-4-(piperidin-4-yl)phenyl]amino}pyrido[2,3-d]pyridazin-5(6H)-one

### Example 108A

### tert-butyl 4-(4-(2-(2,3-dichlorobenzyl)-5-oxo-5,6-dihydropyrido[3,2-d]pyridazin-4-ylamino)-3-methoxy phenyl)piperidine-1-carboxylate

The title compound was obtained following the procedure described in EXAMPLE 107A, using (2,3-dichlorobenzyl)zinc(II) bromide in place of (2-fluorobenzyl)zinc(II) bromide. MS: 610 (M+H⁺).

### Example 108B

### 2-(2,3-dichlorobenzyl)-4-{[2-methoxy-4-(piperidin-4-yl)phenyl]amino}pyrido[2,3-d]pyridazin-5(6H)-one

The title compound was obtained following the procedure described in EXAMPLE 107B, using EXAMPLE 108A in place of EXAMPLE 107A. ¹H NMR (CD₃OD, 300 MHz): δ 8.29 (s, 1H), 7.57-7.54 (m, 1H), 7.33-7.25 (s, 3H), 7.00 (s, 1H), 6.93 (d, J=8.1 Hz, 1H), 6.31 (s, 1H), 4.37 (s, 2H), 3.79 (s, 3H), 3.57-3.53 (m, 2H), 3.21-2.94 (m, 3H), 2.14-1.91 (m, 4H). MS: 510 (M+H⁺).

### Example 109

### 2-(2,6-difluorobenzyl)-4-{[2-methoxy-4-(piperidin-4-yl)phenyl]amino}pyrido[2,3-d]pyridazin-5(6H)-one

### Example 109A

### tert-buty 14-(4-(2-(2,6-difluorobenzyl)-5-oxo-5,6-dihydropyrido[3,2-d]pyridazin-4-ylamino)-3-methoxyphenyl)piperidine-1-carboxylate

The title compound was obtained following the procedure described in EXAMPLE 107A, using (2,6-fluorobenzyl)zinc(II) bromide in place of (2-fluorobenzyl)zinc(II) bromide. MS: 578 (M+H⁺).

### Example 109B

### 2-(2,6-difluorobenzyl)-4-{[2-methoxy-4-(piperidin-4-yl)phenyl]amino}pyrido[2,3-d]pyridazin-5(6H)-one

The title compound was obtained following the procedure described in EXAMPLE 107B, using EXAMPLE 109A in place of EXAMPLE 107A. ¹H NMR (CD₃OD, 300 MHz): δ 8.35 (s, 1H), 7.51-7.41 (m, 1H), 7.29 (d, J=8.1 Hz, 1H), 7.11-7.05 (m, 4H), 6.97 (dd. J=1.5, 8.1 Hz, 1H), 6.38 (s, 1H), 4.30 (s, 2H), 3.80 (s, 3H), 3.59-3.54 (m, 2H), 3.24-3.15 (m, 1H), 3.05-2.97 (m, 1H), 2.14-1.96 (m, 4H). MS: 478 (M+H⁺).

### Example 110

### 2-(2,5-difluorobenzyl)-4-{[2-methoxy-4-(piperidin-4-yl)phenyl]amino}pyrido[2,3-d]pyridazin-5(6H)-one

### Example 110A

### tert-butyl 4-(4-((2-(2,5-difluorobenzyl)-5-oxo-5,6-dihydropyrido[2,3-d]pyridazin-4-yl)amino)-3-methoxyphenyl)piperidine-1-carboxylate

A mixture of EXAMPLE 101A (80.0 mg, 0.165 mmol), 0.25M (2,5-difluorobenzyl)zinc(11) bromide in tetrahydrofuran (6.6 mL, 1.65 mmol) and tetrakis(triphenylphosphine)palladium (19.1 mg, 0.0165 mmol) was degassed with nitrogen and heated in a sealed tube at 80° C. for 15 hours. After cooling to ambient temperature, the mixture was diluted with saturated aqueous ammonium chloride solution (10 mL) and extracted with dichloromethane (3×20 mL). The organic phase was dried over anhydrous sodium sulfate, filtered, concentrated and purified by flash chromatography on silica gel (200-300 mesh) eluting with 50/1 dichloromethane/methanol to give the title compound. MS: 578 (M+H⁺).

### Example 110B

### 2-(2,5-difluorobenzyl)-4-{[2-methoxy-4-(piperidin-4-yl)phenyl]amino}pyrido[2,3-d]pyridazin-5(6H)-one

To a solution of EXAMPLE 110A (90.5 mg, 0.157 mmol) in dichloromethane (5 mL) was added trifluoroacetic acid (3 mL) and the mixture was stirred at ambient temperature for 1 hour. After concentration, the residue was purified by preparative HPLC using a gradient of 10/90 to 90/10 acetronitrile in water (containing 0.1% trifluoroacetic acid) to give the title compound as a solid trifluoroacetate salt. ¹H NMR (DMSO-d₆, 300 MHz): δ 13.15 (s, 1H), 11.15 (s, 1H), 8.90-8.85 (m, 1H), 8.74-8.69 (m, 1H), 8.27 (s, 1H), 7.37 (d, J=8.1 Hz, 1H), 7.29-7.13 (m, 3H), 7.00 (s, 1H), 6.87 (d, J=8.1 Hz, 1H), 6.76 (s, 1H), 4.15 (s, 2H), 3.79 (s, 3H), 3.41-3.36 (m, 2H), 3.06-2.85 (m, 3H), 2.01-1.81 (m, 4H). MS: 478 (M+H⁺).

### Example 111

### 2-(2,3-difluorobenzyl)-4-{[2-methoxy-4-(piperidin-4-yl)phenyl]amino}pyrido[2,3-d]pyridazin-5(6H)-one

### Example 111A

### tert-butyl 4-(4-(2-(2,3-difluorobenzyl)-5-oxo-5,6-dihydropyrido[3,2-d]pyridazin-4-ylamino)-3-methoxyphenyl)piperidine-1-carboxylate

The title compound was obtained following the procedure described in EXAMPLE 107A, using (2,3-fluorobenzyl)zinc(II) bromide in place of (2-fluorobenzyl)zinc(II) bromide. MS: 578 (M+H⁺).

### Example 111B

### 2-(2,3-difluorobenzyl)-4-{[2-methoxy-4-(piperidin-4-yl)phenyl]amino}pyrido[2,3-d]pyridazin-5(6H)-one

The title compound was obtained following the procedure described in EXAMPLE 107B, using EXAMPLE 11A in place of EXAMPLE 107A. ¹H NMR (CD₃OD, 300 MHz): δ 8.34 (s, 1H), 7.35-7.17 (m, 4H), 7.09 (s, 1H), 7.00 (d, J=8.1 Hz, 1H), 6.53 (s, 1H), 4.32 (s, 2H), 3.83 (s, 3H), 3.58-3.54 (m, 2H), 3.23-3.02 (m, 3H), 2.15-1.97 (m, 4H). MS: 478 (M+H⁺).

### Example 112

### 2-(2-chloro-6-fluorobenzyl)-4-{[2-methoxy-4-(piperidin-4-yl)phenyl]amino}pyrido[2,3-d]pyridazin-5(6H)-one

### Example 112A

### tert-butyl 4-(4-(2-(2-chloro-6-fluorobenzyl)-5-oxo-5,6-dihydropyrido[3,2-d]pyridazin-4-ylamino)-3-methoxyphenyl)piperidine-1-carboxylate

The title compound was obtained following the procedure described in EXAMPLE 107A, using (2-chloro-6-fluorobenzyl)zinc(II) bromide in place of (2-fluorobenzyl)zinc(II) bromide. MS: 594 (M+H⁺).

### Example 112B

### 2-(2-chloro-6-fluorobenzyl)-4-{[2-methoxy-4-(piperidin-4-yl)phenyl]amino}pyrido[2,3-d]pyridazin-5(6H)-one

The title compound was obtained following the procedure described in EXAMPLE 107B, using EXAMPLE 112A in place of EXAMPLE 107A. ¹H NMR (CD₃OD, 300 MHz): δ 8.35 (s, 1H), 7.56-7.42 (m, 1H), 7.29 (d, J=8.1 Hz, 1H), 7.11-7.05 (m, 3H), 6.97 (d, J=8.1 Hz, 1H), 6.38 (s, 1H), 4.30 (s, 2H), 3.80 (s, 3H), 3.58-3.54 (m, 2H), 3.21-3.05 (m, 3H), 2.12-2.00 (m, 4H). MS: 494 (M+H⁺).

### Example 113

### 5-[(2-methoxyphenyl)amino]-7-{[4-(piperazin-1-yl)phenyl]amino}pyrido[3,4-d]pyridazin-4-ol

### Example 113A

### 7-chloro-5-(2-methoxyphenylamino)pyrido[3,4-d]pyridazin-4(3H)-one

The title compound was obtained following the procedure described in EXAMPLE 106C, using 2-methoxyaniline in place of EXAMPLE 106B.

### Example 113B

### tert-butyl 4-(4-(5-(2-methoxyphenylamino)-4-oxo-3,4-dihydropyrido[3,4-d]pyridazin-7-ylamino)phenyl)piperazine-1-carboxylate

The title compound was obtained following the procedure described in EXAMPLE 10A, using EXAMPLE 113A in place of EXAMPLE 24A and tert-butyl 4-(4-aminophenyl)piperazine-1-carboxylate in place of 2,6-dichlorobenzenamine.

### Example 113C

### 5-[(2-methoxyphenyl)amino]-7-{[4-(piperazin-1-yl)phenyl]amino}pyrido[3,4-d]pyridazin-4-ol

The title compound was obtained following the procedure described in EXAMPLE 2F, using EXAMPLE 113B in place of EXAMPLE 2E. ¹H NMR (DMSO-d₆, 300 MHz): δ 12.27 (s, 1H), 11.69 (s, 1H), 9.41 (s, 1H), 9.12 (s, 2H), 8.61 (s, 1H), 8.03 (s, 1H), 7.44-7.41 (m, 3H), 7.24 (s, 1H), 7.07-6.99 (m, 6H), 6.88-6.83 (m, 1H), 6.20 (s, 1H), 3.90 (s, 3H), 3.38-3.28 (m, 8H). MS: 445 (M+H⁺).

### Example 114

### 7-(2-chlorophenoxy)-5-{[2-ethoxy-4-(piperazin-1-yl)phenyl]amino}pyrido[3,4-d]pyridazin-4-ol

### Example 114A

### 4-bromo-2-ethoxybenzenamine

To a solution of 2-ethoxybenzenamine (2 g, 14.58 mmol) in acetonitrile (100 mL) at 0° C. was added N-bromosuccinimide (2.72 g, 1.05 mmol) and the mixture was stirred at ambient temperature for 4 hours. The mixture was concentrated and the residue was purified by flash chromatography on silica gel (200-300 mesh) eluting with 6/1 petroleum ether/ethyl acetate to provide the title compound. MS: 216 (M+H⁺).

### Example 114B

### N,N-dibenzyl-4-bromo-2-ethoxybenzenamine

To a solution of EXAMPLE 114A (500 mg, 2.32 mmol) in acetonitrile (20 mL) were added potassium carbonate (963 mg, 6.98 mmol) and benzyl bromide (0.83 mL, 6.98 mmol) and the mixture was heated at 90° C. for 14 hours. The mixture was diluted with water (100 mL) and extracted with dichloromethane (3×100 mL). The combined organic layers were concentrated to provide the title compound. MS: 396 (M+H⁺).

### Example 114C

### tert-butyl 4-(4-(dibenzylamino)-3-ethoxyphenyl)piperazine-1-carboxylate

A mixture of EXAMPLE 114B (500 mg, 1.26 mmol), tert-butyl piperazine-1-carboxylate (260 mg, 1.39 mmol), palladium diacetate (44 mg, 0.19 mmol), (±)-2,2′-bis(diphenylphosphino)-1,1′-binaphthalene (235 mg, 0.38 mmol) and cesium carbonate (823 mg, 2.53 mmol) in 1,4-dioxane (10 mL) was heated at 110° C. for 3 hours. After cooling to ambient temperature, the mixture was filtered and purified by flash chromatography on silica gel eluting with 100/1 dichloromethane/methanol to provide the title compound. MS: 502 (M+H⁺).

### Example 114D

### tert-butyl 4-(4-amino-3-ethoxyphenyl)piperazine-1-carboxylate

To a solution of EXAMPLE 114C (1 g, 1.99 mmol) in methanol (100 mL) was added 10% palladium on carbon (500 mg) and the mixture was stirred under hydrogen for 14 hours. The solid was filtered off and the filtrate was purified by flash chromatography on silica gel (200˜300 mesh) eluting with 100/1 dichloromethane/methanol to provide the title compound. MS: 322 (M+H⁺).

### Example 114E

### tert-butyl 4-(4-(7-chloro-4-oxo-3,4-dihydropyrido[3,4-d]pyridazin-5-ylamino)-3-ethoxyphenyl)piperazine-1-carboxylate

To a solution of EXAMPLE 114D (322 mg, 1 mmol) in 1,4-dioxane (20 mL) was added EXAMPLE 7K (215 mg, 1 mmol) and N,N-diisopropylethylamine (0.4 mL, 2 mmol) and the mixture was heated in a sealed tube at 120° C. for 14 hours. After cooling to ambient temperature, the mixture was filtered and purified by flash chromatography on silica gel eluting with 100/1 dichloromethane/methanol to provide the title compound. MS: 501 (M+H⁺).

### Example 114F

### tert-butyl 4-(4-(7-(2-chlorophenoxy)-4-oxo-3,4-dihydropyrido[3,4-d]pyridazin-5-ylamino)-3-ethoxyphenyl)piperazine-1-carboxylate

To a solution of EXAMPLE 114E (400 mg, 1 mmol) in 1,4-dioxane (20 mL) was added 2-chlorophenol (0.1 ml, 0.96 mmol), cuprous iodide (15 mg, 0.1 mmol), 2-(dimethylamino)acetic acid (25 mg, 0.24 mmol) and cesium carbonate (520 mg, 1.6 mmol) and the mixture was heated in a sealed tube at 100° C. for 14 hours. After cooling to ambient temperature, the mixture was filtered and purified by flash chromatography on silica gel eluting with 100/1 dichloromethane/methanol to provide the title compound. MS: 593 (M+H⁺).

### Example 114G

### 7-(2-chlorophenoxy)-5-{[2-ethoxy-4-(piperazin-1-yl)phenyl]amino}pyrido[3,4-d]pyridazin-4-ol

To a solution of EXAMPLE 14F (410 mg, 0.69 mmol) in dichloromethane (20 mL) was added 2,2,2-trifluoroacetic acid (6 mL) and the mixture was stirred at ambient temperature for 6 hours. The mixture was concentrated and the residue was purified by preparative HPLC using a gradient of 10/90 to 80/20 acetronitrile in water (containing 0.1% trifluoroacetic acid) to give the title compound. ¹H NMR (DMSO-d₆, 300 MHz): δ 12.84 (s, 1H), 11.72 (s, 1H), 8.81-8.78 (br, 2H), 8.26 (s, 1H), 7.73-7.70 (m, 1H), 7.56-7.44 (m, 4H), 6.68-6.64 (m, 2H), 5.98-5.96 (m, 1H), 4.15-4.10 (q, 2H), 3.27 (m, 8H), 1.46-1.42 (t, 3H). MS: 493 (M+H⁺).

### Example 115

### 2-[(5-{[2-methoxy-4-(piperazin-1-yl)phenyl]amino}-4-oxo-3,4-dihydropyrido[3,4-d]pyridazin-7-yl)oxy]benzonitrile

### Example 115A

### tert-butyl 4-(4-(7-(2-cyanophenoxy)-4-oxo-3,4-dihydropyrido[3,4-d]pyridazin-5-ylamino)-3-methoxyphenyl)piperazine-1-carboxylate

The title compound was obtained following the procedure described in EXAMPLE 62A, using 2-hydroxybenzonitrile in place of 2-chlorophenol.

### Example 115B

### 2-[(5-{[2-methoxy-4-(piperazin-1-yl)phenyl]amino}-4-oxo-3,4-dihydropyrido[3,4-d]pyridazin-7-yl)oxy]benzonitrile

The title compound was obtained following the procedure described in EXAMPLE 2F, using EXAMPLE 115A in place of EXAMPLE 2E. ¹H NMR (DMSO-d₆, 300 MHz): δ 12.82 (s, 1H), 11.59 (s, 1H), 9.03 (s, 2H), 8.25 (s, 1H), 8.03 (d, J=7.8 Hz, 1H), 7.79-7.85 (m, 1H), 7.60-7.53 (m, 2H), 7.35 (d, J=8.7 Hz, 1H), 6.70-6.65 (m, 2H), 5.98-5.95 (m, 1H), 3.85 (s, 3H), 3.27-3.21 (m, 8H). MS: 471 (M+H⁺).

### Example 116

### 2-(2-chlorobenzyl)-4-({2-methoxy-4-[(4-methylpiperazin-1-yl)methyl]phenyl}amino)pyrido[2,3-d]pyridazin-5(6H)-one

### Example 116A

### (3-methoxy-4-nitrophenyl)methanol

To a suspension of sodium borohydride (3.85 g, 101.4 mmol) in tetrahydrofuran (20 mL) at 0° C. was added slowly a solution of 3-methoxy-4-nitrobenzoic acid (10.0 g, 50.7 mmol). Boron (tri)fluoride etherate (19.3 mL) was added dropwise and the mixture was stirred at ambient temperature for 4 hours. The mixture was diluted with water (100 mL) and extracted with ethyl acetate (3×150 mL). The combined organic extracts were dried over anhydrous sodium sulfate, filtered and concentrated to give the title compound.

### Example 116B

### (4-amino-3-methoxyphenyl)methanol

To a solution of EXAMPLE 116A (6.25 g, 34 mmol) was added 10% Raney Nickel (625 mg) in methanol (400 mL). After stirring for 8 hours at ambient temperature under hydrogen, the mixture was filtered and concentrated to give the crude title compound. MS: 154 (M+H⁺).

### Example 116C

### 2-chloro-4-(4-(hydroxymethyl)-2-methoxyphenylamino)pyrido[3,2-d]pyridazin-5(6H)-one

A mixture of EXAMPLE 68F (250 mg, 1.17 mmol), EXAMPLE 116B (190 mg, 1.23 mmol) and N,N-diisopropylethylamine (0.5 mL) in 1,4-dioxane (20 mL) was stirred at 100° C. for 16 hours. The mixture was concentrated and the residue was purified by flash chromatography on silica gel (200-300 mesh) eluting with a gradient of 100/1 to 70/1 dichloromethane/methanol to give title compound. MS: 333(M+H⁺).

### Example 116D

### 2-(2-chlorobenzyl)-4-(4-(hydroxymethyl)-2-methoxyphenylamino)pyrido[3,2-d]pyridazin-5(6H)-one

A mixture of EXAMPLE 116C (318 mg, 0.96 mmol), 0.5M 2-chlorobenzyl)zinc(II) bromide in tetrahydrofuran (19.2 mL, 9.6 mmol) and tetrakis(triphenylphosphine)palladium (110 mg, 0.1 mmol) in tetrahydrofuran (8 mL) was heated under nitrogen at 70° C. for 16 hours. The mixture was neutralized with ammonium chloride solution (5 mL) and extracted with ethyl acetate (3×15 mL). The combined organic layers were washed with brine, dried over anhydrous sodium sulfate, filtered, and concentrated. The residue was purified by flash chromatography on silica gel (200-300 mesh) eluting with a gradient of 100/1 to 50/1 dichloromethane/methanol to give the title compound. MS: 423 (M+H⁺).

### Example 116E

### 4-(2-(2-chlorobenzyl)-5-oxo-5,6-dihydropyrido[3,2-d]pyridazin-4-ylamino)-3-methoxybenzaldehyde

To a solution of EXAMPLE 116D (150 mg, 0.35 mmol) in dichloromethane (30 mL) at 0° C. was added Dess-Martin periodinane (180 mg, 0.42 mmol) and the mixture was stirred at ambient temperature for 4 hours. The mixture was quenched with saturated sodium thiosulfate (5 mL), diluted with water (20 mL) and extracted with dichloromethane (3×20 mL). The combined organic extracts were dried over anhydrous sodium sulfate, filtered and concentrated. The residue was purified by flash chromatography on silica gel (200-300 mesh) eluting with a gradient of 100/1 to 30/1 dichloromethane/methanol to give the title compound. MS: 421 (M+H⁺).

### Example 116F

### 2-(2-chlorobenzyl)-4-({2-methoxy-4-[(4-methylpiperazin-1-yl)methyl]phenyl}amino)pyrido[2,3-d]pyridazin-5(6H)-one

To a solution of EXAMPLE 116E (40 mg, 0.1 mmol) and N-methylpiperazine (12 L, 0.11 mmol) in 1,2-dichloroethane (20 mL) was added acetic acid (12 mg, 0.2 mmol) and 4 Å molecular sieves. The mixture was stirred at ambient temperature for 1 hour and sodium triacetoxyborohydride (45 mg, 0.2 mmol) was added. The mixture was stirred at ambient temperature for 14 hours, treated with saturated ammonium chloride aqueous solution (8 mL), and extracted with dichloromethane (3×20 mL). The organic phase was dried over anhydrous sodium sulfate, filtered and concentrated, and the residue was purified by preparative HPLC using a gradient of 10/90 to 90/10 acetronitrile in water (containing 0.1% trifluoroacetic acid) to give the title compound. ¹H NMR (CD₃OD, 300 MHz): δ 8.36 (s, 1H), 7.55-7.26 (m, 5H), 6.56 (s, 1H), 4.50 (s, 2H), 4.40 (s, 2H), 3.87 (s, 3H), 3.66 (brs, 8H), 3.05 (s, 3H). MS: 505 (M+H⁺).

### Example 117

### 7-(2-chlorophenoxy)-5-{[2-(difluoromethoxy)-4-(piperazin-1-yl)phenyl]amino}pyrido[3,4-d]pyridazin-4(3H)-one

### Example 117A

### 4-bromo-2-(difluoromethoxy)-1-nitrobenzene

A suspension of 5-bromo-2-nitrophenol (2.0 g, 9.2 mmol), sodium 2-chloro-2,2-difluoroacetate (3.5 g, 23 mmol) and cesium carbonate (4.5 g, 13.8 mmol) in 1/1 N,N-dimethylformamide/water (40 mL) was heated at 90° C. for 16 hours. The mixture was diluted with water (50 mL) and extracted with ethyl acetate (3×30 mL). The combined organic phase was dried over sodium sulfate, filtered, concentrated and purified by flash chromatography on silica gel (200-300 mesh) eluting with 40/1 petroleum ether/ethyl acetate to give the title compound. MS: 268 (M+H⁺).

### Example 117B

### tert-butyl 4-(3-(difluoromethoxy)-4-nitrophenyl)piperazine-1-carboxylate

A suspension of EXAMPLE 117A (300 mg, 1.1 mmol), tert-butyl piperazine-1-carboxylate (400 mg, 2.1 mmol), palladium(II) acetate (75 mg, 0.32 mmol), (R)-(+)-2,2′-bis(diphenylphosphosino)-1,1′-binaphthyl (399 mg, 0.64 mmol) and cesium carbonate (1.0 g, 3.2 mmol) in toluene (60 mL) was heated in a sealed tube at 100° C. under nitrogen for 16 hours. After concentration, the residue was purified by flash chromatography on silica gel (200-300 mesh) eluting with 20/1 petroleum ether/ethyl acetate to give the title compound. MS: 374 (M+H⁺).

### Example 117C

### tert-buty 14-(4-amino-3-(difluoromethoxy)phenyl)piperazine-1-carboxylate

A suspension of EXAMPLE 117B (150 mg, 0.39 mmol) and 10% palladium on carbon (15 mg) in methanol (20 mL) was stirred under hydrogen for 12 hours. The catalyst was filtered and the filtrate was concentrated to provide the crude title compound which was used in the next step without further purification. MS: 344 (M+H⁺).

### Example 117D

### tert-butyl 4-(4-(7-chloro-4-oxo-3,4-dihydropyrido[3,4-d]pyridazin-5-ylamino)-3-(difluoromethoxy)phenyl)piperazine-1-carboxylate

The title compound was obtained following the procedure described in EXAMPLE 24A, using EXAMPLE 117C in place of EXAMPLE 1G.

### Example 117E

### tert-butyl 4-(4-(7-(2-chlorophenoxy)-4-oxo-3,4-dihydropyrido[3,4-d]pyridazin-5-ylamino)-3-(difluoromethoxy)phenyl)piperazine-1-carboxylate

The title compound was obtained following the procedure described in EXAMPLE 62A, using EXAMPLE 117D in place of EXAMPLE 24A.

### Example 117F

### 7-(2-chlorophenoxy)-5-{[2-(difluoromethoxy)-4-(piperazin-1-yl)phenyl]amino}pyrido[3,4-d]pyridazin-4(3H)-one

The title compound was obtained following the procedure described in EXAMPLE 2F, using EXAMPLE 117E in place of EXAMPLE 2E. ¹H NMR (DMSO-d6, 300 MHz): δ 12.83 (s, 1H), 11.65 (s, 1H), 8.90 (s, 2H), 8.24 (s, 1H), 7.52-7.44 (m, 5H), 7.21 (t, 1H), 7.67 (s, 1H), 6.28 (dd, J=2.7 Hz, 9.3 Hz, 1H), 3.58-3.55 (m, 8H). MS: 516(M+H⁺).

### Example 118

### 7-(2-chlorophenoxy)-5-{[5-(piperidin-4-yl)pyridin-2-yl]amino}pyrido[3,4-d]pyridazin-4(3H)-one

### Example 118A

### tert-butyl 4-(6-aminopyridin-3-yl)-5,6-dihydropyridine-1(2H)-carboxylate

A mixture of 5-bromopyridin-2-amine (1 g, 5.78 mmol), tert-butyl 4-(4,4,5,5-tetramethyl-1,3,2-dioxaborolan-2-yl)-5,6-dihydropyridine-1(2H)-carboxylate (2.14 g, 6.94 mmol), [1,1′-bis(diphenylphosphino)ferrocene]palladium(II) chloride (212 mg, 0.29 mmol) and sodium carbonate (1.84 g, 17.34 mmol) in 1,4-dioxne (30 mL) and water (7 mL) was heated under nitrogen at 80° C. for 16 hours. The mixture was concentrated and the residue was purified by flash chromatography on silica gel (200-300 mesh) eluting with 95/5 dichloromethane/methanol to give the title compound. MS: 276 (M+H⁺).

### Example 118B

### tert-butyl 4-(6-aminopyridin-3-yl)piperidine-1-carboxylate

To a suspension of EXAMPLE 118A (1.6 g, 5.80 mmol) in methanol (100 mL) was added 10% palladium on carbon (200 mg) and the mixture was stirred at ambient temperature under hydrogen for 4 hours. The catalyst was filtered off and the filtrate was concentrated. The residue was purified by flash chromatography on silica gel (200-300 mesh) eluting with 95/5 dichloromethane/methanol to give the title compound. MS: 278 (M+H⁺).

### Example 118C

### tert-butyl 4-(6-(7-chloro-4-oxo-3,4-dihydropyrido[3,4-d]pyridazin-5-ylamino) pyridin-3-yl)piperidine-1-carboxylate

The title compound was obtained following the procedure described in EXAMPLE 3U 57E, using EXAMPLE 118B in place of EXAMPLE 57D. MS: 457 (M+H⁺).

### Example 118D

### tert-butyl 4-(6-(7-(2-chlorophenoxy)-4-oxo-3,4-dihydropyrido[3,4-d]pyridazin-5-ylamino)pyridin-3-yl)piperidine-1-carboxylate

The title compound was obtained following the procedure described in EXAMPLE 62A, using EXAMPLE 118C in place of EXAMPLE 24A. MS: 549 (M+H⁺).

### Example 118E

### 7-(2-chlorophenoxy)-5-{[5-(piperidin-4-yl)pyridin-2-yl]amino}pyrido[3,4-d]pyridazin-4(3H)-one

The title compound was obtained following the procedure described in EXAMPLE 28B, using EXAMPLE 118D in place of EXAMPLE 28A. ¹H NMR (CD₃OD, 300 MHz): δ 8.30 (s, 1H), 7.98-7.96 (m, 2H), 7.80-7.69 (m, 2H), 7.55-7.49 (m, 3H), 6.99 (s, 1H), 3.58-3.54 (m, 2H), 3.32-3.31 (m, 3H), 2.15-1.95 (m, 4H). MS: 449 (M+H⁺).

### Example 119

### 7-(2-chlorophenoxy)-5-{[2-methoxy-4-(piperazin-1-yl)phenyl]amino}-1-methylpyrido[3,4-d]pyridazin-4-ol

### Example 119A

### 2,6-dichloroisonicotinic acid

A solution of 2,6-dihydroxyisonicotinic acid (31.0 g, 200 mmol) in phosphoryl trichloride (150 mL) was stirred in a high-pressure reactor at 130° C. for 6 hours and at 150° C. for 1 hour. After cooling to ambient temperature, the mixture was poured onto ice (1000 g) and stirred for 2 hours. The mixture was filtered to give the title compound.

### Example 119B

### 2,6-dichloro-N-methoxy-N-methylisonicotinamide

To a solution of EXAMPLE 119A (8.0 g, 43.2 mmol) in dichloromethane (150 mL) was added 1,1′-carbonyldiimidazole (12.5 g, 77.2 mmol) in portions over 20 minutes. N,O-dimethylhydroxylamine (12.0 g, 122.4 mmol) was added in portions and the mixture was stirred for 10 hours, filtered and concentrated. The crude residue was purified by flash chromatography on silica gel eluting with 10:1 petroleum ether:ethyl acetate to yield the title compound.

### Example 119C

### 1-(2,6-dichloropyridin-4-yl)ethanone

To a solution of EXAMPLE 119B (5.7 g, 24.3 mmol) in tetrahydrofuran (150 mL) at −70 C was added a solution of methylmagnesium chloride (16.2 mL, 48.6 mmol) in tetrahydrofuran dropwise. The mixture was warmed up to ambient temperature and stirred for 2 hours. The mixture was treated with saturated sodium chloride (10 mL) and extracted with ethyl acetate (2×100 mL). The combined organic phase was dried over magnesium sulfate, filtered, concentrated and purified by flash chromatography on silica gel eluting with 16:1 petroleum ether:ethyl acetate to give the title compound.

### Example 119D

### 2,6-dichloro-4-(2-methyl-1,3-dioxolan-2-yl)pyridine

A mixture of EXAMPLE 119C (4.0 g, 20 mmol), ethane-1,2-diol (6.5 g, 100 mmol) and 4-methylbenzenesulfonic acid (0.3 g, 0.18 mmol) in toluene (80 mL) was heated at 105° C. for 15 hours. After cooling to ambient temperature, the mixture was concentrated and the residue was purified by flash chromatography on silica gel eluting with 10:1 petroleum ether/ethyl acetate to give the title compound.

### Example 119E

### methyl 2,6-dichloro-4-(2-methyl-1,3-dioxolan-2-yl)nicotinate

To a solution of EXAMPLE 119D (3.8 g, 15 mmol) in tetrahydrofuran (150 mL) at −70° C. was added tetramethylethylenediamine (7.3 mL, 50 mmol). After stirring for 10 minutes, 2.5M butyllithium (20 mL, 50 mmol) was added dropwise and the mixture was stirred at −70° C. for 2 hours. Methyl carbonochloridate (3.0 mL, 40 mmol) was added dropwise and the mixture was stirred at −70° C. for 1.5 hours and warmed to ambient temperature. Water (5 mL) was added and the mixture was extracted with ethyl acetate (50 mL). The organic phase was washed with saturated sodium chloride (2×00 mL), dried over magnesium sulfate, filtered, concentrated and purified by flash chromatography on silica gel eluting with 10/1 petroleum ether/ethyl acetate to give the title compound.

### Example 119F

### 5,7-dichloro-1-methylpyrido[3,4-d]pyridazin-4(3H)-one

A mixture of EXAMPLE 119E (3.0 g, 10.3 mmol), hydrazine hydrochloride (3.0 g, 34.5 mmol) and concentrated hydrochloric acid (100 mL) in tetrahydrofuran (100 mL) was heated at 65° C. for 72 hours. The mixture was concentrated and the residue washed with water to afford the title compound.

### Example 119G

### tert-butyl 4-(4-(7-chloro-1-methyl-4-oxo-3,4-dihydropyrido[3,4-d]pyridazin-5-ylamino)-3-methoxy phenyl)piperazine-1-carboxylate

The title compound was obtained following the procedure described in EXAMPLE 2D, using EXAMPLE 119F in place of EXAMPLE 2C.

### Example 119H

### tert-butyl 4-(4-(7-(2-chlorophenoxy)-1-methyl-4-oxo-3,4-dihydropyrido[3,4-d]pyridazin-5-ylamino)-3-methoxyphenyl)piperazine-1-carboxylate

The title compound was obtained following the procedure described in EXAMPLE 62A, using EXAMPLE 119G in place of EXAMPLE 24A. MS: 593(M+H⁺).

### Example 1191

### 7-(2-chlorophenoxy)-5-{[2-methoxy-4-(piperazin-1-yl)phenyl]amino}-1-methylpyrido[3,4-d]pyridazin-4-ol

The title compound was obtained following the procedure described in EXAMPLE 62A, using EXAMPLE 119H in place of EXAMPLE 24A. ¹H NMR (CD₃OD, 300 MHz): δ 7.61-7.34 (m, 5H), 6.67-6.63 (m, 2H), 6.10 (s, 1H), 3.93 (s, 3H), 3.33 (m, 8H), 2.49 (s, 3H). MS: 493 (M+H⁺).

### Example 120

### 2-{[7-(2-chlorophenoxy)-4-hydroxypyrido[3,4-d]pyridazin-5-yl]amino}-5-(piperazin-1-yl)benzonitrile

### Example 120A

### tert-butyl 4-(3-cyano-4-nitrophenyl)piperazine-1-carboxylate

A suspension of 5-bromo-2-nitrobenzonitrile (300 mg, 1.806 mmol), tert-butyl piperazine-1-carboxylate (370 mg, 1.97 mmol) and potassium carbonate (500 g, 3.612 mmol) in tetrahydrofuran (20 mL) was heated at 60° C. for 9 hours. After cooling to ambient temperature, the mixture was diluted with water (50 mL) and extracted with dichloromethane. The organic layer was dried over sodium sulfate, filtered and concentrated. The residue was recrystallized from ethanol to give the title compound.

### Example 120B

### tert-buty 14-(4-amino-3-cyanophenyl)piperazine-1-carboxylate

A mixture of EXAMPLE 120A (620 mg, 1.806 mmol) and 10% palladium on carbon (60 mg) in methanol (10 mL) was stirred under hydrogen at ambient temperature for 1 hour. The mixture was filtered through diatomaceous earth and the filtrate was concentrated. The residue was purified by flash chromatography on silica gel (200-300 mesh) eluting with a gradient of 3/1 to 2/1 petroleum ether/ethyl acetate to give the title compound.

### Example 120C

### tert-buty 4-(4-(7-chloro-4-oxo-3,4-dihydropyrido[3,4-d]pyridazin-5-ylamino)-3-cyanophenyl)piperazine-1-carboxylate

The title compound was obtained following the procedure described in EXAMPLE 24A, using EXAMPLE 120B in place of EXAMPLE 1G. MS: 482 (M+H⁺).

### Example 120D

### tert-butyl 4-(4-(7-(2-chlorophenoxy)-4-oxo-3,4-dihydropyrido[3,4-d]pyridazin-5-ylamino)-3-cyanophenyl)piperazine-1-carboxylate

The title compound was obtained following the procedure described in EXAMPLE 62A, using EXAMPLE 120C in place of EXAMPLE 24A. MS: 738 (M+H⁺).

### Example 120E

### 2-{[7-(2-chlorophenoxy)-4-hydroxypyrido[3,4-d]pyridazin-5-yl]amino}-5-(piperazin-1-yl)benzonitrile

The title compound was obtained following the procedure described in EXAMPLE 26E, using EXAMPLE 120D in place of EXAMPLE 26D. ¹H NMR (CD₃OD, 300 MHz): δ 8.18 (s, 1H), 7.68 (d, J=9.6 Hz, 1H), 7.57 (dd, J=1.8 Hz, J=7.8 Hz, 1H), 7.43 (m, 1H), 7.37 (m, 1H), 7.33 (m, 1H), 7.21 (m, 1H), 6.81 (dd, J=2.7 Hz, J=9.3 Hz, 1H), 6.69 (s, 1H), 3.37 (m, 8H). MS: 474; 476 (M+H⁺).

### Example 121

### Enzyme Inhibition Data

The following procedure is used to determine ALK Activity.

ALK kinase assays were conducted with the indicated final concentrations unless otherwise specified. In 384 well black plates (Axygen), 8 μl of compound (2% DMSO) was incubated with 8 μl Lck-peptide substrate (0.5 μM, biotin-Ahx-GAEEEIYAAFFA-COOH) and 8 μl of a mixture of ALK (3 nM, Millipore) and ATP (50 μM) in reaction buffer (50 mM Hepes, pH 7.4; 10 mM MgCl₂; 2 mM MnCl₂; 0.1 mM sodium orthovanadate; 0.01% BSA and 1 mM DTT (added fresh before assay) for 1 h at room temperature. Reactions were then quenched by the addition of 30 μl quench solution (streptavidin-allophycocyanin and Europium-cryptate PT66 monoclonal antibody in 40 mM Hepes, pH 7.4; 480 mM KF; 66 mM EDTA; 0.01% Tween-20; and 0.1% BSA) at room temperature. Plates were read 1 h after quenching on an Envision Multilaber Reader and IC₅₀ values were calculated using a sigmoidal fit of the concentration/inhibition response curves. These values were converted to apparent K, values using the Cheng-Prusoff relationship.

Alternatively, 4 nM ALK (Millipore) and 50 μM ATP were pre-incubated for 30 min at room temperate in 384 well plates (Corning 3676) in 2.5× reaction buffer (125 nM SEB from Cisbio Bioassays, 12.5 mM MgCl₂, 5 mM MnCl₂, and 2.5 mM DTT). Reactions were initiated by the addition of 4 μl ALK-ATP mixture to 2 μl compounds (2% DMSO) and 4 μl TK-substrate biotin (Cisbio Bioassays). After incubation for 1 h at room temperature, reactions were quenched in 10 μl stop buffer (Cisbio detection buffer containing Streptavididn-XL665 and Eu-Cryptate PT66 monoclonal antibody). Plates were read 1 h after quenching on an Envision Multilaber Reader and IC₅₀ values were calculated using a sigmoidal fit of the concentration/inhibition response curves. These values were converted to apparent Kᵢ values using the Cheng-Prusoff relationship. Results are shown in Table 1.

TABLE 1
ALK Activity

| | HTRF_ALK |
|---|---|
| Example | Human - Kᵢ |
| 1 | 0.011 |
| 2 | 0.0014 |
| 3 | 0.66 |
| 4 | 0.94 |
| 5 | 0.001 |
| 6 | 0.602 |
| 7 | 0.001 |
| 8 | >0.47 |
| 9 | >0.47 |
| 10 | 0.002 |
| 11 | >4.7 |
| 12 | 0.003 |
| 13 | 0.001 |
| 14 | 0.013 |
| 15 | 0.003 |
| 16 | >4.7 |
| 17 | 0.006 |
| 18 | 3.1 |
| 19 | >4.7 |
| 20 | 0.003 |
| 21 | 0.368 |
| 22 | 0.002 |
| 23 | 0.021 |
| 24 | 0.004 |
| 25 | 0.006 |
| 26 | 0.000 |
| 27 | 0.003 |
| 28 | 0.004 |
| 29 | 0.001 |
| 30 | 0.003 |
| 31 | 0.019 |
| 32 | 0.002 |
| 33 | 0.0002 |
| 34 | 0.004 |
| 35 | 0.001 |
| 36 | 0.026 |
| 37 | 0.003 |
| 38 | 0.016 |
| 39 | 0.007 |
| 40 | 0.036 |
| 41 | 0.028 |
| 42 | 0.025 |
| 43 | 0.015 |
| 44 | 0.013 |
| 45 | 0.051 |
| 46 | 0.003 |
| 47 | 0.053 |
| 48 | 0.004 |
| 49 | 0.001 |
| 50 | 0.009 |
| 51 | 0.002 |
| 52 | 0.002 |
| 53 | 0.004 |
| 54 | 0.004 |
| 55 | 0.006 |
| 56 | 0.005 |
| 57 | 0.002 |
| 58 | 0.004 |
| 59 | 0.004 |
| 60 | 0.003 |
| 61 | 0.002 |
| 62 | 0.002 |
| 63 | 0.001 |
| 64 | 0.021 |
| 65 | 0.001 |
| 66 | 0.010 |
| 67 | 0.002 |
| 68 | 0.002 |
| 69 | 0.001 |
| 70 | 0.001 |
| 71 | 0.001 |
| 72 | 0.012 |
| 73 | 0.001 |
| 74 | 0.004 |
| 75 | 0.004 |
| 76 | 0.004 |
| 77 | 0.004 |
| 78 | 0.004 |
| 79 | 0.005 |
| 80 | 0.001 |
| 81 | >0.47 |
| 82 | 0.004 |
| 83 | >0.47 |
| 84 | 0.005 |
| 85 | 0.005 |
| 86 | 0.002 |
| 87 | 0.004 |
| 88 | 0.004 |
| 89 | 0.015 |
| 90 | 0.007 |
| 91 | 0.055 |
| 92 | 0.015 |
| 93 | 0.004 |
| 94 | 0.051 |
| 95 | 0.138 |
| 96 | 0.009 |
| 97 | 0.074 |
| 98 | 0.009 |
| 99 | 0.006 |
| 100 | 0.004 |
| 101 | 0.001 |
| 102 | 0.331 |
| 103 | 0.020 |
| 104 | 0.075 |
| 105 | >0.47 |
| 106 | 0.021 |
| 107 | 0.016 |
| 108 | 0.008 |
| 109 | 0.006 |
| 110 | 0.016 |
| 111 | 0.015 |
| 112 | 0.004 |
| 113 | 0.058 |
| 114 | 0.008 |
| 115 | 0.006 |
| 116 | 0.033 |
| 117 | 0.015 |
| 118 | 0.015 |
| 119 | 0.015 |
| 120 | 0.002 |

Compounds of the present invention assessed by the above-described assays were found to have ALK kinase-inhibiting activity.

All publication and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A compound of formula (I): 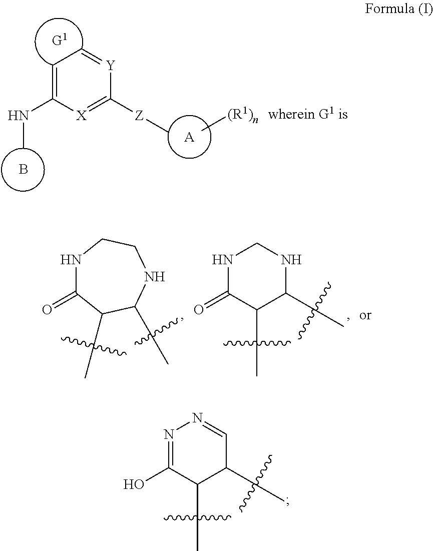
X is CH or N;
Y is CH or N;
wherein at least one of X and Y is N;
A is phenyl, naphthyl, indenyl, C₃₋₈ cycloalkyl, 4-7 membered heterocycloalkyl, 5-7 membered heterocycloalkenyl, or 5-7 membered heteroaryl;
B is
(a) phenyl, naphthyl, tetrahydronaphthyl, indenyl, or indanyl, wherein the phenyl, naphthyl, tetrahydronaphthyl, indenyl, or indanyl is optionally substituted with one, two, three, or four R² and is substituted with R³; or
(b) 5-16 membered monocyclic, bicyclic, or tricyclic heterocyclyl, wherein the heterocyclyl is optionally substituted with one, two, three, four, or five R⁴;
Z is a bond, C₁₋₆ alkylene, C₂₋₆ alkenylene, —O— or —NR⁵—;
R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁶, SR⁶, C(O)R⁶, C(O)NR⁷R⁸, C(O)OR⁶, OC(O)R⁶, OC(O)NR⁷R⁸, NR⁷R⁸, NR⁷C(O)R⁶, S(O)R⁶, S(O)NR⁷R⁸, S(O)₂R⁶, NR⁷S(O)₂R⁶, and S(O)₂NR⁷R⁸; wherein the C₃₋₈ cycloalkyl, aryl, heterocycloalkyl, and heteroaryl are optionally substituted with 1, 2, or 3 substituents independently selected from halo, C₁₋₆ alkyl, C₁₋₆ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c};
R², at each occurrence, is independently selected from the group consisting of halo, CN, OH, C₁₋₆ alkyl, C₁₋₆-haloalkyl, C₁₋₆ alkoxy, C₁₋₆ haloalkoxy, C₁₋₆-thioalkoxy, amino, C₁₋₆ alkylamino, and C₁₋₆ dialkylamino;
R³ is selected from the group consisting of aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁹, C(O)R⁹, —C₁₋₆-alkyl-C(O)R⁹, C(O)NR¹⁰R¹¹, C(O)OR⁹, OC(O)R⁹, OC(O)NR¹⁰R¹¹, NR¹⁰R¹¹, NR¹⁰C(O)R⁹, S(O)R⁹, S(O)NR¹⁰R¹¹, S(O)₂R⁹, NR¹⁰S(O)₂R⁹, and S(O)₂NR¹⁰R¹¹, wherein the C₃₋₈ cycloalkyl, aryl, heterocycloalkyl, and heteroaryl, alone or part of another moiety, are optionally substituted with one, two, or three R¹²;
R⁴ is CN, NO₂, halo, C₁₋₆-alkyl, C₁₋₆-haloalkyl, OR^{j}, SR^{j}, C(O)R^{d}, C(O)NR^{e}R^{f}, C(O)OR^{d}, NR^{e}R^{f}, NR^{e}C(O)R^{d}, S(O)₂R^{d}, NR^{e}S(O)₂R^{d}, or S(O)₂NR^{e}R^{f};
R⁵ is H or C₁₋₆-alkyl;
R⁶, R⁷, and R⁸, at each occurrence, are independently selected from H, C₁₋₆ alkyl, C₁₋₆ haloalkyl, aryl, C₃₋₈ cycloalkyl, heteroaryl, and heterocycloalkyl, wherein the aryl, C₃₋₈ cycloalkyl, heteroaryl, and heterocycloalkyl moiety are optionally substituted with 1, 2, or 3 substituents independently selected from halo, CN, OH, C₁₋₆ alkyl, C₁₋₆-haloalkyl, C₁₋₆ alkoxy, C₁₋₆ haloalkoxy, amino, C₁₋₆ alkylamino, C₁₋₆ dialkylamino, C(O)OH, C(O)C₁₋₆ alkyl, C(O)NH₂, C(O)NH(C₁₋₆ alkyl), or C(O)N(C₁₋₆ alkyl)₂;
R⁹, R¹⁰, and R¹¹, at each occurrence, are independently selected from H, C₁₋₆ alkyl, C₁₋₆ haloalkyl, heteroaryl-C₁₋₆-alkyl-, heterocycloalkyl-C₁₋₆-alkyl-, R¹³R¹⁴N—C₁₋₆-alkyl-, aryl, C₃₋₈ cycloalkyl, heteroaryl, and heterocycloalkyl, wherein the aryl, C₃₋₈ cycloalkyl, heteroaryl, and heterocycloalkyl, alone or as part of another moiety, are optionally substituted with 1, 2, or 3 substituents independently selected from halo, CN, OH, C₁₋₆ alkyl, C₁₋₆-haloalkyl, C₁₋₆ alkoxy, C₁₋₆ haloalkoxy, amino, C₁₋₆ alkylamino, C₁₋₆ dialkylamino, C(O)OH, C(O)C₁₋₆ alkyl, C(O)NH₂, C(O)NH(C₁₋₆ alkyl), or C(O)N(C₁₋₆ alkyl)₂;
R¹², at each occurrence, is independently selected from the group consisting of halo, C₁₋₆ alkyl, C₁₋₆ haloalkyl, amino-C₁₋₆-alkyl-, C₁₋₆ alkylamino-C₁₋₆ alkyl-, C₁₋₆ dialkylamino-C₁₋₆ alkyl-, hydroxy-C₁₋₆-alkyl-, C₁₋₆ alkyl-C₁₋₆ alkoxy, aryl, C₃₋₈ cycloalkyl, heteroaryl, heterocycloalkyl, aryl-(C₁₋₆ alkyl)-, C₃₋₈ cycloalkyl-(C₁₋₆ alkyl)-, heteroaryl-(C₁₋₆ alkyl)-, heterocycloalkyl-(C₁₋₆ alkyl)-, CN, NO₂, OR^{g}, SR^{g}, C(O)R^{g}, C(O)NR^{h}Rⁱ, C(O)OR^{g}, OC(O)R^{g}, OC(O)NR^{h}Rⁱ, NR^{h}Rⁱ, NR^{h}C(O)R^{g}, S(O)R^{g}, S(O)NR^{h}Rⁱ, S(O)₂R^{g}, NR^{h}S(O)₂R^{g}, and S(O)₂NR^{h}Rⁱ, wherein the aryl, C₃₋₈ cycloalkyl, heteroaryl, and heterocycloalkyl, alone or as part of another moiety, are optionally substituted with one, two or three substituents independently selected from halo and C₁₋₆ alkyl;
R¹³ and R¹⁴, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{a}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{b} and R^{c}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{d}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{e} and R^{f}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{g}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{h} and Rⁱ, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂; and
n is 0, 1, 2, or 3;
or a pharmaceutically acceptable salt or solvate thereof.

2. The compound of claim 1 of formula (I) wherein G¹ is 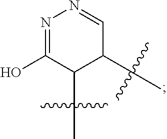 [SMILES: *C1C=NN=C(O)C1*]
X is N; and
Y is CH.

3. The compound of claim 1 of formula (I) wherein G¹ is 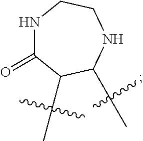 [SMILES: *C1NCCNC(=O)C1*]
X is CH; and
Y is N.

4. The compound of claim 1 of formula (I) wherein G¹ is 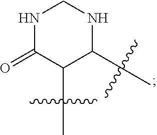 [SMILES: *C1NCNC(=O)C1*]
X is N; and
Y is CH.

5. The compound of claim 1 of formula (I) wherein Z is bond.

6. The compound of claim 1 of formula (I) wherein Z is C₁₋₆ alkylene.

7. The compound of claim 1 of formula (I) wherein Z is —O—.

8. The compound of claim 1 of formula (I) wherein Z is —NR⁵(CH₂)ₚ—.

9. The compound of claim 1 of formula (I) wherein A is phenyl, wherein R¹ is selected from the group consisting of halo, CN, NO₂, C₁₋₆-alkyl, C₁₋₆-haloalkyl, OR⁶, SR⁶, C(O)R⁶, C(O)OR⁶, NR⁷R⁸, and S(O)R⁶.

10. The compound of claim 9 of formula (I) wherein the phenyl is substituted with two R¹, and R¹ is halo.

11. The compound of claim 1 of formula (I) wherein B is phenyl.

12. The compound of claim 13 of formula (I) wherein R³ is heterocycloalkyl.

13. The compound of claim 1 of formula (I) wherein
B is 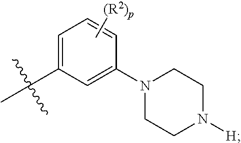
R² is halo, C₁₋₆-alkyl, C₁₋₆ haloalkyl, or OR⁶; and p is 0, 1, or 2.

14. The compound of claim 1 of formula (I), or a pharmaceutically acceptable salt or solvate thereof, selected from the group consisting of
7-(2,6-dichlorobenzyl)-5-{[2-methoxy-4-(piperazin-1-yl)phenyl]amino}-2,3-dihydropyrido[4,3-d]pyrimidin-4(1H)-one;
7-(2,6-dichlorobenzyl)-5-{[2-methoxy-4-(piperazin-1-yl)phenyl]amino}pyrido[3,4-d]pyridazin-4-ol;
8-(2,6-dichlorobenzyl)-6-{[2-methoxy-4-(piperazin-1-yl)phenyl]amino}-1,2,3,4-tetrahydro-5H-pyrido[2,3-e][1,4]diazepin-5-one;
8-(2,6-dichlorobenzyl)-6-{[4-(piperazin-1-yl)phenyl]amino}-1,2,3,4-tetrahydro-5H-pyrido[2,3-e][1,4]diazepin-5-one;
7-(2,6-dichlorobenzyl)-5-{[4-(piperazin-1-yl)phenyl]amino}pyrido[3,4-d]pyridazin-4-ol;
8-(2,6-dichlorobenzyl)-6-{[4-(piperazin-1-yl)phenyl]amino}-1,2,3,4-tetrahydro-5H-pyrido[4,3-e][1,4]diazepin-5-one;
7-(2,6-dichlorobenzyl)-5-{[3-methyl-4-(piperazin-1-yl)phenyl]amino}pyrido[3,4-d]pyridazin-4(3H)-one;
5-{[4-(4-cyclohexylpiperazin-1-yl)-2-methoxyphenyl]amino}-7-(2,6-dichlorobenzyl)pyrido[3,4-d]pyridazin-4(3H)-one;
[4-(4-{[7-(2,6-dichlorobenzyl)-4-hydroxypyrido[3,4-d]pyridazin-5-yl]amino}-3-methoxyphenyl)piperazin-1-yl](phenyl)methanone;
7-[(2,6-dichlorophenyl)amino]-5-{[2-methoxy-4-(piperazin-1-yl)phenyl]amino}pyrido[3,4-d]pyridazin-4-ol;
7-(2,6-dichlorobenzyl)-5-({2-methoxy-4-[4-(propan-2-ylsulfonyl)piperazin-1-yl]phenyl}amino)pyrido[3,4-d]pyridazin-4-ol;
1-[4-(4-{[7-(2,6-dichlorobenzyl)-4-hydroxypyrido[3,4-d]pyridazin-5-yl]amino}-3-methoxyphenyl)piperazin-1-yl]-2-(dimethylamino)ethanone;
5-{[3-chloro-4-(piperazin-1-yl)phenyl]amino}-7-(2,6-dichlorobenzyl)pyrido[3,4-d]pyridazin-4(3H)-one;
7-(2,6-dichlorobenzyl)-5-{[5-(piperazin-1-yl)pyridin-2-yl]amino}pyrido[3,4-d]pyridazin-4(3H)-one;
7-(2,6-dichlorobenzyl)-5-({2-methoxy-4-[4-(1-methylpiperidin-4-yl)piperazin-1-yl]phenyl}amino)pyrido[3,4-d]pyridazin-4(3H)-one;
7-(2,6-dichlorobenzyl)-5-({2-methoxy-4-[4-(3-methylbutyl)piperazin-1-yl]phenyl}amino)pyrido[3,4-d]pyridazin-4(3H)-one;
7-(2,6-dichlorobenzyl)-5-({2-methoxy-4-[2-(pyrrolidin-1-yl)ethyl]phenyl}amino)pyrido[3,4-d]pyridazin-4(3H)-one;
7-(2,6-dichlorobenzyl)-5-({2-methoxy-4-[2-oxo-2-(pyrrolidin-1-yl)ethyl]phenyl}amino)pyrido[3,4-d]pyridazin-4(3H)-one;
7-(2,6-dichlorobenzyl)-5-({2-methoxy-4-[4-(3-methylbutanoyl)piperazin-1-yl]phenyl}amino)pyrido[3,4-d]pyridazin-4(3H)-one;
7-(2,6-dichlorobenzyl)-5-{[2,5-difluoro-4-(piperazin-1-yl)phenyl]amino}pyrido[3,4-d]pyridazin-4(3H)-one;
7-(2,6-dichlorobenzyl)-5-{[2,6-dimethyl-4-(piperazin-1-yl)phenyl]amino}pyrido[3,4-d]pyridazin-4-ol;
5-{[2-methoxy-4-(piperazin-1-yl)phenyl]amino}-7-{[2-(propan-2-ylsulfonyl)phenyl]amino}pyrido[3,4-d]pyridazin-4(3H)-one;
5-{[2-methoxy-4-(piperazin-1-yl)phenyl]amino}-7-{[(4R)-4-(propan-2-yl)-4,5-dihydro-1,3-oxazol-2-yl]amino}pyrido[3,4-d]pyridazin-4-ol;
7-[(2-chlorophenyl)amino]-5-{[2-methoxy-4-(piperazin-1-yl)phenyl]amino}pyrido[3,4-d]pyridazin-4(3H)-one;
7-[(5-chloro-2-fluorophenyl)amino]-5-{[2-methoxy-4-(piperazin-1-yl)phenyl]amino}pyrido[3,4-d]pyridazin-4(3H)-one;
7-(2,6-dichlorobenzyl)-5-{[2-fluoro-4-(piperazin-1-yl)phenyl]amino}pyrido[3,4-d]pyridazin-4(3H)-one;
7-[(2-chloro-5-fluorophenyl)amino]-5-{[2-methoxy-4-(piperazin-1-yl)phenyl]amino}pyrido[3,4-d]pyridazin-4(3H)-one;
7-[(2,5-dichlorophenyl)amino]-5-{[2-methoxy-4-(piperazin-1-yl)phenyl]amino}pyrido[3,4-d]pyridazin-4(3H)-one;
7-[(2-chloro-6-fluorophenyl)amino]-5-{[2-methoxy-4-(piperazin-1-yl)phenyl]amino}pyrido[3,4-d]pyridazin-4(3H)-one;
7-(2,6-dichlorobenzyl)-5-{[2-methoxy-4-(4-methylpiperazin-1-yl)phenyl]amino}pyrido[3,4-d]pyridazin-4(3H)-one;
7-[(3,5-dichlorophenyl)amino]-5-{[2-methoxy-4-(piperazin-1-yl)phenyl]amino}pyrido[3,4-d]pyridazin-4(3H)-one;
7-[(2,6-difluorophenyl)amino]-5-{[2-methoxy-4-(piperazin-1-yl)phenyl]amino}pyrido[3,4-d]pyridazin-4(3H)-one;
7-[(3,5-dichloropyridin-4-yl)amino]-5-{[2-methoxy-4-(piperazin-1-yl)phenyl]amino}pyrido[3,4-d]pyridazin-4(3H)-one;
7-[(2,4-dichlorophenyl)amino]-5-{[2-methoxy-4-(piperazin-1-yl)phenyl]amino}pyrido[3,4-d]pyridazin-4-ol;
7-[(2,6-dichloro-4-fluorophenyl)amino]-5-{[2-methoxy-4-(piperazin-1-yl)phenyl]amino}pyrido[3,4-d]pyridazin-4-ol;
7-[(2,3-difluorophenyl)amino]-5-{[2-methoxy-4-(piperazin-1-yl)phenyl]amino}pyrido[3,4-d]pyridazin-4(3H)-one;
7-[(furan-2-ylmethyl)amino]-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-(piperidin-1-yl)pyrido[3,4-d]pyridazin-4(3H)-one;
7-(benzylamino)-5-{[2-methoxy-4-(piperazin-1-yl)phenyl]amino}pyrido[3,4-d]pyridazin-4(3H)-one;
5-{[2-methoxy-4-(piperazin-1-yl)phenyl]amino}-7-phenylpyrido[3,4-d]pyridazin-4(3H)-one;
7-(2,6-dichlorobenzyl)-5-{[2,6-difluoro-4-(piperazin-1-yl)phenyl]amino}pyrido[3,4-d]pyridazin-4(3H)-one;
5-{[2-methoxy-4-(piperazin-1-yl)phenyl]amino}-7-(pyridin-2-ylamino)pyrido[3,4-d]pyridazin-4(3H)-one;
5-{[2-methoxy-4-(piperazin-1-yl)phenyl]amino}-7-(pyridin-3-ylamino)pyrido[3,4-d]pyridazin-4(3H)-one;
7-(cyclohexylamino)-5-{[2-methoxy-4-(piperazin-1-yl)phenyl]amino}pyrido[3,4-d]pyridazin-4(3H)-one;
5-{[2-methoxy-4-(piperazin-1-yl)phenyl]amino}-7-{[2-(pyridin-2-yl)ethyl]amino}pyrido[3,4-d]pyridazin-4-ol;
7-[(2-fluorophenyl)amino]-5-{[2-methoxy-4-(piperazin-1-yl)phenyl]amino}pyrido[3,4-d]pyridazin-4(3H)-one;
7-[(2,6-dimethylphenyl)amino]-5-{[2-methoxy-4-(piperazin-1-yl)phenyl]amino}pyrido[3,4-d]pyridazin-4(3H)-one;
7-[(3-chloropyridin-2-yl)amino]-5-{[2-methoxy-4-(piperazin-1-yl)phenyl]amino}pyrido[3,4-d]pyridazin-4(3H)-one;
7-[(2,3-dichlorophenyl)amino]-5-{[2-methoxy-4-(piperazin-1-yl)phenyl]amino}pyrido[3,4-d]pyridazin-4(3H)-one;
5-{[2-methoxy-4-(piperazin-1-yl)phenyl]amino}-7-[(2,4,6-trifluorophenyl)amino]pyrido[3,4-d]pyridazin-4(3H)-one;
5-{[2-methoxy-4-(piperazin-1-yl)phenyl]amino}-7-[(2,3,4-trifluorophenyl)amino]pyrido[3,4-d]pyridazin-4(3H)-one;
7-[(3-chloro-2-fluorophenyl)amino]-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-(tetrahydro-2H-pyran-4-ylamino)pyrido[3,4-d]pyridazin-4-ol;
5-{[2-methoxy-4-(piperazin-1-yl)phenyl]amino}-7-(1,3-thiazol-2-ylamino)pyrido[3,4-d]pyridazin-4(3H)-one;
5-{[2-chloro-4-(piperazin-1-yl)phenyl]amino}-7-(2,6-dichlorobenzyl)pyrido[3,4-d]pyridazin-4(3H)-one;
5-{[2-methoxy-4-(piperazin-1-yl)phenyl]amino}-7-[(1-methylpiperidin-4-yl)amino]pyrido[3,4-d]pyridazin-4(3H)-one;
7-(2,6-dichlorobenzyl)-5-{[3-fluoro-2-methoxy-4-(piperazin-1-yl)phenyl]amino}pyrido[3,4-d]pyridazin-4-ol;
7-[(3-chloropyridin-4-yl)amino]-5-{[2-methoxy-4-(piperazin-1-yl)phenyl]amino}pyrido[3,4-d]pyridazin-4(3H)-one;
5-{[2-bromo-4-(piperazin-1-yl)phenyl]amino}-7-(2,6-dichlorobenzyl)pyrido[3,4-d]pyridazin-4(3H)-one;
7-(2-chlorophenoxy)-5-{[2-methoxy-4-(piperazin-1-yl)phenyl]amino}pyrido[3,4-d]pyridazin-4(3H)-one;
7-(2,6-dichlorophenoxy)-5-{[2-methoxy-4-(piperazin-1-yl)phenyl]amino}pyrido[3,4-d]pyridazin-4(3H)-one;
7-(2,6-dichlorobenzyl)-5-{[4-(piperazin-1-yl)-2-(trifluoromethoxy)phenyl]amino}pyrido[3,4-d]pyridazin-4-ol;
7-[(2,3-dichloro-6-fluorophenyl)amino]-5-{[2-methoxy-4-(piperazin-1-yl)iphenyl]amino}pyrido[3,4-d]pyridazin-4(3H)-one;
7-(2,6-dichlorobenzyl)-5-{[4-(hexahydropyrrolo[1,2-a]pyrazin-2(1H)-yl)-2-methoxyphenyl]amino}pyrido[3,4-d]pyridazin-4(3H)-one;
7-[(2,3-dichloro-4-fluorophenyl)amino]-5-{[2-methoxy-4-(piperazin-1-yl)phenyl]amino}pyrido[3,4-d]pyridazin-4-ol;
2-[(2,6-dichlorophenyl)amino]-4-{[2-methoxy-4-(piperazin-1-yl)phenyl]amino}pyrido[2,3-d]pyridazin-5(6H)-one;
7-(2,6-dichlorobenzyl)-5-{[2,3-dimethyl-4-(piperazin-1-yl)phenyl]amino}pyrido[3,4-d]pyridazin-4(3H)-one;
2-(2,6-dichlorobenzyl)-4-{[2-methoxy-4-(piperazin-1-yl)phenyl]amino}pyrido[2,3-d]pyridazin-5(6H)-one;
7-[(2-chloro-4,6-difluorophenyl)amino]-5-{[2-methoxy-4-(piperazin-1-yl)phenyl]amino}pyrido[3,4-d]pyridazin-4-ol;
7-(2,6-dichlorobenzyl)-5-{[2-methoxy-5-methyl-4-(piperazin-1-yl)phenyl]amino}pyrido[3,4-d]pyridazin-4(3H)-one;
7-(2,6-dichlorobenzyl)-5-{[2-fluoro-5-methyl-4-(piperazin-1-yl)phenyl]amino}pyrido[3,4-d]pyridazin-4(3H)-one;
6-(2,6-dichlorobenzyl)-8-[(6-methoxy-1,2,3,4-tetrahydroisoquinolin-7-yl)amino]phthalazin-1(2H)-one;
7-(2,6-difluorophenoxy)-5-{[2-methoxy-4-(piperazin-1-yl)phenyl]amino}pyrido[3,4-d]pyridazin-4(3H)-one;
7-(2-fluorophenoxy)-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-[(2,3,4-trichlorophenyl)amino]pyrido[3,4-d]pyridazin-4(3H)-one;
7-(2,3-difluorophenoxy)-5-{[2-methoxy-4-(piperazin-1-yl)phenyl]amino}pyrido[3,4-d]pyridazin-4-ol;
7-(2,3-dichlorophenoxy)-5-{[2-methoxy-4-(piperazin-1-yl)phenyl]amino}pyrido[3,4-d]pyridazin-4(3H)-one;
7-(2,6-dichloro-4-fluorophenoxy)-5-{[2-methoxy-4-(piperazin-1-yl)phenyl]amino}pyrido[3,4-d]pyridazin-4(3H)-one;
7-[(3-chloropyridin-4-yl)oxy]-5-{[2-methoxy-4-(piperazin-1-yl)phenyl]amino}pyrido[3,4-d]pyridazin-4(3H)-one;
7-(2,3-dimethylphenoxy)-5-{[2-methoxy-4-(piperazin-1-yl)phenyl]amino}pyrido[3,4-d]pyridazin-4-ol;
7-[(3-fluoropyridin-4-yl)oxy]-5-{[2-methoxy-4-(piperazin-1-yl)phenyl]amino}pyrido[3,4-d]pyridazin-4-ol;
5-{[2-methoxy-4-(piperazin-1-yl)phenyl]amino}-7-(2,3,4-trichlorophenoxy)pyrido[3,4-d]pyridazin-4-ol;
7-(2,4-dichlorophenoxy)-5-{[2-methoxy-4-(piperazin-1-yl)phenyl]amino}pyrido[3,4-d]pyridazin-4(3H)-one;
7-[(trans-4-hydroxycyclohexyl)amino]-5-{[2-methoxy-4-(piperazin-1-yl)phenyl]amino}pyrido[3,4-d]pyridazin-4-ol;
7-(cyclopentyloxy)-5-{[2-methoxy-4-(piperazin-1-yl)phenyl]amino}pyrido[3,4-d]pyridazin-4-ol;
7-(2,6-dichlorobenzyl)-5-[(7-methoxy-1,2,3,4-tetrahydroisoquinolin-6-yl)amino]pyrido[3,4-d]pyridazin-4(3H)-one;
2-(2-chlorophenoxy)-4-{[2-methoxy-4-(piperazin-1-yl)phenyl]amino}pyrido[2,3-d]pyridazin-5(6H)-one;
5-{[2-chloro-4-(pyrrolidin-1-ylmethyl)phenyl]amino}-7-(4-fluoropiperidin-1-yl)pyrido[3,4-d]pyridazin-4-ol;
2-(2-chlorobenzyl)-4-[(6-methoxy-1,2,3,4-tetrahydroisoquinolin-7-yl)amino]pyrido[2,3-d]pyridazin-5-ol;
2-(2-chlorobenzyl)-4-{[2-methoxy-4-(piperazin-1-ylmethyl)phenyl]amino}pyrido[2,3-d]pyridazin-5(6H)-one;
5-{[2-chloro-4-(pyrrolidin-1-ylmethyl)phenyl]amino}-7-[(cyclopropylmethyl)amino]pyrido[3,4-d]pyridazin-4-ol;
2-(2-chlorobenzyl)-4-{[2-methoxy-4-(morpholin-4-ylmethyl)phenyl]amino}pyrido[2,3-d]pyridazin-5-ol;
7-(2-chlorophenoxy)-5-{[2-ethoxy-4-(pyrrolidin-1-ylmethyl)phenyl]amino}pyrido[3,4-d]pyridazin-4(3H)-one;
5-{[2-chloro-4-(pyrrolidin-1-ylmethyl)phenyl]amino}-7-(4-hydroxypiperidin-1-yl)pyrido[3,4-d]pyridazin-4(3H)-one;
5-{[2-chloro-4-(pyrrolidin-1-ylmethyl)phenyl]amino}-7-(3-hydroxyazetidin-1-yl)pyrido[3,4-d]pyridazin-4(3H)-one;
7-(2-chlorophenoxy)-5-{[2-methoxy-4-(piperidin-4-yl)phenyl]amino}pyrido[3,4-d]pyridazin-4-ol;
7-[(cyclopropylmethyl)amino]-5-{[2-methoxy-4-(piperidin-4-yl)phenyl]amino}pyrido[3,4-d]pyridazin-4-ol;
2-(2-chlorobenzyl)-4-{[2-methoxy-4-(piperidin-4-yl)phenyl]amino}pyrido[2,3-d]pyridazin-5(6H)-one;
2-(2,6-dichlorobenzyl)-4-{[2-methoxy-4-(piperidin-4-yl)phenyl]amino}pyrido[2,3-d]pyridazin-5(6H)-one;
4-{[2-methoxy-4-(piperidin-4-yl)phenyl]amino}-2-[2-(morpholin-4-yl)ethyl]pyrido[2,3-d]pyridazin-5(6H)-one;
7-(2-chlorophenoxy)-5-{[5-(piperazin-1-yl)pyridin-2-yl]amino}pyrido[3,4-d]pyridazin-4(3H)-one;
2-(2-cyclopropylethyl)-4-{[2-methoxy-4-(piperidin-4-yl)phenyl]amino}pyrido[2,3-d]pyridazin-5(6H)-one;
(4-{[7-(2-chlorophenoxy)-4-hydroxypyrido[3,4-d]pyridazin-5-yl]amino}-3-methoxyphenyl)(pyrrolidin-1-yl)methanone;
7-(2-chlorophenoxy)-5-{[2-ethoxy-4-(piperidin-4-yl)phenyl]amino}pyrido[3,4-d]pyridazin-4(3H)-one;
2-(2-fluorobenzyl)-4-{[2-methoxy-4-(piperidin-4-yl)phenyl]amino}pyrido[2,3-d]pyridazin-5(6H)-one;
2-(2,3-dichlorobenzyl)-4-{[2-methoxy-4-(piperidin-4-yl)phenyl]amino}pyrido[2,3-d]pyridazin-5(6H)-one;
2-(2,6-difluorobenzyl)-4-{[2-methoxy-4-(piperidin-4-yl)phenyl]amino}pyrido[2,3-d]pyridazin-5(6H)-one;
2-(2,5-difluorobenzyl)-4-{[2-methoxy-4-(piperidin-4-yl)phenyl]amino}pyrido[2,3-d]pyridazin-5(6H)-one;
2-(2,3-difluorobenzyl)-4-{[2-methoxy-4-(piperidin-4-yl)phenyl]amino}pyrido[2,3-d]pyridazin-5(6H)-one;
2-(2-chloro-6-fluorobenzyl)-4-{[2-methoxy-4-(piperidin-4-yl)phenyl]amino}pyrido[2,3-d]pyridazin-5(6H)-one;
5-[(2-methoxyphenyl)amino]-7-{[4-(piperazin-1-yl)phenyl]amino}pyrido[3,4-d]pyridazin-4-ol;
7-(2-chlorophenoxy)-5-{[2-ethoxy-4-(piperazin-1-yl)phenyl]amino}pyrido[3,4-d]pyridazin-4-ol;
2-[(5-{[2-methoxy-4-(piperazin-1-yl)phenyl]amino}-4-oxo-3,4-dihydropyrido[3,4-d]pyridazin-7-yl)oxy]benzonitrile;
2-(2-chlorobenzyl)-4-({2-methoxy-4-[(4-methylpiperazin-1-yl)methyl]phenyl}amino)pyrido[2,3-d]pyridazin-5(6H)-one;
7-(2-chlorophenoxy)-5-{[2-(difluoromethoxy)-4-(piperazin-1-yl)phenyl]amino}pyrido[3,4-d]pyridazin-4(3H)-one;
7-(2-chlorophenoxy)-5-{[5-(piperidin-4-yl)pyridin-2-yl]amino}pyrido[3,4-d]pyridazin-4(3H)-one;
7-(2-chlorophenoxy)-5-{[2-methoxy-4-(piperazin-1-yl)phenyl]amino}-1-methylpyrido[3,4-d]pyridazin-4-ol; and
2-{[7-(2-chlorophenoxy)-4-hydroxypyrido[3,4-d]pyridazin-5-yl]amino}-5-(piperazin-1-yl)benzonitrile.

15. A pharmaceutical composition comprising a compound or pharmaceutically acceptable salt of claim 1 and pharmaceutically acceptable excipient.

16. A method of treating cancer in a mammal comprising administering thereto a therapeutically acceptable amount of a compound or pharmaceutically acceptable salt of claim 1.

17. A method for decreasing tumor volume in a mammal comprising administering thereto a therapeutically acceptable amount of a compound or pharmaceutically acceptable salt of claim 1.
